(19) United States
(12) Patent Application Publication
(10) Pub. No.: US 2023/0193305 A1
(43) Pub. Date: Jun. 22, 2023

(54) METHODS FOR INCREASING POWDERY MILDEW RESISTANCE IN CANNABIS

(71) Applicant: BetterSeeds Ltd, Givat Chen (IL)

(72) Inventors: Tal SHERMAN, Rehovot (IL); Ido MARGALIT, Gan Yavne (IL); Shira COREM, Sde Hemed (IL)

(73) Assignee: BetterSeeds Ltd, Givat Chen (IL)

(21) Appl. No.: 17/933,296

(22) Filed: Sep. 19, 2022

Related U.S. Application Data
(63) Continuation-in-part of application No. 17/310,770, filed on Aug. 23, 2021.
(60) Provisional application No. 62/809,584, filed on Feb. 23, 2019.

(51) Int. Cl. C12N 15/82

(52) U.S. Cl. CPC C12N 15/8218

(57) ABSTRACT

The present invention discloses a modified Cannabis plant exhibiting enhanced resistance to powdery mildew (PM). The aforementioned modified Cannabis plant comprises a mutated Cannabis mlo1 (Csmlo1) allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof. The present invention further discloses methods for production of the modified Cannabis plant using genome modification.

### REFERENCE TO AN ELECTRONIC SEQUENCE LISTING

The contents of the electronic sequence listing (6000150040 Sequence listing.xml; Size: 1.11 MB; and Date of Creation: Sep. 13, 2022) is herein incorporated by reference in its entirety.

### FIELD OF THE INVENTION

The present disclosure relates to conferring pathogen resistance in Cannabis plants. More particularly, the current invention pertains to producing fungal resistant Cannabis plants by controlling genes conferring susceptibility to such pathogens.

### BACKGROUND OF THE INVENTION

Cannabis is one of the oldest domesticated plants with evidence of being used by a vast array of ancient cultures. It is thought to have originated from central Asia from which it was spread by humans to China, Europe, the Middle East and the Americas. Thus, Cannabis has been bred by many different cultures for various uses such as food, fiber and medicine since the dawn of agricultural societies. In the last few decades, Cannabis breeding has stopped as it became illegal and non-economic to do so. With the recent legislation converting Cannabis back to legality, there is a growing need for the implementation of new and advanced breeding techniques in future Cannabis breeding programs. This will allow speeding up the long process of classical breeding and accelerate reaching new and genetically improved Cannabis varieties for fiber, food and medicine products. Developing and implementing molecular biology tools to support the breeders, will allow creating new fungal resistant traits and tracking the movement of such desired traits across breeders germplasm.

Currently, breeding of Cannabis plants is mostly done by small Cannabis growers. There is very limited if any molecular tools supporting or leading the breeding process. Traditional Cannabis breeding is done by mixing breeding material with hope to find the desired traits and phenotypes by random crosses. These methods have allowed the construction of the leading Cannabis varieties on the market today. As the cultivation of Cannabis intensifies in protected structures such as greenhouses and closed growth chambers, such an environment encourages the prevalence of certain diseases, with the lead cause being fungi.

Powdery mildew is a fungal disease that affects a wide range of plants. Powdery mildew diseases are caused by many different species of fungi in the order Erysiphales, with Podosphaera xanthii being the most commonly reported cause. Powdery mildew is one of the easier plant diseases to identify, as its symptoms are quite distinctive. Infected plants display white powdery spots on the leaves and stems. The lower leaves are the most affected, but the mildew can appear on any above-ground part of the plant. As the disease progresses, the spots get larger and denser as large numbers of asexual spores are formed, and the mildew may spread up and reduce the length of the plant.

Powdery mildew grows well in environments with high humidity and moderate temperatures. Greenhouses provide an ideal moist, temperate environment for the spread of the disease. This causes harm to agricultural and horticultural practices where powdery mildew may thrive in a greenhouse setting. In an agricultural or horticultural setting, the pathogen can be controlled using chemical methods, bio organic methods, and genetic resistance. It is important to be aware of powdery mildew and its management as the resulting disease can significantly reduce important crop yields.

MLO proteins function as negative regulators of plant defense to powdery mildew disease. Loss-of-function mlo alleles in barley, Arabidopsis and tomato have been reported to lead to broad-spectrum and durable resistance to the fungal pathogen causing powdery mildew.

U.S. Pat. Nos. 6,211,433 and 6,576,814 describe modulating the expression of Mlo genes in Maize by producing transgenic plants comprising mutation-induced recessive alleles of maize Mlo. However, such methods require genetically modifying the plant genome, particularly transforming plants with external foreign genes that enhance disease resistance.

US2018208939 discloses the generation of mutant wheat lines with mutations inactivating MLO alleles which confer heritable resistance to powdery mildew fungus.

Cannabis cultivation has always suffered from fungal diseases due to high humidity growing conditions in growth rooms or greenhouses.

In view of the above there is a heightened immediate need for the development of Cannabis plants that carry genetic resistance to fungal diseases, thereby reducing or eliminating the need for fungicide use in the cultivation of Cannabis. In addition, there is a need for non-GMO, advanced breeding programs of Cannabis for food, medicine and fiber (Hemp) production.

### SUMMARY OF THE INVENTION

It is therefore one object of the present invention to disclose a modified Cannabis plant exhibiting enhanced resistance to powdery mildew (PM), wherein said modified plant comprises a mutated Cannabis mlo1 (Csmlo1) allele, said mutated allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof.

It is a further object of the present invention to disclose the modified Cannabis plant as defined above, wherein said indel comprises a sequence as set forth in SEQ ID NO:883 or a fraction thereof.

It is a further object of the present invention to disclose the modified Cannabis plant as defined in any of the above, wherein said Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof.

It is a further object of the present invention to disclose the modified Cannabis plant as defined in any of the above, wherein said Csmlo1 mutant allele is at least one of: (a) comprises a nucleic acid sequence corresponding to the sequence as set forth in SEQ ID NO:886, or a homologue having at least 80% sequence identity to the nucleic acid sequence of said mutated Csmlo1 allele; (b) confers an enhanced resistance to powdery mildew as compared to a Cannabis plant comprising a wild type CsMLO1 allele having a nucleic acid sequence with at least 80% sequence identity to a nucleic acid sequence as set forth in SEQ ID NO:882 and/or to a nucleic acid sequence as set forth in SEQ ID NO:1; (c) comprising a deletion of 14 bp at position 389 of SEQ ID NO: 1, or a nucleic acid insertion at position 482-483 of SEQ ID NO: 1, or a combination thereof; and (d) generated using genome editing.

It is a further object of the present invention to disclose the modified Cannabis plant as defined in any of the above, wherein said plant has decreased expression levels of Mlo1 protein, relative to a Cannabis plant lacking said mutated Csmlo1 allele.

It is a further object of the present invention to disclose the modified Cannabis plant as defined in any of the above, wherein said genome modification is generated via introduction (a) Cas DNA and gRNA sequence selected from the group consisting of SEQ ID NO:17, SEQ ID NO:43 and SEQ ID NO:50 and any combination thereof, or (b) a ribonucleoprotein (RNP) complex comprising Cas protein and gRNA sequence selected from the group consisting of SEQ ID NO:17, SEQ ID NO:43 and SEQ ID NO:50 and any combination thereof.

It is a further object of the present invention to disclose the modified Cannabis plant as defined in any of the above, wherein said PM is selected from the group consisting of Golovinomyces cichoracearum, Golovinomyces ambrosiae and a mixture thereof.

It is a further object of the present invention to disclose a progeny plant, plant part, plant seed, tissue culture of regenerable cells, protoplasts, callus or plant cell of a modified plant as defined in any of the above.

It is a further object of the present invention to disclose the modified Cannabis plant as defined in any of the above, wherein said modified plant comprises a targeted genome modification conferring reduced expression of a Cannabis MLO1 (CsMLO1) gene as compared to a Cannabis plant lacking said targeted genome modification, said targeted genome modification generates a mutated Cannabis mlo1 (Csmlo1) allele comprising a deletion of a nucleic acid sequence as set forth in SEQ ID NO:883 or a fraction thereof as compared to the wild type CsMLO1 allele comprising a sequence as set forth in SEQ ID NO:1, or a nucleic acid insertion at position 482-483 of SEQ ID NO:1, or a combination thereof.

It is a further object of the present invention to disclose a method for producing a modified Cannabis plant as defined in any of the above, said method comprises introducing using targeted genome modification, at least one genomic modification conferring reduced expression of at least one Cannabis MLO1 (CsMLO1) allele as compared to a Cannabis plant lacking said targeted genome modification, said genomic modification generates a mutated Cannabis mlo1 (Csmlo1) allele comprising an indel of 14 bp at a position corresponding to position 12 of SEQ ID NO: 882 or a fraction thereof, or a nucleic acid insertion at position corresponding to position 104-105 of SEQ ID NO: 882, or a combination thereof.

It is a further object of the present invention to disclose the method as defined in any of the above, comprises at least one step of: (a) introducing a loss of function mutation into said CsMLO1 allele using targeted genome modification; (b) introducing an expression vector comprising a promoter operably linked to a nucleotide sequence encoding a plant optimized Cas9 endonuclease and gRNA targeted to at least one CsMLO1 allele, said gRNA nucleotide sequence targeting said CsMLO1 allele is selected from the group consisting of SEQ ID NO:17, SEQ ID NO:43 and SEQ ID NO:50 or a complementary sequence thereof; (c) introducing and co-expressing in a Cannabis plant Cas9 and gRNA targeted to CsMLO1 gene and screening for induced targeted mutations conferring reduced expression of said CsMLO1 gene; (d) selecting a plant resistant to powdery mildew from plants comprising mutated Csmlo1 allele, said selected plant is characterized by enhanced resistance to powdery mildew as compared to a Cannabis plant comprising a CsMLO1 nucleic acid comprising a nucleic acid sequence as set forth in SEQ ID NO:882; (e) regenerating a plant carrying said genomic modification; and (f) screening said regenerated plants for a plant resistant to powdery mildew.

It is a further object of the present invention to disclose the method as defined in any of the above, wherein at least one of the following holds true: (a) said indel comprises a sequence as set forth in SEQ ID NO:883 or a fraction thereof; (b) said modified plant has decreased levels of at least one Mlo protein as compared to wild type Cannabis plant; (c) said genome modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof; (d) said genetic modification in said CsMLO1 is generated in planta via introduction of a construct comprising (a) Cas DNA and gRNA sequence selected from the group consisting of SEQ ID NO:17, SEQ ID NO:43 and SEQ ID NO:50 or a complementary sequence thereof, and any combination thereof, or (b) a ribonucleoprotein (RNP) complex comprising Cas protein and gRNA sequence selected from the group consisting of SEQ ID NO:17, SEQ ID NO:43 and SEQ ID NO:50 or a complementary sequence thereof, and any combination thereof; (e) said powdery mildew is selected from the group of species consisting of Golovinomyces cichoracearum, Golovinomyces ambrosiae and a mixture thereof; (f) said Cannabis plant is selected from the group of species that includes, but is not limited to, Cannabis sativa (C. sativa), C. indica, C. ruderalis and any hybrid or cultivated variety of the genus Cannabis; (g) said Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof; (h) said Csmlo1 mutant allele comprises a nucleic acid sequence corresponding to the sequence as set forth in SEQ ID NO:886, or a homologue having at least 80% sequence identity to the nucleic acid sequence of said mutated Csmlo1 allele; (i) said mutated Csmlo1 allele confers an enhanced resistance to powdery mildew as compared to a Cannabis plant comprising a wild type CsMLO1 allele having a nucleic acid sequence at least 80% sequence identity to a sequence as set forth in SEQ ID NO:882 and/or to a nucleic acid sequence as set forth in SEQ ID NO:1; and (j) said mutated allele comprising a deletion of 14 bp at position 389 of SEQ ID NO: 1, or a nucleic acid insertion at position 482-483 of SEQ ID NO: 1, or a combination thereof.

It is a further object of the present invention to disclose a method for conferring powdery mildew resistance to a Cannabis plant comprising producing a plant according to the method as defined in any of the above.

It is a further object of the present invention to disclose a plant, plant part, plant cell, tissue culture or a seed obtained or obtainable by the method as defined in any of the above.

It is a further object of the present invention to disclose a method for identifying a Cannabis plant with resistance to powdery mildew, said method comprises steps of: (a) screening the genome of said Cannabis plant for a mutated Csmlo1 allele, said mutated allele comprises a genomic modification selected from an indel of 14 bp at a position corresponding to position 12 of SEQ ID NO: 882 or a fraction thereof, or a nucleic acid insertion at position corresponding to position 104-105 of SEQ ID NO: 882, or a combination thereof; (b) optionally, regenerating plants carrying said genetic modification; and (c) optionally, screening said regenerated plants for a plant resistant to powdery mildew.

It is a further object of the present invention to disclose an isolated polynucleotide sequence having at least 80% sequence identity to a nucleic acid sequence selected from the group consisting of SEQ ID NO:882-886, SEQ ID NO:17, SEQ ID NO:43 and SEQ ID NO:50 or a complementary sequence or any combination thereof.

It is a further object of the present invention to disclose use of the polynucleotide sequence as defined in any of the above, for generating, identifying and/or screening for a Cannabis plant comprising within its genome mutant Csmlo allele conferring resistance to PM, wherein the presence of at least one nucleic acid sequence selected from the group consisting of SEQ ID NO:1, SEQ ID NO:883, SEQ ID NO:882 indicates that the Cannabis plant comprises a wild type CsMLO1 allele, and the presence of at least one nucleic acid sequence selected from the group consisting of SEQ ID NO:884, SEQ ID NO:885 and SEQ ID NO:886 indicates that the Cannabis plant comprises a mutant Csmol1 allele.

It is a further object of the present invention to disclose a detection kit for determining the presence or absence of a mutant Csmlo1 allele in a Cannabis plant, said kit comprising at least one of the isolated polynucleotide sequence as defined in any of the above, said kit is useful for identifying a Cannabis plant with enhanced resistance to powdery mildew, wherein the presence of at least one nucleic acid sequence selected from the group consisting of SEQ ID NO:1, SEQ ID NO:883, SEQ ID NO:882 indicates that the Cannabis plant comprises a wild type CsMLO1 allele, and the presence of at least one nucleic acid sequence selected from the group consisting of SEQ ID NO:884, SEQ ID NO:885 and SEQ ID NO:886 indicates that the Cannabis plant comprises a mutant Csmol1 allele.

It is a further object of the present invention to disclose a method of determining the presence of a mutant Csmlo1 allele in a Cannabis plant using the isolated polynucleotide sequence as defined in any of the above, comprising assaying said Cannabis plant for at least one of the presence of an indel comprising a nucleic acid sequence as set forth in SEQ ID NO:883, an insertion at position 104-105 of SEQ ID NO: 882, a nucleic acid sequence corresponding to the sequence as set forth in SEQ ID NO:884, a nucleic acid sequence corresponding to the sequence as set forth in SEQ ID NO:885, a nucleic acid sequence corresponding to the sequence as set forth in SEQ ID NO:886, or a homologue having at least 80% sequence identity to the nucleic acid sequence of said mutated Csmlo1 allele, a complementary sequence thereof, or any combination thereof.

It is a further object of the present invention to disclose a method for down regulation of Cannabis MLO1 (CsMLO1) gene, which comprises utilizing the isolated polynucleotide sequence as defined in any of the above, by steps of utilizing the nucleotide sequence as set forth in at least one of SEQ ID NO:43 and SEQ ID NO:50 or a complementary sequence thereof, and a combination thereof, for introducing a loss of function mutation into said CsMLO1 gene using targeted genome modification.

### BRIEF DESCRIPTION OF THE FIGURES

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIGS. 1A-C is presenting a photographic illustration of an infected Cannabis plant leaf exhibiting PM symptoms of white powdery spots on the leaves (FIG. 1A), an enlarged view (×4) of fungal asexual spore-carrying bodies (conidia) of Golovinomyces cichoracearum on Cannabis leaf tissue (FIG. 1B), and a microscopic imaging of Golovinomyces cichoracearum spores (FIG. 1C);

FIG. 2A-B is schematically presenting WT plant cell penetrated by the fungal appressorium leading to haustorium establishment and infection by secondary hyphae (FIG. 2A), and mlo knockout plant cell into which the fungal spores are incapable of penetrating (FIG. 2B);

FIG. 3 is schematically presenting CRISPR/Cas9 mode of action as depicted by Xie, Kabin, and Yinong Yang. “RNA-guided genome editing in plants using a CRISPR—Cas system.” Molecular plant 6.6 (2013): 1975-1983;

FIG. 4A-D is photographically presenting GUS staining after transient transformation of Cannabis axillary buds (FIG. 4A), leaves (FIG. 4B), calli (FIG. 4C), and cotyledons (FIG. 4D);

FIG. 5 is presenting regenerated Cannabis tissue;

FIG. 6 is photographically presenting PCR detection of Cas9 DNA in shoots of Cannabis plants transformed using biolistics;

FIG. 7A-B is illustrating in vitro cleavage activity of CRISPR/Cas9; a scheme of genomic area targeted for editing is shown in FIG. 7A, and a gel showing digestion of PCR amplicon containing the gRNA sequence by RNP complex containing Cas9 and gene specific gRNA is shown in FIG. 7B;

FIG. 8 is presenting a schematic illustration of a DNA plasmid containing a plant codon optimized Cas9 nuclease from Streptococcus pyogenes (pcoSpCas9) and sgRNA sequences used for transformation, as embodiments of the present invention;

FIG. 9 schematically presents genomic localization of sgRNAs used for targeting CsMLO1 first exon, as embodiments of the present invention;

FIG. 10 presents genomic nucleotide sequence of the first exon (exon 1) of wild type CsMLO1 targeted by three gRNA sequences;

FIG. 11 presents amino acid sequence of the first exon (exon 1) of wild type CsMLO1;

FIG. 12 photographically presents detection of CsMLO1 PCR products showing length variation as a result of Cas9-mediated genome editing;

FIG. 13 schematically presents genome edited CsMLO1 DNA fragments produced by the present invention; and

FIG. 14 schematically presents nucleic acid sequence comparison of WT CsMLO1 and genome edited Csmlo1_d14i1 fragments produced by the present invention.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a modified Cannabis plant exhibiting enhanced resistance to powdery mildew (PM), wherein the plant comprises a targeted genome modification conferring reduced expression of at least one Cannabis MLO (CsMLO) allele as compared to a Cannabis plant lacking said targeted genome modification.

The present invention is aimed at showing that lack of mildew resistance loci 0 (MLO) genes in Cannabis is correlated with resistance to PM. It is herein disclosed that MLO deletions are likely to increase PM resistance in Cannabis. According to further aspects of the invention, lack of certain MLO genes is used as markers for pathogen resistance and may accelerate breeding for more resistant Cannabis lines.

According to one embodiment, the present invention provides a modified Cannabis plant exhibiting enhanced resistance to powdery mildew (PM), wherein said modified plant comprises a mutated Cannabis mlo1 (Csmlo1) allele, said mutated allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof.

According to a further embodiment of the present invention, the indel comprises a sequence as set forth in SEQ ID NO:883 or a fraction thereof.

According to a further embodiment of the present invention, the Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof.

According to a further embodiment of the present invention, the Csmlo1 mutant allele comprises a nucleic acid sequence corresponding to the sequence as set forth in SEQ ID NO:886, or a homologue having at least 80% sequence identity to the nucleic acid sequence of said mutated Csmlo1 allele.

According to a further embodiment of the present invention, the mutated Csmlo1 allele confers an enhanced resistance to powdery mildew as compared to a Cannabis plant comprising a wild type CsMLO1 allele having a nucleic acid sequence as set forth in SEQ ID NO:882 and/or having a nucleic acid sequence as set forth in SEQ ID NO:1 or a functional variant thereof.

According to a further embodiment of the present invention, the functional variant has at least 80% sequence identity to the corresponding CsMLO1 nucleotide sequence.

According to a further embodiment of the present invention, the mutated allele comprising a deletion of 14 bp at position 389 of SEQ ID NO: 1, or a nucleic acid insertion at position 482-483 of SEQ ID NO: 1, or a combination thereof.

According to a further embodiment of the present invention, the mutated Csmlo1 allele is generated using genome editing.

It is further within the scope of the present invention to provide, a modified Cannabis plant exhibiting enhanced resistance to powdery mildew (PM), wherein said modified plant comprises a targeted genome modification conferring reduced expression of a Cannabis MLO1 (CsMLO1) gene as compared to a Cannabis plant lacking said targeted genome modification, said targeted genome modification generates a mutated Cannabis mlo1 (Csmlo1) allele comprising a deletion of a nucleic acid sequence as set forth in SEQ ID NO:883 or a fraction thereof as compared to the wild type CsMLO1 allele comprising a sequence as set forth in SEQ ID NO:1, or a nucleic acid insertion at position 482-483 of SEQ ID NO:1, or a combination thereof.

According to a further aspect of the present invention, a method for producing a modified Cannabis plant as defined in any of the above is provided. The method comprises introducing using targeted genome modification, at least one genomic modification conferring reduced expression of at least one Cannabis MLO1 (CsMLO1) allele as compared to a Cannabis plant lacking said targeted genome modification, said genomic modification generates a mutated Cannabis mlo1 (Csmlo1) allele comprising an indel of 14 bp at a position corresponding to position 12 of SEQ ID NO: 882 or a fraction thereof, or a nucleic acid insertion at position corresponding to position 104-105 of SEQ ID NO: 882, or a combination thereof.

According to further aspects of the present invention, a method of determining the presence of a mutant Csmlo1 allele in a Cannabis plant is provided. The method comprising assaying the Cannabis plant for at least one of the presence of an indel comprising a nucleic acid sequence as set forth in SEQ ID NO:883, an insertion at position 104-105 of SEQ ID NO: 882, a nucleic acid sequence corresponding to the sequence as set forth in SEQ ID NO:884, a nucleic acid sequence corresponding to the sequence as set forth in SEQ ID NO:885, a nucleic acid sequence corresponding to the sequence as set forth in SEQ ID NO:886, or a homologue having at least 80% sequence identity to the nucleic acid sequence of said mutated Csmlo1 allele, a complementary sequence thereof, or any combination thereof.

It is further within the scope to provide a method for identifying a Cannabis plant with resistance to powdery mildew, said method comprises steps of: (a) screening the genome of said Cannabis plant for a mutated Csmlo1 allele, said mutated allele comprises a genomic modification selected from an indel of 14 bp at a position corresponding to position 12 of SEQ ID NO: 882 or a fraction thereof, or a nucleic acid insertion at position corresponding to position 104-105 of SEQ ID NO: 882, or a combination thereof; (b) optionally, regenerating plants carrying said genetic modification; and (c) optionally, screening said regenerated plants for a plant resistant to powdery mildew.

It is further within the scope of the present invention to provide a method for down regulation of Cannabis MLO1 (CsMLO1) gene, which comprises utilizing the nucleotide sequence as set forth in at least one of SEQ ID NO:43 and SEQ ID NO:50 or a complementary sequence thereof, and a combination thereof, for introducing a loss of function mutation into said CsMLO1 gene using targeted genome modification.

The present invention further provides an isolated amino acid sequence having at least 80% sequence identity to a nucleic acid sequence selected from the group consisting of SEQ ID NO:882-886, SEQ ID NO:17, SEQ ID NO:43 and SEQ ID NO:50 or a complementary sequence or any combination thereof.

It is also within the scope to disclose a use of a nucleotide sequence as set forth in SEQ ID NO: 883-886, SEQ ID NO:17, SEQ ID NO:43 and SEQ ID NO:50 for generating, identifying and/or screening for a Cannabis plant comprising within its genome mutant Csmlo allele conferring resistance to PM.

It is also within the scope to disclose a use of a nucleotide sequence as set forth in at least one of SEQ ID NO:17, SEQ ID NO:43 and SEQ ID NO:50 or a complementary sequence or any combination thereof for targeted genome modification of Cannabis MLO1 (CsMLO1) gene.

According to further aspects, the present invention provides a detection kit for determining the presence or absence of a mutant Csmlo1 allele in a Cannabis plant, comprising a nucleic acid fragment comprising a sequence selected from SEQ ID NO:882-886, SEQ ID NO:17, SEQ ID NO:43 and SEQ ID NO:50 or a complementary sequence or any combination thereof.

According to an embodiment of the present invention, the targeted genome modification is in a CsMLO allele having a wild type genomic nucleotide sequence selected from the group consisting of CsMLO1 having a sequence as set forth in SEQ ID NO:1 or a functional variant thereof, CsMLO2 having a sequence as set forth in SEQ ID NO:4 or a functional variant thereof and CsMLO3 having a sequence as set forth in SEQ ID NO:7 or a functional variant thereof.

According to a further embodiment of the present invention, the functional variant has at least 75%, preferably 80% sequence identity to the corresponding CsMLO nucleotide sequence.

According to a further embodiment of the present invention, the modified Cannabis plant has decreased expression levels of at least one Mlo protein, relative to a Cannabis plant lacking the at least one genome modification.

According to a further embodiment of the present invention, the genome modification is introduced using mutagenesis, small interfering RNA (siRNA), microRNA (miRNA), artificial miRNA (amiRNA), DNA introgression, endonucleases or any combination thereof.

According to a further embodiment of the present invention, the genetic modification is introduced using targeted genome modification, preferably said genetic modification is introduced using an endonuclease.

According to a further embodiment of the present invention, the genome modification is introduced using guide RNA, e.g. single guide RNA (sgRNA) designed and targeted to mutate CsMLO1 gene, said sgRNA nucleotide sequence is selected from the group consisting of SEQ ID NO:17, SEQ ID NO:43 and SEQ ID NO:50.

According to a further embodiment of the present invention, the modified Cannabis plant comprises at least one mutated CsMLO1 allele comprising a nucleotide sequence selected from the group consisting of a nucleotide sequence as set forth in SEQ ID NO:875, a nucleotide sequence as set forth in SEQ ID NO:877, a nucleotide sequence as set forth in SEQ ID NO:880 or a homologue having at least 80% sequence identity to the nucleotide sequence of said at least one mutated CsMLO1 allele or a combination thereof.

According to a further embodiment of the present invention, the modified Cannabis plant comprises at least one silencing mutation, a knockdown mutation, a knockout mutation, a loss of function mutation or any combination thereof in at least one gene or allele selected from the group consisting of CsMLO1, CsMLO2 and CsMLO3.

According to a further embodiment of the present invention the mutated CsMLO1 allele comprises a deletion having a nucleotide sequence as set forth in SEQ ID NO.:876, SEQ ID NO.:879 or SEQ ID NO.:881.

According to a further embodiment of the present invention, the mutated CsMLO1 allele confers an enhanced resistance to powdery mildew as compared to a Cannabis plant comprising a wild type CsMLO1 allele sequence.

According to a further embodiment of the present invention, the wild type CsMLO1 allele comprises a nucleic acid sequence as set forth in at least one of SEQ ID NO:873, SEQ ID NO:876, SEQ ID NO:879 or SEQ ID NO:881. According to a further embodiment of the present invention the present invention provides modified Cannabis plant exhibiting enhanced resistance to powdery mildew (PM) compared to wild type Cannabis plant, wherein the modified plant comprises a genetic modification conferring reduced expression of at least one Cannabis MLO (CsMLO) allele. The present invention further provides methods for producing the aforementioned modified Cannabis plant using genome editing or modification techniques.

Powdery mildew (PM) is a major fungal disease that threatens thousands of plant species. Powdery mildew is commonly controlled by frequent applications of fungicides, having negative effects on the environment, and leading to additional costs for growers. To reduce the amount of chemicals required to control this pathogen, the development of resistant crop varieties is a priority.

It is herein acknowledged that PM pathogenesis is associated with up-regulation of specific MLO genes during early stages of infection, causing down-regulation of plant defense pathways. These up-regulated genes are responsible for PM susceptibility (S-genes) and their knock-out cause durable and broad-spectrum resistance.

As the Cannabis legal market is expanding worldwide, this agricultural crop will gradually move from indoor growing facilities to simple low cost greenhouses to enable mass production at reduced operational costs. One of the major challenges facing this transition is the lack of compatible genetics (strains) adapted for green house growth and more specifically genetic fungal resistances. Cannabis susceptibility to fungal diseases results in damages and losses to the grower and forces the widespread use of fungicides. Excessive fungicide use poses health threats to Cannabis consumers.

To date, there are no fungal disease resistant Cannabis varieties on the market. Classical breeding programs dedicated to the end of creating fungal disease resistant Cannabis varieties are virtually impossible due to limited genetic variation, legal constraints on import and export of genetic material and limited academic knowledge and gene banks involved is such projects. In addition, traditional breeding is a long process with low rates of success and certainty, as it is based on trial and error.

The solution proposed by the current invention is using genome editing such as the CRISPR/Cas system in order to create fungal disease resistant Cannabis varieties. Breeding using genome editing allows a precise and significantly shorter breeding process in order to achieve these goals with a much higher success rate. Thus genome editing, has the potential to generate improved varieties faster and at a lower cost. By using genome editing to generate Powdery Mildew (PM) resistant Cannabis varieties, the current disclosure will allow growers worldwide to supply a safer product to Cannabis consumers.

It is further noted that using genome editing is considered as non GMO by the Israeli regulator and in the US, the USDA has already classified a dozen of genome edited plant as non regulated and non GMO (https://www.usda.gov/media/press-releases/2018/03/28/secretary-perdue-issues-usda-statement-plant-breeding-innovation).

The Cannabis industry's value chain is based on a steady supply of high quality consistent product. Due to lack of suitable genetics adapted for intensive agriculture production, most growing methods are based on cloning as a mean of vegetative propagation in order to ensure genetic consistency of the plant material. These methods are outdated, expensive and not fit for purpose.

The lack of Cannabis strains that are disease resistant, stable and uniform, pose a threat to the ability of supplying the industry with the raw material needed to support itself.

Legal limitations and outdated breeding techniques significantly hamper the efforts of generating new and improved Cannabis varieties fit for intensive agriculture.

Cannabis legalization in certain countries has increased significantly the number of Cannabis growers and area used for growing. One possible solution is moving growing Cannabis into greenhouses (protected growing facilities) like the vegetable industry has been doing for the last few decades. Unlike the vegetable industry, Cannabis is based on vegetative propagation while vegetables are grown through seeds. In addition, Cannabis growers are using Cannabis strains that were bred for indoor cultivation and are now using those for their greenhouse operations. This situation is obviously not ideal and causes many logistic issues for the growers. For example, since Cannabis plants require short days for the induction of flowering, growers install darkening curtains in the greenhouse to control day length for the plants. This artificial darkening results in increased humidity in the greenhouse thus creating optimal conditions for fungal pathogens to spread and thrive. These conditions force growers to intensively use fungicides to control pathogen populations. With strict regulatory constraints in place across the legalized states, these conditions pose a great challenge for sustainable Cannabis production and consumer health.

The next step for the Cannabis industry is the adoption and use of hybrid seeds for propagation, which is common practice in the conventional seed industry (from field crops to vegetables). In addition, breeding for basic agronomic traits that are completely lacking in currently available Cannabis varieties (with an emphasis on disease resistances) will significantly increase grower's productivity. This will allow growing and supplying high quality raw material for the Cannabis industry.

In order to generate a reproducible product, Cannabis growers are currently using vegetative propagation (cloning or tissue culture). However, in conventional agricultural, genetic stability of field crops and vegetables is maintained by using F1 hybrid seeds. These hybrids are generated by crossing homozygous parental lines.

Currently, breeding of Cannabis plants is mostly done by small Cannabis growers. There is very limited if any molecular tools supporting or leading the breeding process. Traditional Cannabis breeding is done by mixing breeding material with hope to find the desired traits and phenotypes by random crosses.

The present invention provides for the first time enhanced resistant Cannabis plants to fungal diseases. The current invention disclose the generation of non-transgenic Cannabis plant resistant to the powdery mildew fungal disease, using the genome editing technology, e.g., the CRISPR/Cas9 tool. The generated mutations can be readily introduced into elite or locally adapted Cannabis lines rapidly, with relatively minimal effort and investment.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. reduced expression of at least one CsMLO gene.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

As used herein the term “genetic modification” or “genomic modification” refers hereinafter to genetic manipulation or modulation, which is the manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with increased resistance to PM are generated using genome editing mechanism. This technique enables to achieve in planta modification of specific genes that relate to and/or control the infection of powdery mildew in the Cannabis plant.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

Genome Editing Glossary

Cas = CRISPR-associated genes
Cas9, Csn1 = a CRISPR-associated protein containing two nuclease
domains, that is programmed by small RNAs to cleave DNA
crRNA = CRISPR RNA
dCAS9 = nuclease-deficient Cas9
DSB = Double-Stranded Break
gRNA = guide RNA
HDR = Homology-Directed Repair
HNH = an endonuclease domain named for characteristic histidine
and asparagine residues
Indel = insertion and/or deletion
NHEJ = Non-Homologous End Joining
PAM = Protospacer-Adjacent Motif
RuvC = an endonuclease domain named for an E. coli protein involved in
DNA repair
sgRNA = single guide RNA
tracrRNA, trRNA = trans-activating crRNA
TALEN = Transcription-Activator Like Effector Nuclease
ZFN = Zinc-Finger Nuclease

It is noted that it is within the scope of the current invention that the term gRNA also refers to or means single guide RNA (sgRNA).

According to specific aspects of the present invention, the CRISPR (Clustered Regularly Interspaced Short Palindromic Repeats) and CRISPR-associated (Cas) genes are used for the first time for generating genome modification in targeted genes in the Cannabis plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-6 nucleotides) known as protospacer-associated motif (PAM), follows immediately 3′- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

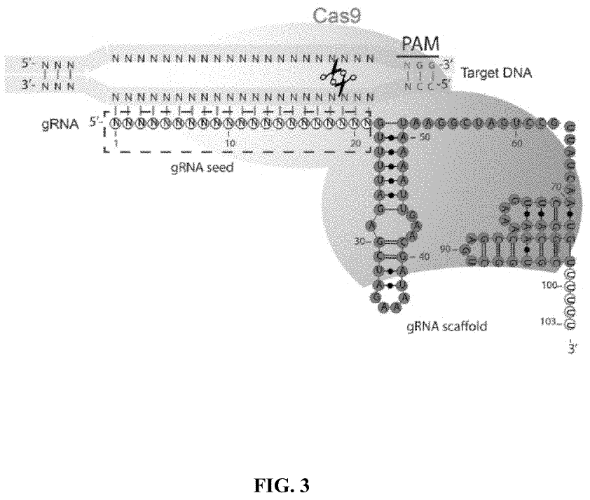

Reference is now made to FIG. 3 schematically presenting an example of CRISPR/Cas9 mechanism of action as depicted by Xie, Kabin, and Yinong Yang. “RNA-guided genome editing in plants using a CRISPR—Cas system.” Molecular plant 6.6 (2013): 1975-1983. As shown in this figure, the Cas9 endonuclease forms a complex with a chimeric RNA (called guide RNA or gRNA), replacing the crRNA—transcrRNA heteroduplex, and the gRNA could be programmed to target specific sites. The gRNA—Cas9 should comprise at least 15-base-pairing (gRNA seed region) without mismatch between the 5′-end of engineered gRNA and targeted genomic site, and a motif called protospacer-adjacent motif or PAM that follows the base-pairing region in the complementary strand of the targeted DNA. The commonly-used Cas9 from Streptococcus pyogenes (SpCas9) recognizes the PAM sequence 5′-NGG-3′ (where “N” can be any nucleotide base).

Other Cas variants and their PAM sequences (5′ to 3′) within the scope of the current invention include NmeCas9 (isolated from Neisseria meningitides) recognizing NNNNGATT, StCas9 (isolated from Streptococcus thermophiles) recognizing NNAGAAW, TdCas9 (isolated from Treponema denticola) recognizing NAAAAC, SaCas9 (isolated from Staphylococcus aureus) recognizing NNGRRT or NGRRT or NGRRN and TBN (Cas-phi).

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” or “silence” or silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it. In some embodiments of the present invention, gene silencing by targeted genome modification results in non-functional gene products, such as transcripts or proteins, for example non-functional CsMLO1 exon 1 fragments.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

As used herein, the term “powdery mildew” or “PM” refers hereinafter to fungi that are obligate, biotrophic parasites of the phylum Ascomycota of Kingdom Fungi. The diseases they cause are common, widespread, and easily recognizable. Infected plants display white powdery spots on the leaves and stems Infection by the fungus is favored by high humidity but not by free water. Powdery mildew fungi tend to grow superficially, or epiphytically, on plant surfaces. During the growing season, hyphae are produced preferably on both upper and lower leaf surfaces. Infections can also occur on stems, flowers, or fruit. Specialized absorption cells, termed haustoria, extend into the plant epidermal cells to obtain nutrition.

Powdery mildew fungi can reproduce both sexually and asexually. Sexual reproduction is via chasmothecia (cleistothecium), a type of ascocarp where the genetic material recombines. Within each ascocarp are several asci. Under optimal conditions, ascospores mature and are released to initiate new infections Conidia (asexual spores) are also produced on plant surfaces during the growing season. They develop either singly or in chains on specialized hyphae called conidiophores. Conidiophores arise from the epiphytic hyphae, or in the case of endophytic hyphae, the conidiophores emerge through leaf stomata. It should be noted that powdery mildew fungi must be adapted to their hosts to be able to infect them. The present invention provides for the first time Cannabis plants with enhanced resistance or tolerance to PM disease. The enhanced resistance to PM is generated by genome editing techniques targeted at silencing at least one Cannabis Mildew Locus O (MLO) gene. The modified resulted Cannabis plant exhibits enhanced resistance to PM as compared to a Cannabis plant lacking the targeted modification.

The term “MLO” or “Mlo” or “mlo” refers hereinafter to the Mildew Locus O (MLO) gene family encoding for plant-specific proteins harboring several transmembrane domains, topologically reminiscent of metazoan G-protein coupled receptors. It is within the scope of the present invention that specific homologs of the MLO family act as susceptibility genes towards PM fungi. It is emphasized that the present invention provides for the first time the identification of MLO orthologous alleles in the Cannabis plant. Three Cannabis MLO alleles or genes (i.e. MLO1, MLO2, MLO3) have been herein identified, namely CsMLO1, CsMLO2 and CsMLO3.

The term “orthologue” as used herein refers hereinafter to one of two or more homologous gene sequences found in different species.

The term “functional variant” or “functional variant of a nucleic acid or protein sequence” as used herein, for example with reference to SEQ ID NOs: 1, 2 or 3 refers to a variant gene sequence or part of the gene sequence which retains the biological function of the full non-variant allele (e.g. CsMLO allele) and hence has the activity of modulating response to PM. A functional variant also comprises a variant of the gene of interest encoding a polypeptide which has sequence alterations that do not affect function of the resulting protein, for example in non-conserved residues. Also encompassed is a variant that is substantially identical, i.e. has only some sequence variations, for example in non-conserved residues, to the wild type nucleic acid sequences of the alleles as shown herein and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the three identified Cannabis MLO genes, namely CsMLO1, CsMLO2 and CsMLO3 having the genomic nucleotide sequence as set forth in SEQ ID NOs: 1, 2 or 3, respectively.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism. In specific embodiments, the tomato plants of the present invention comprise heterozygous configuration of the genetic markers associated with the high yield characteristics.

The term “corresponding” or “corresponding to” or “corresponding to nucleotide sequence” or “corresponding to position” as used herein, refers in the context of the present invention to sequence homology or sequence identity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the term “corresponding to the nucleotide sequence” or “corresponding to position”, refers to variants, homologues and fragments of the indicated nucleotide sequence, which possess or perform the same biological function or correlates with the same phenotypic characteristic of the indicated nucleotide sequence.

Another indication that two nucleic acid sequences are substantially identical or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

In other embodiments of the invention, such substantially identical sequences refer to polynucleotide or amino acid sequences that share at least about 80% similarity, preferably at least about 90% similarity, alternatively, about 95%, 96%, 97%, 98% or 99% similarity to the indicated polynucleotide or amino acid sequences.

According to other aspects of the invention, the term “corresponding” refers also to complementary sequences or base pairing such that when they are aligned antiparallel to each other, the nucleotide bases at each position in the sequences will be complementary. The degree of complementarity between two nucleic acid strands may vary.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a ‘modified” or a “mutant” plant is a plant that has been altered compared to the naturally occurring wild type (WT) plant. Specifically, the endogenous nucleic acid sequences of each of the MLO homologs in Cannabis (nucleic acid sequences CsMLO1, CsMLO2 and CsMLO3) have been altered compared to wild type sequences using mutagenesis and/or genome editing methods as described herein. This causes inactivation of the endogenous Mlo genes and thus disables Mlo function. Such plants have an altered phenotype and show resistance or increased resistance to PM compared to wild type plants. Therefore, the resistance is conferred by the presence of at least one mutated endogenous CsMLO1, CsMLO2 and CsMLO3 genes in the Cannabis plant genome which has been specifically targeted using targeted genome modification.

According to further aspects of the present invention, the increased resistance to PM is not conferred by the presence of transgenes expressed in Cannabis.

It should be noted that nucleic acid sequences of wild type alleles are designated using capital letters namely CsMLO1, CsMLO2 and CsMLO3. Mutant mlo nucleic acid sequences use non-capitalization. Cannabis plants of the invention are modified plants compared to wild type plants which comprise and express mutant mlo alleles.

It is further within the scope of the current invention that mlo mutations that down-regulate or disrupt functional expression of the wild-type Mlo sequence may be recessive, such that they are complemented by expression of a wild-type sequence.

A mlo mutant phenotype according to the invention is characterized by the exhibition of an increased resistance against PM. In other words, a mlo mutant according to the invention confers resistance to the pathogen causing PM, which is identified as described inter alia.

It is further noted that a wild type Cannabis plant is a plant that does not have any mutant Mlo alleles.

Main aspects of the invention involve targeted mutagenesis methods, specifically genome editing, and exclude embodiments that are solely based on generating plants by traditional breeding methods. In a further embodiment of the current invention, as explained herein, the disease resistant trait is not due to the presence of a transgene.

The inventors have generated mutant Cannabis lines with mutations inactivating at least one CsMLO homoeoallele which confer heritable resistance to powdery mildew. In this way no functional CsMLO protein is made. Thus, the invention relates to these mutant Cannabis lines and related methods.

According to one embodiment, the present invention provides a modified Cannabis plant exhibiting enhanced resistance to powdery mildew (PM) compared to wild type Cannabis plant. The Cannabis plant of the present invention comprises a genetic modification conferring reduced expression of at least one Cannabis MLO (CsMLO) allele.

It is within the scope of the present invention that the CsMLO allele is selected from the group consisting of CsMLO1 having a nucleotide sequence as set forth in SEQ ID NO:1 or a fragment or a functional variant thereof, CsMLO2 having a nucleotide sequence as set forth in SEQ ID NO:4 or a fragment or a functional variant thereof and CsMLO3 having a nucleotide sequence as set forth in SEQ ID NO:7 or a fragment or a functional variant thereof.

According to a further embodiment of the present invention, the functional variant has at least 75% sequence identity to the CsMLO nucleotide sequence.

It is within the scope of the current invention that genome editing can be achieved using sequence-specific nucleases (SSNs) and results in chromosomal changes, such as nucleotide deletions, insertions or substitutions at specified genetic loci. Non limiting examples of SSNs include zinc finger nucleases (ZFNs), TAL effector nucleases (TALENs) and, clustered regularly interspaced short palindromic repeats (CRISPR)/CRISPR-associated protein (Cas) system.

Non limiting examples Cas proteins used by the present invention include Csn1, Cpf1 Cas9, Cas12, Cas13, Cas14, CasX and any combination thereof.

According to further aspects of the invention, Cannabis plant resistant to the powdery mildew fungal pathogen using the CRISPR/Cas9 technology is generated, which is based on the Cas9 DNA nuclease guided to a specific DNA target by a single guide RNA (sgRNA).

It is herein acknowledged that wild-type alleles of MILDEW RESISTANT LOCUS 0 (Mlo), which encodes a membrane-associated protein with seven transmembrane domains, confer susceptibility to fungi causing the powdery mildew disease. Therefore, homozygous loss-of-function mutations (mlo) result in resistance to powdery mildew.

According to certain embodiments of the present invention, in planta modification of specific genes that relate to and/or control the infection of powdery mildew in the Cannabis plant is achieved for the first time by the present invention, i.e. the Cannabis MLO genes (CsMLO). More specifically, but not limited to, the use of gene editing technologies, for example the CRISPR/Cas technology (e.g. Cas9 or Cpf1), in order to generate knockout alleles of genes (i.e. MLO genes) controlling the resistance to powdery mildew (PM) is disclosed for the Cannabis plant. The above in planta modification can be based on alternative gene editing technologies such as Zinc Finger Nucleases (ZFN's), Transcription activator-like effector nucleases (TALEN's), RNA silencing (amiRNA etc.) and/or meganucleases.

The loss of function mutation may be a deletion or insertion (“indels”) with reference the wild type CsMLO allele sequence. The deletion may comprise 1-20 or more, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular CsMLO nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant CsMLO nucleotide sequence of the CsMLO allele as shown in SEQ ID NO 1, 2 or 3. Sequence alignment programs to determine sequence identity are well known in the art.

Also, the various aspects of the invention encompass not only a CsMLO nucleic acid sequence or amino acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein and hence act to modulate responses to PM.

According to a further embodiment of the invention, the herein newly identified Cannabis MLO locus (CsMLO) have been targeted using the triple sgRNA strategy.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of PM resistant Cannabis plants, allows the modification of predetermined specific DNA sequences without introducing foreign DNA into the genome by GMO techniques.

According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence (for example see FIG. 3). The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that resistance to PM in Cannabis plants is produced by generating gRNA with homology to a specific site of predetermined genes in the Cannabis genome i.e. MLO genes, sub cloning this gRNA into a plasmid containing the Cas9 gene, and insertion of the plasmid into the Cannabis plant cells. In this way site specific mutations in the MLO genes are generated thus effectively creating non-active molecules, resulting in inability of powdery mildew and similar organisms of infecting the genome edited plant.

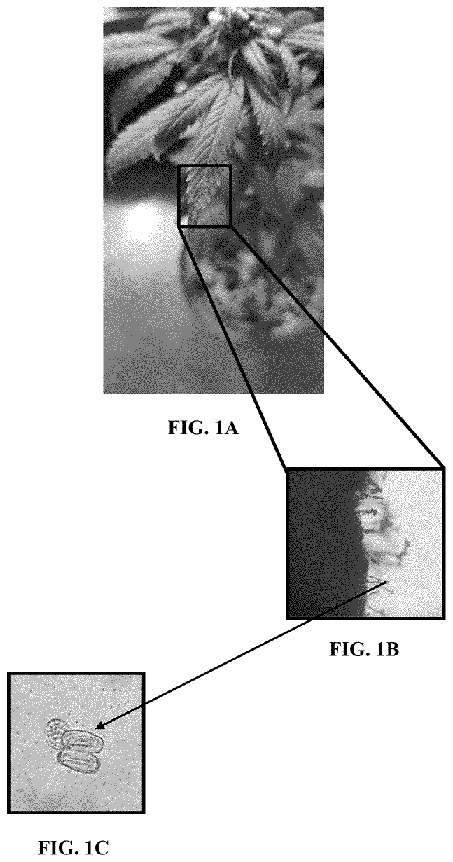

Reference is now made to FIGS. 1A-C schematically present Cannabis plant infected by the fungal pathogen Golovinomyces cichoracearum, causal agent of the Powdery Mildew disease. More specifically this figure shows (A) Cannabis plant leaf exhibiting PM symptoms (B) Fungal asexual spore-carrying bodies (conidia) of Golovinomyces cichoracearum on Cannabis leaf tissue, and (C) microscopic view of Golovinomyces cichoracearum spores.

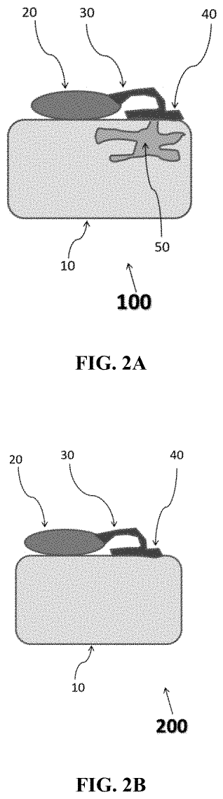

Reference is now made to FIG. 2A-B schematically presenting PM resistance suggested mode of action. This figure shows (A) a WT plant cell penetrated by the PM fungus (100). More particularly, a WT plant cell 10 is infected by PM spore 20 producing germ tubes 30 and penetrated by the PM fungal appressorium 40 which then leads to haustorium 50 establishment and infection by secondary hyphae; and (B) an mlo knockout cell 15 rendering fungal spores incapable of penetrating the plant cell (200).

According to one embodiment, the present invention provides a modified Cannabis plant exhibiting enhanced resistance to powdery mildew (PM), wherein said modified plant comprises a mutated Cannabis mlo1 (Csmlo1) allele, said mutated allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof.

According to a further embodiment of the present invention, the indel comprises a sequence as set forth in SEQ ID NO:883 or a fraction thereof.

According to a further embodiment of the present invention, the Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof.

According to a further embodiment of the present invention, the Csmlo1 mutant allele comprises a nucleic acid sequence corresponding to the sequence as set forth in SEQ ID NO:886, or a homologue having at least 80% sequence identity to the nucleic acid sequence of said mutated Csmlo1 allele.

According to a further embodiment of the present invention, the mutated Csmlo1 allele confers an enhanced resistance to powdery mildew as compared to a Cannabis plant comprising a wild type CsMLO1 allele having a nucleic acid sequence as set forth in SEQ ID NO:882 and/or having a nucleic acid sequence as set forth in SEQ ID NO:1 or a functional variant thereof.

According to a further embodiment of the present invention, the functional variant has at least 80% sequence identity to the corresponding CsMLO1 nucleotide sequence.

According to a further embodiment of the present invention, the mutated allele comprising a deletion of 14 bp at position 389 of SEQ ID NO: 1, or a nucleic acid insertion at position 482-483 of SEQ ID NO: 1, or a combination thereof.

According to a further embodiment of the present invention, the mutated Csmlo1 allele is generated using genome editing.

It is further within the scope of the present invention to provide, a modified Cannabis plant exhibiting enhanced resistance to powdery mildew (PM), wherein said modified plant comprises a targeted genome modification conferring reduced expression of a Cannabis MLO1 (CsMLO1) gene as compared to a Cannabis plant lacking said targeted genome modification, said targeted genome modification generates a mutated Cannabis mlo1 (Csmlo1) allele comprising a deletion of a nucleic acid sequence as set forth in SEQ ID NO:883 or a fraction thereof as compared to the wild type CsMLO1 allele comprising a sequence as set forth in SEQ ID NO:1, or a nucleic acid insertion at position 482-483 of SEQ ID NO:1, or a combination thereof.

According to a further aspect of the present invention, a method for producing a modified Cannabis plant as defined in any of the above is provided. The method comprises introducing using targeted genome modification, at least one genomic modification conferring reduced expression of at least one Cannabis MLO1 (CsMLO1) allele as compared to a Cannabis plant lacking said targeted genome modification, said genomic modification generates a mutated Cannabis mlo1 (Csmlo1) allele comprising an indel of 14 bp at a position corresponding to position 12 of SEQ ID NO: 882 or a fraction thereof, or a nucleic acid insertion at position corresponding to position 104-105 of SEQ ID NO: 882, or a combination thereof.

According to further aspects of the present invention, a method of determining the presence of a mutant Csmlo1 allele in a Cannabis plant is provided. The method comprising assaying the Cannabis plant for at least one of the presence of an indel comprising a nucleic acid sequence as set forth in SEQ ID NO:883, an insertion at position 104-105 of SEQ ID NO: 882, a nucleic acid sequence corresponding to the sequence as set forth in SEQ ID NO:884, a nucleic acid sequence corresponding to the sequence as set forth in SEQ ID NO:885, a nucleic acid sequence corresponding to the sequence as set forth in SEQ ID NO:886, or a homologue having at least 80% sequence identity to the nucleic acid sequence of said mutated Csmlo1 allele, a complementary sequence thereof, or any combination thereof.

It is further within the scope to provide a method for identifying a Cannabis plant with resistance to powdery mildew, said method comprises steps of: (a) screening the genome of said Cannabis plant for a mutated Csmlo1 allele, said mutated allele comprises a genomic modification selected from an indel of 14 bp at a position corresponding to position 12 of SEQ ID NO: 882 or a fraction thereof, or a nucleic acid insertion at position corresponding to position 104-105 of SEQ ID NO: 882, or a combination thereof; (b) optionally, regenerating plants carrying said genetic modification; and (c) optionally, screening said regenerated plants for a plant resistant to powdery mildew.

It is further within the scope of the present invention to provide a method for down regulation of Cannabis MLO1 (CsMLO1) gene, which comprises utilizing the nucleotide sequence as set forth in at least one of SEQ ID NO:43 and SEQ ID NO:50 or a complementary sequence thereof, and a combination thereof, for introducing a loss of function mutation into said CsMLO1 gene using targeted genome modification.

The present invention further provides an isolated amino acid sequence having at least 80% sequence identity to a nucleic acid sequence selected from the group consisting of SEQ ID NO:882-886, SEQ ID NO:17, SEQ ID NO:43 and SEQ ID NO:50 or a complementary sequence or any combination thereof.

It is also within the scope to disclose a use of a nucleotide sequence as set forth in SEQ ID NO: 883-886, SEQ ID NO:17, SEQ ID NO:43 and SEQ ID NO:50 for generating, identifying and/or screening for a Cannabis plant comprising within its genome mutant Csmlo allele conferring resistance to PM.

It is also within the scope to disclose a use of a nucleotide sequence as set forth in at least one of SEQ ID NO:17, SEQ ID NO:43 and SEQ ID NO:50 or a complementary sequence or any combination thereof for targeted genome modification of Cannabis MLO1 (CsMLO1) gene.

According to further aspects, the present invention provides a detection kit for determining the presence or absence of a mutant Csmlo1 allele in a Cannabis plant, comprising a nucleic acid fragment comprising a sequence selected from SEQ ID NO:882-886, SEQ ID NO:17, SEQ ID NO:43 and SEQ ID NO:50 or a complementary sequence or any combination thereof.

It is a further aspect of the present invention to disclose a modified Cannabis plant exhibiting enhanced resistance to powdery mildew (PM), wherein said plant comprises a targeted genome modification conferring reduced expression of at least one Cannabis MLO (CsMLO) allele as compared to a Cannabis plant lacking said targeted genome modification.

It is a further aspect of the present invention to disclose the modified Cannabis plant as defined above, wherein said targeted genome modification is in a CsMLO allele having a wild type genomic nucleotide sequence selected from the group consisting of CsMLO1 having a sequence as set forth in SEQ ID NO:1 or a functional variant thereof, CsMLO2 having a sequence as set forth in SEQ ID NO:4 or a functional variant thereof and CsMLO3 having a sequence as set forth in SEQ ID NO:7 or a functional variant thereof.

It is a further aspect of the present invention to disclose the modified Cannabis plant as defined in any of the above, wherein said functional variant has at least 80% sequence identity to the corresponding CsMLO nucleotide sequence.

It is a further aspect of the present invention to disclose the modified Cannabis plant as defined in any of the above, wherein said genetic modification is introduced using targeted genome modification, preferably said genetic modification is introduced using an endonuclease.

It is a further aspect of the present invention to disclose the modified Cannabis plant as defined in any of the above wherein said targeted genome modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further aspect of the present invention to disclose the modified Cannabis plant as defined in any of the above wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (to (Cas-phi) and any combination thereof.

It is a further aspect of the present invention to disclose the modified Cannabis plant as defined in any of the above, wherein said plant comprises a recombinant DNA construct, said recombinant DNA construct comprising a promoter operably linked to a nucleotide sequence encoding a plant optimized Cas9 endonuclease, wherein said plant optimized Cas9 endonuclease is capable of binding to and creating a double strand break in a genomic target sequence of said plant genome.

It is a further aspect of the present invention to disclose the modified Cannabis plant as defined in any of the above, wherein said DNA construct further comprises sgRNA targeted to at least one CsMLO allele selected from the group consisting of CsMLO1, CsMLO2 and CsMLO3.

It is a further aspect of the present invention to disclose the modified Cannabis plant as defined in any of the above, wherein said sgRNA is targeted to mutate CsMLO1 gene, said sgRNA nucleotide sequence is selected from the group consisting of SEQ ID NO:17, SEQ ID NO:43 and SEQ ID NO:50.

It is a further aspect of the present invention to disclose the modified Cannabis plant as defined in any of the above, wherein said plant comprises at least one mutated CsMLO1 allele comprising a nucleotide sequence selected from the group consisting of a nucleotide sequence as set forth in SEQ ID NO:875, a nucleotide sequence as set forth in SEQ ID NO:877, a nucleotide sequence as set forth in SEQ ID NO:880, a homologue having at least 80% sequence identity to the nucleotide sequence of said at least one mutated CsMLO1 allele and a combination thereof.

It is a further aspect of the present invention to disclose the modified Cannabis plant as defined in any of the above, wherein said mutation is a silencing mutation, a knockdown mutation, a knockout mutation, a loss of function mutation or any combination thereof.

It is a further aspect of the present invention to disclose the modified Cannabis plant as defined in any of the above, wherein said genome modification is an insertion, deletion, indel or substitution.

It is a further aspect of the present invention to disclose the modified Cannabis plant as defined in any of the above, wherein said mutated CsMLO1 allele comprises a deletion having a nucleotide sequence as set forth in SEQ ID NO.:876, SEQ ID NO.:879 or SEQ ID NO.:881.

It is a further aspect of the present invention to disclose the modified Cannabis plant as defined in any of the above, wherein said mutated allele confers an enhanced resistance to powdery mildew as compared to a Cannabis plant comprising a wild type CsMLO1 allele sequence.

It is a further aspect of the present invention to disclose the modified Cannabis plant as defined in any of the above, wherein said wild type CsMLO1 allele comprises a nucleic acid sequence as set forth in at least one of SEQ ID NO:873, SEQ ID NO:876, SEQ ID NO:879 or SEQ ID NO:881.

It is a further aspect of the present invention to disclose the modified Cannabis plant as defined in any of the above wherein said genome modification is an induced mutation in the coding region of said allele, a mutation in the regulatory region of said allele, a mutation in a gene downstream in the MLO pathogen response pathway and/or an epigenetic factor.

It is a further aspect of the present invention to disclose the modified Cannabis plant as defined in any of the above wherein said genome modification is generated in planta.

It is a further aspect of the present invention to disclose the modified Cannabis plant as defined in any of the above wherein said targeted genome modification is generated in planta via introduction of a construct comprising (a) Cas DNA and sgRNA sequence selected from the group consisting of SEQ ID NO:10-SEQ ID NO:870 and any combination thereof, or (b) a ribonucleoprotein (RNP) complex comprising Cas protein and sgRNA sequence selected from the group consisting of SEQ ID NO:10-870 and any combination thereof.

It is a further aspect of the present invention to disclose the modified Cannabis plant as defined in any of the above wherein said targeted genome modification in said CsMLO1 is generated in planta via introduction of a construct comprising (a) Cas DNA and sgRNA sequence selected from the group consisting of SEQ ID NO:10-SEQ ID NO:286 and any combination thereof, or (b) a ribonucleoprotein (RNP) complex comprising Cas protein and sgRNA sequence selected from the group consisting of SEQ ID NO:10-286 and any combination thereof.

It is a further aspect of the present invention to disclose the modified Cannabis plant as defined in any of the above wherein said targeted genome modification in said CsMLO2 is generated in planta via introduction of a construct comprising (a) Cas DNA and sgRNA sequence selected from the group consisting of SEQ ID NO:287-SEQ ID NO:625 and any combination thereof, or (b) a ribonucleoprotein (RNP) complex comprising Cas protein and sgRNA sequence selected from the group consisting of SEQ ID NO:287-625 and any combination thereof.

It is a further aspect of the present invention to disclose the modified Cannabis plant as defined in any of the above, wherein said targeted genome modification in said CsMLO3 is generated in planta via introduction of a construct comprising (a) Cas DNA and sgRNA sequence selected from the group consisting of SEQ ID NO:626-SEQ ID NO:870 and any combination thereof, or (b) a ribonucleoprotein (RNP) complex comprising Cas protein and gRNA sequence selected from the group consisting of SEQ ID NO:626-870 and any combination thereof.

It is a further aspect of the present invention to disclose the modified Cannabis plant as defined in any of the above, wherein said sgRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9) and TBN (Cas-phi),

It is a further aspect of the present invention to disclose the modified Cannabis plant as defined in any of the above, wherein said construct is introduced into the plant cells via Agrobacterium infiltration, virus based plasmids for delivery and/or expression of the genome editing molecules or mechanical insertion such as polyethylene glycol (PEG) mediated DNA transformation, electroporation or gene gun biolistics.

It is a further aspect of the present invention to disclose the modified Cannabis plant as defined in any of the above, wherein said PM is selected from the group consisting of Golovinomyces cichoracearum, Golovinomyces ambrosiae and a mixture thereof.

It is a further aspect of the present invention to disclose the modified Cannabis plant as defined in any of the above, wherein said Cannabis plant is selected from the group of species that includes, but is not limited to, Cannabis sativa (C. sativa), C. indica, C. ruderalis and any hybrid or cultivated variety of the genus Cannabis.

It is a further aspect of the present invention to disclose the modified Cannabis plant as defined in any of the above, wherein said Cannabis plant does not comprise a transgene.

It is a further aspect of the present invention to disclose a modified Cannabis plant, progeny plant, plant part or plant cell as defined in any of the above.

It is a further aspect of the present invention to disclose a plant part, plant cell or plant seed of a modified plant as defined in any of the above.

It is a further aspect of the present invention to disclose a tissue culture of regenerable cells, protoplasts or callus obtained from the modified Cannabis plant as defined in any of the above.

It is a further aspect of the present invention to disclose the modified Cannabis plant as defined in any of the above, wherein said plant genotype is obtainable by deposit under accession number with NCIMB Aberdeen AB21 9YA, Scotland, UK.

It is a further aspect of the present invention to disclose a method for producing a modified Cannabis plant with increased resistance to powdery mildew (PM) comprising introducing using targeted genome modification, at least one genomic modification conferring reduced expression of at least one Cannabis MLO (CsMLO) allele as compared to a Cannabis plant lacking said targeted genome modification.

It is a further aspect of the present invention to disclose the method as defined in any of the above, wherein said method comprises steps of introducing a targeted genome modification to at least one CsMLO allele having a wild type genomic nucleotide sequence selected from the group consisting of CsMLO1 comprising a sequence as set forth in SEQ ID NO:1 or a functional variant thereof, CsMLO2 comprising a sequence as set forth in SEQ ID NO:4 or a functional variant thereof and CsMLO3 comprising a sequence as set forth in SEQ ID NO:7 or a functional variant thereof.

It is a further aspect of the present invention to disclose the method as defined in any of the above, wherein said functional variant has at least 80% sequence identity to the said CsMLO nucleotide sequence.

It is a further aspect of the present invention to disclose the method as defined in any of the above, wherein said method comprises steps of introducing a loss of function mutation into at least one of CsMLO1, CsMLO2 and CsMLO2 nucleic acid sequence.

It is a further aspect of the present invention to disclose the method as defined in any of the above, wherein said method comprises steps of introducing a deletion mutation into the first exon of CsMLO1 genomic sequence to produce a mutated CsMLO1 allele comprising a nucleotide sequence selected from the group consisting of a nucleotide sequence as set forth in SEQ ID NO:875, a nucleotide sequence as set forth in SEQ ID NO:877, a nucleotide sequence as set forth in SEQ ID NO:880, a homologue having at least 80% sequence identity to the nucleotide sequence of said at least one mutated CsMLO1 allele and a combination thereof.

It is a further aspect of the present invention to disclose the method as defined in any of the above, wherein said modified plant has decreased levels of at least one Mlo protein as compared to wild type Cannabis plant.

It is a further aspect of the present invention to disclose the method as defined in any of the above, wherein said modified plant has decreased levels of at least one Mlo protein as compared to a Cannabis plant comprising a wild type CsMLO1 allele sequence comprising a nucleic acid sequence as set forth in at least one of SEQ ID NO:873, SEQ ID NO:876, SEQ ID NO:879 or SEQ ID NO:881.

It is a further aspect of the present invention to disclose the method as defined in any of the above, wherein said genome modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further aspect of the present invention to disclose the method as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is a further aspect of the present invention to disclose the method as defined in any of the above, comprising steps of introducing an expression vector comprising a promoter operably linked to a nucleotide sequence encoding a plant optimized Cas9 endonuclease and sgRNA targeted to at least one CsMLO allele selected from the group consisting of CsMLO1, CsMLO2 and CsMLO3.

It is a further object of the present invention to disclose the method as defined in any of the above, wherein said sgRNA nucleotide sequence targeting CsMLO1 is selected from the group consisting of SEQ ID NO:17, SEQ ID NO:43 and SEQ ID NO:50.

It is a further aspect of the present invention to disclose the method as defined in any of the above, comprising steps of introducing and co-expressing in a Cannabis plant Cas9 and sgRNA targeted to at least one of CsMLO1, CsMLO2 and CsMLO3 genes and screening for induced targeted mutations in at least one of CsMLO1, CsMLO2 and CsMLO3 genes.

It is a further aspect of the present invention to disclose the method as defined in any of the above, comprising steps of screening for induced targeted mutations in at least one of CsMLO1, CsMLO2 and CsMLO3 genes comprising obtaining a nucleic acid sample from a transformed plant and carrying out nucleic acid amplification and optionally restriction enzyme digestion to detect a mutation in at least one of CsMLO1, CsMLO2 and CsMLO3.

It is a further aspect of the present invention to disclose the method as defined in any of the above, wherein said nucleic acid amplification for screening induced targeted mutations in CsMLO1 genomic sequence uses primers having nucleic acid sequence as set forth in SEQ ID NO: 871 and SEQ ID NO: 872.

It is a further aspect of the present invention to disclose the method as defined in any of the above, further comprising steps of assessing PCR fragments or amplicons amplified from the transformed plants using a gel electrophoresis based assay.

It is a further aspect of the present invention to disclose the method as defined in any of the above, further comprising steps of confirming the presence of a mutation by sequencing the at least one of CsMLO1, CsMLO2 and CsMLO3 nucleic acid fragment or amlicon.

It is a further aspect of the present invention to disclose the method as defined in any of the above, wherein said mutation is in the coding region of said allele, a mutation in the regulatory region of said allele, a mutation in a gene downstream in the MLO pathogen response pathway or an epigenetic factor.

It is a further aspect of the present invention to disclose the method as defined in any of the above, wherein said mutation is selected from the group consisting of a silencing mutation, a knockdown mutation, a knockout mutation, a loss of function mutation and any combination thereof.

It is a further aspect of the present invention to disclose the method as defined in any of the above, wherein said mutation is an insertion, deletion, indel or substitution mutation.

It is a further aspect of the present invention to disclose the method as defined in any of the above, wherein said mutation is a deletion in the first exon of CsMLO1, said deletion comprises nucleic acid sequence selected from the group consisting of SEQ ID NO.:876, SEQ ID NO.:879 or SEQ ID NO.:881.

It is a further aspect of the present invention to disclose the method as defined in any of the above, further comprising steps of selecting a plant resistant to powdery mildew from transformed plants comprising mutated at least one of CsMLO1, CsMLO2 and CsMLO3 nucleic acid fragment.

It is a further aspect of the present invention to disclose the method as defined in any of the above, wherein said selected plant is characterized by enhanced resistance to powdery mildew as compared to a Cannabis plant comprising a CsMLO1 nucleic acid comprising a nucleic acid sequence as set forth in SEQ ID NO:873.

It is a further aspect of the present invention to disclose the method as defined in any of the above, wherein said genetic modification in said CsMLO1 is generated in planta via introduction of a construct comprising (a) Cas DNA and gRNA sequence selected from the group consisting of SEQ ID NO:10-SEQ ID NO:286 and any combination thereof, or (b) a ribonucleoprotein (RNP) complex comprising Cas protein and gRNA sequence selected from the group consisting of SEQ ID NO:10-286 and any combination thereof.

It is a further aspect of the present invention to disclose the method as defined in any of the above, wherein said genetic modification in said CsMLO2 is generated in planta via introduction of a construct comprising (a) Cas DNA and gRNA sequence selected from the group consisting of SEQ ID NO:287-SEQ ID NO:625 and any combination thereof, or (b) a ribonucleoprotein (RNP) complex comprising Cas protein and gRNA sequence selected from the group consisting of SEQ ID NO:287-625 and any combination thereof.

It is a further aspect of the present invention to disclose the method as defined in any of the above, wherein said genetic modification in said CsMLO3 is generated in planta via introduction of a construct comprising (a) Cas DNA and gRNA sequence selected from the group consisting of SEQ ID NO:626-SEQ ID NO:870 and any combination thereof, or (b) a ribonucleoprotein (RNP) complex comprising Cas protein and gRNA sequence selected from the group consisting of SEQ ID NO:626-870 and any combination thereof.

It is a further aspect of the present invention to disclose the method as defined in any of the above, wherein said gRNA nucleotide sequence comprises a 3′ Protospacer Adjacent Motif (PAM), said PAM is selected from the group consisting of: NGG (SpCas), NNNNGATT (NmeCas9), NNAGAAW (StCas9), NAAAAC (TdCas9), NNGRRT (SaCas9) and TBN (Cas-phi).

It is a further aspect of the present invention to disclose the method as defined in any of the above, wherein said construct is introduced into the plant cells using Agrobacterium infiltration, virus based plasmids for delivery of the genome editing molecules by or mechanical insertion such as polyethylene glycol (PEG) mediated DNA transformation, electroporation or gene gun biolistics.

It is a further aspect of the present invention to disclose the method as defined in any of the above, further comprising steps of regenerating a plant carrying said genomic modification.

It is a further aspect of the present invention to disclose the method as defined in any of the above, further comprising steps of screening said regenerated plants for a plant resistant to powdery mildew.

It is a further aspect of the present invention to disclose a method for conferring resistance to powdery mildew to a Cannabis plant comprising producing a plant as defined in any of the above.

It is a further aspect of the present invention to disclose a plant, plant part, plant cell, tissue culture or a seed obtained or obtainable by the method as defined in any of the above.

It is a further aspect of the present invention to disclose the method as defined in any of the above, wherein said PM is selected from the group consisting of Golovinomyces cichoracearum, Golovinomyces ambrosiae and a mixture thereof.

It is a further aspect of the present invention to disclose the method as defined in any of the above, wherein said Cannabis plant is selected from the group of species that includes, but is not limited to, Cannabis sativa (C. sativa), C. indica, C. ruderalis and any hybrid or cultivated variety of the genus Cannabis.

It is a further aspect of the present invention to disclose a method for producing a modified Cannabis plant with increased resistance to powdery mildew compared to a Cannabis wild type plant using targeted genome modification comprising introducing at least one genetic modification conferring reduced expression of at least one Cannabis MLO (CsMLO) allele, said method comprises steps of: (a) identifying at least one Cannabis MLO (CsMLO) orthologous allele; (b) sequencing genomic DNA of said at least one identified CsMLO; (c) synthetizing at least one guide RNA (gRNA) comprising a nucleotide sequence complementary to said at least one identified CsMLO; (d) transforming Cannabis plant cells with a construct comprising (a) Cas nucleotide sequence and said gRNA, or (b) a ribonucleoprotein (RNP) complex comprising Cas protein and said gRNA; (e) screening the genome of said transformed plant cells for induced targeted mutations in at least one of said CsMLO alleles comprising obtaining a nucleic acid sample from said transformed plant and carrying out nucleic acid amplification and optionally restriction enzyme digestion to detect a mutation in said at least one of said CsMLO allele; (f) confirming the presence of said genetic mutation in the genome of said plant cells by sequencing said at least one CsMLO allele; (g) regenerating plants carrying said genetic modification; and (h) screening said regenerated plants for a plant resistant to powdery mildew.

It is a further aspect of the present invention to disclose the method as defined in any of the above, wherein said method comprises steps of introducing a targeted genome modification to at least one CsMLO allele having a wild type genomic nucleotide sequence selected from the group consisting of CsMLO1 comprising a sequence as set forth in SEQ ID NO:1 or a functional variant thereof, CsMLO2 comprising a sequence as set forth in SEQ ID NO:4 or a functional variant thereof and CsMLO3 comprising a sequence as set forth in SEQ ID NO:7 or a functional variant thereof.

It is a further aspect of the present invention to disclose the method as defined in any of the above, wherein said functional variant has at least 80% sequence identity to the said CsMLO nucleotide sequence.

It is a further aspect of the present invention to disclose the method as defined in any of the above, wherein said plant has decreased levels of at least one Mlo protein.

It is a further aspect of the present invention to disclose the method as defined in any of the above, further comprising steps of introducing into said plant sgRNA targeted to mutate CsMLO1 gene, said sgRNA nucleotide sequence is selected from the group consisting of SEQ ID NO:17, SEQ ID NO:43 and SEQ ID NO:50.

It is a further aspect of the present invention to disclose the method as defined in any of the above, wherein said nucleic acid amplification for screening induced targeted mutations in CsMLO1 genomic sequence uses primers having nucleic acid sequence as set forth in SEQ ID NO: 871 and SEQ ID NO: 872.

It is a further aspect of the present invention to disclose the method as defined in any of the above, wherein said plant comprises at least one mutated CsMLO1 allele comprising a nucleotide sequence selected from the group consisting of a nucleotide sequence as set forth in SEQ ID NO:875, a nucleotide sequence as set forth in SEQ ID NO:877, a nucleotide sequence as set forth in SEQ ID NO:880, a homologue having at least 80% sequence identity to the nucleotide sequence of said at least one mutated CsMLO1 allele and a combination thereof.

It is a further aspect of the present invention to disclose the method as defined in any of the above, wherein said mutation is a silencing mutation, a knockdown mutation, a knockout mutation, a loss of function mutation or any combination thereof.

It is a further aspect of the present invention to disclose the method as defined in any of the above, wherein said mutated CsMLO1 allele comprises a deletion having a nucleotide sequence as set forth in SEQ ID NO.:876, SEQ ID NO.:879 or SEQ ID NO.:881.

It is a further aspect of the present invention to disclose the method as defined in any of the above, wherein said mutated allele confers an enhanced resistance to powdery mildew as compared to a Cannabis plant comprising a wild type CsMLO1 allele sequence.

It is a further aspect of the present invention to disclose the method as defined in any of the above, wherein said wild type CsMLO1 allele comprises a nucleic acid sequence as set forth in at least one of SEQ ID NO:873, SEQ ID NO:876, SEQ ID NO:879 or SEQ ID NO:881.

It is a further aspect of the present invention to disclose a method of determining the presence of a mutant CsMLO1 nucleic acid in a Cannabis plant comprising assaying said Cannabis plant with primers having nucleic acid sequence as set forth in SEQ ID NO: 871 and SEQ ID NO: 872.

It is a further aspect of the present invention to disclose a method for determining the presence or absence of a mutant CsMLO1 nucleic acid or polypeptide in a Cannabis plant comprising detecting the presence or absence of a deletion of a nucleotide sequence as set forth in SEQ ID NO.:876, SEQ ID NO.:879 or SEQ ID NO.:881.

It is a further aspect of the present invention to disclose a method for identifying a Cannabis plant with resistance to powdery mildew, said method comprises steps of: (a) screening the genome of said Cannabis plant for induced targeted mutations in at least one of CsMLO1, CsMLO2 and/or CsMLO3 alleles having a wild type genomic nucleotide sequence selected from the group consisting of CsMLO1 comprising a sequence as set forth in SEQ ID NO:1 or a functional variant thereof, CsMLO2 comprising a sequence as set forth in SEQ ID NO:4 or a functional variant thereof and CsMLO3 comprising a sequence as set forth in SEQ ID NO:7 or a functional variant thereof; (b) confirming the presence of said genetic mutation in the genome of said plant cells by sequencing said at least one CsMLO allele; (c) regenerating plants carrying said genetic modification; and (d) screening said regenerated plants for a plant resistant to powdery mildew.

It is a further aspect of the present invention to disclose the method as defined in any of the above, wherein said screening for the presence of mutated CsMLO1 allele is carried out using a primer pair having nucleic acid sequence as set forth in SEQ ID NO: 871 and SEQ ID NO: 872.

It is a further aspect of the present invention to disclose the method as defined in any of the above, wherein said method comprises steps of screening for the presence of mutated CsMLO1 allele comprising a nucleic acid sequence selected from the group consisting of a nucleotide sequence as set forth in SEQ ID NO:875, a nucleotide sequence as set forth in SEQ ID NO:877, a nucleotide sequence as set forth in SEQ ID NO:880, a homologue having at least 80% sequence identity to the nucleotide sequence of said at least one mutated CsMLO1 allele and a combination thereof.

It is a further aspect of the present invention to disclose the method as defined in any of the above, wherein said method comprises steps of screening said Cannabis plant for the presence of a deletion in CsMLO1 comprising a nucleotide sequence selected from the group consisting of SEQ ID NO.:876, SEQ ID NO.:879 and SEQ ID NO.:881.

It is a further aspect of the present invention to disclose the method as defined in any of the above, wherein the presence of at least one nucleic acid sequence selected from the group consisting of SEQ ID NO:873, SEQ ID NO:876, SEQ ID NO:879 and SEQ ID NO:881 indicates that the Cannabis plant comprises wild type CsMLO1 nucleic acid, and the presence of at least one nucleic acid sequence selected from the group consisting of SEQ ID NO:875, SEQ ID NO:877 and SEQ ID NO:880, optionally in combination with the absence of at least one nucleic acid sequence selected from the group consisting of SEQ ID NO:876, SEQ ID NO:879 and SEQ ID NO:881 indicates that the Cannabis plant comprises a mutant CsMLO1 nucleic acid.

It is a further aspect of the present invention to disclose the method as defined in any of the above, wherein said Cannabis plant comprising a mutant CsMLO1 nucleic acid is characterized by enhanced resistance to powdery mildew as compared to a Cannabis plant comprising said wild type CsMLO1 nucleic acid.

It is a further aspect of the present invention to disclose an isolated nucleotide sequence of a primer or primer pair having at least 75% sequence identity to a nucleic acid sequence selected from the group consisting of SEQ ID NO:1, 2, 4, 5, 7, 8 and SEQ ID NO:10-873, 875, 876, 877, 879, 880 and 881.

It is a further aspect of the present invention to disclose an isolated amino acid sequence having at least 75% sequence similarity to an amino acid sequence selected from the group consisting of SEQ ID NO:3, SEQ ID NO:6, SEQ ID NO:9, SEQ ID NO:874, SEQ ID NO:878 and SEQ ID NO:887.

It is a further aspect of the present invention to disclose use of a nucleotide sequence as set forth in at least one of SEQ ID NO:871 and SEQ ID NO:872 as a primer or primer pair for identifying or screening for a Cannabis plant comprising within its genome mutant CsMLO1 nucleic acid and/or polypeptide.

It is a further aspect of the present invention to disclose use of a nucleotide sequence as set forth in at least one of SEQ ID NO:871 and SEQ ID NO:872 as a primer or primer pair for identifying or screening for a Cannabis plant resistance to powdery mildew.

It is a further aspect of the present invention to disclose use of a nucleotide sequence as set forth in SEQ ID NO:873, SEQ ID NO:875, SEQ ID NO:876, SEQ ID NO:877, SEQ ID NO:879, SEQ ID NO:880 and SEQ ID NO:881 for identifying and/or screening for a Cannabis plant with comprising within its genome mutant CsMLO1 nucleic acid and/or polypeptide, wherein, the presence of at least one nucleic acid sequence selected from the group consisting of SEQ ID NO:873, SEQ ID NO:876, SEQ ID NO:879 and SEQ ID NO:881 indicates that the Cannabis plant comprises wild type CsMLO1 nucleic acid, and the presence of at least one nucleic acid sequence selected from the group consisting of SEQ ID NO:875, SEQ ID NO:877 and SEQ ID NO:880, optionally in combination with the absence of at least one nucleic acid sequence selected from the group consisting of SEQ ID NO:876, SEQ ID NO:879 and SEQ ID NO:881 indicates that the Cannabis plant comprises a mutant CsMLO1 nucleic acid.

It is a further aspect of the present invention to disclose the use as defined in any of the above, wherein said Cannabis plant comprising a mutant CsMLO1 nucleic acid is characterized by enhanced resistance to powdery mildew as compared to a Cannabis plant comprising said wild type CsMLO1 nucleic acid.

It is a further aspect of the present invention to disclose use of a nucleotide sequence as set forth in at least one of SEQ ID NO:10-870 and any combination thereof for targeted genome modification of at least one Cannabis MLO (CsMLO) allele.

It is a further aspect of the present invention to disclose use of a nucleotide sequence as set forth in at least one of SEQ ID NO:10-286 and any combination thereof for targeted genome modification of Cannabis CsMLO1 allele.

It is a further aspect of the present invention to disclose use of a nucleotide sequence as set forth in at least one of SEQ ID NO:17, SEQ ID NO:43 and SEQ ID NO:50 and any combination thereof for targeted genome modification of Cannabis CsMLO1 allele.

It is a further aspect of the present invention to disclose use of a nucleotide sequence as set forth in at least one of SEQ ID NO:287-625 and any combination thereof for targeted genome modification of Cannabis CsMLO2 allele.

It is a further aspect of the present invention to disclose use of a nucleotide sequence as set forth in at least one of SEQ ID NO:626-870 and any combination thereof for targeted genome modification of Cannabis CsMLO3.

It is a further aspect of the present invention to disclose a detection kit for determining the presence or absence of a mutant CsMLO1 nucleic acid nucleic acid or polypeptide in a Cannabis plant comprising a primer selected from SEQ ID NO:871 and SEQ ID NO:872.

It is a further aspect of the present invention to disclose the detection kit as defined in any of the above, wherein said kit further comprising primers or nucleic acid fragments for detection of a nucleic acid sequence selected from the group consisting of SEQ ID NO:873, SEQ ID NO:875, SEQ ID NO:876, SEQ ID NO:877, SEQ ID NO:879, SEQ ID NO:880 and SEQ ID NO:881.

It is a further aspect of the present invention to disclose the detection kit as defined in any of the above, wherein said kit is useful for identifying a Cannabis plant resistant to powdery mildew.

It is a further aspect of the present invention to disclose a modified Cannabis plant exhibiting enhanced resistance to powdery mildew (PM), wherein the modified plant comprises a mutated Cannabis mlo1 (Csmlo1) allele, the mutated allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof.

It is another object of the present invention to disclose the modified Cannabis plant as defined above, wherein the indel comprises a sequence as set forth in SEQ ID NO:883 or a fraction thereof.

It is another aspect of the present invention to disclose the modified Cannabis plant as defined in any of the above, wherein the Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of the mutated Csmlo1 allele, or a complementary sequence thereof, or any combination thereof.

It is another aspect of the present invention to disclose the modified Cannabis plant as defined in any of the above, wherein the Csmlo1 mutant allele comprises a nucleic acid sequence corresponding to the sequence as set forth in SEQ ID NO:886, or a homologue having at least 80% sequence identity to the nucleic acid sequence of the mutated Csmlo1 allele.

It is another aspect of the present invention to disclose the modified Cannabis plant as defined in any of the above, wherein the mutated Csmlo1 allele confers an enhanced resistance to powdery mildew as compared to a Cannabis plant comprising a wild type CsMLO1 allele having a nucleic acid sequence as set forth in SEQ ID NO:882 and/or having a nucleic acid sequence as set forth in SEQ ID NO:1 or a functional variant thereof.

It is another aspect of the present invention to disclose the modified Cannabis plant as defined in any of the above, wherein the functional variant has at least 80% sequence identity to the corresponding CsMLO1 nucleotide sequence.

It is another aspect of the present invention to disclose the modified Cannabis plant as defined in any of the above, wherein the mutated allele comprising a deletion of 14 bp at position 389 of SEQ ID NO: 1, or a nucleic acid insertion at position 482-483 of SEQ ID NO: 1, or a combination thereof.

It is another aspect of the present invention to disclose the modified Cannabis plant as defined in any of the above, wherein the plant has decreased expression levels of Mlo1 protein, relative to a Cannabis plant lacking the mutated Csmlo1 allele.

It is another aspect of the present invention to disclose the modified Cannabis plant as defined in any of the above, wherein the mutated Csmlo1 allele is generated using mutagenesis, small interfering RNA (siRNA), microRNA (miRNA), artificial miRNA (amiRNA), DNA introgression, endonucleases or any combination thereof.

It is another aspect of the present invention to disclose the modified Cannabis plant as defined in any of the above, wherein the plant comprises a DNA construct, the DNA construct comprising a promoter operably linked to a nucleotide sequence encoding a plant optimized Cas9 endonuclease, wherein the plant optimized Cas9 endonuclease is capable of binding to and creating a double strand break in a genomic target sequence of the plant genome.

It is another aspect of the present invention to disclose the modified Cannabis plant as defined in any of the above, wherein the DNA construct further comprises gRNA targeted to at least one CsMLO1 allele.

It is another aspect of the present invention to disclose the modified Cannabis plant as defined in any of the above, wherein the gRNA has a nucleic acid sequence corresponding to a sequence selected from the group consisting of SEQ ID NO:17, SEQ ID NO:43 and SEQ ID NO:50 or a complementary sequence thereof, or any combination thereof.

It is another aspect of the present invention to disclose the modified Cannabis plant as defined in any of the above, wherein the genome modification is a silencing mutation, a knockdown mutation, a knockout mutation, a loss of function mutation or any combination thereof.

It is another aspect of the present invention to disclose the modified Cannabis plant as defined in any of the above, wherein the genome modification is an insertion, deletion, indel or substitution.

It is another aspect of the present invention to disclose the modified Cannabis plant as defined in any of the above, wherein the genome modification is an induced mutation in the coding region of the allele, a mutation in the regulatory region of the allele, a mutation in a gene downstream in the MLO pathogen response pathway and/or an epigenetic factor.

It is another aspect of the present invention to disclose the modified Cannabis plant as defined in any of the above, wherein the genome modification is generated via introduction (a) Cas DNA and gRNA sequence selected from the group consisting of SEQ ID NO:17, SEQ ID NO:43 and SEQ ID NO:50 and any combination thereof, or (b) a ribonucleoprotein (RNP) complex comprising Cas protein and gRNA sequence selected from the group consisting of SEQ ID NO:17, SEQ ID NO:43 and SEQ ID NO:50 and any combination thereof.

It is another aspect of the present invention to disclose the modified Cannabis plant as defined in any of the above, wherein the Cannabis plant does not comprise a transgene.

It is another aspect of the present invention to disclose a modified Cannabis plant, progeny plant, plant part or plant cell as defined in any of the above.

It is another aspect of the present invention to disclose a plant part, plant cell or plant seed of a modified plant as defined in any of the above.

It is another aspect of the present invention to disclose a tissue culture of regenerable cells, protoplasts or callus obtained from the modified Cannabis plant as defined in any of the above.

It is another aspect of the present invention to disclose the modified Cannabis plant as defined in any of the above, wherein the plant genotype is obtainable by deposit under accession number with NCIMB Aberdeen AB21 9YA, Scotland, UK.

It is another aspect of the present invention to disclose a modified Cannabis plant exhibiting enhanced resistance to powdery mildew (PM), wherein the modified plant comprises a targeted genome modification conferring reduced expression of a Cannabis MLO1 (CsMLO1) gene as compared to a Cannabis plant lacking the targeted genome modification, the targeted genome modification generates a mutated Cannabis mlo1 (Csmlo1) allele comprising a deletion of a nucleic acid sequence as set forth in SEQ ID NO:883 or a fraction thereof as compared to the wild type CsMLO1 allele comprising a sequence as set forth in SEQ ID NO:1, or a nucleic acid insertion at position 482-483 of SEQ ID NO:1, or a combination thereof.

It is another aspect of the present invention to disclose the modified Cannabis plant as defined in any of the above, wherein the mutated allele comprising a deletion of 14 bp at position 389 of SEQ ID NO: 1, or a nucleic acid insertion at position 482-483 of SEQ ID NO: 1, or a combination thereof.

It is another aspect of the present invention to disclose a method for producing a modified Cannabis plant as defined in any of the above, the method comprises introducing using targeted genome modification, at least one genomic modification conferring reduced expression of at least one Cannabis MLO1 (CsMLO1) allele as compared to a Cannabis plant lacking the targeted genome modification, the genomic modification generates a mutated Cannabis mlo1 (Csmlo1) allele comprising an indel of 14 bp at a position corresponding to position 12 of SEQ ID NO: 882 or a fraction thereof, or a nucleic acid insertion at position corresponding to position 104-105 of SEQ ID NO: 882, or a combination thereof.

It is another aspect of the present invention to disclose the method as defined above, comprises steps of introducing a loss of function mutation into the CsMLO1 allele using targeted genome modification.

It is another aspect of the present invention to disclose the method as defined in any of the above, wherein the indel comprises a sequence as set forth in SEQ ID NO:883 or a fraction thereof.

It is another aspect of the present invention to disclose the method as defined in any of the above, wherein the Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of the mutated Csmlo1 allele, or a complementary sequence thereof, or any combination thereof.

It is another aspect of the present invention to disclose the method as defined in any of the above, wherein the Csmlo1 mutant allele comprises a nucleic acid sequence corresponding to the sequence as set forth in SEQ ID NO:886, or a homologue having at least 80% sequence identity to the nucleic acid sequence of the mutated Csmlo1 allele.

It is another aspect of the present invention to disclose the method as defined in any of the above, wherein the mutated Csmlo1 allele confers an enhanced resistance to powdery mildew as compared to a Cannabis plant comprising a wild type CsMLO1 allele having a nucleic acid sequence as set forth in SEQ ID NO:882 and/or having a nucleic acid sequence as set forth in SEQ ID NO:1 or a functional variant thereof.

It is another aspect of the present invention to disclose the method as defined in any of the above, wherein the functional variant has at least 80% sequence identity to the corresponding CsMLO1 nucleotide sequence.

It is another aspect of the present invention to disclose the method as defined in any of the above, wherein the mutated allele comprising a deletion of 14 bp at position 389 of SEQ ID NO: 1, or a nucleic acid insertion at position 482-483 of SEQ ID NO: 1, or a combination thereof.

It is another aspect of the present invention to disclose the method as defined in any of the above, wherein the modified plant has decreased levels of at least one Mlo protein as compared to wild type Cannabis plant.

It is another aspect of the present invention to disclose the method as defined in any of the above, comprising steps of introducing an expression vector comprising a promoter operably linked to a nucleotide sequence encoding a plant optimized Cas9 endonuclease and gRNA targeted to at least one CsMLO1 allele.

It is another aspect of the present invention to disclose the method as defined in any of the above, wherein the gRNA nucleotide sequence targeting the CsMLO1 allele is selected from the group consisting of SEQ ID NO:17, SEQ ID NO:43 and SEQ ID NO:50 or a complementary sequence thereof.

It is another aspect of the present invention to disclose the method as defined in any of the above, comprising steps of introducing and co-expressing in a Cannabis plant Cas9 and gRNA targeted to CsMLO1 gene and screening for induced targeted mutations conferring reduced expression of the CsMLO1 gene.

It is another aspect of the present invention to disclose the method as defined in any of the above, wherein the genomic modification is in the coding region of the allele, a mutation in the regulatory region of the allele, a mutation in a gene downstream in the MLO pathogen response pathway or an epigenetic factor.

It is another aspect of the present invention to disclose the method as defined in any of the above, wherein the genomic modification is selected from the group consisting of a silencing mutation, a knockdown mutation, a knockout mutation, a loss of function mutation and any combination thereof.

It is another aspect of the present invention to disclose the method as defined in any of the above, wherein the genomic modification is an insertion, deletion, indel or substitution mutation.

It is another aspect of the present invention to disclose the method as defined in any of the above, further comprising steps of selecting a plant resistant to powdery mildew from plants comprising mutated Csmlo1 allele.

It is another aspect of the present invention to disclose the method as defined in any of the above, wherein the selected plant is characterized by enhanced resistance to powdery mildew as compared to a Cannabis plant comprising a CsMLO1 nucleic acid comprising a nucleic acid sequence as set forth in SEQ ID NO:882.

It is another aspect of the present invention to disclose the method as defined in any of the above, wherein the genetic modification in the CsMLO1 is generated in planta via introduction of a construct comprising (a) Cas DNA and gRNA sequence selected from the group consisting of SEQ ID NO:17, SEQ ID NO:43 and SEQ ID NO:50 or a complementary sequence thereof, and any combination thereof, or (b) a ribonucleoprotein (RNP) complex comprising Cas protein and gRNA sequence selected from the group consisting of SEQ ID NO:17, SEQ ID NO:43 and SEQ ID NO:50 or a complementary sequence thereof, and any combination thereof.

It is another aspect of the present invention to disclose the method as defined in any of the above, further comprising steps of regenerating a plant carrying the genomic modification.

It is another aspect of the present invention to disclose the method as defined in any of the above, further comprising steps of screening the regenerated plants for a plant resistant to powdery mildew.

It is another aspect of the present invention to disclose the method as defined a method for conferring powdery mildew resistance to a Cannabis plant comprising producing a plant according to the method as defined in any of the above.

It is another aspect of the present invention to disclose a plant, plant part, plant cell, tissue culture or a seed obtained or obtainable by the method as defined in any of the above.

It is another aspect of the present invention to disclose a method of determining the presence of a mutant Csmlo1 allele in a Cannabis plant comprising assaying the Cannabis plant for at least one of the presence of an indel comprising a nucleic acid sequence as set forth in SEQ ID NO:883, an insertion at position 104-105 of SEQ ID NO: 882, a nucleic acid sequence corresponding to the sequence as set forth in SEQ ID NO:884, a nucleic acid sequence corresponding to the sequence as set forth in SEQ ID NO:885, a nucleic acid sequence corresponding to the sequence as set forth in SEQ ID NO:886, or a homologue having at least 80% sequence identity to the nucleic acid sequence of the mutated Csmlo1 allele, a complementary sequence thereof, or any combination thereof.

It is another aspect of the present invention to disclose a method for identifying a Cannabis plant with resistance to powdery mildew, the method comprises steps of: (a) screening the genome of the Cannabis plant for a mutated Csmlo1 allele, the mutated allele comprises a genomic modification selected from an indel of 14 bp at a position corresponding to position 12 of SEQ ID NO: 882 or a fraction thereof, or a nucleic acid insertion at position corresponding to position 104-105 of SEQ ID NO: 882, or a combination thereof; (b) optionally, regenerating plants carrying the genetic modification; and (c) optionally, screening the regenerated plants for a plant resistant to powdery mildew.

It is another aspect of the present invention to disclose the method as defined in any of the above, wherein the genomic modification is a loss of function mutation.

It is another aspect of the present invention to disclose the method as defined in any of the above, wherein the indel comprises a sequence as set forth in SEQ ID NO:883 or a fraction and/or a complementary sequence thereof.

It is another aspect of the present invention to disclose the method as defined in any of the above, wherein the Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of the mutated Csmlo1 allele, or a complementary sequence thereof, or any combination thereof.

It is another aspect of the present invention to disclose the method as defined in any of the above, wherein the method comprises steps of screening the Cannabis plant for the presence of a deletion in CsMLO1 gene comprising a nucleic acid sequence as set forth in SEQ ID NO:1, the deletion comprising a nucleotide sequence as set forth in SEQ ID NO:883.

It is another aspect of the present invention to disclose the method as defined in any of the above, wherein the modified Cannabis plant comprising a mutant Csmlo1 nucleic acid conferring enhanced resistance to powdery mildew as compared to a Cannabis plant comprising a wild type CsMLO1 nucleic acid.

It is another aspect of the present invention to disclose a method for down regulation of Cannabis MLO1 (CsMLO1) gene, which comprises utilizing the nucleotide sequence as set forth in at least one of SEQ ID NO:43 and SEQ ID NO:50 or a complementary sequence thereof, and a combination thereof, for introducing a loss of function mutation into the CsMLO1 gene using targeted genome modification.

It is another aspect of the present invention to disclose an isolated amino acid sequence having at least 80% sequence identity to a nucleic acid sequence selected from the group consisting of SEQ ID NO:882-886, SEQ ID NO:17, SEQ ID NO:43 and SEQ ID NO:50 or a complementary sequence or any combination thereof.

It is another aspect of the present invention to disclose use of a nucleotide sequence as set forth in SEQ ID NO: 883-886, SEQ ID NO:17, SEQ ID NO:43 and SEQ ID NO:50 for generating, identifying and/or screening for a Cannabis plant comprising within its genome mutant Csmlo allele conferring resistance to PM.

It is another aspect of the present invention to disclose the use as defined in any of the above, wherein the presence of at least one nucleic acid sequence selected from the group consisting of SEQ ID NO:1, SEQ ID NO:883, SEQ ID NO:882 indicates that the Cannabis plant comprises a wild type CsMLO1 allele, and the presence of at least one nucleic acid sequence selected from the group consisting of SEQ ID NO:884, SEQ ID NO:885 and SEQ ID NO:886 indicates that the Cannabis plant comprises a mutant Csmol1 allele.

It is another aspect of the present invention to disclose use of a nucleotide sequence as set forth in at least one of SEQ ID NO:17, SEQ ID NO:43 and SEQ ID NO:50 or a complementary sequence or any combination thereof for targeted genome modification of Cannabis MLO1 (CsMLO1) gene.

It is another aspect of the present invention to disclose a detection kit for determining the presence or absence of a mutant Csmlo1 allele in a Cannabis plant, comprising a nucleic acid fragment comprising a sequence selected from SEQ ID NO:882-886, SEQ ID NO:17, SEQ ID NO:43 and SEQ ID NO:50 or a complementary sequence or any combination thereof.

It is another aspect of the present invention to disclose the detection kit as defined above, wherein the kit is useful for identifying a Cannabis plant with enhanced resistance to powdery mildew.

In order to understand the invention and to see how it may be implemented in practice, a plurality of preferred embodiments will now be described, by way of non-limiting example only, with reference to the following examples.

### EXAMPLE 1

### Exemplified Method for Production of Powdery Mildew Resistant Cannabis Plants by Genome Editing

Production of powdery mildew resistant Cannabis lines may be achieved by at least one of the following breeding/cultivation schemes:

Scheme 1:
line stabilization by self pollination
Generation of F6 parental lines
Genome editing of parental lines
Crossing edited parental lines to generate an F1 hybrid PM resistant plant

Scheme 2:
Identifying genes of interest
Designing gRNA
Transformation of plants with Cas9+gRNA constructs
Screening and identifying editing events
Genome editing of parental lines

It is noted that line stabilization may be performed by the following:
Induction of male flowering on female (XX) plants
Self pollination

According to some embodiments of the present invention, line stabilization requires 6 self-crossing (6 generations) and done through a single seed descent (SSD) approach.

F1 hybrid seed production: Novel hybrids are produced by crosses between different Cannabis strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program
Genotyping markers—germplasm used in the current invention is genotyped using molecular markers, in order to allow a more efficient breeding process and identification of the MLO editing event.

It is further within the scope of the current invention that allele and genetic variation is analysed for the Cannabis strains used.

Reference is now made to optional stages that have been used for the production of powdery mildew resistant Cannabis plants by genome editing:

Stage 1: Identifying Cannabis sativa (C. sativa) MLO orthologues, Three MLO orthologues have herein been identified in C. sativa, namely CsMLO1, CsMLO2 and CsMLO3. These homologous genes have been sequenced and mapped. CsMLO1 has been found to be located on chromosome 5 between position 58544241 bp and position 58551241 bp and has a genomic sequence as set forth in SEQ ID NO:1. The CsMLO1 gene has a coding sequence as set forth in SEQ ID NO:2 and it encodes an amino acid sequence as set forth in SEQ ID NO:3.

CsMLO2 has been found to be located on chromosome 3 between position 92616000 bp and position 92629000 bp and has a genomic sequence as set forth in SEQ ID NO:4. The CsMLO2 gene has a coding sequence as set forth in SEQ ID NO:5 and it encodes an amino acid sequence as set forth in SEQ ID NO:6.

CsMLO3 has been found to be located on Chromosome 5 between position 23410000 bp and position 23420000 bp and has a genomic sequence as set forth in SEQ ID NO:7. The CsMLO3 gene has a coding sequence as set forth in SEQ ID NO:8 and it encodes an amino acid sequence as set forth in SEQ ID NO:9.

Stage 2: Designing and synthesizing gRNA molecules corresponding to the sequence targeted for editing, i.e. sequences of each of the genes CsMLO1, CsMLO2 and CsMLO3. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different MLO homologues of different Cannabis strains.

Reference is now made to Tables 1, 2 and 3 presenting gRNA molecules constructed for silencing CsMLO1, CsMLO2 and CsMLO3, respectively. In Tables 1, 2 and 3 the term ‘PAM’ refers to protospacer adjacent motif, which is a 2-6 base pair DNA sequence immediately following the DNA sequence targeted by the Cas9 nuclease in the CRISPR bacterial adaptive immune system. The CsMLO genomic DNA sense strand is marked as “1”, and the antisense strand is marked as “−1”.

TABLE 1
CsMLO1 targeted gRNA sequences

| Position | | | | |
|---|---|---|---|---|
| on | | | | SEQ |
| SEQ ID | | | | ID |
| NO: 1 | Strand | Sequence | PAM | NO |
| 30 | 1 | GTGAGTGAATGAGAGCAAGA | AGG | 10 |
| 59 | -1 | ATTCCGATTTCGAATTCAGA | TGG | 11 |
| 67 | 1 | AATCCATCTGAATTCGAAAT | CGG | 12 |
| 76 | 1 | GAATTCGAAATCGGAATGAG | TGG | 13 |
| 79 | 1 | TTCGAAATCGGAATGAGTGG | CGG | 14 |
| 82 | 1 | GAAATCGGAATGAGTGGCGG | TGG | 15 |
| 88 | 1 | GGAATGAGTGGCGGTGGAGA | AGG | 16 |
| 99 | 1 | CGGTGGAGAAGGTGAGTCCT | TGG | 17 |
| 105 | -1 | CCATGTGGGAGTATACTCCA | AGG | 18 |
| 116 | 1 | CCTTGGAGTATACTCCCACA | TGG | 19 |
| 119 | -1 | ACGACGGCGACGATCCATGT | GGG | 20 |
| 120 | -1 | GACGACGGCGACGATCCATG | TGG | 21 |
| 135 | -1 | GACGATGACAGAGCAGACGA | CGG | 22 |
| 159 | -1 | ACGCTCCGCGGCGAGAGAAA | TGG | 23 |
| 165 | 1 | CGTCGCCATTTCTCTCGCCG | CGG | 24 |
| 171 | -1 | ATAGTGGAGAAGACGCTCCG | CGG | 25 |
| 187 | 1 | GAGCGTCTTCTCCACTATCT | CGG | 26 |
| 187 | -1 | TCAAAACCTGACCGAGATAG | TGG | 27 |
| 192 | 1 | TCTTCTCCACTATCTCGGTC | AGG | 28 |
| 220 | -1 | AGGCCTCGTATAGAGGCTTC | TGG | 29 |
| 227 | -1 | TTCTGCAAGGCCTCGTATAG | AGG | 30 |
| 228 | 1 | GAACCAGAAGCCTCTATACG | AGG | 31 |
| 240 | -1 | CTCCTCCTTGATCTTCTGCA | AGG | 32 |
| 246 | 1 | CGAGGCCTTGCAGAAGATCA | AGG | 33 |
| 249 | 1 | GGCCTTGCAGAAGATCAAGG | AGG | 34 |
| 264 | 1 | CAAGGAGGAGTTGATGCTTT | TGG | 35 |
| 265 | 1 | AAGGAGGAGTTGATGCTTTT | GGG | 36 |
| 292 | -1 | TTGTGTTCTGCGAAACAGTG | AGG | 37 |
| 332 | -1 | AGATTGTCGACCAAAGAAGC | AGG | 38 |
| 333 | 1 | GTTTTGCGTACCTGCTTCTT | TGG | 39 |
| 355 | -1 | GATGAGGGCGCTTACAAGGG | AGG | 40 |
| 358 | -1 | CCTGATGAGGGCGCTTACAA | GGG | 41 |
| 359 | -1 | TCCTGATGAGGGCGCTTACA | AGG | 42 |
| 369 | 1 | CCCTTGTAAGCGCCCTCATC | AGG | 43 |
| 370 | -1 | AATCATTAGCTTCCTGATGA | GGG | 44 |
| 371 | -1 | GAATCATTAGCTTCCTGATG | AGG | 45 |
| 405 | -1 | AGAGCCGGAGATGTGATGAG | AGG | 46 |
| 412 | 1 | TCAACCTCTCATCACATCTC | CGG | 47 |
| 420 | -1 | AAGAAGGCGTCTGAAAGAGC | CGG | 48 |
| 436 | -1 | CAGTGGAAGTTTCTTCAAGA | AGG | 49 |
| 453 | -1 | GCAATAACCCAAATGAGCAG | TGG | 50 |
| 456 | 1 | AGAAACTTCCACTGCTCATT | TGG | 51 |
| 457 | 1 | GAAACTTCCACTGCTCATTT | GGG | 52 |
| 474 | 1 | TTTGGGTTATTGCGCTCATA | AGG | 53 |
| 501 | -1 | ACAACAACAACTAAAGATAT | GGG | 54 |
| 502 | -1 | AACAACAACAACTAAAGATA | TGG | 55 |
| 521 | 1 | TTAGTTGTTGTTGTTTTTTT | AGG | 56 |
| 522 | 1 | TAGTTGTTGTTGTTTTTTTA | GGG | 57 |
| 523 | 1 | AGTTGTTGTTGTTTTTTTAG | GGG | 58 |
| 570 | 1 | TATAAATATACTTTCCCAAA | AGG | 59 |
| 571 | 1 | ATAAATATACTTTCCCAAAA | GGG | 60 |
| 573 | -1 | TAAAGCGAATAGTCCCTTTT | GGG | 61 |
| 574 | -1 | TTAAAGCGAATAGTCCCTTT | TGG | 62 |
| 657 | -1 | ATGCTTCAACGGAATAAAAG | GGG | 63 |
| 658 | -1 | AATGCTTCAACGGAATAAAA | GGG | 64 |
| 659 | -1 | CAATGCTTCAACGGAATAAA | AGG | 65 |
| 668 | -1 | CAAATGGTGCAATGCTTCAA | CGG | 66 |
| 684 | -1 | CGAAGATAAAGATATGCAAA | TGG | 67 |
| 708 | -1 | AAGTGACATGGACAATGGCT | AGG | 68 |
| 713 | -1 | ACAGAAAGTGACATGGACAA | TGG | 69 |
| 720 | -1 | TGAGAACACAGAAAGTGACA | TGG | 70 |
| 744 | 1 | TGTGTTCTCACTGTTGTGTT | TGG | 71 |
| 747 | 1 | GTTCTCACTGTTGTGTTTGG | AGG | 72 |
| 755 | 1 | TGTTGTGTTTGGAGGTGTAA | AGG | 73 |
| 779 | -1 | GAGAGAGAAGCATATGAATT | TGG | 74 |
| 860 | 1 | TACACAACTAGATACGTCAA | TGG | 75 |
| 869 | 1 | AGATACGTCAATGGAAACGT | TGG | 76 |
| 870 | 1 | GATACGTCAATGGAAACGTT | GGG | 77 |
| 873 | 1 | ACGTCAATGGAAACGTTGGG | AGG | 78 |
| 910 | 1 | GAGAGTTATGACACTGAACA | AGG | 79 |
| 937 | -1 | TTCTATAATATTGTCAAAAG | TGG | 80 |
| 978 | -1 | TAAGCGATTCATATGTTAGA | AGG | 81 |
| 1017 | 1 | TGTTCTTAAGTCTAAGAAAA | AGG | 82 |
| 1039 | -1 | CCTTAATGAACGCGTGTTGA | TGG | 83 |
| 1050 | 1 | CCATCAACACGCGTTCATTA | AGG | 84 |
| 1062 | 1 | GTTCATTAAGGACCACTTTT | TGG | 85 |
| 1063 | 1 | TTCATTAAGGACCACTTTTT | GGG | 86 |
| 1063 | -1 | CTTTACCAAAACCCAAAAAG | TGG | 87 |
| 1069 | 1 | AAGGACCACTTTTTGGGTTT | TGG | 88 |
| 1090 | 1 | GGTAAAGACTCAGCTCTACT | AGG | 89 |
| 1094 | 1 | AAGACTCAGCTCTACTAGGC | TGG | 90 |
| 1098 | 1 | CTCAGCTCTACTAGGCTGGC | TGG | 91 |
| 1140 | -1 | ATAATCCTAATTCAGAACTT | TGG | 92 |
| 1146 | 1 | AGTATCCAAAGTTCTGAATT | AGG | 93 |
| 1159 | 1 | CTGAATTAGGATTATTCTTA | TGG | 94 |
| 1174 | -1 | ATATCAAGAGAATAAGAAAA | CGG | 95 |
| 1214 | -1 | AACGTTCTCAAAATAGAAAG | TGG | 96 |
| 1267 | -1 | CTCCAAAGGCTCAGTATCAA | TGG | 97 |
| 1276 | 1 | CTCCATTGATACTGAGCCTT | TGG | 98 |
| 1281 | -1 | GTGATGGAGAAAATCTCCAA | _{A}GG | 99 |
| 1297 | -1 | ATGTCCTAGTTTTCATGTGA | TGG | 100 |
| 1304 | 1 | TTCTCCATCACATGAAAACT | AGG | 101 |
| 1327 | 1 | ACATTTTTGTGCACATGTTA | AGG | 102 |
| 1349 | 1 | GAGCTAGCTAACATTAACAT | TGG | 103 |
| 1356 | 1 | CTAACATTAACATTGGAAAC | AGG | 104 |
| 1379 | -1 | GGGGAAAAAGAATCAAATCA | TGG | 105 |
| 1398 | -1 | AAAAGCATGCTGTTCACAAG | GGG | 106 |
| 1399 | -1 | CAAAAGCATGCTGTTCACAA | GGG | 107 |
| 1400 | -1 | ACAAAAGCATGCTGTTCACA | AGG | 108 |
| 1446 | -1 | CATGGTCGCATAATCTGATT | TGG | 109 |
| 1460 | 1 | AATCAGATTATGCGACCATG | CGG | 110 |
| 1464 | -1 | CATGATGAATCCTAACCGCA | TGG | 111 |
| 1465 | 1 | GATTATGCGACCATGCGGTT | AGG | 112 |
| 1476 | 1 | CATGCGGTTAGGATTCATCA | TGG | 113 |
| 1520 | 1 | TTACTTATAAATTACTAGAA | TGG | 114 |
| 1563 | 1 | CTTTTTTTCTTTTCACTAAA | TGG | 115 |
| 1603 | 1 | TTGTATTCTAGACTCACTGC | AGG | 116 |
| 1604 | 1 | TGTATTCTAGACTCACTGCA | GGG | 117 |
| 1605 | 1 | GTATTCTAGACTCACTGCAG | GGG | 118 |
| 1674 | 1 | GATGATTTCAAGAAAGTTGT | TGG | 119 |
| 1675 | 1 | ATGATTTCAAGAAAGTTGTT | GGG | 120 |
| 1681 | 1 | TCAAGAAAGTTGTTGGGATA | AGG | 121 |
| 1691 | 1 | TGTTGGGATAAGGTAACCCT | TGG | 122 |
| 1696 | -1 | GAAAATAGACTGTCAACCAA | GGG | 123 |
| 1697 | -1 | AGAAAATAGACTGTCAACCA | AGG | 124 |
| 1777 | 1 | TTCTTTTAAGTCTACTGTAT | CGG | 125 |
| 1792 | -1 | AAAAGCTAAAAGGTCAATCT | AGG | 126 |
| 1802 | -1 | ACTGCAGCCAAAAAGCTAAA | AGG | 127 |
| 1806 | 1 | AGATTGACCTTTTAGCTTTT | TGG | 128 |
| 1816 | 1 | TTTAGCTTTTTGGCTGCAGT | TGG | 129 |
| 1825 | 1 | TTGGCTGCAGTTGGTACCTT | TGG | 130 |
| 1826 | 1 | TGGCTGCAGTTGGTACCTTT | GGG | 131 |
| 1830 | -1 | AGATGACCACAAAAACCCAA | AGG | 132 |
| 1835 | 1 | TTGGTACCTTTGGGTTTTTG | TGG | 133 |
| 1863 | 1 | TTCTTGTTGCTGAATGTTAA | TGG | 134 |
| 1910 | -1 | ATCACTTAGATCTTGAGTTA | TGG | 135 |
| 1926 | 1 | ACTCAAGATCTAAGTGATAT | TGG | 136 |
| 1958 | -1 | CAAAGAACCAGACTGATTAC | GGG | 137 |
| 1959 | -1 | ACAAAGAACCAGACTGATTA | CGG | 138 |
| 1962 | 1 | GCAGAATCCCGTAATCAGTC | TGG | 139 |
| 1999 | 1 | CTTCAAGTGTGTCATCTCTT | TGG | 140 |
| 2033 | -1 | GCTTATTTGAAACTATAATT | TGG | 141 |
| 2101 | -1 | GTGGAGGCAGAGTAAGGAAT | TGG | 142 |
| 2107 | -1 | AGAAAAGTGGAGGCAGAGTA | AGG | 143 |
| 2117 | -1 | CTCCAACCATAGAAAAGTGG | AGG | 144 |
| 2120 | -1 | TTTCTCCAACCATAGAAAAG | TGG | 145 |
| 2122 | 1 | ACTCTGCCTCCACTTTTCTA | TGG | 146 |
| 2126 | 1 | TGCCTCCACTTTTCTATGGT | TGG | 147 |
| 2148 | 1 | GAGAAAATTATACTCCAAGT | TGG | 148 |
| 2151 | -1 | TCCAATTCCTTACACCAACT | TGG | 149 |
| 2155 | 1 | TTATACTCCAAGTTGGTGTA | AGG | 150 |
| 2161 | 1 | TCCAAGTTGGTGTAAGGAAT | TGG | 151 |
| 2203 | 1 | TCACTAGAATGCAATCAACA | AGG | 152 |
| 2204 | 1 | CACTAGAATGCAATCAACAA | GGG | 153 |
| 2244 | -1 | AATTTTTTTAATCAGAATTC | TGG | 154 |
| 2293 | -1 | TAAAAGTAAACTAAATTTCT | TGG | 155 |
| 2347 | -1 | ATGTAAATTATGTTCTATAT | AGG | 156 |
| 2380 | -1 | GATCCAATTGAATTATCTTA | AGG | 157 |
| 2388 | 1 | ACACCTTAAGATAATTCAAT | TGG | 158 |
| 2406 | 1 | ATTGGATCTTACTCCTTGTT | TGG | 159 |
| 2408 | -1 | GCCTCATTGTAGTCCAAACA | AGG | 160 |
| 2418 | 1 | TCCTTGTTTGGACTACAATG | AGG | 161 |
| 2453 | -1 | ATCTTGGTTTGAGTATTGAG | AGG | 162 |
| 2469 | -1 | ATATAAATAATGAATGATCT | TGG | 163 |
| 2497 | -1 | CAAAGAAGTTTAATACACAC | TGG | 164 |
| 2516 | 1 | TATTAAACTTCTTTGTTGTA | TGG | 165 |
| 2559 | 1 | TTTTGTCAATGTTTTGTGAT | TGG | 166 |
| 2597 | 1 | TAATAATGTGTTATATTTGC | AGG | 167 |
| 2601 | 1 | AATGTGTTATATTTGCAGGC | TGG | 168 |
| 2616 | 1 | CAGGCTGGCACACATaTTTC | TGG | 169 |
| 2640 | -1 | CTCAATAAACTCACAAAGAA | AGG | 170 |
| 2709 | 1 | CATGTTTCATTGTTCTTGCA | TGG | 171 |
| 2750 | 1 | CATTTTAAGTATCATACTGA | TGG | 172 |
| 2774 | 1 | GAAAGAGATAAAATACAGAG | AGG | 173 |
| 2775 | 1 | AAAGAGATAAAATACAGAGA | GGG | 174 |
| 2783 | 1 | AAAATACAGAGAGGGAGAAT | CGG | 175 |
| 2784 | 1 | AAATACAGAGAGGGAGAATC | GGG | 176 |
| 2817 | 1 | TTTAACACAATTTTGTAAAT | AGG | 177 |
| 2824 | 1 | CAATTTTGTAAATAGGCAAA | TGG | 178 |
| 2837 | 1 | AGGCAAATGGACAGCTAAGA | AGG | 179 |
| 2852 | -1 | TGTTCAATTAATTCTAAATT | TGG | 180 |
| 2875 | 1 | TTAATTGAACAACATGACCT | AGG | 181 |
| 2881 | -1 | AAATTGCACAATATTTACCT | AGG | 182 |
| 2950 | 1 | TAAATGTAGAGTCATGAGTC | AGG | 183 |
| 2951 | 1 | AAATGTAGAGTCATGAGTCA | GGG | 184 |
| 2973 | 1 | GTAGAAATTTGCACCTAGAC | AGG | 185 |
| 2975 | -1 | CACCTTAAAACCACCTGTCT | AGG | 186 |
| 2976 | 1 | GAAATTTGCACCTAGACAGG | TGG | 187 |
| 2984 | 1 | CACCTAGACAGGTGGTTTTA | AGG | 188 |
| 2987 | 1 | CTAGACAGGTGGTTTTAAGG | TGG | 189 |
| 3011 | 1 | ACTTCTCATCTCCAAGTCTT | AGG | 190 |
| 3011 | -1 | CATACATATCACCTAAGACT | TGG | 191 |
| 3046 | -1 | ATGTATATCACAACAGCAAA | AGG | 192 |
| 3083 | -1 | TTAAAAGAAAAAACAACAAG | TGG | 193 |
| 3114 | 1 | TAAATAGCTTCTACTTGCCG | TGG | 194 |
| 3115 | 1 | AAATAGCTTCTACTTGCCGT | GGG | 195 |
| 3120 | -1 | ATGCTCCAGTTTAGTGCCCA | CGG | 196 |
| 3126 | 1 | ACTTGCCGTGGGCACTAAAC | TGG | 197 |
| 3147 | 1 | GGAGCATGTCATTACTCAGT | TGG | 198 |
| 3184 | 1 | GAGAAACATGTAGCAATAGA | AGG | 199 |
| 3212 | -1 | AACCAAAAGTGATCATCTGA | TGG | 200 |
| 3221 | 1 | AGCCATCAGATGATCACTTT | TGG | 201 |
| 3242 | -1 | ATCAGGAAGAGGACAATCTG | GGG | 202 |
| 3243 | -1 | AATCAGGAAGAGGACAATCT | GGG | 203 |
| 3244 | -1 | GAATCAGGAAGAGGACAATC | TGG | 204 |
| 3253 | -1 | TGATGAAATGAATCAGGAAG | AGG | 205 |
| 3259 | -1 | AAAGGATGATGAAATGAATC | AGG | 206 |
| 3277 | -1 | TCTCAAATGAATTTTGGAAA | AGG | 207 |
| 3283 | -1 | ACGCAATCTCAAATGAATTT | TGG | 208 |
| 3305 | 1 | TTGAGATTGCGTTTTTCTTC | TGG | 209 |
| 3312 | 1 | TGCGTTTTTCTTCTGGATAT | TGG | 210 |
| 3320 | 1 | TCTTCTGGATATTGGTAAGC | TGG | 211 |
| 3343 | -1 | TGGTAGAAGTAGAAGCAGAG | TGG | 212 |
| 3363 | -1 | AATAACAATTTGTTCTTTTT | TGG | 213 |
| 3411 | 1 | ATCTTCTTTTCTGTGTATCT | AGG | 214 |
| 3441 | 1 | TTCATTTAACTCCTGTATAA | TGG | 215 |
| 3441 | -1 | ACGAACGTGTCCCATTATAC | AGG | 216 |
| 3442 | 1 | TCATTTAACTCCTGTATAAT | GGG | 217 |
| 3472 | -1 | TTTACCCAATGACAAGTCTT | GGG | 218 |
| 3473 | -1 | TTTTACCCAATGACAAGTCT | TGG | 219 |
| 3478 | 1 | ATTGTCCCAAGACTTGTCAT | TGG | 220 |
| 3479 | 1 | TTGTCCCAAGACTTGTCATT | GGG | 221 |
| 3541 | -1 | TAAAATAAAAGTTTCGTACT | TGG | 222 |
| 3570 | -1 | GAACACCCTAAAGCACAACA | TGG | 223 |
| 3575 | 1 | TTTTTACCATGTTGTGCTTT | AGG | 224 |
| 3576 | 1 | TTTTACCATGTTGTGCTTTA | GGG | 225 |
| 3588 | 1 | GTGCTTTAGGGTGTTCATTC | AGG | 226 |
| 3622 | -1 | GTGTGACAATGGCATAGAGC | GGG | 227 |
| 3623 | -1 | TGTGTGACAATGGCATAGAG | CGG | 228 |
| 3633 | -1 | TCAACGCACCTGTGTGACAA | TGG | 229 |
| 3636 | 1 | GCTCTATGCCATTGTCACAC | AGG | 230 |
| 3681 | 1 | ATAATTTAATAAGTTCTAAA | AGG | 231 |
| 3689 | 1 | ATAAGTTCTAAAAGGAAAGT | AGG | 232 |
| 3720 | -1 | CATTCCACAAGATTTTATTA | TGG | 233 |
| 3727 | 1 | CTGACCATAATAAAATCTTG | TGG | 234 |
| 3743 | 1 | CTTGTGGAATGATTTGAAGA | TGG | 235 |
| 3744 | 1 | TTGTGGAATGATTTGAAGAT | GGG | 236 |
| 3773 | 1 | TTACAAGAAAGCCATATTTG | AGG | 237 |
| 3773 | -1 | TTGCATGCGCTCCTCAAATA | TGG | 238 |
| 3789 | 1 | TTTGAGGAGCGCATGCAAGT | AGG | 239 |
| 3802 | 1 | TGCAAGTAGGAATTGTTAAT | TGG | 240 |
| 3803 | 1 | GCAAGTAGGAATTGTTAATT | GGG | 241 |
| 3812 | 1 | AATTGTTAATTGGGCTCAGA | AGG | 242 |
| 3827 | 1 | TCAGAAGGTCAAGAAAAAGA | AGG | 243 |
| 3828 | 1 | CAGAAGGTCAAGAAAAAGAA | GGG | 244 |
| 3849 | 1 | GGATTTAAAGCAGCCCTCAT | TGG | 245 |
| 3851 | -1 | GCCAGCACCGGAACCAATGA | GGG | 246 |
| 3852 | -1 | AGCCAGCACCGGAACCAATG | AGG | 247 |
| 3855 | 1 | AAAGCAGCCCTCATTGGTTC | CGG | 248 |
| 3861 | 1 | GCCCTCATTGGTTCCGGTGC | TGG | 249 |
| 3863 | -1 | GCCTGAGCCTGAGCCAGCAC | CGG | 250 |
| 3867 | 1 | ATTGGTTCCGGTGCTGGCTC | AGG | 251 |
| 3873 | 1 | TCCGGTGCTGGCTCAGGCTC | AGG | 252 |
| 3879 | 1 | GCTGGCTCAGGCTCAGGCTC | AGG | 253 |
| 3884 | 1 | CTCAGGCTCAGGCTCAGGCT | CGG | 254 |
| 3885 | 1 | TCAGGCTCAGGCTCAGGCTC | GGG | 255 |
| 3891 | 1 | TCAGGCTCAGGCTCGGGATC | AGG | 256 |
| 3903 | 1 | TCGGGATCAGGCTCTACTCC | TGG | 257 |
| 3910 | -1 | GTATCAGAAATTGGTTGACC | AGG | 258 |
| 3919 | -1 | GCAGAACCAGTATCAGAAAT | TGG | 259 |
| 3924 | 1 | GGTCAACCAATTTCTGATAC | TGG | 260 |
| 3938 | 1 | TGATACTGGTTCTGCATCTG | TGG | 261 |
| 3939 | 1 | GATACTGGTTCTGCATCTGT | GGG | 262 |
| 3950 | 1 | TGCATCTGTGGGAATTCAGC | TGG | 263 |
| 3951 | 1 | GCATCTGTGGGAATTCAGCT | GGG | 264 |
| 3973 | -1 | TGCTCTGGCTTTGATGCTTT | GGG | 265 |
| 3974 | -1 | CTGCTCTGGCTTTGATGCTT | TGG | 266 |
| 3988 | -1 | TTAGAGTCATCACTCTGCTC | TGG | 267 |
| 4058 | 1 | GAAGACATAAGTCTACCCTT | AGG | 268 |
| 4062 | -1 | CTAGTAGTAGTATTACCTAA | GGG | 269 |
| 4063 | -1 | ACTAGTAGTAGTATTACCTA | AGG | 270 |
| 4088 | -1 | ATCCCAGCACAGCTGGAAAG | TGG | 271 |
| 4095 | -1 | ATTTCTAATCCCAGCACAGC | TGG | 272 |
| 4096 | 1 | TTGCCACTTTCCAGCTGTGC | TGG | 273 |
| 4097 | 1 | TGCCACTTTCCAGCTGTGCT | GGG | 274 |
| 4132 | 1 | AATTCTTCTGTCATATATTA | TGG | 275 |
| 4138 | 1 | TCTGTCATATATTATGGCTG | TGG | 276 |
| 4141 | 1 | GTCATATATTATGGCTGTGG | TGG | 277 |
| 4142 | 1 | TCATATATTATGGCTGTGGT | GGG | 278 |
| 4160 | -1 | GTCTTGTCCATAAAAGACTT | AGG | 279 |
| 4164 | 1 | GACTGTACCTAAGTCTTTTA | TGG | 280 |
| 4188 | -1 | TTATATAATATATTGATCAA | AGG | 281 |
| 4267 | 1 | CTTCTTTCTTCTTATTATCA | TGG | 282 |
| 4280 | 1 | ATTATCATGGTACATCCTTT | TGG | 283 |
| 4284 | -1 | TTCACTATTCAGTTACCAAA | AGG | 284 |
| 4312 | 1 | AGTGAATACGTGTAGTCTCA | TGG | 285 |
| 4313 | 1 | GTGAATACGTGTAGTCTCAT | GGG | 286 |

TABLE 2
CsMLO2 targeted gRNA sequences

| Position | | | | |
|---|---|---|---|---|
| on | | | | SEQ |
| SEQ ID | | | | ID |
| NO: 4 | Strand | Sequence | PAM | NO |
| 1977 | -1 | GTATGAATATGAAATTAAGT | TGG | 287 |
| 2044 | -1 | AGAGAGAGAGAGACAGAGAG | TGG | 288 |
| 2117 | -1 | TTGAAATTGGGATGGAGATG | TGG | 289 |
| 2125 | -1 | ATTCTGTTTTGAAATTGGGA | TGG | 290 |
| 2129 | -1 | GTAAATTCTGTTTTGAAATT | GGG | 291 |
| 2130 | -1 | TGTAAATTCTGTTTTGAAAT | TGG | 292 |
| 2153 | -1 | GTTAGAATGAAAAGTTTGAT | GGG | 293 |
| 2154 | -1 | AGTTAGAATGAAAAGTTTGA | TGG | 294 |
| 2211 | 1 | TATAATCAATTATTCCCAAG | TGG | 295 |
| 2214 | -1 | TAAATATAAATAGGCCACTT | GGG | 296 |
| 2215 | -1 | ATAAATATAAATAGGCCACT | TGG | 297 |
| 2223 | -1 | TAGTGATCATAAATATAAAT | AGG | 298 |
| 2278 | 1 | AAAATTAAATTAAAAGAAGA | TGG | 299 |
| 2281 | 1 | ATTAAATTAAAAGAAGATGG | CGG | 300 |
| 2284 | 1 | AAATTAAAAGAAGATGGCGG | TGG | 301 |
| 2291 | 1 | AAGAAGATGGCGGTGGCTAG | CGG | 302 |
| 2294 | 1 | AAGATGGCGGTGGCTAGCGG | AGG | 303 |
| 2322 | 1 | CTTTAGAACAAACACCAACA | TGG | 304 |
| 2323 | 1 | TTTAGAACAAACACCAACAT | GGG | 305 |
| 2325 | -1 | ACTACGGCCACAGCCCATGT | TGG | 306 |
| 2329 | 1 | ACAAACACCAACATGGGCTG | TGG | 307 |
| 2341 | -1 | TACCAAAACAAGACAAACTA | CGG | 308 |
| 2350 | 1 | GGCCGTAGTTTGTCTTGTTT | TGG | 309 |
| 2371 | -1 | GATTATGTGCTCAATAATAA | TGG | 310 |
| 2393 | 1 | GAGCACATAATCCATCTCAT | TGG | 311 |
| 2393 | -1 | GGTATACCTTGCCAATGAGA | TGG | 312 |
| 2398 | 1 | CATAATCCATCTCATTGGCA | AGG | 313 |
| 2414 | -1 | TGAGATTAATATATATAATT | GGG | 314 |
| 2415 | -1 | GTGAGATTAATATATATAAT | TGG | 315 |
| 2473 | 1 | CATTTAATTATTTAAATTAA | TGG | 316 |
| 2474 | 1 | ATTTAATTATTTAAATTAAT | GGG | 317 |
| 2495 | 1 | GGTATTTTTTTTTTTTTTAG | TGG | 318 |
| 2535 | 1 | ACGAGCTCTTTATGAATCGT | TGG | 319 |
| 2551 | 1 | TCGTTGGAAAAGATCAAATC | AGG | 320 |
| 2576 | -1 | AAAATGGGTATTCATTAATT | GGG | 321 |
| 2577 | -1 | AAAAATGGGTATTCATTAAT | TGG | 322 |
| 2591 | -1 | TTAAAAAAAAAAACAAAAAT | GGG | 323 |
| 2656 | 1 | TTTGATAGAGCTTATGTTAT | TGG | 324 |
| 2657 | 1 | TTGATAGAGCTTATGTTATT | GGG | 325 |
| 2658 | 1 | TGATAGAGCTTATGTTATTG | GGG | 326 |
| 2680 | 1 | GTTCATATCGTTGTTACTAA | CGG | 327 |
| 2683 | 1 | CATATCGTTGTTACTAACGG | TGG | 328 |
| 2684 | 1 | ATATCGTTGTTACTAACGGT | GGG | 329 |
| 2703 | -1 | GATATACAAATATTTGAGAT | CGG | 330 |
| 2726 | 1 | ATTTGTATATCTGAGAAAAT | TGG | 331 |
| 2729 | 1 | TGTATATCTGAGAAAATTGG | AGG | 332 |
| 2730 | 1 | GTATATCTGAGAAAATTGGA | GGG | 333 |
| 2736 | 1 | CTGAGAAAATTGGAGGGACA | TGG | 334 |
| 2751 | -1 | TCTTCTTGTTCTTTATTACA | AGG | 335 |
| 2777 | 1 | CAAGAAGAGAAATTGAATAA | AGG | 336 |
| 2778 | 1 | AAGAAGAGAAATTGAATAAA | GGG | 337 |
| 2779 | 1 | AGAAGAGAAATTGAATAAAG | GGG | 338 |
| 2817 | 1 | TCGAACATGAAAGTAACAGT | CGG | 339 |
| 2827 | 1 | AAGTAACAGTCGGAGATTGC | TGG | 340 |
| 2839 | -1 | ACCGTCGCCGGACTCTAAAA | AGG | 341 |
| 2843 | 1 | TTGCTGGCCTTTTTAGAGTC | CGG | 342 |
| 2849 | 1 | GCCTTTTTAGAGTCCGGCGA | CGG | 343 |
| 2851 | -1 | GACACTAGCAGCACCGTCGC | CGG | 344 |
| 2865 | 1 | GCGACGGTGCTGCTAGTGTC | CGG | 345 |
| 2873 | -1 | CGGCCGCCGCCAAAATTCGC | CGG | 346 |
| 2875 | 1 | TGCTAGTGTCCGGCGAATTT | TGG | 347 |
| 2878 | 1 | TAGTGTCCGGCGAATTTTGG | CGG | 348 |
| 2881 | 1 | TGTCCGGCGAATTTTGGCGG | CGG | 349 |
| 2885 | 1 | CGGCGAATTTTGGCGGCGGC | CGG | 350 |
| 2886 | 1 | GGCGAATTTTGGCGGCGGCC | GGG | 351 |
| 2893 | -1 | TTCAGCACACTTATCAGTCC | CGG | 352 |
| 2908 | 1 | GACTGATAAGTGTGCTGAAA | AGG | 353 |
| 2978 | 1 | GTCTTTCTTATCCTTTTATT | TGG | 354 |
| 2978 | -1 | GACGAATATGTCCAAATAAA | AGG | 355 |
| 3000 | -1 | CTCCTATAATATTATATGTT | TGG | 356 |
| 3009 | 1 | GTCCAAACATATAATATTAT | AGG | 357 |
| 3051 | -1 | AAATATATAAATTTAAAGGT | TGG | 358 |
| 3055 | -1 | AACTAAATATATAAATTTAA | AGG | 359 |
| 4125 | 1 | AAATTATATACATATATGAA | TGG | 360 |
| 4168 | 1 | ATATATATAATTATAATTTC | AGG | 361 |
| 4169 | 1 | TATATATAATTATAATTTCA | GGG | 362 |
| 4187 | -1 | ATACCATCCGCCGAAACAAA | TGG | 363 |
| 4188 | 1 | AGGGCAAGTTCCATTTGTTT | CGG | 364 |
| 4191 | 1 | GCAAGTTCCATTTGTTTCGG | CGG | 365 |
| 4195 | 1 | GTTCCATTTGTTTCGGCGGA | TGG | 366 |
| 4230 | 1 | GCATATTTTTATCTTTGTGT | TGG | 367 |
| 4249 | -1 | TCATGATGCAGTAGAGAACA | TGG | 368 |
| 4272 | 1 | CTGCATCATGACTATGTTTT | TGG | 369 |
| 4273 | 1 | TGCATCATGACTATGTTTTT | GGG | 370 |
| 4284 | 1 | TATGTTTTTGGGCAGACTTA | AGG | 371 |
| 4404 | -1 | AATTTATATATAATTATTTA | GGG | 372 |
| 4405 | -1 | CAATTTATATATAATTATTT | AGG | 373 |
| 4428 | 1 | TATATAAATTGATTCCCAGA | TGG | 374 |
| 4429 | 1 | ATATAAATTGATTCCCAGAT | GGG | 375 |
| 4431 | -1 | ATGCTTCCAACTTCCCATCT | GGG | 376 |
| 4432 | -1 | AATGCTTCCAACTTCCCATC | TGG | 377 |
| 4436 | 1 | TTGATTCCCAGATGGGAAGT | TGG | 378 |
| 4445 | 1 | AGATGGGAAGTTGGAAGCAT | TGG | 379 |
| 4446 | 1 | GATGGGAAGTTGGAAGCATT | GGG | 380 |
| 4452 | 1 | AAGTTGGAAGCATTGGGAAA | AGG | 381 |
| 4476 | -1 | ACCATGTGAGAATTGATATT | CGG | 382 |
| 4486 | 1 | GCCGAATATCAATTCTCACA | TGG | 383 |
| 4548 | 1 | CTTAATTTTAATTTTTCTAT | AGG | 384 |
| 4551 | 1 | AATTTTAATTTTTCTATAGG | TGG | 385 |
| 4649 | -1 | CTATATGACATATTTGATGG | TGG | 386 |
| 4652 | -1 | TAACTATATGACATATTTGA | TGG | 387 |
| 4742 | 1 | AATTATAAGAGCATCTTTAT | TGG | 388 |
| 4749 | 1 | AGAGCATCTTTATTGGACAC | CGG | 389 |
| 4757 | -1 | TAGAAAGTGTTAAATATCAC | CGG | 390 |
| 4844 | -1 | TATTGGTATAATTAAGTATC | AGG | 391 |
| 4861 | -1 | CTTACCAATTATATTATTAT | TGG | 392 |
| 4868 | 1 | TATACCAATAATAATATAAT | TGG | 393 |
| 4903 | 1 | ATTTATAAGAAGTATATATA | TGG | 394 |
| 4904 | 1 | TTTATAAGAAGTATATATAT | GGG | 395 |
| 4923 | 1 | TGGGAGTTAGAATTAAGTAA | AGG | 396 |
| 4997 | -1 | CTCGCAAATCTGAATCTTTC | TGG | 397 |
| 5009 | 1 | CAGAAAGATTCAGATTTGCG | AGG | 398 |
| 5010 | 1 | AGAAAGATTCAGATTTGCGA | GGG | 399 |
| 5023 | 1 | TTTGCGAGGGACACTTCTTT | TGG | 400 |
| 5045 | 1 | GAAGAAGACATTTAAGTTTC | TGG | 401 |
| 5058 | -1 | CCATATTAGGAAAGGGTGTT | TGG | 402 |
| 5065 | -1 | TTACTATCCATATTAGGAAA | GGG | 403 |
| 5066 | -1 | CTTACTATCCATATTAGGAA | AGG | 404 |
| 5069 | 1 | CCAAACACCCTTTCCTAATA | TGG | 405 |
| 5071 | -1 | GGGATCTTACTATCCATATT | AGG | 406 |
| 5091 | -1 | AAGTAAAAAGTGGGTAAAAA | GGG | 407 |
| 5092 | -1 | AAAGTAAAAAGTGGGTAAAA | AGG | 408 |
| 5100 | -1 | AATATAAAAAAGTAAAAAGT | GGG | 409 |
| 5101 | -1 | GAATATAAAAAAGTAAAAAG | TGG | 410 |
| 5149 | -1 | ATATAAGTGCATGGATATAG | TGG | 411 |
| 5158 | -1 | TATTAATAGATATAAGTGCA | TGG | 412 |
| 5233 | -1 | CATATTTATATGCATGTGAA | AGG | 413 |
| 5253 | 1 | TGCATATAAATATGTTTGCA | TGG | 414 |
| 5269 | 1 | TGCATGGTTTTTATACATCG | TGG | 415 |
| 7159 | 1 | TATATATATAATATTTTTTT | TGG | 416 |
| 7213 | -1 | TTAATTAATAATTAAAGAGC | AGG | 417 |
| 7238 | 1 | ATTAATTAATTATTTTTCGC | AGG | 418 |
| 7282 | -1 | AAAGTTAAATAATCAACTTT | AGG | 419 |
| 7302 | 1 | GATTATTTAACTTTGAGACA | TGG | 420 |
| 7313 | 1 | TTTGAGACATGGATTTATAA | TGG | 421 |
| 7373 | 1 | ATTATAGCTGTAGAGATATT | TGG | 422 |
| 7387 | -1 | TAAGTATTATTAAAAATACA | AGG | 423 |
| 8017 | -1 | GAATGAGAATAGGAATAGAA | TGG | 424 |
| 8027 | -1 | ATAGGAATGGGAATGAGAAT | AGG | 425 |
| 8039 | -1 | ATAGGAATAGAAATAGGAAT | GGG | 426 |
| 8040 | -1 | TATAGGAATAGAAATAGGAA | TGG | 427 |
| 8045 | -1 | GAAAATATAGGAATAGAAAT | AGG | 428 |
| 8057 | -1 | GTTGAGAGGAATGAAAATAT | AGG | 429 |
| 8071 | -1 | CACAGAGGCGTTTGGTTGAG | AGG | 430 |
| 8079 | -1 | AATAGGCCCACAGAGGCGTT | TGG | 431 |
| 8083 | 1 | CTCTCAACCAAACGCCTCTG | TGG | 432 |
| 8084 | 1 | TCTCAACCAAACGCCTCTGT | GGG | 433 |
| 8086 | -1 | ACAAGATAATAGGCCCACAG | AGG | 434 |
| 8096 | -1 | TTAATACATAACAAGATAAT | AGG | 435 |
| 8150 | 1 | ATCAATAACTAAATTAATTG | AGG | 436 |
| 8177 | 1 | TTATAACAATTAATAATTTC | AGG | 437 |
| 8186 | 1 | TTAATAATTTCAGGCACATT | TGG | 438 |
| 8198 | -1 | AAATTTTTGATGGCTTTGAG | GGG | 439 |
| 8199 | -1 | CAAATTTTTGATGGCTTTGA | GGG | 440 |
| 8200 | -1 | TCAAATTTTTGATGGCTTTG | AGG | 441 |
| 8208 | -1 | TTTGAAAGTCAAATTTTTGA | TGG | 442 |
| 8255 | 1 | ATCTCTAGAAGAAGATTTCA | AGG | 443 |
| 8265 | 1 | GAAGATTTCAAGGTCGTTGT | AGG | 444 |
| 8272 | 1 | TCAAGGTCGTTGTAGGAATC | AGG | 445 |
| 8345 | -1 | ATTAAAATAAGTCATCATTT | GGG | 446 |
| 8346 | -1 | AATTAAAATAAGTCATCATT | TGG | 447 |
| 8399 | 1 | TAATAATTATTATTTTGTTT | TGG | 448 |
| 8427 | 1 | TCAATCTCAGTCCTCCTATT | TGG | 449 |
| 8427 | -1 | ACAGCGAAGAACCAAATAGG | AGG | 450 |
| 8430 | -1 | ACCACAGCGAAGAACCAAAT | AGG | 451 |
| 8440 | 1 | TCCTATTTGGTTCTTCGCTG | TGG | 452 |
| 8465 | 1 | TTCTTACTCTTCAATACCCA | TGG | 453 |
| 8470 | -1 | AATAATAAAATGCTCACCAT | GGG | 454 |
| 8471 | -1 | TAATAATAAAATGCTCACCA | TGG | 455 |
| 8500 | -1 | GGATGCATTGAAATAATTAA | TGG | 456 |
| 8521 | -1 | AATCTAAACTGTGATAATTA | GGG | 457 |
| 8522 | -1 | AAATCTAAACTGTGATAATT | AGG | 458 |
| 8566 | -1 | TTGACATATATGCACACGTT | TGG | 459 |
| 8605 | 1 | TATATTTTTGTTTTTATTAT | TGG | 460 |
| 8618 | -1 | AAATGTAAACAAATTCATTA | TGG | 461 |
| 8631 | 1 | ATAATGAATTTGTTTACATT | TGG | 462 |
| 8636 | 1 | GAATTTGTTTACATTTGGAC | AGG | 463 |
| 8640 | 1 | TTGTTTACATTTGGACAGGC | TGG | 464 |
| 8655 | 1 | CAGGCTGGTATTCTTATCTT | TGG | 465 |
| 8670 | -1 | CTTACAATTAGAGGAATAAA | AGG | 466 |
| 8679 | -1 | ATATTAGTACTTACAATTAG | AGG | 467 |
| 8820 | -1 | GATTGTTTGAATTTTATTTT | TGG | 468 |
| 8907 | -1 | GTTTACAGTAAAACTTTAAA | AGG | 469 |
| 8932 | -1 | AATTAGCCCAATTTTTTTCA | CGG | 470 |
| 8936 | 1 | TAAACTACCGTGAAAAAAAT | TGG | 471 |
| 8937 | 1 | AAACTACCGTGAAAAAAATT | GGG | 472 |
| 9001 | -1 | CTCTTTTATTTTTTAAGAAG | AGG | 473 |
| 9053 | 1 | TATTATAAATAAATTATGTT | AGG | 474 |
| 9065 | 1 | ATTATGTTAGGTGATCCTAT | TGG | 475 |
| 9068 | 1 | ATGTTAGGTGATCCTATTGG | TGG | 476 |
| 9069 | 1 | TGTTAGGTGATCCTATTGGT | GGG | 477 |
| 9069 | -1 | GTAATTTCGTCCCCACCAAT | AGG | 478 |
| 9070 | 1 | GTTAGGTGATCCTATTGGTG | GGG | 479 |
| 9101 | 1 | ACAAGTGATTATAACAAAGA | TGG | 480 |
| 9102 | 1 | CAAGTGATTATAACAAAGAT | GGG | 481 |
| 9103 | 1 | AAGTGATTATAACAAAGATG | GGG | 482 |
| 9123 | 1 | GGGCTAAGAATTCAAGAAAG | AGG | 483 |
| 9138 | 1 | GAAAGAGGAGAAGTTGTAAA | AGG | 484 |
| 9149 | 1 | AGTTGTAAAAGGAGTGCCTG | TGG | 485 |
| 9154 | -1 | TCGTCCCCAGGTTGGACCAC | AGG | 486 |
| 9159 | 1 | GGAGTGCCTGTGGTCCAACC | TGG | 487 |
| 9160 | 1 | GAGTGCCTGTGGTCCAACCT | GGG | 488 |
| 9161 | 1 | AGTGCCTGTGGTCCAACCTG | GGG | 489 |
| 9162 | -1 | AGAAAAGGTCGTCCCCAGGT | TGG | 490 |
| 9166 | -1 | AACCAGAAAAGGTCGTCCCC | AGG | 491 |
| 9175 | 1 | AACCTGGGGACGACCTTTTC | TGG | 492 |
| 9177 | -1 | GTGGGCGGTTGAACCAGAAA | AGG | 493 |
| 9192 | -1 | GGTAGAGAATAAGGCGTGGG | CGG | 494 |
| 9195 | -1 | TAAGGTAGAGAATAAGGCGT | GGG | 495 |
| 9196 | -1 | ATAAGGTAGAGAATAAGGCG | TGG | 496 |
| 9201 | -1 | AGTTAATAAGGTAGAGAATA | AGG | 497 |
| 9213 | -1 | GGAAGAGGACGAAGTTAATA | AGG | 498 |
| 9227 | 1 | TATTAACTTCGTCCTCTTCC | AGG | 499 |
| 9228 | -1 | ATTGATTATGTACCTGGAAG | AGG | 500 |
| 9234 | -1 | ATTTTGATTGATTATGTACC | TGG | 501 |
| 9254 | 1 | TAATCAATCAAAATCAGCCT | TGG | 502 |
| 9260 | -1 | GGTGCATTATAGAATTTCCA | AGG | 503 |
| 9281 | -1 | TCAATGTATTCATTTTAAGG | GGG | 504 |
| 9282 | -1 | ATCAATGTATTCATTTTAAG | GGG | 505 |
| 9283 | -1 | CATCAATGTATTCATTTTAA | GGG | 506 |
| 9284 | -1 | GCATCAATGTATTCATTTTA | AGG | 507 |
| 9308 | -1 | TTGAGTGCTAAAACAAGTAA | GGG | 508 |
| 9309 | -1 | TTTGAGTGCTAAAACAAGTA | AGG | 509 |
| 9350 | 1 | TTTAGTCAAATTTTTTCTCA | TGG | 510 |
| 10632 | -1 | TCCATGCAAAGAACGCAAGC | TGG | 511 |
| 10642 | 1 | TCCAGCTTGCGTTCTTTGCA | TGG | 512 |
| 10648 | 1 | TTGCGTTCTTTGCATGGACT | TGG | 513 |
| 10649 | 1 | TGCGTTCTTTGCATGGACTT | GGG | 514 |
| 10753 | 1 | TTAATTTTTCAGTATGAATT | TGG | 515 |
| 10785 | 1 | TTGCTTTCATGAACATGTTG | AGG | 516 |
| 10791 | 1 | TCATGAACATGTTGAGGATG | TGG | 517 |
| 10809 | 1 | TGTGGTTATCAGAATCACCA | TGG | 518 |
| 10810 | 1 | GTGGTTATCAGAATCACCAT | GGG | 519 |
| 10811 | 1 | TGGTTATCAGAATCACCATG | GGG | 520 |
| 10815 | -1 | ATATCTGTATACAGACCCCA | TGG | 521 |
| 10922 | -1 | AAATAAAAATTAAATATTAA | TGG | 522 |
| 10958 | 1 | GTAAAAATTTCTAACACCGT | TGG | 523 |
| 10963 | -1 | CCCTGATGATCATGATCCAA | CGG | 524 |
| 10973 | 1 | ACCGTTGGATCATGATCATC | AGG | 525 |
| 10974 | 1 | CCGTTGGATCATGATCATCA | GGG | 526 |
| 10993 | -1 | TGACGTAGCTGCACAGAATC | TGG | 527 |
| 11016 | -1 | AACAAGGGCGTAGAGAGGGA | GGG | 528 |
| 11017 | -1 | TAACAAGGGCGTAGAGAGGG | AGG | 529 |
| 11020 | -1 | GTGTAACAAGGGCGTAGAGA | GGG | 530 |
| 11021 | -1 | TGTGTAACAAGGGCGTAGAG | AGG | 531 |
| 11031 | -1 | TGTAATTACTTGTGTAACAA | GGG | 532 |
| 11032 | -1 | GTGTAATTACTTGTGTAACA | AGG | 533 |
| 11159 | -1 | AGATTTTATATATTTAATTA | GGG | 534 |
| 11160 | -1 | TAGATTTTATATATTTAATT | AGG | 535 |
| 11524 | -1 | CGGACTATATTTTAATTAAA | AGG | 536 |
| 11544 | -1 | TAATTAAATAAAATTCTAAA | CGG | 537 |
| 11580 | 1 | TAAAAAATATTGTCATAGTT | TGG | 538 |
| 11581 | 1 | AAAAAATATTGTCATAGTTT | GGG | 539 |
| 11782 | 1 | TATATATATGACACAACAGA | TGG | 540 |
| 11783 | 1 | ATATATATGACACAACAGAT | GGG | 541 |
| 11800 | -1 | GTTGAATATAGTTGGTTTCA | TGG | 542 |
| 11808 | -1 | ACTTTGTCGTTGAATATAGT | TGG | 543 |
| 11824 | 1 | TATATTCAACGACAAAGTAG | CGG | 544 |
| 11827 | 1 | ATTCAACGACAAAGTAGCGG | AGG | 545 |
| 11839 | -1 | TGAGTGGTGCCAGTTGCGGA | GGG | 546 |
| 11840 | -1 | CTGAGTGGTGCCAGTTGCGG | AGG | 547 |
| 11841 | 1 | TAGCGGAGGCCCTCCGCAAC | TGG | 548 |
| 11843 | -1 | GGGCTGAGTGGTGCCAGTTG | CGG | 549 |
| 11855 | -1 | TGATGTGCTTTCGGGCTGAG | TGG | 550 |
| 11863 | -1 | TTGGTGTTTGATGTGCTTTC | GGG | 551 |
| 11864 | -1 | TTTGGTGTTTGATGTGCTTT | CGG | 552 |
| 11881 | 1 | GCACATCAAACACCAAAACA | AGG | 553 |
| 11882 | -1 | CTGACCCCGCCGCCTTGTTT | TGG | 554 |
| 11884 | 1 | CATCAAACACCAAAACAAGG | CGG | 555 |
| 11887 | 1 | CAAACACCAAAACAAGGCGG | CGG | 556 |
| 11888 | 1 | AAACACCAAAACAAGGCGGC | GGG | 557 |
| 11889 | 1 | AACACCAAAACAAGGCGGCG | GGG | 558 |
| 11910 | -1 | GTCGTCGGCCGGCTTGACAG | CGG | 559 |
| 11913 | 1 | CAGTGACGCCGCTGTCAAGC | CGG | 560 |
| 11921 | -1 | GATGTGTGGGTGTCGTCGGC | CGG | 561 |
| 11925 | -1 | ATGTGATGTGTGGGTGTCGT | CGG | 562 |
| 11934 | -1 | ACCGGGGACATGTGATGTGT | GGG | 563 |
| 11935 | -1 | GACCGGGGACATGTGATGTG | TGG | 564 |
| 11944 | 1 | ACCCACACATCACATGTCCC | CGG | 565 |
| 11950 | -1 | GTGGCGCAAGAGGTGGACCG | GGG | 566 |
| 11951 | -1 | AGTGGCGCAAGAGGTGGACC | GGG | 567 |
| 11952 | -1 | TAGTGGCGCAAGAGGTGGAC | CGG | 568 |
| 11957 | -1 | TGCGGTAGTGGCGCAAGAGG | TGG | 569 |
| 11960 | -1 | CACTGCGGTAGTGGCGCAAG | AGG | 570 |
| 11969 | -1 | CTGCTGCCTCACTGCGGTAG | TGG | 571 |
| 11974 | 1 | CTTGCGCCACTACCGCAGTG | AGG | 572 |
| 11975 | -1 | GGCTGTCTGCTGCCTCACTG | CGG | 573 |
| 11996 | -1 | AGCGCCTTGGGGAGTTTTGG | AGG | 574 |
| 11999 | -1 | TTGAGCGCCTTGGGGAGTTT | TGG | 575 |
| 12003 | 1 | ACAGCCTCCAAAACTCCCCA | AGG | 576 |
| 12007 | -1 | ATCAAAGTTTGAGCGCCTTG | GGG | 577 |
| 12008 | -1 | CATCAAAGTTTGAGCGCCTT | GGG | 578 |
| 12009 | -1 | CCATCAAAGTTTGAGCGCCT | TGG | 579 |
| 12020 | 1 | CCAAGGCGCTCAAACTTTGA | TGG | 580 |
| 12033 | 1 | ACTTTGATGGCGCCACTGAA | CGG | 581 |
| 12034 | -1 | ATCTGTCTCCCACCGTTCAG | TGG | 582 |
| 12036 | 1 | TTGATGGCGCCACTGAACGG | TGG | 583 |
| 12037 | 1 | TGATGGCGCCACTGAACGGT | GGG | 584 |
| 12059 | -1 | TGGTGGTGGTGAGATGGAGA | TGG | 585 |
| 12065 | -1 | CGGCCGTGGTGGTGGTGAGA | TGG | 586 |
| 12073 | 1 | TCTCCATCTCACCACCACCA | CGG | 587 |
| 12073 | -1 | TCGCGAAGCGGCCGTGGTGG | TGG | 588 |
| 12076 | -1 | CGGTCGCGAAGCGGCCGTGG | TGG | 589 |
| 12079 | -1 | CCTCGGTCGCGAAGCGGCCG | TGG | 590 |
| 12085 | -1 | AGGAACCCTCGGTCGCGAAG | CGG | 591 |
| 12090 | 1 | CCACGGCCGCTTCGCGACCG | AGG | 592 |
| 12091 | 1 | CACGGCCGCTTCGCGACCGA | GGG | 593 |
| 12096 | -1 | ATGATGAGAGGAGGAACCCT | CGG | 594 |
| 12105 | -1 | ATTATTACTATGATGAGAGG | AGG | 595 |
| 12108 | -1 | ATTATTATTACTATGATGAG | AGG | 596 |
| 12150 | 1 | TAAAAATCAGCAAATTGAAT | TGG | 597 |
| 12151 | 1 | AAAAATCAGCAAATTGAATT | GGG | 598 |
| 12162 | 1 | AATTGAATTGGGACAAATAA | TGG | 599 |
| 12181 | 1 | ATGGAACAACATCATCTTCA | TGG | 600 |
| 12188 | 1 | AACATCATCTTCATGGAGAT | CGG | 601 |
| 12204 | -1 | GGTTTGAGGAGGAAGCTCAT | TGG | 602 |
| 12215 | -1 | CTTAATGTAGTGGTTTGAGG | AGG | 603 |
| 12218 | -1 | TTTCTTAATGTAGTGGTTTG | AGG | 604 |
| 12225 | -1 | AGCTTGATTTCTTAATGTAG | TGG | 605 |
| 12267 | 1 | TGATCAATCAGCAGCAGCAC | AGG | 606 |
| 12272 | 1 | AATCAGCAGCAGCACAGGTG | AGG | 607 |
| 12284 | -1 | TTAATTTCATGGTGGGGCGG | CGG | 608 |
| 12287 | -1 | ATATTAATTTCATGGTGGGG | CGG | 609 |
| 12290 | -1 | CCAATATTAATTTCATGGTG | GGG | 610 |
| 12291 | -1 | TCCAATATTAATTTCATGGT | GGG | 611 |
| 12292 | -1 | GTCCAATATTAATTTCATGG | TGG | 612 |
| 12295 | -1 | TGTGTCCAATATTAATTTCA | TGG | 613 |
| 12301 | 1 | CCCCACCATGAAATTAATAT | TGG | 614 |
| 12326 | 1 | ACAGAGATTTCTCTTTTGAA | CGG | 615 |
| 12350 | -1 | CTCTCTCGTCATCAAACGCT | GGG | 616 |
| 12351 | -1 | TCTCTCTCGTCATCAAACGC | TGG | 617 |
| 12376 | 1 | CGAGAGAGAATTCCGTTATT | TGG | 618 |
| 12377 | -1 | TTAACATTATAACCAAATAA | CGG | 619 |
| 12392 | 1 | TATTTGGTTATAATGTTAAT | CGG | 620 |
| 12396 | 1 | TGGTTATAATGTTAATCGGA | CGG | 621 |
| 12411 | 1 | TCGGACGGTTCTCATTGTCT | CGG | 622 |
| 12423 | -1 | TCTAGCTCGTTGATCATCAG | AGG | 623 |
| 12499 | -1 | ATAATTAAACCGCTCATTAT | TGG | 624 |
| 12501 | 1 | TAAGCAGCTCCAATAATGAG | CGG | 625 |

TABLE 3
CsMLO3 targeted gRNA sequences

| Position | | | | |
|---|---|---|---|---|
| on | | | | SEQ |
| SEQ ID | | | | ID |
| NO: 7 | Strand | Sequence | PAM | NO |
| 777 | 1 | TGAAACTCAAACTAAAATCA | AGG | 626 |
| 801 | -1 | TCTAACAGTTGGTATCAGAG | CGG | 627 |
| 812 | -1 | ATATATAAATGTCTAACAGT | TGG | 628 |
| 860 | 1 | ATATGTTTAAGTATTAACTG | CGG | 629 |
| 894 | 1 | TATATACACTATATAACTTA | AGG | 630 |
| 915 | -1 | GCTCAAGAATCAATGGCTGG | AGG | 631 |
| 918 | -1 | GAAGCTCAAGAATCAATGGC | TGG | 632 |
| 922 | -1 | GTTTGAAGCTCAAGAATCAA | TGG | 633 |
| 944 | -1 | TTGCAGATCAAAGCTTATGT | GGG | 634 |
| 945 | -1 | CTTGCAGATCAAAGCTTATG | TGG | 635 |
| 957 | 1 | CACATAAGCTTTGATCTGCA | AGG | 636 |
| 958 | 1 | ACATAAGCTTTGATCTGCAA | GGG | 637 |
| 965 | 1 | CTTTGATCTGCAAGGGAAAC | TGG | 638 |
| 974 | 1 | GCAAGGGAAACTGGTTGATG | TGG | 639 |
| 975 | 1 | CAAGGGAAACTGGTTGATGT | GGG | 640 |
| 982 | 1 | AACTGGTTGATGTGGGTAAT | CGG | 641 |
| 983 | 1 | ACTGGTTGATGTGGGTAATC | GGG | 642 |
| 998 | -1 | TAAAGAGAGTTGAGAGAGCG | AGG | 643 |
| 1014 | 1 | CTCTCTCAACTCTCTTTAGA | TGG | 644 |
| 1044 | 1 | TGTTATGAACAGAATGAGTG | AGG | 645 |
| 1051 | 1 | AACAGAATGAGTGAGGAGCT | CGG | 646 |
| 1052 | 1 | ACAGAATGAGTGAGGAGCTC | GGG | 647 |
| 1053 | 1 | CAGAATGAGTGAGGAGCTCG | GGG | 648 |
| 1066 | -1 | CACCTATAAATATAGGGTCT | CGG | 649 |
| 1072 | -1 | GTATCTCACCTATAAATATA | GGG | 650 |
| 1073 | -1 | AGTATCTCACCTATAAATAT | AGG | 651 |
| 1075 | 1 | GACCGAGACCCTATATTTAT | AGG | 652 |
| 1096 | -1 | TAATGTGGCACAGATACTGA | TGG | 653 |
| 1111 | -1 | AAATATTCTGACAATTAATG | TGG | 654 |
| 1138 | 1 | AATATTTTGACAATTAATTC | AGG | 655 |
| 1151 | 1 | TTAATTCAGGAAATCAAATC | AGG | 656 |
| 1183 | -1 | ATTATGTAATATTCTATATA | TGG | 657 |
| 4585 | 1 | GTTCTCACTATCAGTTATTA | TGG | 658 |
| 4595 | 1 | TCAGTTATTATGGTTATTTA | TGG | 659 |
| 4615 | 1 | TGGTTATTTATCTTTTTTAG | TGG | 660 |
| 4634 | -1 | CCTGAAGGGCTTTTTGTGTT | TGG | 661 |
| 4645 | 1 | CCAAACACAAAAAGCCCTTC | AGG | 662 |
| 4648 | -1 | CTTCTCAAGCGCTTCCTGAA | GGG | 663 |
| 4649 | -1 | TCTTCTCAAGCGCTTCCTGA | AGG | 664 |
| 4670 | 1 | GCGCTTGAGAAGATTAAATT | AGG | 665 |
| 4736 | 1 | TTATTAGTATTTTTTTTTTT | TGG | 666 |
| 4751 | 1 | TTTTTTGGTCTAATTTTAAT | TGG | 667 |
| 4752 | 1 | TTTTTGGTCTAATTTTAATT | GGG | 668 |
| 4802 | 1 | TGTTGCAGAGCTTATGCTAT | TGG | 669 |
| 4803 | 1 | GTTGCAGAGCTTATGCTATT | GGG | 670 |
| 4842 | -1 | ATATGTCAGCAATGTAATCT | TGG | 671 |
| 4870 | -1 | CAAGTGTTTGCTGCACTTTT | TGG | 672 |
| 4882 | 1 | CAAAAAGTGCAGCAAACACT | TGG | 673 |
| 4897 | -1 | TCTTCATTTTGGTATGGGCA | AGG | 674 |
| 4902 | -1 | TTTTCTCTTCATTTTGGTAT | GGG | 675 |
| 4903 | -1 | TTTTTCTCTTCATTTTGGTA | TGG | 676 |
| 4908 | -1 | TAGCCTTTTTCTCTTCATTT | TGG | 677 |
| 4916 | 1 | ATACCAAAATGAAGAGAAAA | AGG | 678 |
| 4922 | 1 | AAATGAAGAGAAAAAGGCTA | AGG | 679 |
| 4945 | -1 | TAATCAATTGTTTTTGATTT | TGG | 680 |
| 5012 | 1 | TGTAATTATGTCTTAATGAT | AGG | 681 |
| 5033 | 1 | GGACGTATACTAAAAGTGTG | TGG | 682 |
| 5078 | 1 | AATGAGTTCTGAATTTTTGA | AGG | 683 |
| 5098 | 1 | AGGACTTTTTGAATATTGTA | TGG | 684 |
| 5139 | 1 | TAATATAAAATTAATATATA | TGG | 685 |
| 5181 | 1 | TGATTTGTGTGTTTTGTGTG | AGG | 686 |
| 5187 | 1 | GTGTGTTTTGTGTGAGGTGC | AGG | 687 |
| 5188 | 1 | TGTGTTTTGTGTGAGGTGCA | GGG | 688 |
| 5214 | 1 | AGTTCTTTAGTGTCTAAATA | TGG | 689 |
| 5215 | 1 | GTTCTTTAGTGTCTAAATAT | GGG | 690 |
| 5232 | -1 | CAAATATGAAGATATGAAGC | TGG | 691 |
| 5249 | 1 | TCATATCTTCATATTTGTCT | TGG | 692 |
| 5268 | -1 | TAGTAATGCAATATATAATA | TGG | 693 |
| 5285 | 1 | TATATATTGCATTACTACCT | TGG | 694 |
| 5291 | -1 | TTTGGTTCTGCCAATAGCCA | AGG | 695 |
| 5292 | 1 | TGCATTACTACCTTGGCTAT | TGG | 696 |
| 5309 | -1 | AACTTAAAAACTACTCACTT | TGG | 697 |
| 5361 | 1 | CATATTCTATAAAATTAATA | TGG | 698 |
| 5401 | 1 | TTGAATTGCAGATGAGAAAA | TGG | 699 |
| 5410 | 1 | AGATGAGAAAATGGAAAGTT | TGG | 700 |
| 5411 | 1 | GATGAGAAAATGGAAAGTTT | GGG | 701 |
| 5414 | 1 | GAGAAAATGGAAAGTTTGGG | AGG | 702 |
| 5450 | 1 | ATTGAGTACATATATAGTAA | CGG | 703 |
| 5537 | 1 | TTGTATAATTAATTATTTTT | TGG | 704 |
| 5563 | 1 | CACTACAACTTATCTAACTC | AGG | 705 |
| 6711 | -1 | ATCTTTACATTCTTACTTTT | TGG | 706 |
| 6785 | 1 | TATATAAATATTCAATCAAA | TGG | 707 |
| 6789 | 1 | TAAATATTCAATCAAATGGT | TGG | 708 |
| 6811 | -1 | CTTGTAAATCTAAATCTCTC | AGG | 709 |
| 6837 | 1 | TTTACAAGAGACACATCATT | TGG | 710 |
| 6859 | 1 | GAAGAAGACATTTGAACATT | TGG | 711 |
| 6873 | -1 | TCCAAAGTGAAATTGGTGAT | TGG | 712 |
| 6880 | -1 | CTTACAATCCAAAGTGAAAT | TGG | 713 |
| 6883 | 1 | GCCAATCACCAATTTCACTT | TGG | 714 |
| 6927 | -1 | TTGTTTTCTTCTCTATAATA | AGG | 715 |
| 6973 | 1 | TCAAAAGTTTTTTATTATAT | AGG | 716 |
| 7030 | 1 | TTCTTGTTTATCAAATGATC | AGG | 717 |
| 7055 | 1 | TGCTTTTTCAGACAATTCTT | CGG | 718 |
| 7056 | 1 | GCTTTTTCAGACAATTCTTC | GGG | 719 |
| 7069 | 1 | ATTCTTCGGGTCAGTCACTA | AGG | 720 |
| 7089 | 1 | AGGTTGATTACATGACACTG | AGG | 721 |
| 7094 | 1 | GATTACATGACACTGAGGCA | TGG | 722 |
| 7105 | 1 | ACTGAGGCATGGATTTGTAA | TGG | 723 |
| 7126 | 1 | GGTATGTTGCACAATGATCT | TGG | 724 |
| 7137 | 1 | CAATGATCTTGGCCTGAAAA | TGG | 725 |
| 7138 | -1 | TGTAATTTGAAGCCATTTTC | AGG | 726 |
| 7203 | 1 | AGCTATGCTTTTCCCATTTC | AGG | 727 |
| 7204 | -1 | GAGCCAAATGTGCCTGAAAT | GGG | 728 |
| 7205 | -1 | GGAGCCAAATGTGCCTGAAA | TGG | 729 |
| 7212 | 1 | TTTCCCATTTCAGGCACATT | TGG | 730 |
| 7226 | -1 | TCAAATCTTGTTTCACTTTC | TGG | 731 |
| 7272 | 1 | CATCAGCAAATCACTTGATC | AGG | 732 |
| 7291 | 1 | CAGGATTTTGTAGTAATTGT | TGG | 733 |
| 7292 | 1 | AGGATTTTGTAGTAATTGTT | GGG | 734 |
| 7323 | -1 | ATATTATAAGCTGATTTCAA | AGG | 735 |
| 7419 | -1 | CGGCAACGAACCAAATTACT | GGG | 736 |
| 7420 | 1 | ATATATGCAGCCCAGTAATT | TGG | 737 |
| 7420 | -1 | ACGGCAACGAACCAAATTAC | TGG | 738 |
| 7439 | -1 | GTTGGACAGTAGAAACAATA | CGG | 739 |
| 7457 | -1 | CAATAACTTACCATATGTGT | TGG | 740 |
| 7458 | 1 | TTTCTACTGTCCAACACATA | TGG | 741 |
| 7519 | -1 | CAACATTTCAGTCACTGAAA | TGG | 742 |
| 7549 | 1 | GTTGTTCTTTTTTAATTAAC | AGG | 743 |
| 7568 | 1 | CAGGAATATACTCTTATTTG | TGG | 744 |
| 7583 | -1 | CTTACAATCAAAGGTAGAAA | TGG | 745 |
| 7592 | -1 | TGTGTTGTACTTACAATCAA | AGG | 746 |
| 7660 | -1 | TTCCACACATTAGCAAATGT | GGG | 747 |
| 7661 | -1 | TTTCCACACATTAGCAAATG | TGG | 748 |
| 7669 | 1 | GTCCCACATTTGCTAATGTG | TGG | 749 |
| 7699 | 1 | TTGTGATATATAAGATGAAT | AGG | 750 |
| 7715 | 1 | GAATAGGCTACTCCTTTTAT | AGG | 751 |
| 7716 | 1 | AATAGGCTACTCCTTTTATA | GGG | 752 |
| 7716 | -1 | CCATTTGAAAACCCTATAAA | AGG | 753 |
| 7727 | 1 | CCTTTTATAGGGTTTTCAAA | TGG | 754 |
| 7741 | -1 | ATTTAGGAATAAGATGAATG | GGG | 755 |
| 7742 | -1 | AATTTAGGAATAAGATGAAT | GGG | 756 |
| 7743 | -1 | GAATTTAGGAATAAGATGAA | TGG | 757 |
| 7757 | -1 | GACATACCATGTTAGAATTT | AGG | 758 |
| 7762 | 1 | CTTATTCCTAAATTCTAACA | TGG | 759 |
| 7788 | -1 | AAAAACCCAACACTGGAAAG | TGG | 760 |
| 7793 | 1 | TGTGTGCCACTTTCCAGTGT | TGG | 761 |
| 7794 | 1 | GTGTGCCACTTTCCAGTGTT | GGG | 762 |
| 7795 | -1 | ACAGGTCAAAAACCCAACAC | TGG | 763 |
| 7813 | -1 | AAATTTGTAGATTTTGAAAC | AGG | 764 |
| 7849 | -1 | CCAAATATCGGAAAATTTGT | GGG | 765 |
| 7850 | -1 | GCCAAATATCGGAAAATTTG | TGG | 766 |
| 7860 | 1 | CCCACAAATTTTCCGATATT | TGG | 767 |
| 7861 | -1 | AATCTCACAAGGCCAAATAT | CGG | 768 |
| 7872 | -1 | ACATTTGAAAGAATCTCACA | AGG | 769 |
| 7892 | 1 | ATTCTTTCAAATGTCACGTT | CGG | 770 |
| 7900 | 1 | AAATGTCACGTTCGGTCCTG | TGG | 771 |
| 7905 | -1 | AACGACCTTTCAGAGACCAC | AGG | 772 |
| 7911 | 1 | TCGGTCCTGTGGTCTCTGAA | AGG | 773 |
| 7935 | -1 | CGTTTGGGCCTGAAAAGTGT | GGG | 774 |
| 7936 | -1 | ACGTTTGGGCCTGAAAAGTG | TGG | 775 |
| 7938 | 1 | TCGTTATACCCACACTTTTC | AGG | 776 |
| 7950 | -1 | TTAATACACTCCTCACGTTT | GGG | 777 |
| 7951 | 1 | ACTTTTCAGGCCCAAACGTG | AGG | 778 |
| 7951 | -1 | CTTAATACACTCCTCACGTT | TGG | 779 |
| 7991 | 1 | AGTCTCACATTGCTAATGTA | TGG | 780 |
| 8020 | 1 | ATTGTGATATATAAAATGAA | TGG | 781 |
| 8021 | 1 | TTGTGATATATAAAATGAAT | GGG | 782 |
| 8038 | -1 | TAAAACTAATTGGCTGTGGG | AGG | 783 |
| 8041 | -1 | TCTTAAAACTAATTGGCTGT | GGG | 784 |
| 8042 | -1 | ATCTTAAAACTAATTGGCTG | TGG | 785 |
| 8048 | -1 | GGTTTTATCTTAAAACTAAT | TGG | 786 |
| 8069 | -1 | ATTTAGGGATAAGATGAATG | GGG | 787 |
| 8070 | -1 | AATTTAGGGATAAGATGAAT | GGG | 788 |
| 8071 | -1 | GAATTTAGGGATAAGATGAA | TGG | 789 |
| 8084 | -1 | ATTAAGCATGTTAGAATTTA | GGG | 790 |
| 8085 | -1 | GATTAAGCATGTTAGAATTT | AGG | 791 |
| 8144 | 1 | CAAATTGCAGATAATATTAC | TGG | 792 |
| 8147 | 1 | ATTGCAGATAATATTACTGG | TGG | 793 |
| 8148 | 1 | TTGCAGATAATATTACTGGT | GGG | 794 |
| 8180 | 1 | TCAAGTAATCATAACAAAGA | TGG | 795 |
| 8181 | 1 | CAAGTAATCATAACAAAGAT | GGG | 796 |
| 8202 | 1 | GGATTAAGCATTCAAGAGAG | AGG | 797 |
| 8210 | 1 | CATTCAAGAGAGAGGAGATG | TGG | 798 |
| 8217 | 1 | GAGAGAGGAGATGTGGTAAA | AGG | 799 |
| 8228 | 1 | TGTGGTAAAAGGTGCACCAT | TGG | 800 |
| 8233 | -1 | TCATCTCCTGGTTGAACCAA | TGG | 801 |
| 8238 | 1 | GGTGCACCATTGGTTCAACC | AGG | 802 |
| 8245 | -1 | AACCAGAAGAGGTCATCTCC | TGG | 803 |
| 8254 | 1 | AACCAGGAGATGACCTCTTC | TGG | 804 |
| 8256 | -1 | TAGGCCGTCCGAACCAGAAG | AGG | 805 |
| 8259 | 1 | GGAGATGACCTCTTCTGGTT | CGG | 806 |
| 8263 | 1 | ATGACCTCTTCTGGTTCGGA | CGG | 807 |
| 8275 | -1 | ATGAGAAAGAGCATTAATTT | AGG | 808 |
| 8301 | -1 | TAAGTACCTGAAAGAGAACA | AGG | 809 |
| 8306 | 1 | CATTCACCTTGTTCTCTTTC | AGG | 810 |
| 8413 | 1 | AAAATGATATCTTTTCTGCT | TGG | 811 |
| 8429 | 1 | TGCTTGGTACTAATTAATGC | TGG | 812 |
| 8487 | -1 | TACTGTACTCCATGCAAAAA | AGG | 813 |
| 8489 | 1 | TTCAACTTGCCTTTTTTGCA | TGG | 814 |
| 8531 | -1 | TGCCTTGAAACCAAAAATCA | AGG | 815 |
| 8532 | 1 | ATTTGACTTTCCTTGATTTT | TGG | 816 |
| 8540 | 1 | TTCCTTGATTTTTGGTTTCA | AGG | 817 |
| 8559 | 1 | AAGGCAATAAAATTATTACA | TGG | 818 |
| 8624 | -1 | TTCGTGGAAGCAAGTGTTCA | AGG | 819 |
| 8640 | -1 | TGATATCTTCAATTTTTTCG | TGG | 820 |
| 8669 | 1 | TATCATCATAAGAATTTCAA | TGG | 821 |
| 8670 | 1 | ATCATCATAAGAATTTCAAT | GGG | 822 |
| 8671 | 1 | TCATCATAAGAATTTCAATG | GGG | 823 |
| 8805 | 1 | TTCTCTTTTTCTTTCTTACT | AGG | 824 |
| 8819 | -1 | AACTGCATAGAACTTGTATG | AGG | 825 |
| 8853 | -1 | TGTGTGACAAGAGCATATAG | AGG | 826 |
| 8866 | 1 | TCTATATGCTCTTGTCACAC | AGG | 827 |
| 8893 | -1 | GATGATAATGATGATTTAGA | AGG | 828 |
| 8954 | 1 | ATTTGATCATATATTACAGA | TGG | 829 |
| 8955 | 1 | TTTGATCATATATTACAGAT | GGG | 830 |
| 8956 | 1 | TTGATCATATATTACAGATG | GGG | 831 |
| 8980 | -1 | ACTCTGTCATTGAAAATTAC | TGG | 832 |
| 9013 | 1 | TAGCAACAGCATTAAAGAAC | TGG | 833 |
| 9027 | -1 | TGTTCTTGGTTTTGGCTGAA | TGG | 834 |
| 9035 | -1 | GTGTTTTTTGTTCTTGGTTT | TGG | 835 |
| 9041 | -1 | TCGGTTGTGTTTTTTGTTCT | TGG | 836 |
| 9059 | 1 | CAAAAAACACAACCGAAATT | CGG | 837 |
| 9060 | -1 | GCGAGTTTGTCTCCGAATTT | CGG | 838 |
| 9082 | -1 | GTTGCAGGCCTACTTGAGAA | TGG | 839 |
| 9085 | 1 | CAAACTCGCCATTCTCAAGT | AGG | 840 |
| 9097 | -1 | ATGCCATATGTTGGAGTTGC | AGG | 841 |
| 9105 | 1 | AGGCCTGCAACTCCAACATA | TGG | 842 |
| 9106 | -1 | ACTGGAGACATGCCATATGT | TGG | 843 |
| 9124 | -1 | TAATTTTGCAGCAGATGAAC | TGG | 844 |
| 9156 | 1 | TACAGAAGCACAGCAACTGA | TGG | 845 |
| 9165 | 1 | ACAGCAACTGATGGATACTA | TGG | 846 |
| 9175 | 1 | ATGGATACTATGGTTCTCCG | AGG | 847 |
| 9181 | -1 | TTTTCGACATTAGACATCCT | CGG | 848 |
| 9213 | 1 | AACGATTACTATGAGCCTGA | AGG | 849 |
| 9214 | 1 | ACGATTACTATGAGCCTGAA | GGG | 850 |
| 9217 | -1 | TTGGGAGATGGTGTCCCTTC | AGG | 851 |
| 9229 | -1 | GATGGTCCATTGTTGGGAGA | TGG | 852 |
| 9234 | 1 | GGGACACCATCTCCCAACAA | TGG | 853 |
| 9235 | -1 | GCTGCAGATGGTCCATTGTT | GGG | 854 |
| 9236 | -1 | TGCTGCAGATGGTCCATTGT | TGG | 855 |
| 9247 | -1 | TGTATTTCACTTGCTGCAGA | TGG | 856 |
| 9284 | 1 | GAATAACTATGAAGTTGAGA | AGG | 857 |
| 9296 | 1 | AGTTGAGAAGGATATAAGTG | AGG | 858 |
| 9300 | 1 | GAGAAGGATATAAGTGAGGA | AGG | 859 |
| 9311 | 1 | AAGTGAGGAAGGACAGCCAA | TGG | 860 |
| 9316 | -1 | GAGCTTGGTTCCTGAACCAT | TGG | 861 |
| 9317 | 1 | GGAAGGACAGCCAATGGTTC | AGG | 862 |
| 9331 | -1 | TTTTGCTGTGAGGAGGAGCT | TGG | 863 |
| 9338 | -1 | GACCTCATTTTGCTGTGAGG | AGG | 864 |
| 9341 | -1 | CTTGACCTCATTTTGCTGTG | AGG | 865 |
| 9347 | 1 | CTCCTCCTCACAGCAAAATG | AGG | 866 |
| 9368 | -1 | CCTAAATGAGAAGTGAGATA | AGG | 867 |
| 9379 | 1 | CCTTATCTCACTTCTCATTT | AGG | 868 |
| 9450 | 1 | CTTTATTTCTTATTATCTTT | TGG | 869 |
| 9498 | 1 | AATATGTATAAGCTTGAATT | TGG | 870 |

Reference is made to Table 4 summarizing sequences relating to WT CsMLO within the scope of the current invention.

TABLE 4
WT CsMLO sequence table

| Sequence type | | | |
|---|---|---|---|
| characterization | CsMLO1 | CsMLO2 | CsMLO3 |
| Genomic | SEQ ID NO: 1 | SEQ ID NO: 4 | SEQ ID NO: 7 |
| sequence | | | |
| Coding | SEQ ID NO: 2 | SEQ ID NO: 5 | SEQ ID NO: 8 |
| sequence | | | |
| (CDS) | | | |
| Amino acid | SEQ ID NO: 3 | SEQ ID NO: 6 | SEQ ID NO: 9 |
| sequence | | | |
| gRNA | SEQ ID NO: 10- | SEQ ID NO: 287- | SEQ ID NO: 626- |
| sequence | SEQ ID NO: 286 | SEQ ID NO: 625 | SEQ ID NO: 870 |
| | (Table 1) | (Table 2) | (Table 3) |

The above gRNA molecules have been cloned into suitable vectors and their sequence has been verified. In addition different Cas9 versions have been analyzed for optimal compatibility between the Cas9 protein activity and the gRNA molecule in the Cannabis plant.

Stage 3: Transforming Cannabis plants using Agrobacterium or biolistics (gene gun) methods. For Agrobacterium and bioloistics a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various Cannabis tissues was performed using particle bombardment of:
DNA vectors
Ribonucleoprotein complex (RNP's) According to further embodiments of the present invention, transformation of various Cannabis tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

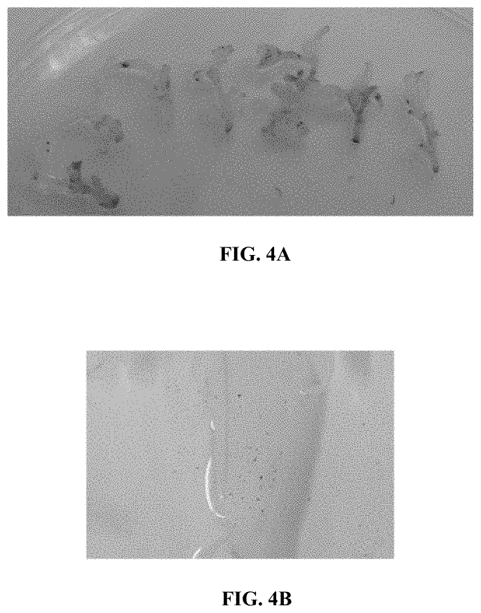

Reference is now made to FIG. 4A-D photographically presenting GUS staining after transient transformation of the following Cannabis tissues (A) axillary buds (B) leaf (C) calli, and (D) cotyledons.

FIG. 4 demonstrates that various Cannabis tissues have been successfully transiently transformed using biolistics system. Transformation has been performed into calli, leaves, axillary buds and cotyledons of Cannabis.

According to further embodiments of the present invention, additional transformation tools were used in Cannabis, including, but not limited to:
Protoplast PEG transformation
Extend RNP use
Directed editing screening using fluorescent tags
Electroporation

Stage 4: Regeneration in tissue-culture. When transforming DNA constructs into the plant, antibiotics is used for selection of positive transformed plants. An improved regeneration protocol was herein established for the Cannabis plant.

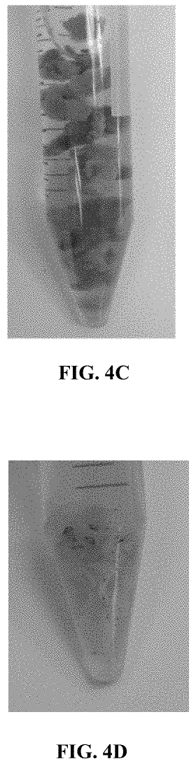

Reference is now made to FIG. 5 presenting regeneration of Cannabis tissue. In this figure, arrows indicate new meristem emergence.

Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

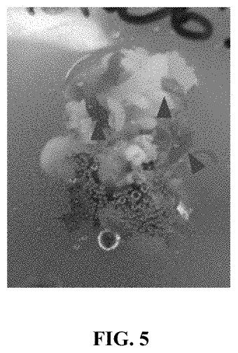

Reference is now made to FIG. 6 showing PCR detection of Cas9 DNA in shoots of transformed Cannabis plants. DNA extracted from shoots of plants transformed with Cas9 using biolistics. This figure shows that three weeks post transformation, Cas9 DNA was detected in shoots of transformed plants.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

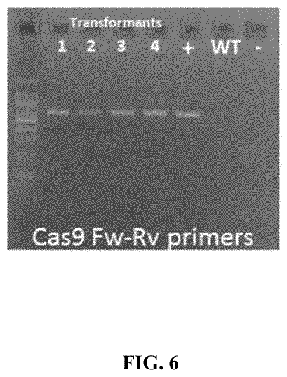

Reference is now made to FIG. 7 presenting results of in vitro analysis of CRISPR/Cas9 cleavage activity. FIG. 7A schematically shows the genomic area targeted for editing (PAM is marked in red) and amplified by the reverse and forward designed primers FIG. 7B photographically presents a gel showing successful digestion of the resulted PCR amplicon containing the gene specific gRNA sequence, by RNP complex containing Cas9. The analysis included the following steps:
1) Amplicon was isolated from two exemplified Cannabis strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Stage 6: Selection of transformed Cannabis plants presenting resistance to PM by establishing a protocol adapted for Cannabis. It is within the scope that different gRNA promoters were tested in order to maximize editing efficiency.

### Example 2

Identifying powdery mildew (PM) pathogen specific for Cannabis Powdery Mildew is one of the most destructive fungal pathogens infecting Cannabis. It is an obligate biotroph that can vascularize into the plant tissue and remain invisible to a grower. Under ideal conditions, powdery mildew has a 4-7 days post inoculation (dpi) window where it remains invisible as it builds a network internally in the plant. It is herein acknowledged that the powdery mildew vascularized network in Cannabis is detectable with a PCR DNA based test prior to conidiospore generation. At later stages, powdery mildew infection and conidiospore generation results in rapid spreading of the fungus to other plants. This tends to emerge and sporulate within 2 weeks into flowering thus destroying very mature crops with severe economic consequences. DNA based tools could facilitate early detection and rapid removal of infected plant materials or screening of incoming clones.

To date, there are no fungal disease resistant Cannabis varieties on the market. Golovinomyces cichoracearum is known for causing PM on several Cucurbits and on Cannabis (Pepin et al., 2018). In order to identify the specific fungi type affecting Cannabis, a molecular analysis has been performed. Internal Transcribed Spacer (ITS) DNA of PM samples obtained from Cannabis strains growing in our greenhouse has been isolated and sequenced. The term Internal transcribed spacer (ITS) as used hereinafter refers to the spacer DNA region situated between the small-subunit ribosomal RNA (rRNA) and large-subunit rRNA genes in the chromosome or the corresponding transcribed region in the polycistronic rRNA precursor transcript. It is herein acknowledged that the internal transcribed spacer (ITS) region is considered to have the highest probability of successful identification for the broadest range of fungi, with the most clearly defined barcode gap between inter- and intraspecific variation. Thus ITS is proposed for adoption as the primary fungal barcode marker, namely as potential DNA marker or finger print for fungi (Schoch C. L. et al, PNAS, 2012 109 (16) 6241-6246). The results of the molecular analysis of PM isolated from Cannabis revealed that Golovinomyces ambrosiae or Golovinomyces cichoracearum are the cause of the disease.

A further achievement of the present invention is the establishment of an inoculation assay and index for Cannabis, or in other words establishment of bio-assay for powdery mildew inoculation in Cannabis. Such an assay establishment may include:
Development of susceptibility index
Designing a protocol by testing different inoculation approaches at several plant developmental stages

### Example 3

Production of Genome-Edited Cannabis MLO (CsMLO) Genes

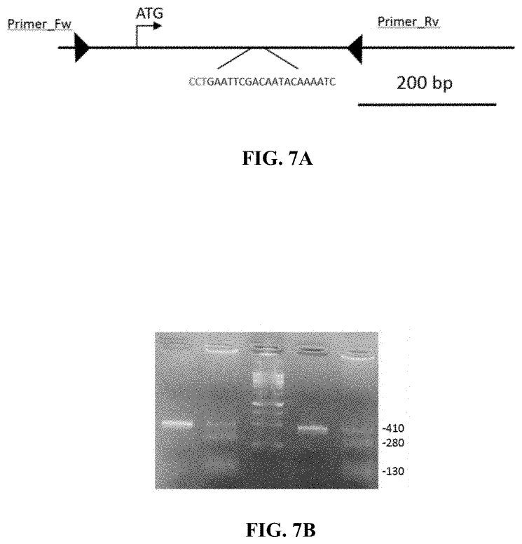

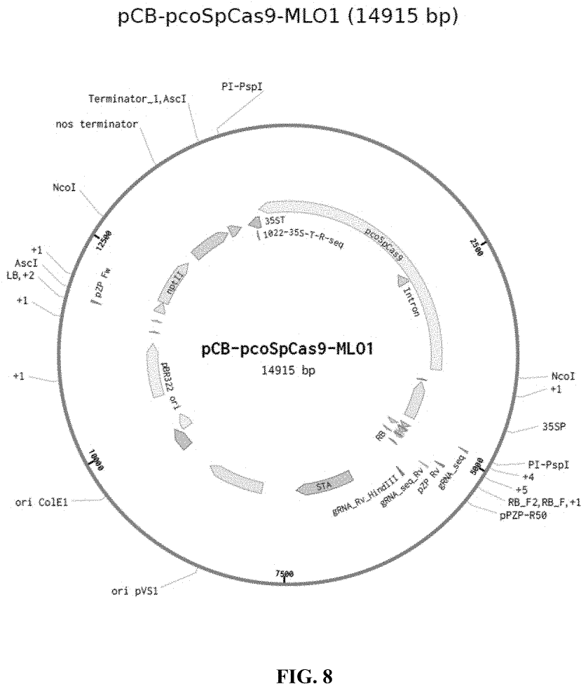

Three single guide RNAs (sgRNA) targeting the first exon (exon 1) of the CsMLO1 gene were designed and synthesized. These sgRNAs include sgRNA having nucleotide sequence as set forth in SEQ ID NO:17 (first guide), SEQ ID NO:43 (second guide) and SEQ ID NO:50 (third guide) starting at position 99, 369 and 453 of SEQ ID NO:1. The predicted Cas9 cleavage sites directed by these guide RNAs were designed to overlap with the nucleic acid recognition site of the restriction enzymes: Hinf1, BseLI and BtsI for the first, second and third gRNA, respectively (see FIG. 9). Transformation was performed using a DNA plasmid such as a plant codon optimized Streptococcus pyogenes Cas9 (pcoSpCas9) plasmid presented in FIG. 8. The plasmid contained the plant codon optimized SpCas9 and the above mentioned at least one sgRNA.

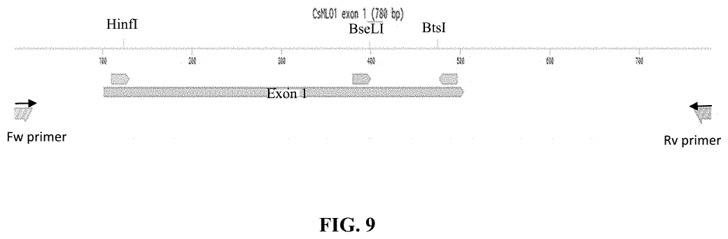

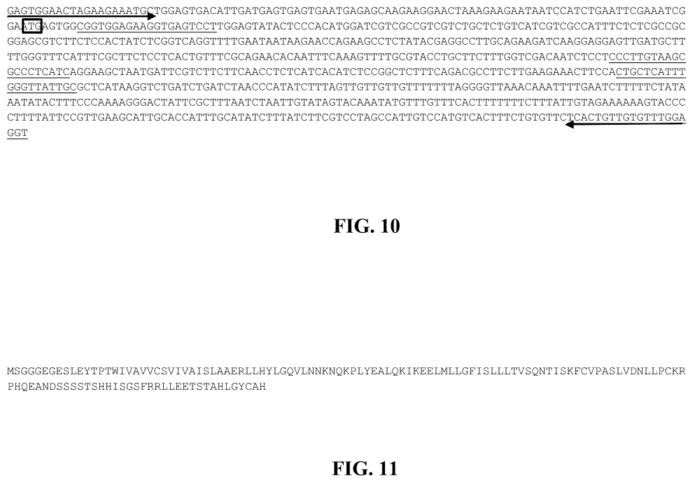

About two months post transformation, leaves from mature plants were sampled, and their DNA was extracted and digested with the suitable enzymes. Digested genomic DNA was used as a template for PCR using a primer pair flanking the 5′ and 3′ ends of the first exon of CsMLO1. The forward primer (fwd) (5-GAGTGGAACTAGAAGAAATGC-3) comprises a nucleotide sequence as set forth in SEQ ID NO:871, and the reverse primer (rev) (5-CCCTCCAAACACAACAGTGA-3) comprises a nucleotide sequence as set forth in SEQ ID NO:872 (see FIG. 9 and FIG. 10). As shown in FIG. 10, the aforementioned primer pair (marked with arrows) generates a 778 bp amplicon comprising the entire exon 1 of CsMLO1, having a nucleotide sequence as set forth in SEQ ID NO:873 (nucleotide positions 4-782 of SEQ ID NO:1). In FIG. 10 the three gRNA sequences used to target exon 1 of CsMLO1 genomic sequence are underlined. The translation initiation codon ATG (encoding Methionine amino acid) is marked with a square. FIG. 11 presents the amino acid sequence of CsMLO1 first exon as set forth in SEQ ID NO:874.

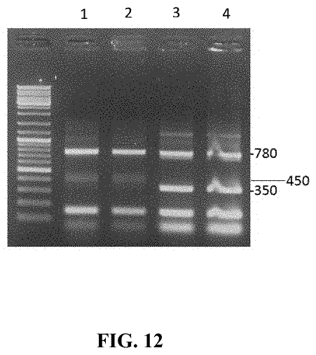

Reference is now made to FIG. 12 photographically presenting detection of CsMLO1 PCR products showing length variation (i.e. truncated fragments) as a result of Cas9-mediated genome editing. DNA from plants two months post transformation was used as a template for the PCR using primers having nucleic acid sequence as set forth in SEQ ID NO:871 and SEQ ID NO:872. DNA fragments shorter than the expected WT 780 bp amplicon were obtained by the PCR reaction and subcloned into a sequencing plasmid and sequenced. The sequencing results are described below.

It can be seen in FIG. 12 that WT or non-edited PCR products result in a 780 bp band, while DNA extracted from edited plants exhibit a shorter band than the expected 780 bp WT exon 1 length, i.e. samples 1 and 2 show a 450 bp fragment and samples 3 and 4 show a 350 bp fragment.

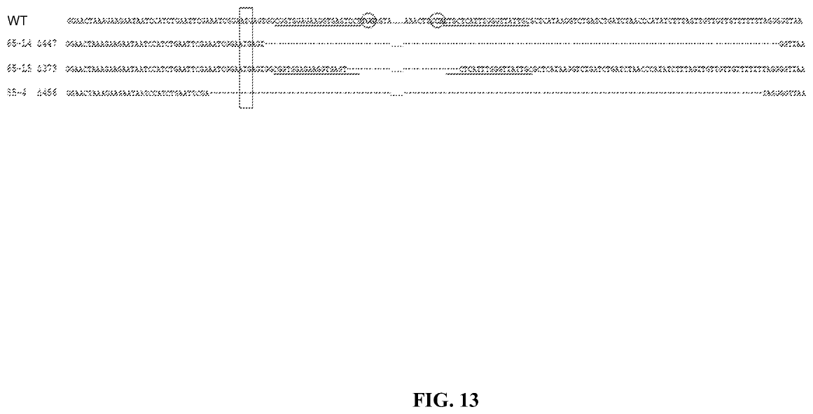

FIG. 13 schematically presents sequences of WT and genome edited CsMLO1 DNA fragments obtained for the first time by the present invention. In this figure, sgRNA sequences are underlined. sgRNA having nucleotide sequence as set forth in SEQ ID NO:17 (first guide) with Hinf1 restriction site appears on the left hand of exon 1, and sgRNA having nucleotide sequence as set forth in SEQ ID NO:50 (third guide) with BtsI restriction site appears on the right hand of exon 1 fragments. PAM sequences (NGG) are in marked italics and bold and are circled. ATG codon position is marked with a square.

The sequencing results show that three CsMLO1 exon 1 genome edited fragments were achieved by the present invention.

Reference is now made to Table 5 summarizing sequences relating to mutated (genome edited) exon 1 fragments of CsMLO1 achieved by the current invention.

TABLE 5
Sequences of mutated CsMLO1 exon 1

| | | 65-L4 (Δ447) | 65-L5 (Δ373) | 85-4 (Δ456) |
|---|---|---|---|---|
| | Exon 1 of WT | fragment of | fragment of | fragment of |
| Sequence type | CsMLO1 | CsMLO1 | CsMLO1 | CsMLO1 |
| Genomic sequence | SEQ ID NO: 873 | SEQ ID NO: 875 | SEQ ID NO: 877 | SEQ ID NO: 880 |
| (Position in SEQ | (nucleic acid 4-782 | (deletion of nucleic | (deletion of nucleic | (deletion of nucleic |
| ID NO: 1) | in SEQ ID NO: 1) | acid 109-556 in | acid 128-501 in | acid 96-552 in |
| | (FIG. 10) | SEQ ID NO: 1) | SEQ ID NO: 1) | SEQ ID NO: 1) |
| | | (FIG. 13) | (FIG. 13) | (FIG. 13) |
| Deleted nucleic | | SEQ ID NO: 876 | SEQ ID NO: 879 | SEQ ID NO: 881 |
| acid sequence | | | | |
| Amino acid | SEQ ID NO: 874 | SEQ ID NO: 887 | SEQ ID NO: 878 | No amino-acid |
| sequence | (FIG. 11) | MS | MSGGGEGE | sequence is |
| | | | | produced |
| gRNA sequence | SEQ ID NO: 17, | | | |
| targeted to Exon | SEQ ID NO: 43 | | | |
| 1 of CsMLO1 | and SEQ ID NO: | | | |
| | 50 (Table 1) | | | |

The resulted mutated CsMLO1 fragments include the following:
(1) Fragment 1: CsMLO1 fragment marked as 65-L4 Δ447 comprises a nucleotide sequence as set forth in SEQ ID NO:875 (about 330 bp). This fragment contains a deletion of 447 bp (position 109-556 of SEQ ID NO:1) having a nucleotide sequence as set forth in SEQ ID NO:876. It should be noted that this fragment encodes a two amino acid peptide (SEQ ID NO:887, as shown in Table 5). The short CsMLO1 exon 1 peptide generated by the targeted genome editing is expected to result is a non-functional, silenced CsMLO1 gene or allele.
(2) Fragment 2: CsMLO1 fragment marked as 65-L5 Δ373 comprises a nucleotide sequence as set forth in SEQ ID NO:877 (about 405 bp). This fragment contains a deletion of 373 bp (position 128-501 of SEQ ID NO:1) having a nucleotide sequence as set forth in SEQ ID NO:879. It should be noted that this fragment encodes a short peptide of eight amino acids (SEQ ID NO:878, as shown in Table 5). Such a short exon 1 fragment is expected to result in a non-functional CsMLO1 allele.
(3) Fragment 3: CsMLO1 fragment marked as 85-4 Δ456 comprises a nucleotide sequence as set forth in SEQ ID NO:880 (about 320 bp). This fragment contains a deletion of 456 bp (position 96-552 of SEQ ID NO:1) having a nucleotide sequence as set forth in SEQ ID NO:881. It is emphasized that fragment 3 was edited such that it lacks the ATG translation start codon, therefore no translated protein is generated. The resulted truncated CsMLO1 gene/protein is expected to be non-functional.

The genome-edited CsMLO1 truncated fragments of the present invention are characterized by deletion of significant parts of the first exon sequence of CsMLO1 gene. Thus these genome edited fragments produce truncated CsMLO1 proteins. The truncated proteins lack significant part of the Open Reading Frame (ORF), e.g. absent of the translation start codon or significant part of exon-1 protein encoding sequence, and therefore would be non-functional.

### Example 4

Production of Mutated Csmlo1 Gene by Genome Editing Events

This example presents the production of new genome editing events within CsMLO1 gene. A mutated Csmlo1 allele has been generated encompassing at least one of the following genome editing events within CsMLO1 gene:
1. indel_d14-14 pb deletion (bp 389-402 of SEQ ID NO:1) having SEQ ID NO:883
2. i1-1 bp insertion of A (bp 482-483 of SEQ ID NO:1)

As compared to the WT CsMLO1_378-500 fragment of SEQ ID NO:1 having a nucleic acid sequence as set forth in SEQ ID NO:882, the mutated Csmlo1 allele may comprise one or more of the following mutated DNA fragments:
Csmlo1_d14i1 encompassing the above identified deletion and insertion events, comprising a nucleic acid sequence corresponding to the sequence as set forth in SEQ ID NO:886.
Csmlo1_d14 encompassing the above identified deletion event, comprising a nucleic acid sequence corresponding to the sequence as set forth in SEQ ID NO:884.
Csmlo1_i1 encompassing the above identified insertion event, comprising a nucleic acid sequence corresponding to the sequence as set forth in SEQ ID NO:885.

The sequence of the mutated DNA fragments Csmlo1_d14i, Csmlo1_d14, Csmlo1_i1, as well as the presence of a deletion indel_d14 and/or insertion i1 provided by the present invention, is useful to identify and generate Cannabis plants with mutated alleles of MLO1 gene, desirable for the production of Cannabis plants with PM resistance.

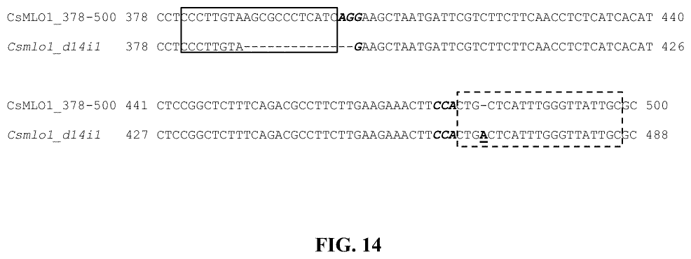

Reference is now made to FIG. 14, schematically presenting sequence comparison (homology) between WT CsMLO1_378-500 fragment of SE ID NO:1, having a nucleic acid sequence as set forth in SEQ ID NO:882, and genome edited Csmlo1_d14i1 fragment, having a nucleic acid sequence as set forth in SEQ ID NO:886. In this figure, the deleted nucleic acids within Csmlo1_d14i1 fragment are marked with dashed line, and the inserted nucleotide (A) is underlined and marked in bold. The gRNA designed to target the deletion region (having a sequence as set forth in SEQ ID NO:43) comprises BslI restriction site and is marked with a square having a continuous line. The gRNA designed to target the insertion region (having a nucleotide sequence complementary to the nucleotide sequence as set forth in SEQ ID NO:50) comprises BtsI restriction site and is marked with a square having a dashed line. PAM sequence regions are marked in bold and italics.

As shown in this figure, specific genome editing events within CsMLO1 gene are generated by the present invention resulting in mutated Csmlo1 alleles (such as alleles comprising Csmlo1_d14, Csmlo1_i1 and Csmlo1_d14i1 fragments). According to one embodiment, a mutated Csmlo allele comprising a deletion of 14 bp (having SEQ ID NO:883) at position corresponding to position 12 of SEQ ID NO: 882 is produced (Csmlo allele containing Csmlo1_d14 fragment). According to a further embodiment, a mutated Csmlo allele comprising a nucleic acid insertion of A at position corresponding to position 104-105 of SEQ ID NO: 882 is produced (Csmlo allele containing Csmlo1_i1 fragment). According to yet another embodiment, a mutated Csmlo allele comprising a deletion of 14 bp (having SEQ ID NO:883) at position corresponding to position 12 of SEQ ID NO: 882 in combination with a nucleic acid insertion of A at position corresponding to position 104-105 of SEQ ID NO: 882 (Csmlo allele containing Csmlo1_d14i1 fragment) is produced.

The genome editing events herein described introduce mutations that silence or significantly reduce CsMLO1 gene expression or function in the plant.

By silencing genes encoding MLO proteins (e.g. CsMLO1, CsMLO2 and/or CsMLO3) in Cannabis, plants with enhanced resistance to Powdery Mildew disease, can be produced. These PM resistant plants are highly desirable for the medical Cannabis industry since usage of chemical agents to control pathogen diseases is significantly reduced or avoided.

1. A modified Cannabis plant exhibiting enhanced resistance to powdery mildew (PM), wherein said modified plant comprises a mutated Cannabis mlo1 (Csmlo1) allele, said mutated allele comprising a genomic modification selected from an indel of 14 bp at position corresponding to position 12 of SEQ ID NO: 882, or a fraction thereof, or a nucleic acid insertion at position corresponding to position 104-105 of SEQ ID NO: 882, or a combination thereof.

2. The modified Cannabis plant according to claim 1, wherein said indel comprises a sequence as set forth in SEQ ID NO:883 or a fraction thereof.

3. The modified Cannabis plant according to claim 1, wherein said Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof.

4. The modified Cannabis plant according to claim 1, wherein said Csmlo1 mutant allele is at least one of:
a. comprises a nucleic acid sequence corresponding to the sequence as set forth in SEQ ID NO:886, or a homologue having at least 80% sequence identity to the nucleic acid sequence of said mutated Csmlo1 allele;
b. confers an enhanced resistance to powdery mildew as compared to a Cannabis plant comprising a wild type CsMLO1 allele having a nucleic acid sequence with at least 80% sequence identity to a nucleic acid sequence as set forth in SEQ ID NO:882 and/or to a nucleic acid sequence as set forth in SEQ ID NO:1;
c. comprising a deletion of 14 bp at position 389 of SEQ ID NO: 1, or a nucleic acid insertion at position 482-483 of SEQ ID NO: 1, or a combination thereof; and
d. generated using genome editing.

5. The modified Cannabis plant according to claim 1, wherein said plant has decreased expression levels of Mlo1 protein, relative to a Cannabis plant lacking said mutated Csmlo1 allele.

6. The modified Cannabis plant according to claim 1, wherein said genome modification is generated via introduction (a) Cas DNA and gRNA sequence selected from the group consisting of SEQ ID NO:17, SEQ ID NO:43 and SEQ ID NO:50 and any combination thereof, or (b) a ribonucleoprotein (RNP) complex comprising Cas protein and gRNA sequence selected from the group consisting of SEQ ID NO:17, SEQ ID NO:43 and SEQ ID NO:50 and any combination thereof.

7. The modified Cannabis plant according to claim 1, wherein said PM is selected from the group consisting of Golovinomyces cichoracearum, Golovinomyces ambrosiae and a mixture thereof.

8. A progeny plant, plant part, plant seed, tissue culture of regenerable cells, protoplasts, callus or plant cell of a modified plant according to claim 1.

9. The modified Cannabis plant according to claim 1, wherein said modified plant comprises a targeted genome modification conferring reduced expression of a Cannabis MLO1 (CsMLO1) gene as compared to a Cannabis plant lacking said targeted genome modification, said targeted genome modification generates a mutated Cannabis mlo1 (Csmlo1) allele comprising a deletion of a nucleic acid sequence as set forth in SEQ ID NO:883 or a fraction thereof as compared to the wild type CsMLO1 allele comprising a sequence as set forth in SEQ ID NO:1, or a nucleic acid insertion at position 482-483 of SEQ ID NO:1, or a combination thereof.

10. A method for producing a modified Cannabis plant according to claim 1, said method comprises introducing using targeted genome modification, at least one genomic modification conferring reduced expression of at least one Cannabis MLO1 (CsMLO1) allele as compared to a Cannabis plant lacking said targeted genome modification, said genomic modification generates a mutated Cannabis mlo1 (Csmlo1) allele comprising an indel of 14 bp at a position corresponding to position 12 of SEQ ID NO: 882 or a fraction thereof, or a nucleic acid insertion at position corresponding to position 104-105 of SEQ ID NO: 882, or a combination thereof.

11. The method according to claim 10, comprises at least one step of:
a. introducing a loss of function mutation into said CsMLO1 allele using targeted genome modification;
b. introducing an expression vector comprising a promoter operably linked to a nucleotide sequence encoding a plant optimized Cas9 endonuclease and gRNA targeted to at least one CsMLO1 allele, said gRNA nucleotide sequence targeting said CsMLO1 allele is selected from the group consisting of SEQ ID NO:17, SEQ ID NO:43 and SEQ ID NO:50 or a complementary sequence thereof;
c. introducing and co-expressing in a Cannabis plant Cas9 and gRNA targeted to CsMLO1 gene and screening for induced targeted mutations conferring reduced expression of said CsMLO1 gene;
d. selecting a plant resistant to powdery mildew from plants comprising mutated Csmlo1 allele, said selected plant is characterized by enhanced resistance to powdery mildew as compared to a Cannabis plant comprising a CsMLO1 nucleic acid comprising a nucleic acid sequence as set forth in SEQ ID NO:882;
e. regenerating a plant carrying said genomic modification; and
f. screening said regenerated plants for a plant resistant to powdery mildew.

12. The method according to claim 10, wherein at least one of the following holds true:
a. said indel comprises a sequence as set forth in SEQ ID NO:883 or a fraction thereof;
b. said modified plant has decreased levels of at least one Mlo protein as compared to wild type Cannabis plant;
c. said genome modification is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof;
d. said genetic modification in said CsMLO1 is generated in planta via introduction of a construct comprising (a) Cas DNA and gRNA sequence selected from the group consisting of SEQ ID NO:17, SEQ ID NO:43 and SEQ ID NO:50 or a complementary sequence thereof, and any combination thereof, or (b) a ribonucleoprotein (RNP) complex comprising Cas protein and gRNA sequence selected from the group consisting of SEQ ID NO:17, SEQ ID NO:43 and SEQ ID NO:50 or a complementary sequence thereof, and any combination thereof;
e. said powdery mildew is selected from the group of species consisting of Golovinomyces cichoracearum, Golovinomyces ambrosiae and a mixture thereof;
f. said Cannabis plant is selected from the group of species that includes, but is not limited to, Cannabis sativa (C. sativa), C. indica, C. ruderalis and any hybrid or cultivated variety of the genus Cannabis;
g. said Csmlo1 mutant allele comprises a nucleic acid sequence corresponding to the sequence as set forth in SEQ ID NO:884, or a nucleic acid sequence corresponding to the sequence as set forth in SEQ ID NO:885, or a nucleic acid sequence corresponding to the sequence as set forth in SEQ ID NO:886, or a homologue having at least 80% sequence identity to the nucleic acid sequence of said mutated Csmlo1 allele, or a complementary sequence thereof, or any combination thereof;
h. said Csmlo1 mutant allele comprises a nucleic acid sequence corresponding to the sequence as set forth in SEQ ID NO:886, or a homologue having at least 80% sequence identity to the nucleic acid sequence of said mutated Csmlo1 allele;
i. said mutated Csmlo1 allele confers an enhanced resistance to powdery mildew as compared to a Cannabis plant comprising a wild type CsMLO1 allele having a nucleic acid sequence at least 80% sequence identity to a sequence as set forth in SEQ ID NO:882 and/or to a nucleic acid sequence as set forth in SEQ ID NO:1; and
j. said mutated allele comprising a deletion of 14 bp at position 389 of SEQ ID NO: 1, or a nucleic acid insertion at position 482-483 of SEQ ID NO: 1, or a combination thereof.

13. A method for conferring powdery mildew resistance to a Cannabis plant comprising producing a plant according to the method of claim 10.

14. A plant, plant part, plant cell, tissue culture or a seed obtained or obtainable by the method of claim 10.

15. A method for identifying a Cannabis plant with resistance to powdery mildew, said method comprises steps of:
a. screening the genome of said Cannabis plant for a mutated Csmlo1 allele, said mutated allele comprises a genomic modification selected from an indel of 14 bp at a position corresponding to position 12 of SEQ ID NO: 882 or a fraction thereof, or a nucleic acid insertion at position corresponding to position 104-105 of SEQ ID NO: 882, or a combination thereof;
b. optionally, regenerating plants carrying said genetic modification; and
c. optionally, screening said regenerated plants for a plant resistant to powdery mildew.

16. An isolated polynucleotide sequence having at least 80% sequence identity to a nucleic acid sequence selected from the group consisting of SEQ ID NO:882-886, SEQ ID NO:17, SEQ ID NO:43 and SEQ ID NO:50 or a complementary sequence or any combination thereof.

17. Use of the polynucleotide sequence according to claim 16, for generating, identifying and/or screening for a Cannabis plant comprising within its genome mutant Csmlo allele conferring resistance to PM, wherein the presence of at least one nucleic acid sequence selected from the group consisting of SEQ ID NO:1, SEQ ID NO:883, SEQ ID NO:882 indicates that the Cannabis plant comprises a wild type CsMLO1 allele, and the presence of at least one nucleic acid sequence selected from the group consisting of SEQ ID NO:884, SEQ ID NO:885 and SEQ ID NO:886 indicates that the Cannabis plant comprises a mutant Csmol1 allele.

18. A detection kit for determining the presence or absence of a mutant Csmlo1 allele in a Cannabis plant, said kit comprising at least one of the isolated polynucleotide sequence according to claim 16, said kit is useful for identifying a Cannabis plant with enhanced resistance to powdery mildew, wherein the presence of at least one nucleic acid sequence selected from the group consisting of SEQ ID NO:1, SEQ ID NO:883, SEQ ID NO:882 indicates that the Cannabis plant comprises a wild type CsMLO1 allele, and the presence of at least one nucleic acid sequence selected from the group consisting of SEQ ID NO:884, SEQ ID NO:885 and SEQ ID NO:886 indicates that the Cannabis plant comprises a mutant Csmol1 allele.

19. A method of determining the presence of a mutant Csmlo1 allele in a Cannabis plant using the isolated polynucleotide sequence according to claim 16, comprising assaying said Cannabis plant for at least one of the presence of an indel comprising a nucleic acid sequence as set forth in SEQ ID NO:883, an insertion at position 104-105 of SEQ ID NO: 882, a nucleic acid sequence corresponding to the sequence as set forth in SEQ ID NO:884, a nucleic acid sequence corresponding to the sequence as set forth in SEQ ID NO:885, a nucleic acid sequence corresponding to the sequence as set forth in SEQ ID NO:886, or a homologue having at least 80% sequence identity to the nucleic acid sequence of said mutated Csmlo1 allele, a complementary sequence thereof, or any combination thereof.

20. A method for down regulation of Cannabis MLO1 (CsMLO1) gene, which comprises utilizing the isolated polynucleotide sequence according to claim 16, by steps of utilizing the nucleotide sequence as set forth in at least one of SEQ ID NO:43 and SEQ ID NO:50 or a complementary sequence thereof, and a combination thereof, for introducing a loss of function mutation into said CsMLO1 gene using targeted genome modification.


SEQUENCE LISTING (886 sequences)

SEQ ID NO: 1 - DNA, 4363 bp, Cannabis sativa
  misc_feature 1..4363: note = Genomic sequence of CsMLO1; note = The gRNA sequences SEQ ID NO: 10 - SEQ ID NO: 286 are the respective CsMLO1 positions targeted gRNA
  source 1..4363: mol_type = genomic DNA
agtgagtggaactagaagaaatgctggagtgacattgatgagtgagtgaatgagagcaagaaggaactaaagaagaataatccatctgaattcgaaatcggaatgagtggcggtggagaaggtgagtccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcataaggtctgatctgatctaacccatatctttagttgttgttgtttttttaggggttaaacaaaattttgaatctttttctataaatatactttcccaaaagggactattcgctttaatctaattgtaatagtacaaatatgtttgtttcactttttttctttattgtagaaaaaagtaccccttttatccgttgaagcattgcaccatttgcatatctttatcttcgtctagccattgtccatgtcactttctgtgttctcactgttgtgtttggaggggtaaaggtaagtatatcttccaaattcatatgcttctctctcttttatactctactactttgtttttgttcgtttatgagaaatatttctacacaactagatacgtcaatggaaacgttgggaggattctattgcaaatgagagttatgacactgaacaaggtaagtattctcagtaccacttttgacaatattatagaaaatttgtctgactgtattccttctaacatatgaatcgcttaacagctgttcttaagtctaagaaaaaggtaacaagcgtccatcaacacgcgttcattaaggaccactttttgggttttggtaaagactcagctctactaggctggctggtaagtaactctttgatttgtacgtttagtatccaaagttctgaattaggattattcttatggtctccgttttcttattctcttgatattatattcttcttttattccactttctattttgagaacgttgttgcttagttaagaaacgtgagaagaactccattgatactgagcctttggagattttctccatcacatgaaaactaggacatttttgtgcacatgttaaggagctagctaacattaacattggaaacaggacatcgctaaaccatgatttgattctttttccccttgtgaacagcatgcttttgttaagcaattttatgcatctgtgaccaaatcagattatgcgaccatgcggttaggattcatcatggtaagaatcagagcttgagtttttacttataaattactagaatggttatgacaattgttatctttctttttttcttttcactaaatggtgtttatgcttctttatttgtattctagactcactgcaggggaaatcttaagtttaattttcacaaatatatgatacgtgctctcgaagatgatttcaagaaagttgttgggataaggtaacccttggttgacagtctattttctgatatttgatatttatgcttctttttctgtgcatatatatgtgtaattcttttaagtctactgtatcggtctcctagattgaccttttagctttttggctgcagttggtacctttgggtttttgtggtcatcttcttgttgctgaatgttaatggtaagatgcttctgtctctagtttataattgcaagaccataactcaagatctaagtgatattggattgttttctgtagcagaatcccgtaatcagtctaggttctttgttttgaacttcaagtgtgtcatctctttggtgcttttacttaaaattgacttccaaattatagtttcaaataagcatctttagttacaacgttttctagtgaaattagtaatatatttttccaattccttactctgcctccacttttctatggtggagaaaattatactccaagttggtgtaaggaattggaaaaatatatttactaatttcactagaatgcaatcaacaaggggtcataatctgattgtatgacagaagaaaccagaattctgattaaaaaaattctttctttatttttctttttcttgccaagaaatttagtttacttttattttattttgcttgaaattaatttacattaaacctatatagaacataattttacatataagtaacaccttaagataattcaattggatcttactccttgtttggactacaatgaggcaatgtctatattgattattaatgcctctcaatactcaaaccaagatcattcattatttatataaaagaccagtgtgtattaaacttctttgttgtatggaacatattagtctttttttttttttttgtcaatgttttgtgattggacgactaagctcttctgaataatgtgttatatttgcaggctggcacacatatttctggatagcattcattcctttctttgtgagttgtattgagattatgctatctactttgtgattattgtttgcatgcatgtttcattgttcttgcatggtgttaatagttcatttcacattttaagtatcatactgatggagaaagagataaaatacagagagggagaatcgggttgaaaaaagtttaacacaattttgtaaataggcaaatggacagctaagaaggaaaccaaatttagaattaattgaacaacatgacctaggtaaatattgtgcaatttgttttgtgataagttacacatgcatcatgcacttgtaaatgtagagtcatgagtcagggtagaaatttgcacctagacaggtggttttaaggtggtacttctcatctccaagtcttaggtgatatgtatgatagtatgagtgccttttgctgttgtgatatacatatcacaaattgtaaccacttgttgttttttcttttaaatagcttctacttgccgtgggcactaaactggagcatgtcattactcagttggctcatgaagttgctgagaaacatgtagcaatagaaggagatctaatagttcagccatcagatgatcacttttggtttcatcgtccccagattgtcctcttcctgattcatttcatccttttccaaaattcatttgagattgcgtttttcttctggatattggtaagctggtttctaactcaccactctgcttctacttctaccaaaaaaagaacaaattgttattatgaacacaaacttatcttcttttctgtgtatctaggttcaatattcatttaactcctgtataatgggacacgttcgttacattgtcccaagacttgtcattgggtaaaagcaatagtctttatattctcattacaaaaattttaatcatcgcttccaagtacgaaacttttattttatttttaccatgttgtgctttagggtgttcattcaggtgctctgtagttacagcactctcccgctctatgccattgtcacacaggtgcgttgatatgatgctgatctataatttaataagttctaaaaggaaagtaggaacttaaataaaatgctgaccataataaaatcttgtggaatgatttgaagatgggaacttcttacaagaaagccatatttgaggagcgcatgcaagtaggaattgttaattgggctcagaaggtcaagaaaaagaagggatttaaagcagccctcattggttccggtgctggctcaggctcaggctcaggctcgggatcaggctctactcctggtcaaccaatttctgatactggttctgcatctgtgggaattcagctgggaagatttgtgcccaaagcatcaaagccagagcagagtgatgactctaagtgatctcttttcaacgtgtcagtgttcatgacactgaagacataagtctacccttaggtaatactactactagttgccactttccagctgtgctgggattagaaattttaattcttctgtcatatattatggctgtggtgggactgtacctaagtcttttatggacaagacacaagcctttgatcaatatattatataatcttatatatacataagagaaattaagaaatctgatcttctcatgcttctttcttcttattatcatggtacatacttttggtaactgaatagtgaatacgtgtagtctcatgggaaaagatat

SEQ ID NO: 2 - DNA, 1560 bp, Cannabis sativa
  source 1..1560: mol_type = genomic DNA
  CDS 1..1560: note = CDS of CsMLO1
atgagtggcggtggagaaggtgagtccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcattattgtagaaaaaagtaccccttttattccgttgaagcattgcaccatttgcatatctttatcttcgtcctagccattgtccatgtcactttctgtgttctcactgttgtgtttggaggtgtaaaggtaatacgtcaatggaaacgttgggaggattctattgcaaatgagagttatgacactgaacaagctgttcttaagtctaagaaaaaggtaacaagcgtccatcaacacgcgttcattaaggaccactttttgggttttggtaaagactcagctctactaggctggctgcatgcttttgttaagcaattttatgcatctgtgaccaaatcagattatgcgaccatgcggttaggattcatcactcactgcaggggaaatcttaagtttaattttcacaaatatatgatacgtgctctcgaagatgatttcaagaaagttgttgggataaggctttgggtttttgtggtcatcttcttgttgctgaatgttggctggcacacatatttctggatagcattcattcctttctttgtgcttctacttgccgtgggcactaaactggagcatgtcattactcagttggctcatgaagttgctgagaaacatgtagcaatagaaggagatctaatagttcagccatcagatgatcacttttggtttcatcgtccccagattgtcctcttcctgattcatttcatcatccttttccaaaattcatttgagattgcgtttttcttctggatattggtaagctggtttctaactcaccactctttccaagtacgaaacttttattttatttttaccatgttgtgctttagggtgttcattcaggtgctctgtagttacagcactctcccgctctatgccattgtcacacaggtgaagatgggaacttcttacaagaaagccatatttgaggagcgcatgcaagtaggaattgttaattgggctcagaaggtcaagaaaaagaagggatttaaagcagccctcattggttccggtgctggctcaggctcaggctcaggctcgggatcaggctctactcctggtcaaccaatttctgatactggttctgcatctgtgggaattcagctgggaagatttgtgcccaaagcatcaaagccagagcagagtgatgactctaagtga

SEQ ID NO: 3 - protein, 519 aa, Cannabis sativa
  source 1..519: note = This sequence is a translation of SEQ ID NO:2
MSGGGEGESLEYTPTWIVAVVCSVIVAISLAAERLLHYLGQVLNNKNQKPLYEALQKIKEELMLLGFISLLLTVSQNTISKFCVPASLVDNLLPCKRPHQEANDSSSSTSHHISGSFRRLLEETSTAHLGYCAHYCRKKYPFYSVEALHHLHIFIFVLAIVHVTFCVLTVVFGGVKVIRQWKRWEDSIANESYDTEQAVLKSKKKVTSVHQHAFIKDHFLGFGKDSALLGWLHAFVKQFYASVTKSDYATMRLGFITHCRGNLKFNFHKYMIRALEDDFKKVVGIRLWVFVVIFLLLNVGWHTYFWIAFIPFFVLLLAVGTKLEHVITQLAHEVAEKHVAIEGDLIVQPSDDHFWFHRPQIVLFLIHFIILFQNSFEIAFFFWILVSWFLTHHSFQVRNFYFIFTMLCFRVFIQVLCSYSTLPLYAIVTQVKMGTSYKKAIFEERMQVGIVNWAQKVKKKKGFKAALIGSGAGSGSGSGSGSGSTPGQPISDTGSASVGIQLGRFVPKASKPEQSDDSK

SEQ ID NO: 4 - DNA, 13001 bp, Cannabis sativa
  misc_feature 1..13001: note = Genomic sequence of CsMLO2; note = The gRNA sequences SEQ ID NO: 287 - SEQ ID NO: 625 are the respective CsMLO2 positions targeted gRNA
  source 1..13001: mol_type = genomic DNA
ttatatttataatactatgtaatctcttaattcttatggtcttttgaaaaatttaaggcaccacctagcatttctcatatattaggcactaataattctaataattaacaaaataaactataatgtgaacgaaaacatgtgagacatatctctaatattacaagcaaattaattaggagccttttataatagttattgaacgaaaacatgtgagacatatatctaatattaaattaccaaaaataatataaaatattattttaaattttgtgttttacaaaatttaccaattgaatattctattttgttaaaagacgattcgaaccttaaatttttgaaaattttaccttgagtttaatttttgtcaattttatattaacataaccaacttgaaactatagtttttagtataaaaagatgcaaaaattgtaattagctacatacaccttcaaatcaagttattattaaattttattttgataaaaattgaagttatggtaccattttatacaattttaaaaaatacagaatctaatttatcatttaataaaatatattagagtttaattggagagccttttagtctattttttttttcgttgtagctatttaagaccacttgtaaatttttaaaaaattctaaataatttacaatgtcgaaaataaactttaaacagttacctaccacgtgtataagataaatagtcgtgtgagtaataaaatatgtaaatcatattttcaaatatttttaactgtttgaatttttttaaagttatacaagtagtattaaataattacgacgtatgcaattaaaaaaaataaactaaaagctatctaaataccaaacaaataaaaatctaccaaaacatccaaaggccgttagtaatatttactcatattattataatatttttaactagagccaaaagccgttagtaattattaggaaggatattcagatccaaaaaaaaaagtacttttattaggaaaggaatattaaaagtgaagggtagaatttggtggtggaagtatataagcaagtggttgacttaattggaagactaattcatccaaaggttgatatctcttattaaaaccatcatcatcaaattccaattgtttttctagtaggtcaaataaaagctttgattgaacttttttttcttttagagaacattgttattggtaatacttaagaaattttttaataagagaacacttatagttattatgggtaaattgtggcataaataattaatatttcagattttatacatttaaatacttaatgttttttttaaaggcaaaaatacctaatgttacaattctcttacactgttactaccacttccgttaactcataaatatggacacgtgaagggttcagatgtcttacatttatttatattttattttaaaatttaatattcttaatcagaaactaaatgctacttgtttaaaaaaaaaacttgttctcattacatatactatactacttcactcagttatggtaagttagaattgcatctctctttttttttttaaaaaaaaaaaaaagtaatgattaataattctaaaagttgtctagtatgatcttacgtgctactatttaaattttattaaataccataaaataattttactttgaaaaaaaaaagtttaaaaataataaccacattgcatatgtagtaaaaactttatgggtcacctctattggaaatgcttttaacactttcacaatattttttaaaaaattttttcttaagttatataagaccgagacatatttacactcatacaatatatacatatatattatggaagtcaaatattccatctcaatctctttctacgacgcgtaatcagctaattggaagtcaaagtcgtccatttctcaatggttcctcccaatggaccgtatacaacatatatcttatgtatatatatgtatattttataacacctcatttccaacttaatttcatattcatacgtacatacatacattgtgtgtatatatatacatataattaatatccactctctgtctctctctctctctctctctctctctatatcactattcacaatcagtctcaaattttttcaaccacatctccatcccaatttcaaaacagaatttacacccatcaaacttttcattctaacttattacaataactcataaatatatataatcaattattcccaagtggcctatttatatttatgatcactatatatatattttcttagagagaaaattaaattaaaagaagatggcggtggctagcggaggaagatctttagaacaaacaccaacatgggctgtggccgtagtttgtcttgttttggtatttgtttccattattattgagcacataatccatctcattggcaaggtatacccaattatatatattaatctcactttgaaattgttttttaaattaaacatttaattatttaaattaatgggtattttttttttttttagtggtttaagaagaaacataaacgagctctttatgaatcgttggaaaagatcaaatcaggtaaagactaaagacccaattaatgaatacccatttttgtttttttttttaatttaaaattgaaaaatatcttataatttttttttgatagagcttatgttattggggttcatatcgttgttactaacggtgggacaaagtccgatctcaaatatttgtatatctgagaaaattggagggacatggcatccttgtaataaagaacaagaagagaaattgaataaaggggctgaagaatctgatgtcgaacatgaaagtaacagtcggagattgctggcctttttagagtccggcgacggtgctgctagtgtccggcgaattttggcggcggccgggactgataagtgtgctgaaaaggtaaataattatataaaaaatctttttctttcttcttttcttcttcttgtctttcttatccttttatttggacatattcgtccaaacatataatattataggagttttatttgtattttttttaattataaaccaacctttaaatttatatatttagttttttttttgttttttgagaaaaagtcttatatatttagttataaatatatatattttaaaaaatattattatattttaaatattataaaaaattaaaattaaaattacaagaaattttattgaaaaaaattatgaaagtaaattaattaaatatttaatttaatttattagattttattttttaaaatatttaattgtatttattatttttatatatttatgtatataccccttagagagtggtggtagcattttctagaattttttttttaaattatattaaaaaatattctcacacacatttttatttacaggccgaccttgtttggccccaatttatttttcttttaagaattctcttttacattatctcataaaaaggcccacacaaaatatttttgcttgaaaaacagtttaaaaactatttaattttattcttttcttaaataaaaaatctatattattttttttaaaaattcctaagcgcatataagctagagatgatccaataatattaccattaattgaacaaaaaaaaaacctcacctaaaaacacaaactctttaattcttaatatttaattagtttgacattctcattcttgccactttaattatgattttaatttatatcgaatttgtaaaaaatgtttataattcataattaaggtaatattttatgtgtatagtattaggcgtattgtctctttcgagttaaaccatttgtcaaatatacccaactaatattattaatgattaatattaaaatatctaaaattaattaaatataattatataattagtccatgacaaaggcaaggattttccgcgtgaaagaacaaacaatagatttcaatgaggatttatatatatatatatatatatatatatatataatatgacaaattaatgtcatattattaatattcttatatactttttattttggctatctaaccatttaacacatatattattattaatgaagtgttttttcagagtacaaaattcgatcttaattattaagtactgttctaaaattatatacatatatgaatggttttaattattatttgtattatatatataattataatttcagggcaagttccatttgtttcggcggatggtattcatcagttgcatatttttatctttgtgttggctgttttccatgttctctactgcatcatgactatgtttttgggcagacttaaggtacttaattacttaattaattgcactaatttatatgatttacatttttaattaataagtttgaattaattaatctcttttgagatatgactttattaattaattaattccctaaataattatatataaattgattcccagatgggaagttggaagcattgggaaaaggagactaaaacagccgaatatcaattctcacatggtaactattattattattattattattattatacttttatcttaattttaatttttctataggtggtgtaattaattaattagattgttagtcaaaatgagttaagattatagattaatactaaagattaagaacattcataatgtctaataccaccatcaaatatgtcatatagttactaacataattcaatatcaaaatttattttaattattaaatatatatgtgagtataaattataagagcatctttattggacaccggtgatatttaacactttctaaatgtgtcgctttacaattagctagcaatactctctaaaaattattacattaaattatatagaacctgatacttaattataccaataataatataattggtaagagtattatatttataagaagtatatatatgggagttagaattaagtaaaggagcgttgacattaattattagattaattatacaactgtgttgttatatatatttttacagatccagaaagattcagatttgcgagggacacttcttttggaagaagacatttaagtttctggaccaaacaccctttcctaatatggatagtaagatccctttttacccactttttacttttttatattcacttaataattaattattatatataccactatatccatgcacttatatctattaatagtctacgtattacattataattaattattataagtgaatttttactttctagcctttcacatgcatataaatatgtttgcatggtttttatacatcgtggaatttgtttatatatatttaaatcaagagaaatgctaaagggcaccactggtgcctagcaccctcttacgtgtcaatatcgctattggtacaattaagtatcgggtcccatatagtttaatataataatttttatagagtatcgctagccattgcaagtcgacacatttagaaggtgctaagcaccactggtgcccaataacaatactcttaaatcaatgtaaaaagtgctagttgattaaattaatcattaaagaattattgggtcttaaaaaatgaattacaataataataataatcattgcttgcttttaagtgaagtaaatagctagctcgaaaatcagtttacacattgatttaaataatttttatcaccatcgaaaataatgacgcgtgtccttttaatgttacaatacaacaaattaaataataatgtggccccacttgtgatattagtgcttactttaaacccattaatttcttctttaattacctttaattatgacattatgaaattccaatatagtaccttgatgatggtatataatatatatatagagagagtttgttagatctacctttcatgattgatcatcttatctgaattaactacctttaataaagttttttgttcattattgtaaaatgttttttaacaaatgaaaagttgcatgtagattcacgtgaatatactatattttacaagctaaggctaatataggaatgaataatgttattgagtacaatactctttagcactttctacatgtgttttcttattactattagtaggctcggattttggtatagacagactaaacttaaggtccacttagctcagcggcttaaaaaagttttcttgatattatatatatatatttttctaaataagagtttaaaaatttttttttctatttattatattttaagaccaatcctaactattagttaataaaaattattatattaaattatataaaattcaataattaattataccaatatcaatacgacgttaaataaatattattgctatctatactttttaacatttatcggtaggaatatcaattttttttttttttttggttttttcttcttttttagagggattgggctgagaagtcataagctactatatcaagctatggtgtcctattatatatataaaaagtgttgacttttattttttcttttcaaacactaaatattcattactctatatagatgaatttaattgggacttatcaaaccacatatggtggtatattcacaaaatgttaaatgtgctttattaatttattctcccatgaaaaagacccaacaagattattttggtcaatggttaaaatggctttgaaatattctcatcatatataattagtcaaaccattcatattcaatttatagtttgcttcccaagttatattaaacaagcttaaactctatctcttattcctcttaacaaaatgattaaaacacattaaaatatattaatcatagtgcatttatataatggtgacgtgttaaggttaatatatatatatatatatataacaacggtctgtatatggcaaggtgtgaaaatgacaaggcctttactccatatttatggctcagctcaaattttctttctctctctacatatatataattggtttttgactttatattttaattttattccaccaataattcatctttgtctagagcataataatgtgttttatattattttgtttttacatttaataacatatttatatttaatatttggtgaacactactttttccttaattttgtattattagtttgtcaaattatatgtttaaggtaagagaatgattgatcatgatatctagtattttatatatataatattttttttggaaaagttataatatataatttattaattattgattattgtagcctgctctttaattattaattaattatttttcgcaggtttgtttcttcagacaatttgttagatcagttcctaaagttgattatttaactttgagacatggatttataatggtgagattttcttgcactaattatatatatacaattgaattatagctgtagagatatttggtaccttgtatttttaataatacttatatttagtactctatattttaaaatcatatatatttagtaatctaaatttaaaatttttgataaaattttattaatacaatcaatttatctaaaattttctatattttgcaatcaaatctaattattttgatacatataactgaaaattgtctggtcaaattaataaaattgtgttgattaaattttattttaaaatactaaatatgtatagtttaaaaatataaagtaaaaggtattgactttaattattaatgcttcaacataatttaattaaataaggaaaaattaactatttattgacatttcacctatatatatattttaaacaatgcaccgtatagagttaagtttaagttaataaacttaggtggcgtttggttggaggtaatgaaatggaatggaatggaaagaaaataattttcattccattcatttgtttggttgcattttaaagtattagaatggcattccaatggaatgctctttccaccattttggtggaatgactattccattttaaaaaggaatgaatgaccattccaatgtaacaagaaaaaaaatttaattattttttatcaattttttttatccattttaaattttattccattctattcctattctcattcccattcctatttctattcctatattttcattcctctcaaccaaacgcctctgtgggcctattatcttgttatgtattaactatttaatattcataaaagatcaataactaaattaattgaggtttattataacaattaataatttcaggcacatttggcccctcaaagccatcaaaaatttgactttcaaaagtacattaacagatctctagaagaagatttcaaggtcgttgtaggaatcaggtaaattattaatataatataatctttaaaaattaatttgttaattatatattattaatattcccaaatgatgacttattttaattcattctaataataatataataataattattattttgttttggcttgatcaatctcagtcctcctatttggttcttcgctgtggtgttcttactcttcaatacccatggtgagcattttattattaattataccattaattatttcaatgcatccctaattatcacagtttagatttttaattaaagaatgataagatccaaacgtgtgcatatatgtcaaattattatatttttgtttttattattggaccataatgaatttgtttacatttggacaggctggtattcttatctttggctaccttttattcctctaattgtaagtactaatatactgctctatttatttcgatatttgagagaattttttaatttattttttataactatttatttgtagttatttaagatatcttgtaaaattttaaaaaattcagaaagatttaacacgccaaaaataaaattcaaacaatctatttcacacgtgtgcagtaataaactgtttgaattttattttcgcttattaaatttttttaaaccttttaaagttttactgtaaactaccgtgaaaaaaattgggctaatttttttttttttaaagtatcaaaacaaataaaaaatgtattaatgaacctcttcttaaaaaataaaagagtgcattattttaatttattattataaataaattatgttaggtgatcctattggtggggacgaaattacaagtgattataacaaagatggggctaagaattcaagaaagaggagaagttgtaaaaggagtgcctgtggtccaacctggggacgaccttttctggttcaaccgcccacgccttattctctaccttattaacttcgtcctcttccaggtacataatcaatcaaaatcagccttggaaattctataatgcacccccttaaaatgaatacattgatgcacccttacttgttttagcactcaaaataatattttagtcaaattttttctcatggtcatgtacgttatagttatttaagacaccctacaaaattttaagaaatttagaaaaatttaacacgtcgaaaaactacgtacaaatagtgtgttgcacgcgtgactattttattttatacgcgtgtaaaatagattgttcgaacattgttttctatattgtaaattattcgaaatttctcaaaattttgtaggttatcttaactagctataacgtacataaatatgaaagaaaattagactaaaaaatttttctggatgccgaaacaagtgatgtgtatcaatacatcccttttaaggagatgcattgtagaatcaccccaattttatacgtcctttaataaaaagaaagtgatatttttaaaattattaaagaatatgtcatttctaaaaattaaaaaaagttaagtattgggctgagtgtgcctaggtgaaaacttagcccaccttagtcataataatagttagggataaattgaaaaatatctttttttgtccattaattaaaaataccttatattatattttgttaaatatacccactttcgtgagtgagttgtcaaatgtactccaaccttccacttaagtttagcatatattttacattaacttaatggtataatagggacatcatctataatagtgggtatctcttaaagaaaataaaaatagaggtaaagatcaaaataaataaagtaaagtgaatacaatgtaatttcctctaatagttatttggagattttttacttttatgattttaaggtataatgtgtaaaatatagttttttcttgaaaaaatattttatatgaaaattaaaaaaaaaatatgagacaaacaatataagataactctaattatctctctggttatcatacaagtacttagttacccttatttggtttctcatcatttttcaacaatttcttaattttttttcataaatactataaaaattcataaaacttataatttccaatttaattattatgacttaatcaatggggataattattttatttttaagaaaaataattaatatccatgttctaaacaaaataaacaactaacaccggtcaattgagttgatgtatgatgacttgactatttattttcataaatacttaatgttctatattataaaatttatagatagactagtattattataatttcatatataacatcaatgtatattttttcatgtgttcaaacaataaattaataaattttctaatatatttcatttattttttgtcattgcagaatgcattccagcttgcgttctttgcatggacttgggtaattatctatataattatacacatatcttaataattagtattattctaattttgtgaatttcatctaaatatttttttaattaatttttcagtatgaatttggacgcaaatcttgctttcatgaacatgttgaggatgtggttatcagaatcaccatggggtctgtatacagatataaatatatatatatatatatatatatatatatatatatattataaatttcatttaattatttatttatgttcaatcattaagtgccattaatatttaatttttatttttgtaaaaatttctaacaccgttggatcatgatcatcagggtgctcatccagattctgtgcagctacgtcaccctccctctctacgcccttgttacacaagtaattacactctaaacactaaataacacaatttcattatatatttctctttatatagtttgcaaaaaaaattattatctagtatttattattgcacatataattataactacaccctaattaaatatataaaatctatttaaaaataacttttaatttattattaaaaaattcaagtagaattaatttagactaattataaataaaaataatctctaaatagtaattagttatgcgttttttaagtactgtattaaaaaaaatatctcgatataagaataattatatcttaataaaataaatacatataatttttgtgtttatttatgtaaaattaataatttcattctaaattataaaacatgtataaatttactaaaaaataattttattatgatacagtattaagaataatttatcgtcattaatattttattgtgtttcaattttttttagttaaactctacttatagttataaccttttaattaaaatatagtccgtttagaattttatttaattacttaaaaaatattgtcatagtttgggagaacacattattatagtgttttacataattattagatatatttttttatactaataataacaatatgagaaatataacaaatttgtgtgaacatatcactaatcatattttaattgttatattgtatgattaacaataacataattaatgatcatatattgttattaacttaattactatatatatgacacaacagatgggtacatccatgaaaccaactatattcaacgacaaagtagcggaggccctccgcaactggcaccactcagcccgaaagcacatcaaacaccaaaacaaggcggcggggtcagtgacgccgctgtcaagccggccgacgacacccacacatcacatgtccccggtccacctcttgcgccactaccgcagtgaggcagcagacagcctccaaaactccccaaggcgctcaaactttgatggcgccactgaacggtgggagacagatcgccatctccatctcaccaccaccacggccgcttcgcgaccgagggttcctcctctcatcatagtaataataataataaaaataaaaatcagcaaattgaattgggacaaataatggaacaacatcatcttcatggagatcggttcgccaatgagcttcctcctcaaaccactacattaagaaatcaagctgctgctgatgatcaatcagcagcagcacaggtgaggccgccgccccaccatgaaattaatattggacacagagatttctcttttgaacggtttgaacgacagcccagcgtttgatgacgagagagaattccgttatttggttataatgttaatcggacggttctcattgtctcggcctctgatgatcaacgagctagaatgattgagatgatttattactgatcagcatgcatgattatatataagcagctccaataatgagcggtttaattatatgttatttatgtataattaattattatgtttatataatttttttgtttatttgtttttcattagtatttttaatttataaatattaatttcttttaagaaacgaatttggcgcacgtggagatatggagctggttttggcatcccatcccatgctaagcttcatgatcagttttattaattaattattactgcttaattagtttaatttgtacgtaagtgcattttctatatatgtatataaaatgcatgtataattaggaatattttcgacaattttctttttcattttatatcgacctaatcactttatattattataattatataagcaccaatttatattgttgttacctcccaataaattccatgcaataattcaaactatatcatgattaattattaacaatagaaatgctggaccaaaccaacctacaaagttgaaataagaaacaacccaaatagtattaatcactcaaacatgtta

SEQ ID NO: 5 - DNA, 1758 bp, Cannabis sativa
  source 1..1758: mol_type = genomic DNA
  CDS 1..1758: note = CDS CsMLO2
atggcggtggctagcggaggaagatctttagaacaaacaccaacatgggctgtggccgtagtttgtcttgttttggtatttgtttccattattattgagcacataatccatctcattggctggtttaagaagaaacataaacgagctctttatgaatcgttggaaaagatcaaattgatagagcttatgttattggggttcatatcgttgttactaacggtgggacaaagtccgatctcaaatatttgtatatctgagaaaattggagggacatggcatccttgtaataaagaacaagaagagaaattgaataaaggggctgaagaatctgatgtcgaacatgaaagtaacagtcggagattgctggcctttttagagtccggcgacggtgctgctagtgtccggcgaattttggcggcggccgggactgataagtgtgctgaaaagtcagggcaagttccatttgtttcggcggatggtattcatcagttgcatatttttatctttgtgttggctgttttccatgttctctactgcatcatgactatgtttttgggcagacttaagatgggaagttggaagcattgggaaaaggagactaaaacagccgaatatcaattctcacatacagatccagaaagattcagatttgcgagggacacttcttttggaagaagacatttaagtttctggaccaaacaccctttcctaatatggatagtagtttgtttcttcagacaatttgttagatcagttcctaaagttgattatttaactttgagacatggatttataatgtttcaggcacatttggcccctcaaagccatcaaaaatttgactttcaaaagtacattaacagatctctagaagaagatttcaaggtcgttgtaggattgatcaatctcagtcctcctatttggttcttcgctgtggtgttcttactcttcaatacccatggtgggacgaaattacaagtgattataacaaagatggggctaagaattcaagaaagaggagaagttgtaaaaggagtgcctgtggtccaacctggggacgaccttttctggttcaaccgcccacgccttattctctaccttattaacttcgtcctcttccaggtacataatcaatcaaaatcagccttggaaattctatatgaatttggacgcaaatcttgctttcatgaacatgttgaggatgtggttatcagaatcaccatggggtgcagctacgtcaccctccctctctacgcccttgttacaatcactaatcatattttaattgttatattgtatgatcagatgggtacatccatgaaaccaactatattcaacgacaaagtagcggaggccctccgcaactggcaccactcagcccgaaagcacatcaaacaccaaaacaaggcggcggggtcagtgacgccgctgtcaagccggccgacgacacccacacatcacatgtccccggtccacctcttgcgccactaccgcagtgaggcagcagacagcctccaaaactccccaaggcgctcaaactttgatggcgccactgaacggtgggagacagatatggaacaacatcatcttcatggagatcggttcgccaatgagcttcctcctcaaaccactacattaagaaatcaagctgctgctgatgatcaatcagcagcagcacaggtgaggccgccgccccaccatgaaattaatattggacacagagatttctcttttgaacggtttgaacgacagcccagcgtt

SEQ ID NO: 6 - protein, 586 aa, Cannabis sativa
  source 1..586: note = This sequence is a translation of SEQ ID NO:5
MAVASGGRSLEQTPTWAVAVVCLVLVFVSIIIEHIIHLIGWFKKKHKRALYESLEKIKLIELMLLGFISLLLTVGQSPISNICISEKIGGTWHPCNKEQEEKLNKGAEESDVEHESNSRRLLAFLESGDGAASVRRILAAAGTDKCAEKSGQVPFVSADGIHQLHIFIFVLAVFHVLYCIMTMFLGRLKMGSWKHWEKETKTAEYQFSHTDPERFRFARDTSFGRRHLSFWTKHPFLIWIVVCFFRQFVRSVPKVDYLTLRHGFIMFQAHLAPQSHQKFDFQKYINRSLEEDFKVVVGLINLSPPIWFFAVVFLLFNTHGGTKLQVIITKMGLRIQERGEVVKGVPVVQPGDDLFWFNRPRLILYLINFVLFQVHNQSKSALEILYEFGRKSCFHEHVEDVVIRITMGCSYVTLPLYALVTITNHILIVILYDQMGTSMKPTIFNDKVAEALRNWHHSARKHIKHQNKAAGSVTPLSSRPTTPTHHMSPVHLLRHYRSEAADSLQNSPRRSNFDGATERWETDMEQHHLHGDRFANELPPQTTTLRNQAAADDQSAAAQVRPPPHHEINIGHRDFSFERFERQPSV

SEQ ID NO: 7 - DNA, 10001 bp, Cannabis sativa
  misc_feature 1..10001: note = Genomic sequence of CsMLO3; note = The gRNA sequences SEQ ID NO: 626 - SEQ ID NO: 870 are the respective CsMLO3 positions targeted gRNA
  source 1..10001: mol_type = genomic DNA
attctaaatatatgtattaaattaaaatggaaattaatgaatgtaatttattgactaaattaacaataggagaattttatatttccaaataaaaaatatataaaaaaaaaatcgaaattttttttttccctttttttttttttttttttgaaaaatacactgccacaaacgacatgataaatttccttaaacttccaaaatcatgaaatcaattccaaatgaatctaaatgcaaaaattaagacattcatatagattttatgcatcgaaaatacataaaacaaagactttaaaaatcaccaaaattttctgaaaaataattttgagatttctttgttttttcaaagtggattttcatgcttagaacaatctatatatatatgaatgtattaatacatatacaatatatatataatatatatacatatatatacagaaaaataaaaatactgtatgttatataataaacatgaaatgtatatatatgtatatttgtatatagcatttagatatataaataatatatatatacaacatatacgtaatgaaatgaagaagaaaaatataatatatatgtgtgaatatattatgaactgaagaataaacaagtatatatgtatgaaaatatatatgtatatatataattgggattgaatgaatatgaatataaatatatacacaaacgaaaataaatatatatatcaacactgaattaatatatataatatatataaacactgtatatacgagtatatatatatgaaactcaaactaaaatcaaggcagagataaaaaccgctctgataccaactgttagacatttatatatataaactgaaatgtatatgtttaagtattaactgcggaattaaacaaatatatacactatataacttaaggatcgaaatacctccagccattgattcttgagcttcaaacccacataagctttgatctgcaagggaaactggttgatgtgggtaatcgggcttcctcgctctctcaactctctttagatggaatttgctgttatgaacagaatgagtgaggagctcggggaccgagaccctatatttataggtgagatactccatcagtatctgtgccacattaattgtcagaatattttgacaattaattcaggaaatcaaatcaggtaatgaatatagaaatctgaccatatatagaatattacataattgattttgtccagattcaatgaaataaaatattcatttaacagaaaatcaattatttatttatttccttaaatagaaaatcatttccttaattagaaaataatttccataaataaaatattctaatatgcattatatatcgaaattagttttttccaaaatttgaagttttttttggctgtatatagtaaaattatatctaagacaacaaaagacaataaaatatttttaaaatattataaataataataaaacataaaccatttaaactctagatcattctgggtagtcaatgaatacggcaaaatattattggtttttacgaagtcaagcaaattgaatatgtatgtgttgcgctcccaaaatttctggacaaaattaaatatttggtgactttattttgtctaattgtttatatacttcacgaattacgatatatacaatattgcactgaagtatgtggtccaaaatggtacgaaaaatggtctttaattaacacaaattttaggaggactttttccctaaactaattggaattaagctcgtttttgctatgacccttagaatgccgtatagacatgaagatgcttcatctcgttgactttttatatatatgatttcttgagttcaacaaatattattatattattattattattattattatttattattatttcaacttagtatgacagtgtttttcttgctcctgctaaattattaacacaatttagatctacctattatatagctatttttttgacgatttagctcctaataaatagatctacgtatatatattacaacgcttttgacactatatatgtatatttttttttgacaaataataatattaaaatttaattatcgtttttactaataataaataaaatatttagatcgatttccttaaataatttcattaaatgttatactaatcgtatataagatatatagacgttcggtatatatatattctatataaagtgtacttatataattgaaattcttgatttataagaaatttatcgtgactttttatttttatttaataaaataataaaatattatttataataataaaataataataaaataaatttcattaatatttaaactgatatttaatcctaaatattagagaatgataacccatttaaaaagaaaacaaccggggatttgaggcaataccctaatagcgaagctcctcatatacatgtgatggtcgtccatctccattgctgacaacttggtgaattttctatccaatcaggcaataaccacaaaatcaaagctttgtagtctattcttggtgagttatcattttttttttggtttagtttgttctttcttttattatttaccacttgtttctttttattttttgtaacagttttatgtatgtgatttagtcattagagttgtttgtgattcttcacttgcgataaaagatatgttgatagtggcgatagtggggtaaacttgtatgcctataaagtgtttgatataattcttcaatgacttcttcaaattgcctagagcagcggtacccatacaccaacaatacaaggtatacgatttatataagcattttaggttgtatcagttagtctatgatgtcaagaattatcattagattgtattactcttgctttatcagctctaaaagccattgaagctgggaaattgaaacttaaacttggaggttaaaatcttcaattctcataaccaatctttacaattaattaatcaataaatttatgttggggtttcaaatttcaatatatgtaggagttgcattgggagatagttggatatctcttgaagattttgtggtaaaaaaactatgatctgaattttcaatttctttttgattttacaattatgggaatcaagtcatttcaatgtttgattttacaacttgaaggtgttataagctctaacagcaatgaagtagtacttatgaacataaaatgtggaatcatttgtatgaacattatatgacgtcatgaccttaattcttttcttctttttttgttgttgttgtgatcaatttcaagacttttataactttctattggattcaggaatggactcaatttcatcatcttcaacaacagtacttcaattgtcaaaagagagtgcatacaagagatactcaagatatcttagtgtattgagatcatcatatattgcattcattatttacttttaattgtcaaaattataaattatattatttaaatatacataataataatctcacctaataattaataataatttttttaatttttaattgtatcacttctaaataaaaaaaaaatacctaacgtaaaatttaatatacgtgcctcgcacgtaaatttttactagtatatatatatatatccaacacgtttgacaaatatatgtatcttatattagtataatattaacatctaactttaataatactaaaaaaattaataataaataaaatatttagatcgatttcctaataatttcattgaatgtttactaattgtataattagagttctagttttagtggaagaagtaatattataactcaatacatattgattgaaaattaatgaatagtgcgcgtttggaggtcaaaacaaaacaattaaaacattttattttttaaatgcataataacattttattaaatctatatttatacatatgtgacgagagtcgttcaattcaagtaaacctacagatcgacttgtcaatatgctttgaaaggtacaacattacttggtctacatcactacgtggatgaataactaataaaatatgcaaaaaaaacctcaattaaataatatatttagtcttcacaagagatcaaaattttcttaattcctttgttcttatatacatattttttttatttcttcataaaaaataaaaaataacaaatctctaatcaaaattcttggtgatagctacttttttcttttggggtttgaaaagtcaaagtcttaaatgtttttcttaattgttttctttaaaaggaacactaaaatgaaattatcattcgtacttggaaaaaaaagggcatttcagctttgctctctttccattcttctccatatcatttctgtacattttctgagactctttttgtatacttcattatatcattatattataatatattaattgatatatatatttatacagtgataaactctttattataatggctgatctgcaagaagagcgtagtttggaggaaactccaacctgggctgttgctgtagtggtttttgtgtttgattatcatatctataatcattgaacatgttattcatctcataggaaaagtaagtttcattacaattatgtatatatattttaagttctcactatcagttattatggttatttatggttatttatcttttttagtggttagaatccaaacacaaaaagcccttcaggaagcgcttgagaagattaaattaggtacatacatctactttatttattttatatattcttttgtgcatttattagtattttttttttttggtctaattttaattgggtttgttttcattttttttttttttgtgtgttgcagagcttatgctattgggattcatttcgctgctcttaacaatattccaagattacattgctgacatatgtattccaaaaagtgcagcaaacacttggcatccttgcccataccaaaatgaagagaaaaaggctaaggaatcagataaaccaaaatcaaaaacaattgattattgttcagaaaatgtaagaaaacaattaattaatgtaattatgtcttaatgataggacgtatactaaaagtgtgtggtagtagtagtacttttgcttttaatgagttctgaatttttgaaggactttttgaatattgtatggtgtcatgtcatgaagtaataatataaaattaatatatatggtttaatgattattgaaacatgatttgtgtgttttgtgtgaggtgcagggtaaagttctttagtgtctaaatatgggattcaccagcttcatatcttcatatttgtcttggctcttttccatattatatattgcattactaccttggctattggcagaaccaaagtgagtagtttttaagtttatcactttagtttaacacatattctataaaattaatatggtctaatgattgattttattgaattgcagatgagaaaatggaaagtttgggaggatgagacaaagaaattgagtacatatatagtaacggtaaattttttttttttattattatattattataacttatattatcaatcattttgctgtattttttgtataattaattattttttggaaacactacaacttatctaactcaggaaagtcaattaagtgtgaggatatttttaattccttcccagccgtttctttgttgtaaacactgattggtttttatgtgggtcatgtgaattaatatttgtttgactttaattttttaatatgatgttaagtattgagtcattgacctttatacaacttgttcatctgatcatcacaatcctcatccacatcctcacatttctcatctttaatatccaaaatttttgaaagctctctcttatatatcacactcatatatctttcactaaagtaaaatctagaaatatcatggaccttgcagaaagagttaaaattacatacgattataacaactaattatatgtacacattataaaagatgatattattaattttttctgtgtaaaatttatgaaacaatataaaatatttatatttctttaaaattatatataaaagaatatattcatattaatggataaatgtttatataataatattaatcgcaaatgggaaatacctagtaaaaaaagcatgaaaatactatgtaattttaattaataataaaattaaaatattattaattaatactctatcatgggtttgactacaattgacttcttacttcttcttcttattgttattattttgaaataaaaaatcagattagttttggaatattttatagaaaggcagtatataatattttatacaataattatgtagggaactaaaataatattttctttcgataatttaatgtatacttttttgtttgaattttcattatattatattttaatttaatatgtaatattaatggaaactcttaagcacttaattttattttaaaattaattttttttgagtgaattttaaaattaaatttaattataattgctaaataattatttaattaaaaaataatttactttaagtataattaattaaattaatttaactctaaaagcatattttttttaagaatagcctgttgagaacatattataatagattataaaaaatgtattattatagctacatttgtaatatattcattgagtagttttaatgatctttgactccaaaacaaactttaatagcatgaaataaatagcatagcattgaaatcaattaaagcatgtaaataatttgtaccaaaaagtaagaatgtaaagattatagtaattatactttcactgatatagttatatattatttatataaatattcaatcaaatggttggttttgcagatcctgagagatttagatttacaagagacacatcatttggaagaagacatttgaacatttggagccaatcaccaatttcactttggattgtaagtactgtatatttttactataagaaccttattatagagaagaaaacaacaattaattttgtcaaaagttttttattatataggtctataatataatataataataacatttttctttttcttgtttatcaaatgatcaggtatgctttttcagacaattcttcgggtcagtcactaaggttgattacatgacactgaggcatggatttgtaatggtatgttgcacaatgatcttggcctgaaaatggcttcaaattacacatatattgatcactaaagctcatgaatattagctatgcttttcccatttcaggcacatttggctccagaaagtgaaacaagatttgattttcagaagtacatcagcaaatcacttgatcaggattttgtagtaattgttgggataacgttagtgttttctgcctttgaaatcagcttataatataaatgtatatatataaatatatatatattgataataatatctaaactttgttttgtataatttatatatgcagcccagtaatttggttcgttgccgtattgtttctactgtccaacacatatggtaagttattgacagtttgttctgttattattaaaacttattatatattaccatttcagtgactgaaatgttgttcttttttaattaacaggaatatactcttatttgtggctgccatttctacctttgattgtaagtacaacacaattcacgaataattgatagatagtatatatgttttattaattagtcccacatttgctaatgtgtggaaataaattgtgatatataagatgaataggctactccttttatagggttttcaaatggaaccccattcatcttattcctaaattctaacatggtatgtcaatgtgtgccactttccagtgttgggtttttgacctgtttcaaaatctacaaatttctcgacgtgtacacccacaaattttccgatatttggccttgtgagattctttcaaatgtcacgttcggtcctgtggtctctgaaaggtcgttcgttatacccacacttttcaggcccaaacgtgaggagtgtattaagatagttagtctcacattgctaatgtatggaaataaattgtgatatataaaatgaatgggctactcctcccacagccaattagttttaagataaaaccccattcatcttatccctaaattctaacatgcttaatcatattattctaattaagttattttacaaattgcagataatattactggtgggaacaaaacttcaagtaatcataacaaagatgggattaagcattcaagagagaggagatgtggtaaaaggtgcaccattggttcaaccaggagatgacctcttctggttcggacggcctaaattaatgctctttctcattcaccttgttctctttcaggtacttatgttgtctatctatatatatgtctctttaatattataatcttaaaactatttgcactaatatatatatgttaacaaacaaaatgatatcttttctgcttggtactaattaatgctggctgtttctgttgcatcttattcatttgcagaatgcatttcaacttgccttttttgcatggagtacagtaagtgattcttcatttgactttccttgatttttggtttcaaggcaataaaattattacatggtcagtcaatttctatgtgctgattaatcttttctttctttcagtatgaatttaccttgaacacttgcttccacgaaaaaattgaagatatcatcataagaatttcaatggggtgagttgtatgagattgagaaattataatatatgcttattactatatgaattcatcatatcatacatgttcaaagttttataattaattactaatacacacaaaacttgtgttctctttttctttcttactaggatcctcatacaagttctatgcagttatgtgactttgcctctatatgctcttgtcacacaggtcagtgacataatcaccttctaaatcatcattatcatctttgattcaattaactttttctaatttatttgatcatatattacagatggggtctacaatgagaccagtaattttcaatgacagagtagcaacagcattaaagaactggcaccattcagccaaaaccaagaacaaaaaacacaaccgaaattcggagacaaactcgccattctcaagtaggcctgcaactccaacatatggcatgtctccagttcatctgctgcaaaattacagaagcacagcaactgatggatactatggttctccgaggatgtctaatgtcgaaaacgattactatgagcctgaagggacaccatctcccaacaatggaccatctgcagcaagtgaaatacaagtgaataactatgaagttgagaaggatataagtgaggaaggacagccaatggttcaggaaccaagctcctcctcacagcaaaatgaggtcaagatttccttatctcacttctcatttaggaagtgaaatagataacactactgtttttgtcttataataacttcttttctttatttcttattatcttttggttatgtacattttgttgaagatgataatatgtataagcttgaatttggtagaaattgcttaatacatatccaataccaaatacatgttgcattgatagattatcttttttattctttaggtattatgtactcataaaccaacttaattttactttttgaagtaagttttgtttaatagataaataccattttggttcaaatgcttagaagtaataagaagagctctacatttatttggcttcttaaatttttgactttgcccctagttgtagtgcttctaatatgcttagtggaattattcaaatcatcaatattgctttatcgtttctagatattccacctatactataatagcattagctgtcactattagaaaagaaagaaagaaagaaagaaagaaagaaaaaaaaaaggtttgtagaactattcatggtgcgagttgggtgggtgactatggaaaaagatttcaccaaattctacatgtgcggtttaaacttgaatgtaatgtatttatttccctttcaaaaaggaaaaagaaaaagttga

SEQ ID NO: 8 - DNA, 1467 bp, Cannabis sativa
  source 1..1467: mol_type = genomic DNA
  CDS 1..1467: note = CDS CsMLO3
atggaatttgctgttatgaacagaatgagtgaggagctcggggaccgagaccctatatttgcagagcttatgctattgggattcatttcgctgctcttaacaatattccaagattacattgctgacatatgtattccaaaaagtgcagcaaacacttggcatccttgcccataccaaaatgaagagaaaaaggctaaggaatcagataaaccaaaatcaaaaacaattgattattgttcagaaaatgtaagaaaacaattaagttctttagtgtctaaatatgggattcaccagcttcatatcttcatatttgtcttggctcttttccatattatatattgcattactaccttggctattggcagaaccaaaatgagaaaatggaaagtttgggaggatgagacaaagaaattggatcctgagagatttagatttacaagagacacatcatttggaagaagacatttgaacatttggagccaatcaccaatttcactttggattgtaagtgtatgctttttcagacaattcttcgggtcagtcactaaggttgattacatgacactgaggcatggatttgtaatggtagcacatttggctccagaaagtgaaacaagatttgattttcagaagtacatcagcaaatcacttgatcaggattttgtagtaattgttgggataacgttagtgtttccagtaatttggttcgttgccgtattgtttctactgtccaacacatatggtgtgggaacaaaacttcaagtaatcataacaaagatgggattaagcattcaagagagaggagatgtggtaaaaggtgcaccattggttcaaccaggagatgacctcttctggttcggacggcctaaattaatgctctttctcattcactatgaatttaccttgaacacttgcttccacgaaaaaattgaagatatcatcataagaatttcaatggggatcctcatacaagttctatgcagttatgtgactttgcctctatatgctcttgtcttatttgatcatatattacagatggggtctacaatgagaccagtaattttcaatgacagagtagcaacagcattaaagaactggcaccattcagccaaaaccaagaacaaaaaacacaaccgaaattcggagacaaactcgccattctcaagtaggcctgcaactccaacatatggcatgtctccagttcatctgctgcaaaattacagaagcacagcaactgatggatactatggttctccgaggatgtctaatgtcgaaaacgattactatgagcctgaagggacaccatctcccaacaatggaccatctgcagcaagtgaaatacaagtgaataactatgaagttgagaaggatataagtgaggaaggacagccaatggttcaggaaccaagctcctcctcacagcaaaatgaggtcaagatttccttatctcacttctcatttaggaag

SEQ ID NO: 9 - protein, 489 aa, Cannabis sativa
MEFAVMNRMSEELGDRDPIFAELMLLGFISLLLTIFQDYIADICIPKSAANTWHPCPYQNEEKKAKESDKPKSKTIDYCSENVRKQLSSLVSKYGIHQLHIFIFVLALFHIIYCITTLAIGRTKMRKWKVWEDETKKLDPERFRFTRDTSFGRRHLNIWSQSPISLWIVSVCFFRQFFGSVTKVDYMTLRHGFVMVAHLAPESETRFDFQKYISKSLDQDFVVIVGITLVFPVIWFVAVLFLLSNTYGVGTKLQVIITKMGLSIQERGDVVKGAPLVQPGDDLFWFGRPKLMLFLIHYEFTLNTCFHEKIEDIIIRISMGILIQVLCSYVTLPLYALVLFDHILQMGSTMRPVIFNDRVATALKNWHHSAKTKNKKHNRNSETNSPFSSRPATPTYGMSPVHLLQNYRSTATDGYYGSPRMSNVENDYYEPEGTPSPNNGPSAASEIQVNNYEVEKDISEEGQPMVQEPSSSSQQNEVKISLSHFSFRK

SEQ ID NO: 10 - DNA, 20 bp, Cannabis sativa
  misc_feature 1..20: note = CsMLO1 position 30 targeted gRNA
  source 1..20: mol_type = genomic DNA
gtgagtgaatgagagcaaga

SEQ ID NO: 11 - DNA, 20 bp, Cannabis sativa
  misc_feature 1..20: note = CsMLO1 position 59 targeted gRNA
  source 1..20: mol_type = genomic DNA
attccgatttcgaattcaga

SEQ ID NO: 12 - DNA, 20 bp, Cannabis sativa
  misc_feature 1..20: note = CsMLO1 position 67 targeted gRNA
  source 1..20: mol_type = genomic DNA
aatccatctgaattcgaaat

SEQ ID NO: 13 - DNA, 20 bp, Cannabis sativa
  misc_feature 1..20: note = CsMLO1 76 targeted gRNA
  source 1..20: mol_type = genomic DNA
gaattcgaaatcggaatgag

SEQ ID NO: 14 - DNA, 20 bp, Cannabis sativa
  misc_feature 1..20: note = CsMLO1 position 79 targeted gRNA
  source 1..20: mol_type = genomic DNA
ttcgaaatcggaatgagtgg

SEQ ID NO: 15 - DNA, 20 bp, Cannabis sativa
  misc_feature 1..20: note = CsMLO1 position 82 targeted gRNA
  source 1..20: mol_type = genomic DNA
gaaatcggaatgagtggcgg

SEQ ID NO: 16 - DNA, 20 bp, Cannabis sativa
  misc_feature 1..20: note = CsMLO1 position 88 targeted gRNA
  source 1..20: mol_type = genomic DNA
ggaatgagtggcggtggaga

SEQ ID NO: 17 - DNA, 20 bp, Cannabis sativa
  misc_feature 1..20: note = CsMLO1 position 99 targeted gRNA
  source 1..20: mol_type = genomic DNA
cggtggagaaggtgagtcct

SEQ ID NO: 18 - DNA, 20 bp, Cannabis sativa
  misc_feature 1..20: note = CsMLO1 position 105 targeted gRNA
  source 1..20: mol_type = genomic DNA
ccatgtgggagtatactcca

SEQ ID NO: 19 - DNA, 20 bp, Cannabis sativa
  misc_feature 1..20: note = CsMLO1 position 116 targeted gRNA
  source 1..20: mol_type = genomic DNA
ccttggagtatactcccaca

SEQ ID NO: 20 - DNA, 20 bp, Cannabis sativa
  misc_feature 1..20: note = CsMLO1 position 119 targeted gRNA
  source 1..20: mol_type = genomic DNA
acgacggcgacgatccatgt

SEQ ID NO: 21 - DNA, 20 bp, Cannabis sativa
  misc_feature 1..20: note = CsMLO1 position 120 targeted gRNA
  source 1..20: mol_type = genomic DNA
gacgacggcgacgatccatg

SEQ ID NO: 22 - DNA, 20 bp, Cannabis sativa
  misc_feature 1..20: note = CsMLO1 position 135 targeted gRNA
  source 1..20: mol_type = genomic DNA
gacgatgacagagcagacga

SEQ ID NO: 23 - DNA, 20 bp, Cannabis sativa
  misc_feature 1..20: note = CsMLO1 position 159 targeted gRNA
  source 1..20: mol_type = genomic DNA
acgctccgcggcgagagaaa

SEQ ID NO: 24 - DNA, 20 bp, Cannabis sativa
  misc_feature 1..20: note = CsMLO1 position 165 targeted gRNA
  source 1..20: mol_type = genomic DNA
cgtcgccatttctctcgccg

SEQ ID NO: 25 - DNA, 20 bp, Cannabis sativa
  misc_feature 1..20: note = CsMLO1 position 171 targeted gRNA
  source 1..20: mol_type = genomic DNA
atagtggagaagacgctccg

SEQ ID NO: 26 - DNA, 20 bp, Cannabis sativa
  misc_feature 1..20: note = CsMLO1 position 187 targeted gRNA
  source 1..20: mol_type = genomic DNA
gagcgtcttctccactatct

SEQ ID NO: 27 - DNA, 20 bp, Cannabis sativa
  misc_feature 1..20: note = CsMLO1 position 187, strand -1, targeted gRNA
  source 1..20: mol_type = genomic DNA
tcaaaacctgaccgagatag

SEQ ID NO: 28 - DNA, 20 bp, Cannabis sativa
  misc_feature 1..20: note = CsMLO1 position 192 targeted gRNA
  source 1..20: mol_type = genomic DNA
tcttctccactatctcggtc

SEQ ID NO: 29 - DNA, 20 bp, Cannabis sativa
  misc_feature 1..20: note = CsMLO1 position 220 targeted gRNA
  source 1..20: mol_type = genomic DNA
aggcctcgtatagaggcttc

SEQ ID NO: 30 - DNA, 20 bp, Cannabis sativa
  misc_feature 1..20: note = CsMLO1 position 227 targeted gRNA
  source 1..20: mol_type = genomic DNA
ttctgcaaggcctcgtatag

SEQ ID NO: 31 - DNA, 20 bp, Cannabis sativa
  misc_feature 1..20: note = CsMLO1 position 228 targeted gRNA
  source 1..20: mol_type = genomic DNA
gaaccagaagcctctatacg

SEQ ID NO: 32 - DNA, 20 bp, Cannabis sativa
  misc_feature 1..20: note = CsMLO1 position 240 targeted gRNA
  source 1..20: mol_type = genomic DNA
ctcctccttgatcttctgca

SEQ ID NO: 33 - DNA, 20 bp, Cannabis sativa
  misc_feature 1..20: note = CsMLO1 position 246 targeted gRNA
  source 1..20: mol_type = genomic DNA
cgaggccttgcagaagatca

SEQ ID NO: 34 - DNA, 20 bp, Cannabis sativa
  misc_feature 1..20: note = CsMLO1 position 249 targeted gRNA
  source 1..20: mol_type = genomic DNA
ggccttgcagaagatcaagg

SEQ ID NO: 35 - DNA, 20 bp, Cannabis sativa
  misc_feature 1..20: note = CsMLO1 position 264 targeted gRNA
  source 1..20: mol_type = genomic DNA
caaggaggagttgatgcttt

SEQ ID NO: 36 - DNA, 20 bp, Cannabis sativa
  misc_feature 1..20: note = CsMLO1 position 265 targeted gRNA
  source 1..20: mol_type = genomic DNA
aaggaggagttgatgctttt

SEQ ID NO: 37 - DNA, 20 bp, Cannabis sativa
  misc_feature 1..20: note = CsMLO1 position 292 targeted gRNA
  source 1..20: mol_type = genomic DNA
ttgtgttctgcgaaacagtg

SEQ ID NO: 38 - DNA, 20 bp, Cannabis sativa
  misc_feature 1..20: note = CsMLO1 position 332 targeted gRNA
  source 1..20: mol_type = genomic DNA
agattgtcgaccaaagaagc

SEQ ID NO: 39 - DNA, 20 bp, Cannabis sativa
  misc_feature 1..20: note = CsMLO1 position 333 targeted gRNA
  source 1..20: mol_type = genomic DNA
gttttgcgtacctgcttctt

SEQ ID NO: 40 - DNA, 20 bp, Cannabis sativa
  misc_feature 1..20: note = CsMLO1 position 355 targeted gRNA
  source 1..20: mol_type = genomic DNA
gatgagggcgcttacaaggg

SEQ ID NO: 41 - DNA, 20 bp, Cannabis sativa
  misc_feature 1..20: note = CsMLO1 position 358 targeted gRNA
  source 1..20: mol_type = genomic DNA
cctgatgagggcgcttacaa

SEQ ID NO: 42 - DNA, 20 bp, Cannabis sativa
  misc_feature 1..20: note = CsMLO1 position 359 targeted gRNA
  source 1..20: mol_type = genomic DNA
tcctgatgagggcgcttaca

SEQ ID NO: 43 - DNA, 20 bp, Cannabis sativa
  misc_feature 1..20: note = CsMLO1 position 369 targeted gRNA
  source 1..20: mol_type = genomic DNA
cccttgtaagcgccctcatc

SEQ ID NO: 44 - DNA, 20 bp, Cannabis sativa
  misc_feature 1..20: note = CsMLO1 position 370 targeted gRNA
  source 1..20: mol_type = genomic DNA
aatcattagcttcctgatga

SEQ ID NO: 45 - DNA, 20 bp, Cannabis sativa
  misc_feature 1..20: note = CsMLO1 position 371targeted gRNA
  source 1..20: mol_type = genomic DNA
gaatcattagcttcctgatg

SEQ ID NO: 46 - DNA, 20 bp, Cannabis sativa
  misc_feature 1..20: note = CsMLO1 position 405 targeted gRNA
  source 1..20: mol_type = genomic DNA
agagccggagatgtgatgag

SEQ ID NO: 47 - DNA, 20 bp, Cannabis sativa
  misc_feature 1..20: note = CsMLO1 412targeted gRNA
  source 1..20: mol_type = genomic DNA
tcaacctctcatcacatctc

SEQ ID NO: 48 - DNA, 20 bp, Cannabis sativa
  misc_feature 1..20: note = CsMLO1 position 420 targeted gRNA
  source 1..20: mol_type = genomic DNA
aagaaggcgtctgaaagagc

SEQ ID NO: 49 - DNA, 20 bp, Cannabis sativa
  misc_feature 1..20: note = CsMLO1 position 436 targeted gRNA
  source 1..20: mol_type = genomic DNA
cagtggaagtttcttcaaga

SEQ ID NO: 50 - DNA, 20 bp, Cannabis sativa
  misc_feature 1..20: note = CsMLO1 position 453 targeted gRNA
  source 1..20: mol_type = genomic DNA
gcaataacccaaatgagcag

SEQ ID NO: 51 - DNA, 20 bp, Cannabis sativa
  misc_feature 1..20: note = CsMLO1 position 456 targeted gRNA
  source 1..20: mol_type = genomic DNA
agaaacttccactgctcatt

SEQ ID NO: 52 - DNA, 20 bp, Cannabis sativa
  misc_feature 1..20: note = CsMLO1 position 457 targeted gRNA
  source 1..20: mol_type = genomic DNA
gaaacttccactgctcattt

SEQ ID NO: 53 - DNA, 20 bp, Cannabis sativa
  misc_feature 1..20: note = CsMLO1 position 474 targeted gRNA
  source 1..20: mol_type = genomic DNA
tttgggttattgcgctcata

SEQ ID NO: 54 - DNA, 20 bp, Cannabis sativa
  misc_feature 1..20: note = CsMLO1 position 501 targeted gRNA
  source 1..20: mol_type = genomic DNA
acaacaacaactaaagatat

SEQ ID NO: 55 - DNA, 20 bp, Cannabis sativa
  misc_feature 1..20: note = CsMLO1 position 502 targeted gRNA
  source 1..20: mol_type = genomic DNA
aacaacaacaactaaagata

SEQ ID NO: 56 - DNA, 20 bp, Cannabis sativa
  misc_feature 1..20: note = CsMLO1 position 521 targeted gRNA
  source 1..20: mol_type = genomic DNA
ttagttgttgttgttttttt

SEQ ID NO: 57 - DNA, 20 bp, Cannabis sativa
  misc_feature 1..20: note = CsMLO1 position 522 targeted gRNA
  source 1..20: mol_type = genomic DNA
tagttgttgttgttttttta

SEQ ID NO: 58 - DNA, 20 bp, Cannabis sativa
  misc_feature 1..20: note = CsMLO1 position 523 targeted gRNA
  source 1..20: mol_type = genomic DNA
agttgttgttgtttttttag

SEQ ID NO: 59 - DNA, 20 bp, Cannabis sativa
  misc_feature 1..20: note = CsMLO1 position 570 targeted gRNA
  source 1..20: mol_type = genomic DNA
tataaatatactttcccaaa

SEQ ID NO: 60 - DNA, 20 bp, Cannabis sativa
  misc_feature 1..20: note = CsMLO1 position 571 targeted gRNA
  source 1..20: mol_type = genomic DNA
ataaatatactttcccaaaa

SEQ ID NO: 61 - DNA, 20 bp, Cannabis sativa
  misc_feature 1..20: note = CsMLO1 position 573 targeted gRNA
  source 1..20: mol_type = genomic DNA
taaagcgaatagtccctttt

SEQ ID NO: 62 - DNA, 20 bp, Cannabis sativa
  misc_feature 1..20: note = CsMLO1 position 574 targeted gRNA
  source 1..20: mol_type = genomic DNA
ttaaagcgaatagtcccttt

SEQ ID NO: 63 - DNA, 20 bp, Cannabis sativa
  misc_feature 1..20: note = CsMLO1 position 657 targeted gRNA
  source 1..20: mol_type = genomic DNA
atgcttcaacggaataaaag

SEQ ID NO: 64 - DNA, 20 bp, Cannabis sativa
  misc_feature 1..20: note = CsMLO1 position 658 targeted gRNA
  source 1..20: mol_type = genomic DNA
aatgcttcaacggaataaaa

SEQ ID NO: 65 - DNA, 20 bp, Cannabis sativa
  misc_feature 1..20: note = CsMLO1 position 659 targeted gRNA
  source 1..20: mol_type = genomic DNA
caatgcttcaacggaataaa

SEQ ID NO: 66 - DNA, 20 bp, Cannabis sativa
  misc_feature 1..20: note = CsMLO1 position 668 targeted gRNA
  source 1..20: mol_type = genomic DNA
caaatggtgcaatgcttcaa

SEQ ID NO: 67 - DNA, 20 bp, Cannabis sativa
  misc_feature 1..20: note = CsMLO1 position 684 targeted gRNA
  source 1..20: mol_type = genomic DNA
cgaagataaagatatgcaaa

SEQ ID NO: 68 - DNA, 20 bp, Cannabis sativa
  misc_feature 1..20: note = CsMLO1 position 708 targeted gRNA
  source 1..20: mol_type = genomic DNA
aagtgacatggacaatggct

SEQ ID NO: 69 - DNA, 20 bp, Cannabis sativa
  misc_feature 1..20: note = CsMLO1 position 713 targeted gRNA
  source 1..20: mol_type = genomic DNA
acagaaagtgacatggacaa

SEQ ID NO: 70 - DNA, 20 bp, Cannabis sativa
  misc_feature 1..20: note = CsMLO1 720 targeted gRNA
  source 1..20: mol_type = genomic DNA
tgagaacacagaaagtgaca

SEQ ID NO: 71 - DNA, 20 bp, Cannabis sativa
  misc_feature 1..20: note = CsMLO1 744 targeted gRNA
  source 1..20: mol_type = genomic DNA
tgtgttctcactgttgtgtt

SEQ ID NO: 72 - DNA, 20 bp, Cannabis sativa
  misc_feature 1..20: note = CsMLO1 position 747 targeted gRNA
  source 1..20: mol_type = genomic DNA
gttctcactgttgtgtttgg

SEQ ID NO: 73 - DNA, 20 bp, Cannabis sativa
  misc_feature 1..20: note = CsMLO1 position 755 targeted gRNA
  source 1..20: mol_type = genomic DNA
tgttgtgtttggaggtgtaa

SEQ ID NO: 74 - DNA, 20 bp, Cannabis sativa
  misc_feature 1..20: note = CsMLO1 position 779 targeted gRNA
  source 1..20: mol_type = genomic DNA
gagagagaagcatatgaatt

SEQ ID NO: 75 - DNA, 20 bp, Cannabis sativa
  misc_feature 1..20: note = CsMLO1 position 860 targeted gRNA
  source 1..20: mol_type = genomic DNA
tacacaactagatacgtcaa

SEQ ID NO: 76 - DNA, 20 bp, Cannabis sativa
  misc_feature 1..20: note = CsMLO1 position 869 targeted gRNA
  source 1..20: mol_type = genomic DNA
agatacgtcaatggaaacgt

SEQ ID NO: 77 - DNA, 20 bp, Cannabis sativa
  misc_feature 1..20: note = CsMLO1 position 870 targeted gRNA
  source 1..20: mol_type = genomic DNA
gatacgtcaatggaaacgtt

SEQ ID NO: 78 - DNA, 20 bp, Cannabis sativa
  misc_feature 1..20: note = CsMLO1 position 873 targeted gRNA
  source 1..20: mol_type = genomic DNA
acgtcaatggaaacgttggg

SEQ ID NO: 79 - DNA, 20 bp, Cannabis sativa
  misc_feature 1..20: note = CsMLO1 position 910 targeted gRNA
  source 1..20: mol_type = genomic DNA
gagagttatgacactgaaca

SEQ ID NO: 80 - DNA, 20 bp, Cannabis sativa
  misc_feature 1..20: note = CsMLO1 position 937 targeted gRNA
  source 1..20: mol_type = genomic DNA
ttctataatattgtcaaaag

SEQ ID NO: 81 - DNA, 20 bp, Cannabis sativa
  misc_feature 1..20: note = CsMLO1 position 978 targeted gRNA
  source 1..20: mol_type = genomic DNA
taagcgattcatatgttaga

SEQ ID NO: 82 - DNA, 20 bp, Cannabis sativa
  misc_feature 1..20: note = CsMLO1 position 1017 targeted gRNA
  source 1..20: mol_type = genomic DNA
tgttcttaagtctaagaaaa

SEQ ID NO: 83 - DNA, 20 bp, Cannabis sativa
  misc_feature 1..20: note = CsMLO1 position 1039 targeted gRNA
  source 1..20: mol_type = genomic DNA
ccttaatgaacgcgtgttga

SEQ ID NO: 84 - DNA, 20 bp, Cannabis sativa
  misc_feature 1..20: note = CsMLO1 position 1050 targeted gRNA
  source 1..20: mol_type = genomic DNA
ccatcaacacgcgttcatta

SEQ ID NO: 85 - DNA, 20 bp, Cannabis sativa
  misc_feature 1..20: note = CsMLO1 position 1062 targeted gRNA
  source 1..20: mol_type = genomic DNA
gttcattaaggaccactttt

SEQ ID NO: 86 - DNA, 20 bp, Cannabis sativa
  misc_feature 1..20: note = CsMLO1 position 1063 targeted gRNA
  source 1..20: mol_type = genomic DNA
ttcattaaggaccacttttt

SEQ ID NO: 87 - DNA, 20 bp, Cannabis sativa
  misc_feature 1..20: note = CsMLO1 position 1063, strand -1,targeted gRNA
  source 1..20: mol_type = genomic DNA
ctttaccaaaacccaaaaag

SEQ ID NO: 88 - DNA, 20 bp, Cannabis sativa
  misc_feature 1..20: note = CsMLO1 position 1069 targeted gRNA
  source 1..20: mol_type = genomic DNA
aaggaccactttttgggttt

SEQ ID NO: 89 - DNA, 20 bp, Cannabis sativa
  misc_feature 1..20: note = CsMLO1 position 1090 targeted gRNA
  source 1..20: mol_type = genomic DNA
ggtaaagactcagctctact

SEQ ID NO: 90 - DNA, 20 bp, Cannabis sativa
  misc_feature 1..20: note = CsMLO1 position 1094 targeted gRNA
  source 1..20: mol_type = genomic DNA
aagactcagctctactaggc

SEQ ID NO: 91 - DNA, 20 bp, Cannabis sativa
  misc_feature 1..20: note = CsMLO1 position 1098 targeted gRNA
  source 1..20: mol_type = genomic DNA
ctcagctctactaggctggc

SEQ ID NO: 92 - DNA, 20 bp, Cannabis sativa
  misc_feature 1..20: note = CsMLO1 position 1140 targeted gRNA
  source 1..20: mol_type = genomic DNA
ataatcctaattcagaactt

SEQ ID NO: 93 - DNA, 20 bp, Cannabis sativa
  misc_feature 1..20: note = CsMLO1 position 1146 targeted gRNA
  source 1..20: mol_type = genomic DNA
agtatccaaagttctgaatt

SEQ ID NO: 94 - DNA, 20 bp, Cannabis sativa
  misc_feature 1..20: note = CsMLO1 position 1159 targeted gRNA
  source 1..20: mol_type = genomic DNA
ctgaattaggattattctta

SEQ ID NO: 95 - DNA, 20 bp, Cannabis sativa
  misc_feature 1..20: note = CsMLO1 position 1174 targeted gRNA
  source 1..20: mol_type = genomic DNA
atatcaagagaataagaaaa

SEQ ID NO: 96 - DNA, 20 bp, Cannabis sativa
  misc_feature 1..20: note = CsMLO1 position 1214 targeted gRNA
  source 1..20: mol_type = genomic DNA
aacgttctcaaaatagaaag

SEQ ID NO: 97 - DNA, 20 bp, Cannabis sativa
  misc_feature 1..20: note = CsMLO1 position 1267 targeted gRNA
  source 1..20: mol_type = genomic DNA
ctccaaaggctcagtatcaa

SEQ ID NO: 98 - DNA, 20 bp, Cannabis sativa
  misc_feature 1..20: note = CsMLO1 position 1276 targeted gRNA
  source 1..20: mol_type = genomic DNA
ctccattgatactgagcctt

SEQ ID NO: 99 - DNA, 20 bp, Cannabis sativa
  misc_feature 1..20: note = CsMLO1 position 1281 targeted gRNA
  source 1..20: mol_type = genomic DNA
gtgatggagaaaatctccaa

SEQ ID NO: 100 - DNA, 20 bp, Cannabis sativa
  misc_feature 1..20: note = CsMLO1 position 1297 targeted gRNA
  source 1..20: mol_type = genomic DNA
atgtcctagttttcatgtga

SEQ ID NO: 101 - DNA, 20 bp, Cannabis sativa
  misc_feature 1..20: note = CsMLO1 position 1304 targeted gRNA
  source 1..20: mol_type = genomic DNA
ttctccatcacatgaaaact

SEQ ID NO: 102 - DNA, 20 bp, Cannabis sativa
  misc_feature 1..20: note = CsMLO1 position 1327 targeted gRNA
  source 1..20: mol_type = genomic DNA
acatttttgtgcacatgtta

SEQ ID NO: 103 - DNA, 20 bp, Cannabis sativa
  misc_feature 1..20: note = CsMLO1 position 1349 targeted gRNA
  source 1..20: mol_type = genomic DNA
gagctagctaacattaacat

SEQ ID NO: 104 - DNA, 20 bp, Cannabis sativa
  misc_feature 1..20: note = CsMLO1 position 1356 targeted gRNA
  source 1..20: mol_type = genomic DNA
ctaacattaacattggaaac

SEQ ID NO: 105 - DNA, 20 bp, Cannabis sativa
  misc_feature 1..20: note = CsMLO1 position 1379 targeted gRNA
  source 1..20: mol_type = genomic DNA
ggggaaaaagaatcaaatca

SEQ ID NO: 106 - DNA, 20 bp, Cannabis sativa
  misc_feature 1..20: note = CsMLO1 position 1398 targeted gRNA
  source 1..20: mol_type = genomic DNA
aaaagcatgctgttcacaag

SEQ ID NO: 107 - DNA, 20 bp, Cannabis sativa
  misc_feature 1..20: note = CsMLO1 position 1399 targeted gRNA
  source 1..20: mol_type = genomic DNA
caaaagcatgctgttcacaa

SEQ ID NO: 108 - DNA, 20 bp, Cannabis sativa
  misc_feature 1..20: note = CsMLO1 position 1400 targeted gRNA
  source 1..20: mol_type = genomic DNA
acaaaagcatgctgttcaca

SEQ ID NO: 109 - DNA, 20 bp, Cannabis sativa
  misc_feature 1..20: note = CsMLO1 position 1446 targeted gRNA
  source 1..20: mol_type = genomic DNA
catggtcgcataatctgatt

SEQ ID NO: 110 - DNA, 20 bp, Cannabis sativa
  misc_feature 1..20: note = CsMLO1 position 1460 targeted gRNA
  source 1..20: mol_type = genomic DNA
aatcagattatgcgaccatg

SEQ ID NO: 111 - DNA, 20 bp, Cannabis sativa
  misc_feature 1..20: note = CsMLO1 position 1464 targeted gRNA
  source 1..20: mol_type = genomic DNA
catgatgaatcctaaccgca

SEQ ID NO: 112 - DNA, 20 bp, Cannabis sativa
  misc_feature 1..20: note = CsMLO1 position 1465 targeted gRNA
  source 1..20: mol_type = genomic DNA
gattatgcgaccatgcggtt

SEQ ID NO: 113 - DNA, 20 bp, Cannabis sativa
  misc_feature 1..20: note = CsMLO1 position 1476 targeted gRNA
  source 1..20: mol_type = genomic DNA
catgcggttaggattcatca

SEQ ID NO: 114 - DNA, 20 bp, Cannabis sativa
  misc_feature 1..20: note = CsMLO1 position 1520 targeted gRNA
  source 1..20: mol_type = genomic DNA
ttacttataaattactagaa

SEQ ID NO: 115 - DNA, 20 bp, Cannabis sativa
  misc_feature 1..20: note = CsMLO1 position 1563 targeted gRNA
  source 1..20: mol_type = genomic DNA
ctttttttcttttcactaaa

SEQ ID NO: 116 - DNA, 20 bp, Cannabis sativa
  misc_feature 1..20: note = CsMLO1 position 1603 targeted gRNA
  source 1..20: mol_type = genomic DNA
ttgtattctagactcactgc

SEQ ID NO: 117 - DNA, 20 bp, Cannabis sativa
  misc_feature 1..20: note = CsMLO1 position 1604 targeted gRNA
  source 1..20: mol_type = genomic DNA
tgtattctagactcactgca

SEQ ID NO: 118 - DNA, 20 bp, Cannabis sativa
  misc_feature 1..20: note = CsMLO1 position 1605 targeted gRNA
  source 1..20: mol_type = genomic DNA
gtattctagactcactgcag

SEQ ID NO: 119 - DNA, 20 bp, Cannabis sativa
  misc_feature 1..20: note = CsMLO1 position 1674 targeted gRNA
  source 1..20: mol_type = genomic DNA
gatgatttcaagaaagttgt

SEQ ID NO: 120 - DNA, 20 bp, Cannabis sativa
  misc_feature 1..20: note = CsMLO1 position 1675 targeted gRNA
  source 1..20: mol_type = genomic DNA
atgatttcaagaaagttgtt

SEQ ID NO: 121 - DNA, 20 bp, Cannabis sativa
  misc_feature 1..20: note = CsMLO1 position 1681 targeted gRNA
  source 1..20: mol_type = genomic DNA
tcaagaaagttgttgggata

SEQ ID NO: 122 - DNA, 20 bp, Cannabis sativa
  misc_feature 1..20: note = CsMLO1 position 1691 targeted gRNA
  source 1..20: mol_type = genomic DNA
tgttgggataaggtaaccct

SEQ ID NO: 123 - DNA, 20 bp, Cannabis sativa
  misc_feature 1..20: note = CsMLO1 position 1696 targeted gRNA
  source 1..20: mol_type = genomic DNA
gaaaatagactgtcaaccaa

SEQ ID NO: 124 - DNA, 20 bp, Cannabis sativa
  misc_feature 1..20: note = CsMLO1 position 1697 targeted gRNA
  source 1..20: mol_type = genomic DNA
agaaaatagactgtcaacca

SEQ ID NO: 125 - DNA, 20 bp, Cannabis sativa
  misc_feature 1..20: note = CsMLO1 position 1777 targeted gRNA
  source 1..20: mol_type = genomic DNA
ttcttttaagtctactgtat

SEQ ID NO: 126 - DNA, 20 bp, Cannabis sativa
  misc_feature 1..20: note = CsMLO1 position 1792 targeted gRNA
  source 1..20: mol_type = genomic DNA
aaaagctaaaaggtcaatct

SEQ ID NO: 127 - DNA, 20 bp, Cannabis sativa
  misc_feature 1..20: note = CsMLO1 position 1802 targeted gRNA
  source 1..20: mol_type = genomic DNA
actgcagccaaaaagctaaa

SEQ ID NO: 128 - DNA, 20 bp, Cannabis sativa
  misc_feature 1..20: note = CsMLO1 position 1806 targeted gRNA
  source 1..20: mol_type = genomic DNA
agattgaccttttagctttt

SEQ ID NO: 129 - DNA, 20 bp, Cannabis sativa
  misc_feature 1..20: note = CsMLO1 position 1816 targeted gRNA
  source 1..20: mol_type = genomic DNA
tttagctttttggctgcagt

SEQ ID NO: 130 - DNA, 20 bp, Cannabis sativa
  misc_feature 1..20: note = CsMLO1 position 1825 targeted gRNA
  source 1..20: mol_type = genomic DNA
ttggctgcagttggtacctt

SEQ ID NO: 131 - DNA, 20 bp, Cannabis sativa
  misc_feature 1..20: note = CsMLO1 position 1826 targeted gRNA
  source 1..20: mol_type = genomic DNA
tggctgcagttggtaccttt

SEQ ID NO: 132 - DNA, 20 bp, Cannabis sativa
  misc_feature 1..20: note = CsMLO1 position 1830 targeted gRNA
  source 1..20: mol_type = genomic DNA
agatgaccacaaaaacccaa

SEQ ID NO: 133 - DNA, 20 bp, Cannabis sativa
  misc_feature 1..20: note = CsMLO1 position 1835 targeted gRNA
  source 1..20: mol_type = genomic DNA
ttggtacctttgggtttttg

SEQ ID NO: 134 - DNA, 20 bp, Cannabis sativa
  misc_feature 1..20: note = CsMLO1 position 1863 targeted gRNA
  source 1..20: mol_type = genomic DNA
ttcttgttgctgaatgttaa

SEQ ID NO: 135 - DNA, 20 bp, Cannabis sativa
  misc_feature 1..20: note = CsMLO1 position 1910 targeted gRNA
  source 1..20: mol_type = genomic DNA
atcacttagatcttgagtta

SEQ ID NO: 136 - DNA, 20 bp, Cannabis sativa
  misc_feature 1..20: note = CsMLO1 position 1926 targeted gRNA
  source 1..20: mol_type = genomic DNA
actcaagatctaagtgatat

SEQ ID NO: 137 - DNA, 20 bp, Cannabis sativa
  misc_feature 1..20: note = CsMLO1 position 1958 targeted gRNA
  source 1..20: mol_type = genomic DNA
caaagaaccagactgattac

SEQ ID NO: 138 - DNA, 20 bp, Cannabis sativa
  misc_feature 1..20: note = CsMLO1 position 1959 targeted gRNA
  source 1..20: mol_type = genomic DNA
acaaagaaccagactgatta

SEQ ID NO: 139 - DNA, 20 bp, Cannabis sativa
  misc_feature 1..20: note = CsMLO1 position 1962 targeted gRNA
  source 1..20: mol_type = genomic DNA
gcagaatcccgtaatcagtc

SEQ ID NO: 140 - DNA, 20 bp, Cannabis sativa
  misc_feature 1..20: note = CsMLO1 position 1999 targeted gRNA
  source 1..20: mol_type = genomic DNA
cttcaagtgtgtcatctctt

SEQ ID NO: 141 - DNA, 20 bp, Cannabis sativa
  misc_feature 1..20: note = CsMLO1 position 2033 targeted gRNA
  source 1..20: mol_type = genomic DNA
gcttatttgaaactataatt

SEQ ID NO: 142 - DNA, 20 bp, Cannabis sativa
  misc_feature 1..20: note = CsMLO1 position 2101 targeted gRNA
  source 1..20: mol_type = genomic DNA
gtggaggcagagtaaggaat

SEQ ID NO: 143 - DNA, 20 bp, Cannabis sativa
  misc_feature 1..20: note = CsMLO1 position 2107 targeted gRNA
  source 1..20: mol_type = genomic DNA
agaaaagtggaggcagagta

SEQ ID NO: 144 - DNA, 20 bp, Cannabis sativa
  misc_feature 1..20: note = CsMLO1 position 2117targeted gRNA
  source 1..20: mol_type = genomic DNA
ctccaaccatagaaaagtgg

SEQ ID NO: 145 - DNA, 20 bp, Cannabis sativa
  misc_feature 1..20: note = CsMLO1 position 2120 targeted gRNA
  source 1..20: mol_type = genomic DNA
tttctccaaccatagaaaag

SEQ ID NO: 146 - DNA, 20 bp, Cannabis sativa
  misc_feature 1..20: note = CsMLO1 position 2122 targeted gRNA
  source 1..20: mol_type = genomic DNA
actctgcctccacttttcta

SEQ ID NO: 147 - DNA, 20 bp, Cannabis sativa
  misc_feature 1..20: note = CsMLO1 position 2126 targeted gRNA
  source 1..20: mol_type = genomic DNA
tgcctccacttttctatggt

SEQ ID NO: 148 - DNA, 20 bp, Cannabis sativa
  misc_feature 1..20: note = CsMLO1 position 2148 targeted gRNA
  source 1..20: mol_type = genomic DNA
gagaaaattatactccaagt

SEQ ID NO: 149 - DNA, 20 bp, Cannabis sativa
  misc_feature 1..20: note = CsMLO1 position 2151 targeted gRNA
  source 1..20: mol_type = genomic DNA
tccaattccttacaccaact

SEQ ID NO: 150 - DNA, 20 bp, Cannabis sativa
  misc_feature 1..20: note = CsMLO1 position 2155 targeted gRNA
  source 1..20: mol_type = genomic DNA
ttatactccaagttggtgta

SEQ ID NO: 151 - DNA, 20 bp, Cannabis sativa
  misc_feature 1..20: note = CsMLO1 position 2161 targeted gRNA
  source 1..20: mol_type = genomic DNA
tccaagttggtgtaaggaat

SEQ ID NO: 152 - DNA, 20 bp, Cannabis sativa
  misc_feature 1..20: note = CsMLO1 position 2203targeted gRNA
  source 1..20: mol_type = genomic DNA
tcactagaatgcaatcaaca

SEQ ID NO: 153 - DNA, 20 bp, Cannabis sativa
  misc_feature 1..20: note = CsMLO1 position 2204 targeted gRNA
  source 1..20: mol_type = genomic DNA
cactagaatgcaatcaacaa

SEQ ID NO: 154 - DNA, 20 bp, Cannabis sativa
  misc_feature 1..20: note = CsMLO1 position 2244 targeted gRNA
  source 1..20: mol_type = genomic DNA
aatttttttaatcagaattc

SEQ ID NO: 155 - DNA, 20 bp, Cannabis sativa
  misc_feature 1..20: note = CsMLO1 position 2293 targeted gRNA
  source 1..20: mol_type = genomic DNA
taaaagtaaactaaatttct

SEQ ID NO: 156 - DNA, 20 bp, Cannabis sativa
  misc_feature 1..20: note = CsMLO1 position 2347 targeted gRNA
  source 1..20: mol_type = genomic DNA
atgtaaattatgttctatat

SEQ ID NO: 157 - DNA, 20 bp, Cannabis sativa
  misc_feature 1..20: note = CsMLO1 position 2380 targeted gRNA
  source 1..20: mol_type = genomic DNA
gatccaattgaattatctta

SEQ ID NO: 158 - DNA, 20 bp, Cannabis sativa
  misc_feature 1..20: note = CsMLO1 position 2388 targeted gRNA
  source 1..20: mol_type = genomic DNA
acaccttaagataattcaat

SEQ ID NO: 159 - DNA, 20 bp, Cannabis sativa
  misc_feature 1..20: note = CsMLO1 position 2406 targeted gRNA
  source 1..20: mol_type = genomic DNA
attggatcttactccttgtt

SEQ ID NO: 160 - DNA, 20 bp, Cannabis sativa
  misc_feature 1..20: note = CsMLO1 position 2408 targeted gRNA
  source 1..20: mol_type = genomic DNA
gcctcattgtagtccaaaca

SEQ ID NO: 161 - DNA, 20 bp, Cannabis sativa
  misc_feature 1..20: note = CsMLO1 position 2418 targeted gRNA
  source 1..20: mol_type = genomic DNA
tccttgtttggactacaatg

SEQ ID NO: 162 - DNA, 20 bp, Cannabis sativa
  misc_feature 1..20: note = CsMLO1 position 2453 targeted gRNA
  source 1..20: mol_type = genomic DNA
atcttggtttgagtattgag

SEQ ID NO: 163 - DNA, 20 bp, Cannabis sativa
  misc_feature 1..20: note = CsMLO1 position 2469 targeted gRNA
  source 1..20: mol_type = genomic DNA
atataaataatgaatgatct

SEQ ID NO: 164 - DNA, 20 bp, Cannabis sativa
  misc_feature 1..20: note = CsMLO1 position 2497 targeted gRNA
  source 1..20: mol_type = genomic DNA
caaagaagtttaatacacac

SEQ ID NO: 165 - DNA, 20 bp, Cannabis sativa
  misc_feature 1..20: note = CsMLO1 position 2516 targeted gRNA
  source 1..20: mol_type = genomic DNA
tattaaacttctttgttgta

SEQ ID NO: 166 - DNA, 20 bp, Cannabis sativa
  misc_feature 1..20: note = CsMLO1 position 2559 targeted gRNA
  source 1..20: mol_type = genomic DNA
ttttgtcaatgttttgtgat

SEQ ID NO: 167 - DNA, 20 bp, Cannabis sativa
  misc_feature 1..20: note = CsMLO1 position 2597 targeted gRNA
  source 1..20: mol_type = genomic DNA
taataatgtgttatatttgc

SEQ ID NO: 168 - DNA, 20 bp, Cannabis sativa
  misc_feature 1..20: note = CsMLO1 position 2601 targeted gRNA
  source 1..20: mol_type = genomic DNA
aatgtgttatatttgcaggc

SEQ ID NO: 169 - DNA, 20 bp, Cannabis sativa
  misc_feature 1..20: note = CsMLO1 position 2616 targeted gRNA
  source 1..20: mol_type = genomic DNA
caggctggcacacatatttc

SEQ ID NO: 170 - DNA, 20 bp, Cannabis sativa
  misc_feature 1..20: note = CsMLO1 position 2640 targeted gRNA
  source 1..20: mol_type = genomic DNA
ctcaataaactcacaaagaa

SEQ ID NO: 171 - DNA, 20 bp, Cannabis sativa
  misc_feature 1..20: note = CsMLO1 position 2709 targeted gRNA
  source 1..20: mol_type = genomic DNA
catgtttcattgttcttgca

SEQ ID NO: 172 - DNA, 20 bp, Cannabis sativa
  misc_feature 1..20: note = CsMLO1 position 2750 targeted gRNA
  source 1..20: mol_type = genomic DNA
cattttaagtatcatactga

SEQ ID NO: 173 - DNA, 20 bp, Cannabis sativa
  misc_feature 1..20: note = CsMLO1 position 2774 targeted gRNA
  source 1..20: mol_type = genomic DNA
gaaagagataaaatacagag

SEQ ID NO: 174 - DNA, 20 bp, Cannabis sativa
  misc_feature 1..20: note = CsMLO1 position 2775 targeted gRNA
  source 1..20: mol_type = genomic DNA
aaagagataaaatacagaga

SEQ ID NO: 175 - DNA, 20 bp, Cannabis sativa
  misc_feature 1..20: note = CsMLO1 position 2783 targeted gRNA
  source 1..20: mol_type = genomic DNA
aaaatacagagagggagaat

SEQ ID NO: 176 - DNA, 20 bp, Cannabis sativa
  misc_feature 1..20: note = CsMLO1 position 2784 targeted gRNA
  source 1..20: mol_type = genomic DNA
aaatacagagagggagaatc

SEQ ID NO: 177 - DNA, 20 bp, Cannabis sativa
  misc_feature 1..20: note = CsMLO1 position 2817 targeted gRNA
  source 1..20: mol_type = genomic DNA
tttaacacaattttgtaaat

SEQ ID NO: 178 - DNA, 20 bp, Cannabis sativa
  misc_feature 1..20: note = CsMLO1 position 2824 targeted gRNA
  source 1..20: mol_type = genomic DNA
caattttgtaaataggcaaa

SEQ ID NO: 179 - DNA, 20 bp, Cannabis sativa
  misc_feature 1..20: note = CsMLO1 position 2837 targeted gRNA
  source 1..20: mol_type = genomic DNA
aggcaaatggacagctaaga

SEQ ID NO: 180 - DNA, 20 bp, Cannabis sativa
  misc_feature 1..20: note = CsMLO1 position 2852 targeted gRNA
  source 1..20: mol_type = genomic DNA
tgttcaattaattctaaatt

SEQ ID NO: 181 - DNA, 20 bp, Cannabis sativa
  misc_feature 1..20: note = CsMLO1 position 2875 targeted gRNA
  source 1..20: mol_type = genomic DNA
ttaattgaacaacatgacct

SEQ ID NO: 182 - DNA, 20 bp, Cannabis sativa
  misc_feature 1..20: note = CsMLO1 position 2881 targeted gRNA
  source 1..20: mol_type = genomic DNA
aaattgcacaatatttacct

SEQ ID NO: 183 - DNA, 20 bp, Cannabis sativa
  misc_feature 1..20: note = CsMLO1 position 2950 targeted gRNA
  source 1..20: mol_type = genomic DNA
taaatgtagagtcatgagtc

SEQ ID NO: 184 - DNA, 20 bp, Cannabis sativa
  misc_feature 1..20: note = CsMLO1 position 2951 targeted gRNA
  source 1..20: mol_type = genomic DNA
aaatgtagagtcatgagtca

SEQ ID NO: 185 - DNA, 20 bp, Cannabis sativa
  misc_feature 1..20: note = CsMLO1 position 2973 targeted gRNA
  source 1..20: mol_type = genomic DNA
gtagaaatttgcacctagac

SEQ ID NO: 186 - DNA, 20 bp, Cannabis sativa
  misc_feature 1..20: note = CsMLO1 position 2975 targeted gRNA
  source 1..20: mol_type = genomic DNA
caccttaaaaccacctgtct

SEQ ID NO: 187 - DNA, 20 bp, Cannabis sativa
  misc_feature 1..20: note = CsMLO1 position 2976 targeted gRNA
  source 1..20: mol_type = genomic DNA
gaaatttgcacctagacagg

SEQ ID NO: 188 - DNA, 20 bp, Cannabis sativa
  misc_feature 1..20: note = CsMLO1 position 2984targeted gRNA
  source 1..20: mol_type = genomic DNA
cacctagacaggtggtttta

SEQ ID NO: 189 - DNA, 20 bp, Cannabis sativa
  misc_feature 1..20: note = CsMLO1 position 2987 targeted gRNA
  source 1..20: mol_type = genomic DNA
ctagacaggtggttttaagg

SEQ ID NO: 190 - DNA, 20 bp, Cannabis sativa
  misc_feature 1..20: note = CsMLO1 position 3011 targeted gRNA
  source 1..20: mol_type = genomic DNA
acttctcatctccaagtctt

SEQ ID NO: 191 - DNA, 20 bp, Cannabis sativa
  misc_feature 1..20: note = CsMLO1 position 3011, strand -1, targeted gRNA
  source 1..20: mol_type = genomic DNA
catacatatcacctaagact

SEQ ID NO: 192 - DNA, 20 bp, Cannabis sativa
  misc_feature 1..20: note = CsMLO1 position 3011, strand -1, targeted gRNA
  source 1..20: mol_type = genomic DNA
atgtatatcacaacagcaaa

SEQ ID NO: 193 - DNA, 20 bp, Cannabis sativa
  misc_feature 1..20: note = CsMLO1 position 3083 targeted gRNA
  source 1..20: mol_type = genomic DNA
ttaaaagaaaaaacaacaag

SEQ ID NO: 194 - DNA, 20 bp, Cannabis sativa
  misc_feature 1..20: note = CsMLO1 position 3114 targeted gRNA
  source 1..20: mol_type = genomic DNA
taaatagcttctacttgccg

SEQ ID NO: 195 - DNA, 20 bp, Cannabis sativa
  misc_feature 1..20: note = CsMLO1 position 3115 targeted gRNA
  source 1..20: mol_type = genomic DNA
aaatagcttctacttgccgt

SEQ ID NO: 196 - DNA, 20 bp, Cannabis sativa
  misc_feature 1..20: note = CsMLO1 position 3120 targeted gRNA
  source 1..20: mol_type = genomic DNA
atgctccagtttagtgccca

SEQ ID NO: 197 - DNA, 20 bp, Cannabis sativa
  misc_feature 1..20: note = CsMLO1 position 3126 targeted gRNA
  source 1..20: mol_type = genomic DNA
acttgccgtgggcactaaac

SEQ ID NO: 198 - DNA, 20 bp, Cannabis sativa
  misc_feature 1..20: note = CsMLO1 position 3147 targeted gRNA
  source 1..20: mol_type = genomic DNA
ggagcatgtcattactcagt

SEQ ID NO: 199 - DNA, 20 bp, Cannabis sativa
  misc_feature 1..20: note = CsMLO1 position 3184 targeted gRNA
  source 1..20: mol_type = genomic DNA
gagaaacatgtagcaataga

SEQ ID NO: 200 - DNA, 20 bp, Cannabis sativa
  misc_feature 1..20: note = CsMLO1 position 3212 targeted gRNA
  source 1..20: mol_type = genomic DNA
aaccaaaagtgatcatctga

SEQ ID NO: 201 - DNA, 20 bp, Cannabis sativa
  misc_feature 1..20: note = CsMLO1 position 3221 targeted gRNA
  source 1..20: mol_type = genomic DNA
agccatcagatgatcacttt

SEQ ID NO: 202 - DNA, 20 bp, Cannabis sativa
  misc_feature 1..20: note = CsMLO1 position 3242 targeted gRNA
  source 1..20: mol_type = genomic DNA
atcaggaagaggacaatctg

SEQ ID NO: 203 - DNA, 20 bp, Cannabis sativa
  misc_feature 1..20: note = CsMLO1 position 3243 targeted gRNA
  source 1..20: mol_type = genomic DNA
aatcaggaagaggacaatct

SEQ ID NO: 204 - DNA, 20 bp, Cannabis sativa
  misc_feature 1..20: note = CsMLO1 position 3244 targeted gRNA
  source 1..20: mol_type = genomic DNA
gaatcaggaagaggacaatc

SEQ ID NO: 205 - DNA, 20 bp, Cannabis sativa
  misc_feature 1..20: note = CsMLO1 position 3253 targeted gRNA
  source 1..20: mol_type = genomic DNA
tgatgaaatgaatcaggaag

SEQ ID NO: 206 - DNA, 20 bp, Cannabis sativa
  misc_feature 1..20: note = CsMLO1 position 3259 targeted gRNA
  source 1..20: mol_type = genomic DNA
aaaggatgatgaaatgaatc

SEQ ID NO: 207 - DNA, 20 bp, Cannabis sativa
  misc_feature 1..20: note = CsMLO1 position 3277 targeted gRNA
  source 1..20: mol_type = genomic DNA
tctcaaatgaattttggaaa

SEQ ID NO: 208 - DNA, 20 bp, Cannabis sativa
  misc_feature 1..20: note = CsMLO1 position 3283 targeted gRNA
  source 1..20: mol_type = genomic DNA
acgcaatctcaaatgaattt

SEQ ID NO: 209 - DNA, 20 bp, Cannabis sativa
  misc_feature 1..20: note = CsMLO1 position 3305 targeted gRNA
  source 1..20: mol_type = genomic DNA
ttgagattgcgtttttcttc

SEQ ID NO: 210 - DNA, 20 bp, Cannabis sativa
  misc_feature 1..20: note = CsMLO1 position 3312 targeted gRNA
  source 1..20: mol_type = genomic DNA
tgcgtttttcttctggatat

SEQ ID NO: 211 - DNA, 20 bp, Cannabis sativa
  misc_feature 1..20: note = CsMLO1 position 3320 targeted gRNA
  source 1..20: mol_type = genomic DNA
tcttctggatattggtaagc

SEQ ID NO: 212 - DNA, 20 bp, Cannabis sativa
  misc_feature 1..20: note = CsMLO1 position 3343 targeted gRNA
  source 1..20: mol_type = genomic DNA
tggtagaagtagaagcagag

SEQ ID NO: 213 - DNA, 20 bp, Cannabis sativa
  misc_feature 1..20: note = CsMLO1 position 3363 targeted gRNA
  source 1..20: mol_type = genomic DNA
aataacaatttgttcttttt

SEQ ID NO: 214 - DNA, 20 bp, Cannabis sativa
  misc_feature 1..20: note = CsMLO1 position 3411 targeted gRNA
  source 1..20: mol_type = genomic DNA
atcttcttttctgtgtatct

SEQ ID NO: 215 - DNA, 20 bp, Cannabis sativa
  misc_feature 1..20: note = CsMLO1 position 3441 targeted gRNA
  source 1..20: mol_type = genomic DNA
ttcatttaactcctgtataa

SEQ ID NO: 216 - DNA, 20 bp, Cannabis sativa
  misc_feature 1..20: note = CsMLO1 position 3441, strand -1, targeted gRNA
  source 1..20: mol_type = genomic DNA
acgaacgtgtcccattatac

SEQ ID NO: 217 - DNA, 20 bp, Cannabis sativa
  misc_feature 1..20: note = CsMLO1 position 3441 targeted gRNA
  source 1..20: mol_type = genomic DNA
tcatttaactcctgtataat

SEQ ID NO: 218 - DNA, 20 bp, Cannabis sativa
  misc_feature 1..20: note = CsMLO1 position 3472 targeted gRNA
  source 1..20: mol_type = genomic DNA
tttacccaatgacaagtctt

SEQ ID NO: 219 - DNA, 20 bp, Cannabis sativa
  misc_feature 1..20: note = CsMLO1 position 3473 targeted gRNA
  source 1..20: mol_type = genomic DNA
ttttacccaatgacaagtct

SEQ ID NO: 220 - DNA, 20 bp, Cannabis sativa
  misc_feature 1..20: note = CsMLO1 position 3478 targeted gRNA
  source 1..20: mol_type = genomic DNA
attgtcccaagacttgtcat

SEQ ID NO: 221 - DNA, 20 bp, Cannabis sativa
  misc_feature 1..20: note = CsMLO1 position 3479 targeted gRNA
  source 1..20: mol_type = genomic DNA
ttgtcccaagacttgtcatt

SEQ ID NO: 222 - DNA, 20 bp, Cannabis sativa
  misc_feature 1..20: note = CsMLO1 position 3541 targeted gRNA
  source 1..20: mol_type = genomic DNA
taaaataaaagtttcgtact

SEQ ID NO: 223 - DNA, 20 bp, Cannabis sativa
  misc_feature 1..20: note = CsMLO1 position 3570 targeted gRNA
  source 1..20: mol_type = genomic DNA
gaacaccctaaagcacaaca

SEQ ID NO: 224 - DNA, 20 bp, Cannabis sativa
  misc_feature 1..20: note = CsMLO1 position 1098 targeted gRNA
  source 1..20: mol_type = genomic DNA
tttttaccatgttgtgcttt

SEQ ID NO: 225 - DNA, 20 bp, Cannabis sativa
  misc_feature 1..20: note = CsMLO1 position 3576 targeted gRNA
  source 1..20: mol_type = genomic DNA
ttttaccatgttgtgcttta

SEQ ID NO: 226 - DNA, 20 bp, Cannabis sativa
  misc_feature 1..20: note = CsMLO1 position 3588 targeted gRNA
  source 1..20: mol_type = genomic DNA
gtgctttagggtgttcattc

SEQ ID NO: 227 - DNA, 20 bp, Cannabis sativa
  misc_feature 1..20: note = CsMLO1 position 3622 targeted gRNA
  source 1..20: mol_type = genomic DNA
gtgtgacaatggcatagagc

SEQ ID NO: 228 - DNA, 20 bp, Cannabis sativa
  misc_feature 1..20: note = CsMLO1 position 3623 targeted gRNA
  source 1..20: mol_type = genomic DNA
tgtgtgacaatggcatagag

SEQ ID NO: 229 - DNA, 20 bp, Cannabis sativa
  misc_feature 1..20: note = CsMLO1 position 3633 targeted gRNA
  source 1..20: mol_type = genomic DNA
tcaacgcacctgtgtgacaa

SEQ ID NO: 230 - DNA, 20 bp, Cannabis sativa
  misc_feature 1..20: note = CsMLO1 position 3636 targeted gRNA
  source 1..20: mol_type = genomic DNA
gctctatgccattgtcacac

SEQ ID NO: 231 - DNA, 20 bp, Cannabis sativa
  misc_feature 1..20: note = CsMLO1 position 3681 targeted gRNA
  source 1..20: mol_type = genomic DNA
ataatttaataagttctaaa

SEQ ID NO: 232 - DNA, 20 bp, Cannabis sativa
  misc_feature 1..20: note = CsMLO1 position 3689 targeted gRNA
  source 1..20: mol_type = genomic DNA
ataagttctaaaaggaaagt

SEQ ID NO: 233 - DNA, 20 bp, Cannabis sativa
  misc_feature 1..20: note = CsMLO1 position 3720 targeted gRNA
  source 1..20: mol_type = genomic DNA
cattccacaagattttatta

SEQ ID NO: 234 - DNA, 20 bp, Cannabis sativa
  misc_feature 1..20: note = CsMLO1 position 3727 targeted gRNA
  source 1..20: mol_type = genomic DNA
ctgaccataataaaatcttg

SEQ ID NO: 235 - DNA, 20 bp, Cannabis sativa
  misc_feature 1..20: note = CsMLO1 position 3743 targeted gRNA
  source 1..20: mol_type = genomic DNA
cttgtggaatgatttgaaga

SEQ ID NO: 236 - DNA, 20 bp, Cannabis sativa
  misc_feature 1..20: note = CsMLO1 position 3744 targeted gRNA
  source 1..20: mol_type = genomic DNA
ttgtggaatgatttgaagat

SEQ ID NO: 237 - DNA, 20 bp, Cannabis sativa
  misc_feature 1..20: note = CsMLO1 position 3773 targeted gRNA
  source 1..20: mol_type = genomic DNA
ttacaagaaagccatatttg

SEQ ID NO: 238 - DNA, 20 bp, Cannabis sativa
  misc_feature 1..20: note = CsMLO1 position 3773, strand -1, targeted gRNA
  source 1..20: mol_type = genomic DNA
ttgcatgcgctcctcaaata

SEQ ID NO: 239 - DNA, 20 bp, Cannabis sativa
  misc_feature 1..20: note = CsMLO1 position 3789 targeted gRNA
  source 1..20: mol_type = genomic DNA
tttgaggagcgcatgcaagt

SEQ ID NO: 240 - DNA, 20 bp, Cannabis sativa
  misc_feature 1..20: note = CsMLO1 position 3802 targeted gRNA
  source 1..20: mol_type = genomic DNA
tgcaagtaggaattgttaat

SEQ ID NO: 241 - DNA, 20 bp, Cannabis sativa
  misc_feature 1..20: note = CsMLO1 position 3803 targeted gRNA
  source 1..20: mol_type = genomic DNA
gcaagtaggaattgttaatt

SEQ ID NO: 242 - DNA, 20 bp, Cannabis sativa
  misc_feature 1..20: note = CsMLO1 position 3812 targeted gRNA
  source 1..20: mol_type = genomic DNA
aattgttaattgggctcaga

SEQ ID NO: 243 - DNA, 20 bp, Cannabis sativa
  misc_feature 1..20: note = CsMLO1 position 3827targeted gRNA
  source 1..20: mol_type = genomic DNA
tcagaaggtcaagaaaaaga

SEQ ID NO: 244 - DNA, 20 bp, Cannabis sativa
  misc_feature 1..20: note = CsMLO1 position 3828 targeted gRNA
  source 1..20: mol_type = genomic DNA
cagaaggtcaagaaaaagaa

SEQ ID NO: 245 - DNA, 20 bp, Cannabis sativa
  misc_feature 1..20: note = CsMLO1 position 3849 targeted gRNA
  source 1..20: mol_type = genomic DNA
ggatttaaagcagccctcat

SEQ ID NO: 246 - DNA, 20 bp, Cannabis sativa
  misc_feature 1..20: note = CsMLO1 position 3851 targeted gRNA
  source 1..20: mol_type = genomic DNA
gccagcaccggaaccaatga

SEQ ID NO: 247 - DNA, 20 bp, Cannabis sativa
  misc_feature 1..20: note = CsMLO1 position 3852 targeted gRNA
  source 1..20: mol_type = genomic DNA
agccagcaccggaaccaatg

SEQ ID NO: 248 - DNA, 20 bp, Cannabis sativa
  misc_feature 1..20: note = CsMLO1 position 3855 targeted gRNA
  source 1..20: mol_type = genomic DNA
aaagcagccctcattggttc

SEQ ID NO: 249 - DNA, 20 bp, Cannabis sativa
  misc_feature 1..20: note = CsMLO1 position 3861 targeted gRNA
  source 1..20: mol_type = genomic DNA
gccctcattggttccggtgc

SEQ ID NO: 250 - DNA, 20 bp, Cannabis sativa
  misc_feature 1..20: note = CsMLO1 position 3863 targeted gRNA
  source 1..20: mol_type = genomic DNA
gcctgagcctgagccagcac

SEQ ID NO: 251 - DNA, 20 bp, Cannabis sativa
  misc_feature 1..20: note = CsMLO1 position 3867 targeted gRNA
  source 1..20: mol_type = genomic DNA
attggttccggtgctggctc

SEQ ID NO: 252 - DNA, 20 bp, Cannabis sativa
  misc_feature 1..20: note = CsMLO1 position 3873 targeted gRNA
  source 1..20: mol_type = genomic DNA
tccggtgctggctcaggctc

SEQ ID NO: 253 - DNA, 20 bp, Cannabis sativa
  misc_feature 1..20: note = CsMLO1 position 3879 targeted gRNA
  source 1..20: mol_type = genomic DNA
gctggctcaggctcaggctc

SEQ ID NO: 254 - DNA, 20 bp, Cannabis sativa
  misc_feature 1..20: note = CsMLO1 position 3884 targeted gRNA
  source 1..20: mol_type = genomic DNA
ctcaggctcaggctcaggct

SEQ ID NO: 255 - DNA, 20 bp, Cannabis sativa
  misc_feature 1..20: note = CsMLO1 position 3885 targeted gRNA
  source 1..20: mol_type = genomic DNA
tcaggctcaggctcaggctc

SEQ ID NO: 256 - DNA, 20 bp, Cannabis sativa
  misc_feature 1..20: note = CsMLO1 position 3891 targeted gRNA
  source 1..20: mol_type = genomic DNA
tcaggctcaggctcgggatc

SEQ ID NO: 257 - DNA, 20 bp, Cannabis sativa
  misc_feature 1..20: note = CsMLO1 position 3903 targeted gRNA
  source 1..20: mol_type = genomic DNA
tcgggatcaggctctactcc

SEQ ID NO: 258 - DNA, 20 bp, Cannabis sativa
  misc_feature 1..20: note = CsMLO1 position 3910 targeted gRNA
  source 1..20: mol_type = genomic DNA
gtatcagaaattggttgacc

SEQ ID NO: 259 - DNA, 20 bp, Cannabis sativa
  misc_feature 1..20: note = CsMLO1 position 3919 targeted gRNA
  source 1..20: mol_type = genomic DNA
gcagaaccagtatcagaaat

SEQ ID NO: 260 - DNA, 20 bp, Cannabis sativa
  misc_feature 1..20: note = CsMLO1 position 3924 targeted gRNA
  source 1..20: mol_type = genomic DNA
ggtcaaccaatttctgatac

SEQ ID NO: 261 - DNA, 20 bp, Cannabis sativa
  misc_feature 1..20: note = CsMLO1 position 3938 targeted gRNA
  source 1..20: mol_type = genomic DNA
tgatactggttctgcatctg

SEQ ID NO: 262 - DNA, 20 bp, Cannabis sativa
  misc_feature 1..20: note = CsMLO1 position 3939 targeted gRNA
  source 1..20: mol_type = genomic DNA
gatactggttctgcatctgt

SEQ ID NO: 263 - DNA, 20 bp, Cannabis sativa
  misc_feature 1..20: note = CsMLO1 position 3950 targeted gRNA
  source 1..20: mol_type = genomic DNA
tgcatctgtgggaattcagc

SEQ ID NO: 264 - DNA, 20 bp, Cannabis sativa
  misc_feature 1..20: note = CsMLO1 position 3951 targeted gRNA
  source 1..20: mol_type = genomic DNA
gcatctgtgggaattcagct

SEQ ID NO: 265 - DNA, 20 bp, Cannabis sativa
  misc_feature 1..20: note = CsMLO1 position 3973 targeted gRNA
  source 1..20: mol_type = genomic DNA
tgctctggctttgatgcttt

SEQ ID NO: 266 - DNA, 20 bp, Cannabis sativa
  misc_feature 1..20: note = CsMLO1 position 3974 targeted gRNA
  source 1..20: mol_type = genomic DNA
ctgctctggctttgatgctt

SEQ ID NO: 267 - DNA, 20 bp, Cannabis sativa
  misc_feature 1..20: note = CsMLO1 position 3988 targeted gRNA
  source 1..20: mol_type = genomic DNA
ttagagtcatcactctgctc

SEQ ID NO: 268 - DNA, 20 bp, Cannabis sativa
  misc_feature 1..20: note = CsMLO1 position 4058 targeted gRNA
  source 1..20: mol_type = genomic DNA
gaagacataagtctaccctt

SEQ ID NO: 269 - DNA, 20 bp, Cannabis sativa
  misc_feature 1..20: note = CsMLO1 position 4062 targeted gRNA
  source 1..20: mol_type = genomic DNA
ctagtagtagtattacctaa

SEQ ID NO: 270 - DNA, 20 bp, Cannabis sativa
  misc_feature 1..20: note = CsMLO1 position 4063 targeted gRNA
  source 1..20: mol_type = genomic DNA
actagtagtagtattaccta

SEQ ID NO: 271 - DNA, 20 bp, Cannabis sativa
  misc_feature 1..20: note = CsMLO1 position 4088 targeted gRNA
  source 1..20: mol_type = genomic DNA
atcccagcacagctggaaag

SEQ ID NO: 272 - DNA, 20 bp, Cannabis sativa
  misc_feature 1..20: note = CsMLO1 position 4095 targeted gRNA
  source 1..20: mol_type = genomic DNA
atttctaatcccagcacagc

SEQ ID NO: 273 - DNA, 20 bp, Cannabis sativa
  misc_feature 1..20: note = CsMLO1 position 4096 targeted gRNA
  source 1..20: mol_type = genomic DNA
ttgccactttccagctgtgc

SEQ ID NO: 274 - DNA, 20 bp, Cannabis sativa
  misc_feature 1..20: note = CsMLO1 position 4097 targeted gRNA
  source 1..20: mol_type = genomic DNA
tgccactttccagctgtgct

SEQ ID NO: 275 - DNA, 20 bp, Cannabis sativa
  misc_feature 1..20: note = CsMLO1 position 4132 targeted gRNA
  source 1..20: mol_type = genomic DNA
aattcttctgtcatatatta

SEQ ID NO: 276 - DNA, 20 bp, Cannabis sativa
  misc_feature 1..20: note = CsMLO1 position 4138 targeted gRNA
  source 1..20: mol_type = genomic DNA
tctgtcatatattatggctg

SEQ ID NO: 277 - DNA, 20 bp, Cannabis sativa
  misc_feature 1..20: note = CsMLO1 position 4141 targeted gRNA
  source 1..20: mol_type = genomic DNA
gtcatatattatggctgtgg

SEQ ID NO: 278 - DNA, 20 bp, Cannabis sativa
  misc_feature 1..20: note = CsMLO1 position 4142 targeted gRNA
  source 1..20: mol_type = genomic DNA
tcatatattatggctgtggt

SEQ ID NO: 279 - DNA, 20 bp, Cannabis sativa
  misc_feature 1..20: note = CsMLO1 position 4160 targeted gRNA
  source 1..20: mol_type = genomic DNA
gtcttgtccataaaagactt

SEQ ID NO: 280 - DNA, 20 bp, Cannabis sativa
  misc_feature 1..20: note = CsMLO1 position 4164 targeted gRNA
  source 1..20: mol_type = genomic DNA
gactgtacctaagtctttta

SEQ ID NO: 281 - DNA, 20 bp, Cannabis sativa
  misc_feature 1..20: note = CsMLO1 position 4188 targeted gRNA
  source 1..20: mol_type = genomic DNA
ttatataatatattgatcaa

SEQ ID NO: 282 - DNA, 20 bp, Cannabis sativa
  misc_feature 1..20: note = CsMLO1 position 4267 targeted gRNA
  source 1..20: mol_type = genomic DNA
cttctttcttcttattatca

SEQ ID NO: 283 - DNA, 20 bp, Cannabis sativa
  misc_feature 1..20: note = CsMLO1 position 4280 targeted gRNA
  source 1..20: mol_type = genomic DNA
attatcatggtacatccttt

SEQ ID NO: 284 - DNA, 20 bp, Cannabis sativa
  misc_feature 1..20: note = CsMLO1 position 4284 targeted gRNA
  source 1..20: mol_type = genomic DNA
ttcactattcagttaccaaa

SEQ ID NO: 285 - DNA, 20 bp, Cannabis sativa
  misc_feature 1..20: note = CsMLO1 position 4312 targeted gRNA
  source 1..20: mol_type = genomic DNA
agtgaatacgtgtagtctca

SEQ ID NO: 286 - DNA, 20 bp, Cannabis sativa
  misc_feature 1..20: note = CsMLO1 position 4313 targeted gRNA
  source 1..20: mol_type = genomic DNA
gtgaatacgtgtagtctcat

SEQ ID NO: 287 - DNA, 20 bp, Cannabis sativa
  misc_feature 1..20: note = CsMLO2 position 1977 targeted gRNA
  source 1..20: mol_type = genomic DNA
gtatgaatatgaaattaagt

SEQ ID NO: 288 - DNA, 20 bp, Cannabis sativa
  misc_feature 1..20: note = CsMLO2 position 2044 targeted gRNA
  source 1..20: mol_type = genomic DNA
agagagagagagacagagag

SEQ ID NO: 289 - DNA, 20 bp, Cannabis sativa
  misc_feature 1..20: note = CsMLO2 position 2117 targeted gRNA
  source 1..20: mol_type = genomic DNA
ttgaaattgggatggagatg

SEQ ID NO: 290 - DNA, 20 bp, Cannabis sativa
  misc_feature 1..20: note = CsMLO2 position 2125 targeted gRNA
  source 1..20: mol_type = genomic DNA
attctgttttgaaattggga

SEQ ID NO: 291 - DNA, 20 bp, Cannabis sativa
  misc_feature 1..20: note = CsMLO2 position 2129 targeted gRNA
  source 1..20: mol_type = genomic DNA
gtaaattctgttttgaaatt

SEQ ID NO: 292 - DNA, 20 bp, Cannabis sativa
  misc_feature 1..20: note = CsMLO2 position 2130 targeted gRNA
  source 1..20: mol_type = genomic DNA
tgtaaattctgttttgaaat

SEQ ID NO: 293 - DNA, 20 bp, Cannabis sativa
  misc_feature 1..20: note = CsMLO2 position 2153 targeted gRNA
  source 1..20: mol_type = genomic DNA
gttagaatgaaaagtttgat

SEQ ID NO: 294 - DNA, 20 bp, Cannabis sativa
  misc_feature 1..20: note = CsMLO2 position 2154 targeted gRNA
  source 1..20: mol_type = genomic DNA
agttagaatgaaaagtttga

SEQ ID NO: 295 - DNA, 20 bp, Cannabis sativa
  misc_feature 1..20: note = CsMLO2 position 2211 targeted gRNA
  source 1..20: mol_type = genomic DNA
tataatcaattattcccaag

SEQ ID NO: 296 - DNA, 20 bp, Cannabis sativa
  misc_feature 1..20: note = CsMLO2 position 2214 targeted gRNA
  source 1..20: mol_type = genomic DNA
taaatataaataggccactt

SEQ ID NO: 297 - DNA, 20 bp, Cannabis sativa
  misc_feature 1..20: note = CsMLO2 position 2215targeted gRNA
  source 1..20: mol_type = genomic DNA
ataaatataaataggccact

SEQ ID NO: 298 - DNA, 20 bp, Cannabis sativa
  misc_feature 1..20: note = CsMLO2 position 2223 targeted gRNA
  source 1..20: mol_type = genomic DNA
tagtgatcataaatataaat

SEQ ID NO: 299 - DNA, 20 bp, Cannabis sativa
  misc_feature 1..20: note = CsMLO2 position 2278 targeted gRNA
  source 1..20: mol_type = genomic DNA
aaaattaaattaaaagaaga

SEQ ID NO: 300 - DNA, 20 bp, Cannabis sativa
  source 1..20: mol_type = genomic DNA
attaaattaaaagaagatgg

SEQ ID NO: 301 - DNA, 20 bp, Cannabis sativa
  misc_feature 1..20: note = CsMLO2 position 2284 targeted gRNA
  source 1..20: mol_type = genomic DNA
aaattaaaagaagatggcgg

SEQ ID NO: 302 - DNA, 20 bp, Cannabis sativa
  misc_feature 1..20: note = CsMLO2 position 2291 targeted gRNA
  source 1..20: mol_type = genomic DNA
aagaagatggcggtggctag

SEQ ID NO: 303 - DNA, 20 bp, Cannabis sativa
  misc_feature 1..20: note = CsMLO2 position 2294 targeted gRNA
  source 1..20: mol_type = genomic DNA
aagatggcggtggctagcgg

SEQ ID NO: 304 - DNA, 20 bp, Cannabis sativa
  misc_feature 1..20: note = CsMLO2 position 2322 targeted gRNA
  source 1..20: mol_type = genomic DNA
ctttagaacaaacaccaaca

SEQ ID NO: 305 - DNA, 20 bp, Cannabis sativa
  misc_feature 1..20: note = CsMLO2 position 2323 targeted gRNA
  source 1..20: mol_type = genomic DNA
tttagaacaaacaccaacat

SEQ ID NO: 306 - DNA, 20 bp, Cannabis sativa
  misc_feature 1..20: note = CsMLO2 position 2325 targeted gRNA
  source 1..20: mol_type = genomic DNA
actacggccacagcccatgt

SEQ ID NO: 307 - DNA, 20 bp, Cannabis sativa
  misc_feature 1..20: note = CsMLO2 position 2329 targeted gRNA
  source 1..20: mol_type = genomic DNA
acaaacaccaacatgggctg

SEQ ID NO: 308 - DNA, 20 bp, Cannabis sativa
  misc_feature 1..20: note = CsMLO2 position 2341 targeted gRNA
  source 1..20: mol_type = genomic DNA
taccaaaacaagacaaacta

SEQ ID NO: 309 - DNA, 20 bp, Cannabis sativa
  misc_feature 1..20: note = CsMLO2 position 2350 targeted gRNA
  source 1..20: mol_type = genomic DNA
ggccgtagtttgtcttgttt

SEQ ID NO: 310 - DNA, 20 bp, Cannabis sativa
  misc_feature 1..20: note = CsMLO2 position 2371 targeted gRNA
  source 1..20: mol_type = genomic DNA
gattatgtgctcaataataa

SEQ ID NO: 311 - DNA, 20 bp, Cannabis sativa
  misc_feature 1..20: note = CsMLO2 position 2393 targeted gRNA
  source 1..20: mol_type = genomic DNA
gagcacataatccatctcat

SEQ ID NO: 312 - DNA, 20 bp, Cannabis sativa
  misc_feature 1..20: note = CsMLO2 position 2393, strand -1, targeted gRNA
  source 1..20: mol_type = genomic DNA
ggtataccttgccaatgaga

SEQ ID NO: 313 - DNA, 20 bp, Cannabis sativa
  misc_feature 1..20: note = CsMLO2 position 2398 targeted gRNA
  source 1..20: mol_type = genomic DNA
cataatccatctcattggca

SEQ ID NO: 314 - DNA, 20 bp, Cannabis sativa
  misc_feature 1..20: note = CsMLO2 position 2414 targeted gRNA
  source 1..20: mol_type = genomic DNA
tgagattaatatatataatt

SEQ ID NO: 315 - DNA, 20 bp, Cannabis sativa
  misc_feature 1..20: note = CsMLO2 position 2415 targeted gRNA
  source 1..20: mol_type = genomic DNA
gtgagattaatatatataat

SEQ ID NO: 316 - DNA, 20 bp, Cannabis sativa
  misc_feature 1..20: note = CsMLO2 position 2473 targeted gRNA
  source 1..20: mol_type = genomic DNA
catttaattatttaaattaa

SEQ ID NO: 317 - DNA, 20 bp, Cannabis sativa
  misc_feature 1..20: note = CsMLO2 position 2474 targeted gRNA
  source 1..20: mol_type = genomic DNA
atttaattatttaaattaat

SEQ ID NO: 318 - DNA, 20 bp, Cannabis sativa
  misc_feature 1..20: note = CsMLO2 position 2495 targeted gRNA
  source 1..20: mol_type = genomic DNA
ggtattttttttttttttag

SEQ ID NO: 319 - DNA, 20 bp, Cannabis sativa
  misc_feature 1..20: note = CsMLO2 position 2535 targeted gRNA
  source 1..20: mol_type = genomic DNA
acgagctctttatgaatcgt

SEQ ID NO: 320 - DNA, 20 bp, Cannabis sativa
  misc_feature 1..20: note = CsMLO2 position 2551 targeted gRNA
  source 1..20: mol_type = genomic DNA
tcgttggaaaagatcaaatc

SEQ ID NO: 321 - DNA, 20 bp, Cannabis sativa
  misc_feature 1..20: note = CsMLO2 position 2576 targeted gRNA
  source 1..20: mol_type = genomic DNA
aaaatgggtattcattaatt

SEQ ID NO: 322 - DNA, 20 bp, Cannabis sativa
  misc_feature 1..20: note = CsMLO2 position 2577 targeted gRNA
  source 1..20: mol_type = genomic DNA
aaaaatgggtattcattaat

SEQ ID NO: 323 - DNA, 20 bp, Cannabis sativa
  misc_feature 1..20: note = CsMLO2 position 2591 targeted gRNA
  source 1..20: mol_type = genomic DNA
ttaaaaaaaaaaacaaaaat

SEQ ID NO: 324 - DNA, 20 bp, Cannabis sativa
  misc_feature 1..20: note = CsMLO2 position 2656 targeted gRNA
  source 1..20: mol_type = genomic DNA
tttgatagagcttatgttat

SEQ ID NO: 325 - DNA, 20 bp, Cannabis sativa
  misc_feature 1..20: note = CsMLO2 position 2657 targeted gRNA
  source 1..20: mol_type = genomic DNA
ttgatagagcttatgttatt

SEQ ID NO: 326 - DNA, 20 bp, Cannabis sativa
  misc_feature 1..20: note = CsMLO2 position 2658 targeted gRNA
  source 1..20: mol_type = genomic DNA
tgatagagcttatgttattg

SEQ ID NO: 327 - DNA, 20 bp, Cannabis sativa
  misc_feature 1..20: note = CsMLO2 position 2680 targeted gRNA
  source 1..20: mol_type = genomic DNA
gttcatatcgttgttactaa

SEQ ID NO: 328 - DNA, 20 bp, Cannabis sativa
  misc_feature 1..20: note = CsMLO2 position 2683 targeted gRNA
  source 1..20: mol_type = genomic DNA
catatcgttgttactaacgg

SEQ ID NO: 329 - DNA, 20 bp, Cannabis sativa
  misc_feature 1..20: note = CsMLO2 position 2684 targeted gRNA
  source 1..20: mol_type = genomic DNA
atatcgttgttactaacggt

SEQ ID NO: 330 - DNA, 20 bp, Cannabis sativa
  misc_feature 1..20: note = CsMLO2 position 2703 targeted gRNA
  source 1..20: mol_type = genomic DNA
gatatacaaatatttgagat

SEQ ID NO: 331 - DNA, 20 bp, Cannabis sativa
  misc_feature 1..20: note = CsMLO2 position 2726 targeted gRNA
  source 1..20: mol_type = genomic DNA
atttgtatatctgagaaaat

SEQ ID NO: 332 - DNA, 20 bp, Cannabis sativa
  misc_feature 1..20: note = CsMLO2 position 2729 targeted gRNA
  source 1..20: mol_type = genomic DNA
tgtatatctgagaaaattgg

SEQ ID NO: 333 - DNA, 20 bp, Cannabis sativa
  misc_feature 1..20: note = CsMLO2 position 2730 targeted gRNA
  source 1..20: mol_type = genomic DNA
gtatatctgagaaaattgga

SEQ ID NO: 334 - DNA, 20 bp, Cannabis sativa
  misc_feature 1..20: note = CsMLO2 position 2736 targeted gRNA
  source 1..20: mol_type = genomic DNA
ctgagaaaattggagggaca

SEQ ID NO: 335 - DNA, 20 bp, Cannabis sativa
  misc_feature 1..20: note = CsMLO2 position 2751 targeted gRNA
  source 1..20: mol_type = genomic DNA
tcttcttgttctttattaca

SEQ ID NO: 336 - DNA, 20 bp, Cannabis sativa
  misc_feature 1..20: note = CsMLO2 position 2777 targeted gRNA
  source 1..20: mol_type = genomic DNA
caagaagagaaattgaataa

SEQ ID NO: 337 - DNA, 20 bp, Cannabis sativa
  misc_feature 1..20: note = CsMLO2 position 2778 targeted gRNA
  source 1..20: mol_type = genomic DNA
aagaagagaaattgaataaa

SEQ ID NO: 338 - DNA, 20 bp, Cannabis sativa
  misc_feature 1..20: note = CsMLO2 position 2779 targeted gRNA
  source 1..20: mol_type = genomic DNA
agaagagaaattgaataaag

SEQ ID NO: 339 - DNA, 20 bp, Cannabis sativa
  misc_feature 1..20: note = CsMLO2 position 2817 targeted gRNA
  source 1..20: mol_type = genomic DNA
tcgaacatgaaagtaacagt

SEQ ID NO: 340 - DNA, 20 bp, Cannabis sativa
  misc_feature 1..20: note = CsMLO2 position 2827 targeted gRNA
  source 1..20: mol_type = genomic DNA
aagtaacagtcggagattgc

SEQ ID NO: 341 - DNA, 20 bp, Cannabis sativa
  misc_feature 1..20: note = CsMLO2 position 2839 targeted gRNA
  source 1..20: mol_type = genomic DNA
accgtcgccggactctaaaa

SEQ ID NO: 342 - DNA, 20 bp, Cannabis sativa
  misc_feature 1..20: note = CsMLO2 position 2843 targeted gRNA
  source 1..20: mol_type = genomic DNA
ttgctggcctttttagagtc

SEQ ID NO: 343 - DNA, 20 bp, Cannabis sativa
  misc_feature 1..20: note = CsMLO2 position 2849 targeted gRNA
  source 1..20: mol_type = genomic DNA
gcctttttagagtccggcga

SEQ ID NO: 344 - DNA, 20 bp, Cannabis sativa
  misc_feature 1..20: note = CsMLO2 position 2851 targeted gRNA
  source 1..20: mol_type = genomic DNA
gacactagcagcaccgtcgc

SEQ ID NO: 345 - DNA, 20 bp, Cannabis sativa
  misc_feature 1..20: note = CsMLO2 position 2865 targeted gRNA
  source 1..20: mol_type = genomic DNA
gcgacggtgctgctagtgtc

SEQ ID NO: 346 - DNA, 20 bp, Cannabis sativa
  misc_feature 1..20: note = CsMLO2 position 2873 targeted gRNA
  source 1..20: mol_type = genomic DNA
cggccgccgccaaaattcgc

SEQ ID NO: 347 - DNA, 20 bp, Cannabis sativa
  misc_feature 1..20: note = CsMLO2 position 2875 targeted gRNA
  source 1..20: mol_type = genomic DNA
tgctagtgtccggcgaattt

SEQ ID NO: 348 - DNA, 20 bp, Cannabis sativa
  misc_feature 1..20: note = CsMLO2 position 2878 targeted gRNA
  source 1..20: mol_type = genomic DNA
tagtgtccggcgaattttgg

SEQ ID NO: 349 - DNA, 20 bp, Cannabis sativa
  misc_feature 1..20: note = CsMLO2 position 2881 targeted gRNA
  source 1..20: mol_type = genomic DNA
tgtccggcgaattttggcgg

SEQ ID NO: 350 - DNA, 20 bp, Cannabis sativa
  misc_feature 1..20: note = CsMLO2 position 2885 targeted gRNA
  source 1..20: mol_type = genomic DNA
cggcgaattttggcggcggc

SEQ ID NO: 351 - DNA, 20 bp, Cannabis sativa
  misc_feature 1..20: note = CsMLO2 position 2886 targeted gRNA
  source 1..20: mol_type = genomic DNA
ggcgaattttggcggcggcc

SEQ ID NO: 352 - DNA, 20 bp, Cannabis sativa
  misc_feature 1..20: note = CsMLO2 position 2893 targeted gRNA
  source 1..20: mol_type = genomic DNA
ttcagcacacttatcagtcc

SEQ ID NO: 353 - DNA, 20 bp, Cannabis sativa
  misc_feature 1..20: note = CsMLO2 position 2908 targeted gRNA
  source 1..20: mol_type = genomic DNA
gactgataagtgtgctgaaa

SEQ ID NO: 354 - DNA, 20 bp, Cannabis sativa
  misc_feature 1..20: note = CsMLO2 position 2978 targeted gRNA
  source 1..20: mol_type = genomic DNA
gtctttcttatccttttatt

SEQ ID NO: 355 - DNA, 20 bp, Cannabis sativa
  misc_feature 1..20: note = CsMLO2 position 2978, strand -1, targeted gRNA
  source 1..20: mol_type = genomic DNA
gacgaatatgtccaaataaa

SEQ ID NO: 356 - DNA, 20 bp, Cannabis sativa
  misc_feature 1..20: note = CsMLO2 position 3000 targeted gRNA
  source 1..20: mol_type = genomic DNA
ctcctataatattatatgtt

SEQ ID NO: 357 - DNA, 20 bp, Cannabis sativa
  misc_feature 1..20: note = CsMLO2 position 3009 targeted gRNA
  source 1..20: mol_type = genomic DNA
gtccaaacatataatattat

SEQ ID NO: 358 - DNA, 20 bp, Cannabis sativa
  misc_feature 1..20: note = CsMLO2 position 3051 targeted gRNA
  source 1..20: mol_type = genomic DNA
aaatatataaatttaaaggt

SEQ ID NO: 359 - DNA, 20 bp, Cannabis sativa
  misc_feature 1..20: note = CsMLO2 position 3055 targeted gRNA
  source 1..20: mol_type = genomic DNA
aactaaatatataaatttaa

SEQ ID NO: 360 - DNA, 20 bp, Cannabis sativa
  misc_feature 1..20: note = CsMLO2 position 4125 targeted gRNA
  source 1..20: mol_type = genomic DNA
aaattatatacatatatgaa

SEQ ID NO: 361 - DNA, 20 bp, Cannabis sativa
  misc_feature 1..20: note = CsMLO2 position 4168 targeted gRNA
  source 1..20: mol_type = genomic DNA
atatatataattataatttc

SEQ ID NO: 362 - DNA, 20 bp, Cannabis sativa
  misc_feature 1..20: note = CsMLO2 position 4169 targeted gRNA
  source 1..20: mol_type = genomic DNA
tatatataattataatttca

SEQ ID NO: 363 - DNA, 20 bp, Cannabis sativa
  misc_feature 1..20: note = CsMLO2 position 4187 targeted gRNA
  source 1..20: mol_type = genomic DNA
ataccatccgccgaaacaaa

SEQ ID NO: 364 - DNA, 20 bp, Cannabis sativa
  misc_feature 1..20: note = CsMLO2 position 4188 targeted gRNA
  source 1..20: mol_type = genomic DNA
agggcaagttccatttgttt

SEQ ID NO: 365 - DNA, 20 bp, Cannabis sativa
  misc_feature 1..20: note = CsMLO2 position 4191 targeted gRNA
  source 1..20: mol_type = genomic DNA
gcaagttccatttgtttcgg

SEQ ID NO: 366 - DNA, 20 bp, Cannabis sativa
  misc_feature 1..20: note = CsMLO2 position 4195 targeted gRNA
  source 1..20: mol_type = genomic DNA
gttccatttgtttcggcgga

SEQ ID NO: 367 - DNA, 20 bp, Cannabis sativa
  misc_feature 1..20: note = CsMLO2 position 4230 targeted gRNA
  source 1..20: mol_type = genomic DNA
gcatatttttatctttgtgt

SEQ ID NO: 368 - DNA, 20 bp, Cannabis sativa
  misc_feature 1..20: note = CsMLO2 position 4249 targeted gRNA
  source 1..20: mol_type = genomic DNA
tcatgatgcagtagagaaca

SEQ ID NO: 369 - DNA, 20 bp, Cannabis sativa
  misc_feature 1..20: note = CsMLO2 position 4272 targeted gRNA
  source 1..20: mol_type = genomic DNA
ctgcatcatgactatgtttt

SEQ ID NO: 370 - DNA, 20 bp, Cannabis sativa
  misc_feature 1..20: note = CsMLO2 position 4273 targeted gRNA
  source 1..20: mol_type = genomic DNA
tgcatcatgactatgttttt

SEQ ID NO: 371 - DNA, 20 bp, Cannabis sativa
  misc_feature 1..20: note = CsMLO2 position 4284 targeted gRNA
  source 1..20: mol_type = genomic DNA
tatgtttttgggcagactta

SEQ ID NO: 372 - DNA, 20 bp, Cannabis sativa
  misc_feature 1..20: note = CsMLO2 position 4404 targeted gRNA
  source 1..20: mol_type = genomic DNA
aatttatatataattattta

SEQ ID NO: 373 - DNA, 20 bp, Cannabis sativa
  misc_feature 1..20: note = CsMLO2 position 4405 targeted gRNA
  source 1..20: mol_type = genomic DNA
caatttatatataattattt

SEQ ID NO: 374 - DNA, 20 bp, Cannabis sativa
  misc_feature 1..20: note = CsMLO2 position 4428 targeted gRNA
  source 1..20: mol_type = genomic DNA
tatataaattgattcccaga

SEQ ID NO: 375 - DNA, 20 bp, Cannabis sativa
  misc_feature 1..20: note = CsMLO2 position 4429 targeted gRNA
  source 1..20: mol_type = genomic DNA
atataaattgattcccagat

SEQ ID NO: 376 - DNA, 20 bp, Cannabis sativa
  misc_feature 1..20: note = CsMLO2 position 4431 targeted gRNA
  source 1..20: mol_type = genomic DNA
atgcttccaacttcccatct

SEQ ID NO: 377 - DNA, 20 bp, Cannabis sativa
  misc_feature 1..20: note = CsMLO2 position 4432 targeted gRNA
  source 1..20: mol_type = genomic DNA
aatgcttccaacttcccatc

SEQ ID NO: 378 - DNA, 20 bp, Cannabis sativa
  misc_feature 1..20: note = CsMLO2 position 4436 targeted gRNA
  source 1..20: mol_type = genomic DNA
ttgattcccagatgggaagt

SEQ ID NO: 379 - DNA, 20 bp, Cannabis sativa
  misc_feature 1..20: note = CsMLO2 position 4445 targeted gRNA
  source 1..20: mol_type = genomic DNA
agatgggaagttggaagcat

SEQ ID NO: 380 - DNA, 20 bp, Cannabis sativa
  misc_feature 1..20: note = CsMLO2 position 4446 targeted gRNA
  source 1..20: mol_type = genomic DNA
gatgggaagttggaagcatt

SEQ ID NO: 381 - DNA, 20 bp, Cannabis sativa
  misc_feature 1..20: note = CsMLO2 position 4452 targeted gRNA
  source 1..20: mol_type = genomic DNA
aagttggaagcattgggaaa

SEQ ID NO: 382 - DNA, 20 bp, Cannabis sativa
  misc_feature 1..20: note = CsMLO2 position 4476 targeted gRNA
  source 1..20: mol_type = genomic DNA
accatgtgagaattgatatt

SEQ ID NO: 383 - DNA, 20 bp, Cannabis sativa
  misc_feature 1..20: note = CsMLO2 position 4486 targeted gRNA
  source 1..20: mol_type = genomic DNA
gccgaatatcaattctcaca

SEQ ID NO: 384 - DNA, 20 bp, Cannabis sativa
  misc_feature 1..20: note = CsMLO2 position 4548 targeted gRNA
  source 1..20: mol_type = genomic DNA
cttaattttaatttttctat

SEQ ID NO: 385 - DNA, 20 bp, Cannabis sativa
  misc_feature 1..20: note = CsMLO2 position 4551 targeted gRNA
  source 1..20: mol_type = genomic DNA
aattttaatttttctatagg

SEQ ID NO: 386 - DNA, 20 bp, Cannabis sativa
  misc_feature 1..20: note = CsMLO2 position 4649 targeted gRNA
  source 1..20: mol_type = genomic DNA
ctatatgacatatttgatgg

SEQ ID NO: 387 - DNA, 20 bp, Cannabis sativa
  misc_feature 1..20: note = CsMLO2 position 4652 targeted gRNA
  source 1..20: mol_type = genomic DNA
taactatatgacatatttga

SEQ ID NO: 388 - DNA, 20 bp, Cannabis sativa
  misc_feature 1..20: note = CsMLO2 position 4742 targeted gRNA
  source 1..20: mol_type = genomic DNA
aattataagagcatctttat

SEQ ID NO: 389 - DNA, 20 bp, Cannabis sativa
  misc_feature 1..20: note = CsMLO2 position 4749 targeted gRNA
  source 1..20: mol_type = genomic DNA
agagcatctttattggacac

SEQ ID NO: 390 - DNA, 20 bp, Cannabis sativa
  misc_feature 1..20: note = CsMLO2 position 4757 targeted gRNA
  source 1..20: mol_type = genomic DNA
tagaaagtgttaaatatcac

SEQ ID NO: 391 - DNA, 20 bp, Cannabis sativa
  misc_feature 1..20: note = CsMLO2 position 4844 targeted gRNA
  source 1..20: mol_type = genomic DNA
tattggtataattaagtatc

SEQ ID NO: 392 - DNA, 20 bp, Cannabis sativa
  misc_feature 1..20: note = CsMLO2 position 4861 targeted gRNA
  source 1..20: mol_type = genomic DNA
cttaccaattatattattat

SEQ ID NO: 393 - DNA, 20 bp, Cannabis sativa
  misc_feature 1..20: note = CsMLO2 position 4868 targeted gRNA
  source 1..20: mol_type = genomic DNA
tataccaataataatataat

SEQ ID NO: 394 - DNA, 20 bp, Cannabis sativa
  misc_feature 1..20: note = CsMLO2 position 4903 targeted gRNA
  source 1..20: mol_type = genomic DNA
atttataagaagtatatata

SEQ ID NO: 395 - DNA, 20 bp, Cannabis sativa
  misc_feature 1..20: note = CsMLO2 position 4904 targeted gRNA
  source 1..20: mol_type = genomic DNA
tttataagaagtatatatat

SEQ ID NO: 396 - DNA, 20 bp, Cannabis sativa
  misc_feature 1..20: note = CsMLO2 position 4923 targeted gRNA
  source 1..20: mol_type = genomic DNA
tgggagttagaattaagtaa

SEQ ID NO: 397 - DNA, 20 bp, Cannabis sativa
  misc_feature 1..20: note = CsMLO2 position 4997 targeted gRNA
  source 1..20: mol_type = genomic DNA
ctcgcaaatctgaatctttc

SEQ ID NO: 398 - DNA, 20 bp, Cannabis sativa
  misc_feature 1..20: note = CsMLO2 position 5009 targeted gRNA
  source 1..20: mol_type = genomic DNA
cagaaagattcagatttgcg

SEQ ID NO: 399 - DNA, 20 bp, Cannabis sativa
  misc_feature 1..20: note = CsMLO2 position 5010 targeted gRNA
  source 1..20: mol_type = genomic DNA
agaaagattcagatttgcga

SEQ ID NO: 400 - DNA, 20 bp, Cannabis sativa
  misc_feature 1..20: note = CsMLO2 position 5023 targeted gRNA
  source 1..20: mol_type = genomic DNA
tttgcgagggacacttcttt

SEQ ID NO: 401 - DNA, 20 bp, Cannabis sativa
  misc_feature 1..20: note = CsMLO2 position 5045 targeted gRNA
  source 1..20: mol_type = genomic DNA
gaagaagacatttaagtttc

SEQ ID NO: 402 - DNA, 20 bp, Cannabis sativa
  misc_feature 1..20: note = CsMLO2 position 5058 targeted gRNA
  source 1..20: mol_type = genomic DNA
ccatattaggaaagggtgtt

SEQ ID NO: 403 - DNA, 20 bp, Cannabis sativa
  misc_feature 1..20: note = CsMLO2 position 5065 targeted gRNA
  source 1..20: mol_type = genomic DNA
ttactatccatattaggaaa

SEQ ID NO: 404 - DNA, 20 bp, Cannabis sativa
  misc_feature 1..20: note = CsMLO2 position 5066 targeted gRNA
  source 1..20: mol_type = genomic DNA
cttactatccatattaggaa

SEQ ID NO: 405 - DNA, 20 bp, Cannabis sativa
  misc_feature 1..20: note = CsMLO2 position 5069 targeted gRNA
  source 1..20: mol_type = genomic DNA
ccaaacaccctttcctaata

SEQ ID NO: 406 - DNA, 20 bp, Cannabis sativa
  misc_feature 1..20: note = CsMLO2 position 5071 targeted gRNA
  source 1..20: mol_type = genomic DNA
gggatcttactatccatatt

SEQ ID NO: 407 - DNA, 20 bp, Cannabis sativa
  misc_feature 1..20: note = CsMLO2 position 5091 targeted gRNA
  source 1..20: mol_type = genomic DNA
aagtaaaaagtgggtaaaaa

SEQ ID NO: 408 - DNA, 20 bp, Cannabis sativa
  misc_feature 1..20: note = CsMLO2 position 5092targeted gRNA
  source 1..20: mol_type = genomic DNA
aaagtaaaaagtgggtaaaa

SEQ ID NO: 409 - DNA, 20 bp, Cannabis sativa
  misc_feature 1..20: note = CsMLO2 position 5100 targeted gRNA
  source 1..20: mol_type = genomic DNA
aatataaaaaagtaaaaagt

SEQ ID NO: 410 - DNA, 20 bp, Cannabis sativa
  misc_feature 1..20: note = CsMLO2 position 5101 targeted gRNA
  source 1..20: mol_type = genomic DNA
gaatataaaaaagtaaaaag

SEQ ID NO: 411 - DNA, 20 bp, Cannabis sativa
  misc_feature 1..20: note = CsMLO2 position 5149 targeted gRNA
  source 1..20: mol_type = genomic DNA
atataagtgcatggatatag

SEQ ID NO: 412 - DNA, 20 bp, Cannabis sativa
  misc_feature 1..20: note = CsMLO2 position 5158 targeted gRNA
  source 1..20: mol_type = genomic DNA
tattaatagatataagtgca

SEQ ID NO: 413 - DNA, 20 bp, Cannabis sativa
  misc_feature 1..20: note = CsMLO2 position 5233 targeted gRNA
  source 1..20: mol_type = genomic DNA
catatttatatgcatgtgaa

SEQ ID NO: 414 - DNA, 20 bp, Cannabis sativa
  misc_feature 1..20: note = CsMLO2 position 5253 targeted gRNA
  source 1..20: mol_type = genomic DNA
tgcatataaatatgtttgca

SEQ ID NO: 415 - DNA, 20 bp, Cannabis sativa
  misc_feature 1..20: note = CsMLO2 position 5269 targeted gRNA
  source 1..20: mol_type = genomic DNA
tgcatggtttttatacatcg

SEQ ID NO: 416 - DNA, 20 bp, Cannabis sativa
  misc_feature 1..20: note = CsMLO2 position 7159 targeted gRNA
  source 1..20: mol_type = genomic DNA
tatatatataatattttttt

SEQ ID NO: 417 - DNA, 20 bp, Cannabis sativa
  misc_feature 1..20: note = CsMLO2 position 7213 targeted gRNA
  source 1..20: mol_type = genomic DNA
ttaattaataattaaagagc

SEQ ID NO: 418 - DNA, 20 bp, Cannabis sativa
  misc_feature 1..20: note = CsMLO2 position 7238 targeted gRNA
  source 1..20: mol_type = genomic DNA
attaattaattatttttcgc

SEQ ID NO: 419 - DNA, 20 bp, Cannabis sativa
  misc_feature 1..20: note = CsMLO2 position 7282 targeted gRNA
  source 1..20: mol_type = genomic DNA
aaagttaaataatcaacttt

SEQ ID NO: 420 - DNA, 20 bp, Cannabis sativa
  misc_feature 1..20: note = CsMLO2 position 7302 targeted gRNA
  source 1..20: mol_type = genomic DNA
gattatttaactttgagaca

SEQ ID NO: 421 - DNA, 20 bp, Cannabis sativa
  misc_feature 1..20: note = CsMLO2 position 7313 targeted gRNA
  source 1..20: mol_type = genomic DNA
tttgagacatggatttataa

SEQ ID NO: 422 - DNA, 20 bp, Cannabis sativa
  misc_feature 1..20: note = CsMLO2 position 7373 targeted gRNA
  source 1..20: mol_type = genomic DNA
attatagctgtagagatatt

SEQ ID NO: 423 - DNA, 20 bp, Cannabis sativa
  misc_feature 1..20: note = CsMLO2 position 7387 targeted gRNA
  source 1..20: mol_type = genomic DNA
taagtattattaaaaataca

SEQ ID NO: 424 - DNA, 20 bp, Cannabis sativa
  misc_feature 1..20: note = CsMLO2 position 8017 targeted gRNA
  source 1..20: mol_type = genomic DNA
gaatgagaataggaatagaa

SEQ ID NO: 425 - DNA, 20 bp, Cannabis sativa
  misc_feature 1..20: note = CsMLO2 position 8027 targeted gRNA
  source 1..20: mol_type = genomic DNA
ataggaatgggaatgagaat

SEQ ID NO: 426 - DNA, 20 bp, Cannabis sativa
  misc_feature 1..20: note = CsMLO2 position 8039 targeted gRNA
  source 1..20: mol_type = genomic DNA
ataggaatagaaataggaat

SEQ ID NO: 427 - DNA, 20 bp, Cannabis sativa
  misc_feature 1..20: note = CsMLO2 position 8040 targeted gRNA
  source 1..20: mol_type = genomic DNA
tataggaatagaaataggaa

SEQ ID NO: 428 - DNA, 20 bp, Cannabis sativa
  misc_feature 1..20: note = CsMLO2 position 8045 targeted gRNA
  source 1..20: mol_type = genomic DNA
gaaaatataggaatagaaat

SEQ ID NO: 429 - DNA, 20 bp, Cannabis sativa
  misc_feature 1..20: note = CsMLO2 position 8057 targeted gRNA
  source 1..20: mol_type = genomic DNA
gttgagaggaatgaaaatat

SEQ ID NO: 430 - DNA, 20 bp, Cannabis sativa
  misc_feature 1..20: note = CsMLO2 position 8071 targeted gRNA
  source 1..20: mol_type = genomic DNA
cacagaggcgtttggttgag

SEQ ID NO: 431 - DNA, 20 bp, Cannabis sativa
  misc_feature 1..20: note = CsMLO2 position 8079 targeted gRNA
  source 1..20: mol_type = genomic DNA
aataggcccacagaggcgtt

SEQ ID NO: 432 - DNA, 20 bp, Cannabis sativa
  misc_feature 1..20: note = CsMLO2 position 8083 targeted gRNA
  source 1..20: mol_type = genomic DNA
ctctcaaccaaacgcctctg

SEQ ID NO: 433 - DNA, 20 bp, Cannabis sativa
  misc_feature 1..20: note = CsMLO2 position 8084 targeted gRNA
  source 1..20: mol_type = genomic DNA
tctcaaccaaacgcctctgt

SEQ ID NO: 434 - DNA, 20 bp, Cannabis sativa
  misc_feature 1..20: note = CsMLO2 position 8086 targeted gRNA
  source 1..20: mol_type = genomic DNA
acaagataataggcccacag

SEQ ID NO: 435 - DNA, 20 bp, Cannabis sativa
  misc_feature 1..20: note = CsMLO2 position 8096 targeted gRNA
  source 1..20: mol_type = genomic DNA
ttaatacataacaagataat

SEQ ID NO: 436 - DNA, 20 bp, Cannabis sativa
  misc_feature 1..20: note = CsMLO2 position 8150 targeted gRNA
  source 1..20: mol_type = genomic DNA
atcaataactaaattaattg

SEQ ID NO: 437 - DNA, 20 bp, Cannabis sativa
  misc_feature 1..20: note = CsMLO2 position 8177 targeted gRNA
  source 1..20: mol_type = genomic DNA
ttataacaattaataatttc

SEQ ID NO: 438 - DNA, 20 bp, Cannabis sativa
  misc_feature 1..20: note = CsMLO2 position 8186 targeted gRNA
  source 1..20: mol_type = genomic DNA
ttaataatttcaggcacatt

SEQ ID NO: 439 - DNA, 20 bp, Cannabis sativa
  misc_feature 1..20: note = CsMLO2 position 8198 targeted gRNA
  source 1..20: mol_type = genomic DNA
aaatttttgatggctttgag

SEQ ID NO: 440 - DNA, 20 bp, Cannabis sativa
  misc_feature 1..20: note = CsMLO2 position 8199 targeted gRNA
  source 1..20: mol_type = genomic DNA
caaatttttgatggctttga

SEQ ID NO: 441 - DNA, 20 bp, Cannabis sativa
  misc_feature 1..20: note = CsMLO2 position 8200 targeted gRNA
  source 1..20: mol_type = genomic DNA
tcaaatttttgatggctttg

SEQ ID NO: 442 - DNA, 20 bp, Cannabis sativa
  misc_feature 1..20: note = CsMLO2 position 8208 targeted gRNA
  source 1..20: mol_type = genomic DNA
tttgaaagtcaaatttttga

SEQ ID NO: 443 - DNA, 20 bp, Cannabis sativa
  misc_feature 1..20: note = CsMLO2 position 8255 targeted gRNA
  source 1..20: mol_type = genomic DNA
atctctagaagaagatttca

SEQ ID NO: 444 - DNA, 20 bp, Cannabis sativa
  misc_feature 1..20: note = CsMLO2 position 8265 targeted gRNA
  source 1..20: mol_type = genomic DNA
gaagatttcaaggtcgttgt

SEQ ID NO: 445 - DNA, 20 bp, Cannabis sativa
  misc_feature 1..20: note = CsMLO2 position 8272 targeted gRNA
  source 1..20: mol_type = genomic DNA
tcaaggtcgttgtaggaatc

SEQ ID NO: 446 - DNA, 20 bp, Cannabis sativa
  misc_feature 1..20: note = CsMLO2 position 8345 targeted gRNA
  source 1..20: mol_type = genomic DNA
attaaaataagtcatcattt

SEQ ID NO: 447 - DNA, 20 bp, Cannabis sativa
  misc_feature 1..20: note = CsMLO2 position 8346 targeted gRNA
  source 1..20: mol_type = genomic DNA
aattaaaataagtcatcatt

SEQ ID NO: 448 - DNA, 20 bp, Cannabis sativa
  misc_feature 1..20: note = CsMLO2 position 8399 targeted gRNA
  source 1..20: mol_type = genomic DNA
taataattattattttgttt

SEQ ID NO: 449 - DNA, 20 bp, Cannabis sativa
  misc_feature 1..20: note = CsMLO2 position 8427 targeted gRNA
  source 1..20: mol_type = genomic DNA
tcaatctcagtcctcctatt

SEQ ID NO: 450 - DNA, 20 bp, Cannabis sativa
  misc_feature 1..20: note = CsMLO2 position 8427, strand -1, targeted gRNA
  source 1..20: mol_type = genomic DNA
acagcgaagaaccaaatagg

SEQ ID NO: 451 - DNA, 20 bp, Cannabis sativa
  misc_feature 1..20: note = CsMLO2 position 8430 targeted gRNA
  source 1..20: mol_type = genomic DNA
accacagcgaagaaccaaat

SEQ ID NO: 452 - DNA, 20 bp, Cannabis sativa
  misc_feature 1..20: note = CsMLO2 position 8440 targeted gRNA
  source 1..20: mol_type = genomic DNA
tcctatttggttcttcgctg

SEQ ID NO: 453 - DNA, 20 bp, Cannabis sativa
  misc_feature 1..20: note = CsMLO2 position 8465 targeted gRNA
  source 1..20: mol_type = genomic DNA
ttcttactcttcaataccca

SEQ ID NO: 454 - DNA, 20 bp, Cannabis sativa
  misc_feature 1..20: note = CsMLO2 position 8470 targeted gRNA
  source 1..20: mol_type = genomic DNA
aataataaaatgctcaccat

SEQ ID NO: 455 - DNA, 20 bp, Cannabis sativa
  misc_feature 1..20: note = CsMLO2 position 8471 targeted gRNA
  source 1..20: mol_type = genomic DNA
taataataaaatgctcacca

SEQ ID NO: 456 - DNA, 20 bp, Cannabis sativa
  misc_feature 1..20: note = CsMLO2 position 8500 targeted gRNA
  source 1..20: mol_type = genomic DNA
ggatgcattgaaataattaa

SEQ ID NO: 457 - DNA, 20 bp, Cannabis sativa
  misc_feature 1..20: note = CsMLO2 position 8521 targeted gRNA
  source 1..20: mol_type = genomic DNA
aatctaaactgtgataatta

SEQ ID NO: 458 - DNA, 20 bp, Cannabis sativa
  misc_feature 1..20: note = CsMLO2 position 8522 targeted gRNA
  source 1..20: mol_type = genomic DNA
aaatctaaactgtgataatt

SEQ ID NO: 459 - DNA, 20 bp, Cannabis sativa
  misc_feature 1..20: note = CsMLO2 position 8566 targeted gRNA
  source 1..20: mol_type = genomic DNA
ttgacatatatgcacacgtt

SEQ ID NO: 460 - DNA, 20 bp, Cannabis sativa
  misc_feature 1..20: note = CsMLO2 position 8605 targeted gRNA
  source 1..20: mol_type = genomic DNA
tatatttttgtttttattat

SEQ ID NO: 461 - DNA, 20 bp, Cannabis sativa
  misc_feature 1..20: note = CsMLO2 position 8618 targeted gRNA
  source 1..20: mol_type = genomic DNA
aaatgtaaacaaattcatta

SEQ ID NO: 462 - DNA, 20 bp, Cannabis sativa
  misc_feature 1..20: note = CsMLO2 position 8631 targeted gRNA
  source 1..20: mol_type = genomic DNA
ataatgaatttgtttacatt

SEQ ID NO: 463 - DNA, 20 bp, Cannabis sativa
  misc_feature 1..20: note = CsMLO2 position 8636 targeted gRNA
  source 1..20: mol_type = genomic DNA
gaatttgtttacatttggac

SEQ ID NO: 464 - DNA, 20 bp, Cannabis sativa
  misc_feature 1..20: note = CsMLO2 position 8640 targeted gRNA
  source 1..20: mol_type = genomic DNA
ttgtttacatttggacaggc

SEQ ID NO: 465 - DNA, 20 bp, Cannabis sativa
  misc_feature 1..20: note = CsMLO2 position 8655 targeted gRNA
  source 1..20: mol_type = genomic DNA
caggctggtattcttatctt

SEQ ID NO: 466 - DNA, 20 bp, Cannabis sativa
  misc_feature 1..20: note = CsMLO2 position 8670 targeted gRNA
  source 1..20: mol_type = genomic DNA
cttacaattagaggaataaa

SEQ ID NO: 467 - DNA, 20 bp, Cannabis sativa
  misc_feature 1..20: note = CsMLO2 position 8679 targeted gRNA
  source 1..20: mol_type = genomic DNA
atattagtacttacaattag

SEQ ID NO: 468 - DNA, 20 bp, Cannabis sativa
  misc_feature 1..20: note = CsMLO2 position 8820 targeted gRNA
  source 1..20: mol_type = genomic DNA
gattgtttgaattttatttt

SEQ ID NO: 469 - DNA, 20 bp, Cannabis sativa
  misc_feature 1..20: note = CsMLO2 position 8907 targeted gRNA
  source 1..20: mol_type = genomic DNA
gtttacagtaaaactttaaa

SEQ ID NO: 470 - DNA, 20 bp, Cannabis sativa
  misc_feature 1..20: note = CsMLO2 position 8932 targeted gRNA
  source 1..20: mol_type = genomic DNA
aattagcccaatttttttca

SEQ ID NO: 471 - DNA, 20 bp, Cannabis sativa
  misc_feature 1..20: note = CsMLO2 position 8936 targeted gRNA
  source 1..20: mol_type = genomic DNA
taaactaccgtgaaaaaaat

SEQ ID NO: 472 - DNA, 20 bp, Cannabis sativa
  misc_feature 1..20: note = CsMLO2 position 8937 targeted gRNA
  source 1..20: mol_type = genomic DNA
aaactaccgtgaaaaaaatt

SEQ ID NO: 473 - DNA, 20 bp, Cannabis sativa
  misc_feature 1..20: note = CsMLO2 position 9001 targeted gRNA
  source 1..20: mol_type = genomic DNA
ctcttttattttttaagaag

SEQ ID NO: 474 - DNA, 20 bp, Cannabis sativa
  misc_feature 1..20: note = CsMLO2 position 9053 targeted gRNA
  source 1..20: mol_type = genomic DNA
tattataaataaattatgtt

SEQ ID NO: 475 - DNA, 20 bp, Cannabis sativa
  misc_feature 1..20: note = CsMLO2 position 9065 targeted gRNA
  source 1..20: mol_type = genomic DNA
attatgttaggtgatcctat

SEQ ID NO: 476 - DNA, 20 bp, Cannabis sativa
  misc_feature 1..20: note = CsMLO2 position 9068 targeted gRNA
  source 1..20: mol_type = genomic DNA
atgttaggtgatcctattgg

SEQ ID NO: 477 - DNA, 20 bp, Cannabis sativa
  misc_feature 1..20: note = CsMLO2 position 9069 targeted gRNA
  source 1..20: mol_type = genomic DNA
tgttaggtgatcctattggt

SEQ ID NO: 478 - DNA, 20 bp, Cannabis sativa
  misc_feature 1..20: note = CsMLO2 position 9069, strand -1, targeted gRNA
  source 1..20: mol_type = genomic DNA
gtaatttcgtccccaccaat

SEQ ID NO: 479 - DNA, 20 bp, Cannabis sativa
  misc_feature 1..20: note = CsMLO2 position 9070 targeted gRNA
  source 1..20: mol_type = genomic DNA
gttaggtgatcctattggtg

SEQ ID NO: 480 - DNA, 20 bp, Cannabis sativa
  misc_feature 1..20: note = CsMLO2 position 9101 targeted gRNA
  source 1..20: mol_type = genomic DNA
acaagtgattataacaaaga

SEQ ID NO: 481 - DNA, 20 bp, Cannabis sativa
  misc_feature 1..20: note = CsMLO2 position 9102targeted gRNA
  source 1..20: mol_type = genomic DNA
caagtgattataacaaagat

SEQ ID NO: 482 - DNA, 20 bp, Cannabis sativa
  misc_feature 1..20: note = CsMLO2 position 9103 targeted gRNA
  source 1..20: mol_type = genomic DNA
aagtgattataacaaagatg

SEQ ID NO: 483 - DNA, 20 bp, Cannabis sativa
  misc_feature 1..20: note = CsMLO2 position 9123 targeted gRNA
  source 1..20: mol_type = genomic DNA
gggctaagaattcaagaaag

SEQ ID NO: 484 - DNA, 20 bp, Cannabis sativa
  misc_feature 1..20: note = CsMLO2 position 9138 targeted gRNA
  source 1..20: mol_type = genomic DNA
gaaagaggagaagttgtaaa

SEQ ID NO: 485 - DNA, 20 bp, Cannabis sativa
  misc_feature 1..20: note = CsMLO2 position 9149 targeted gRNA
  source 1..20: mol_type = genomic DNA
agttgtaaaaggagtgcctg

SEQ ID NO: 486 - DNA, 20 bp, Cannabis sativa
  misc_feature 1..20: note = CsMLO2 position 9154 targeted gRNA
  source 1..20: mol_type = genomic DNA
tcgtccccaggttggaccac

SEQ ID NO: 487 - DNA, 20 bp, Cannabis sativa
  misc_feature 1..20: note = CsMLO2 position 9159 targeted gRNA
  source 1..20: mol_type = genomic DNA
ggagtgcctgtggtccaacc

SEQ ID NO: 488 - DNA, 20 bp, Cannabis sativa
  misc_feature 1..20: note = CsMLO2 position 9160 targeted gRNA
  source 1..20: mol_type = genomic DNA
gagtgcctgtggtccaacct

SEQ ID NO: 489 - DNA, 20 bp, Cannabis sativa
  misc_feature 1..20: note = CsMLO2 position 9161 targeted gRNA
  source 1..20: mol_type = genomic DNA
agtgcctgtggtccaacctg

SEQ ID NO: 490 - DNA, 20 bp, Cannabis sativa
  misc_feature 1..20: note = CsMLO2 position 9162 targeted gRNA
  source 1..20: mol_type = genomic DNA
agaaaaggtcgtccccaggt

SEQ ID NO: 491 - DNA, 20 bp, Cannabis sativa
  misc_feature 1..20: note = CsMLO2 position 9166 targeted gRNA
  source 1..20: mol_type = genomic DNA
aaccagaaaaggtcgtcccc

SEQ ID NO: 492 - DNA, 20 bp, Cannabis sativa
  misc_feature 1..20: note = CsMLO2 position 9175 targeted gRNA
  source 1..20: mol_type = genomic DNA
aacctggggacgaccttttc

SEQ ID NO: 493 - DNA, 20 bp, Cannabis sativa
  misc_feature 1..20: note = CsMLO2 position 9177 targeted gRNA
  source 1..20: mol_type = genomic DNA
gtgggcggttgaaccagaaa

SEQ ID NO: 494 - DNA, 20 bp, Cannabis sativa
  misc_feature 1..20: note = CsMLO2 position 9192 targeted gRNA
  source 1..20: mol_type = genomic DNA
ggtagagaataaggcgtggg

SEQ ID NO: 495 - DNA, 20 bp, Cannabis sativa
  misc_feature 1..20: note = CsMLO2 position 9195 targeted gRNA
  source 1..20: mol_type = genomic DNA
taaggtagagaataaggcgt

SEQ ID NO: 496 - DNA, 20 bp, Cannabis sativa
  misc_feature 1..20: note = CsMLO2 position 9196 targeted gRNA
  source 1..20: mol_type = genomic DNA
ataaggtagagaataaggcg

SEQ ID NO: 497 - DNA, 20 bp, Cannabis sativa
  misc_feature 1..20: note = CsMLO2 position 9201 targeted gRNA
  source 1..20: mol_type = genomic DNA
agttaataaggtagagaata

SEQ ID NO: 498 - DNA, 20 bp, Cannabis sativa
  misc_feature 1..20: note = CsMLO2 position 9213 targeted gRNA
  source 1..20: mol_type = genomic DNA
ggaagaggacgaagttaata

SEQ ID NO: 499 - DNA, 20 bp, Cannabis sativa
  misc_feature 1..20: note = CsMLO2 position 9227 targeted gRNA
  source 1..20: mol_type = genomic DNA
tattaacttcgtcctcttcc

SEQ ID NO: 500 - DNA, 20 bp, Cannabis sativa
  misc_feature 1..20: note = CsMLO2 position 9228 targeted gRNA
  source 1..20: mol_type = genomic DNA
attgattatgtacctggaag

SEQ ID NO: 501 - DNA, 20 bp, Cannabis sativa
  misc_feature 1..20: note = CsMLO2 position 9234 targeted gRNA
  source 1..20: mol_type = genomic DNA
attttgattgattatgtacc

SEQ ID NO: 502 - DNA, 20 bp, Cannabis sativa
  misc_feature 1..20: note = CsMLO2 position 9254 targeted gRNA
  source 1..20: mol_type = genomic DNA
taatcaatcaaaatcagcct

SEQ ID NO: 503 - DNA, 20 bp, Cannabis sativa
  misc_feature 1..20: note = CsMLO2 position 9260 targeted gRNA
  source 1..20: mol_type = genomic DNA
ggtgcattatagaatttcca

SEQ ID NO: 504 - DNA, 20 bp, Cannabis sativa
  misc_feature 1..20: note = CsMLO2 position 9281 targeted gRNA
  source 1..20: mol_type = genomic DNA
tcaatgtattcattttaagg

SEQ ID NO: 505 - DNA, 20 bp, Cannabis sativa
  misc_feature 1..20: note = CsMLO2 position 9282 targeted gRNA
  source 1..20: mol_type = genomic DNA
atcaatgtattcattttaag

SEQ ID NO: 506 - DNA, 20 bp, Cannabis sativa
  misc_feature 1..20: note = CsMLO2 position 9283 targeted gRNA
  source 1..20: mol_type = genomic DNA
catcaatgtattcattttaa

SEQ ID NO: 507 - DNA, 20 bp, Cannabis sativa
  misc_feature 1..20: note = CsMLO2 position 9284 targeted gRNA
  source 1..20: mol_type = genomic DNA
gcatcaatgtattcatttta

SEQ ID NO: 508 - DNA, 20 bp, Cannabis sativa
  misc_feature 1..20: note = CsMLO2 position 9308 targeted gRNA
  source 1..20: mol_type = genomic DNA
ttgagtgctaaaacaagtaa

SEQ ID NO: 509 - DNA, 20 bp, Cannabis sativa
  misc_feature 1..20: note = CsMLO2 position 9309 targeted gRNA
  source 1..20: mol_type = genomic DNA
tttgagtgctaaaacaagta

SEQ ID NO: 510 - DNA, 20 bp, Cannabis sativa
  misc_feature 1..20: note = CsMLO2 position 9350 targeted gRNA
  source 1..20: mol_type = genomic DNA
tttagtcaaattttttctca

SEQ ID NO: 511 - DNA, 20 bp, Cannabis sativa
  misc_feature 1..20: note = CsMLO2 position 10632 targeted gRNA
  source 1..20: mol_type = genomic DNA
tccatgcaaagaacgcaagc

SEQ ID NO: 512 - DNA, 20 bp, Cannabis sativa
  misc_feature 1..20: note = CsMLO2 position 10642 targeted gRNA
  source 1..20: mol_type = genomic DNA
tccagcttgcgttctttgca

SEQ ID NO: 513 - DNA, 20 bp, Cannabis sativa
  misc_feature 1..20: note = CsMLO2 position 10648 targeted gRNA
  source 1..20: mol_type = genomic DNA
ttgcgttctttgcatggact

SEQ ID NO: 514 - DNA, 20 bp, Cannabis sativa
  misc_feature 1..20: note = CsMLO2 position 10649 targeted gRNA
  source 1..20: mol_type = genomic DNA
tgcgttctttgcatggactt

SEQ ID NO: 515 - DNA, 20 bp, Cannabis sativa
  misc_feature 1..20: note = CsMLO2 position 10753 targeted gRNA
  source 1..20: mol_type = genomic DNA
ttaatttttcagtatgaatt

SEQ ID NO: 516 - DNA, 20 bp, Cannabis sativa
  misc_feature 1..20: note = CsMLO2 position 10785targeted gRNA
  source 1..20: mol_type = genomic DNA
ttgctttcatgaacatgttg

SEQ ID NO: 517 - DNA, 20 bp, Cannabis sativa
  misc_feature 1..20: note = CsMLO2 position 10791 targeted gRNA
  source 1..20: mol_type = genomic DNA
tcatgaacatgttgaggatg

SEQ ID NO: 518 - DNA, 20 bp, Cannabis sativa
  misc_feature 1..20: note = CsMLO2 position 10809 targeted gRNA
  source 1..20: mol_type = genomic DNA
tgtggttatcagaatcacca

SEQ ID NO: 519 - DNA, 20 bp, Cannabis sativa
  misc_feature 1..20: note = CsMLO2 position 10810 targeted gRNA
  source 1..20: mol_type = genomic DNA
gtggttatcagaatcaccat

SEQ ID NO: 520 - DNA, 20 bp, Cannabis sativa
  misc_feature 1..20: note = CsMLO2 position 10811 targeted gRNA
  source 1..20: mol_type = genomic DNA
tggttatcagaatcaccatg

SEQ ID NO: 521 - DNA, 20 bp, Cannabis sativa
  misc_feature 1..20: note = CsMLO2 position 10815 targeted gRNA
  source 1..20: mol_type = genomic DNA
atatctgtatacagacccca

SEQ ID NO: 522 - DNA, 20 bp, Cannabis sativa
  misc_feature 1..20: note = CsMLO2 position 10922 targeted gRNA
  source 1..20: mol_type = genomic DNA
aaataaaaattaaatattaa

SEQ ID NO: 523 - DNA, 20 bp, Cannabis sativa
  misc_feature 1..20: note = CsMLO2 position 10958 targeted gRNA
  source 1..20: mol_type = genomic DNA
gtaaaaatttctaacaccgt

SEQ ID NO: 524 - DNA, 20 bp, Cannabis sativa
  misc_feature 1..20: note = CsMLO2 position 10963 targeted gRNA
  source 1..20: mol_type = genomic DNA
ccctgatgatcatgatccaa

SEQ ID NO: 525 - DNA, 20 bp, Cannabis sativa
  misc_feature 1..20: note = CsMLO2 position 10973 targeted gRNA
  source 1..20: mol_type = genomic DNA
accgttggatcatgatcatc

SEQ ID NO: 526 - DNA, 20 bp, Cannabis sativa
  misc_feature 1..20: note = CsMLO2 position 10974 targeted gRNA
  source 1..20: mol_type = genomic DNA
ccgttggatcatgatcatca

SEQ ID NO: 527 - DNA, 20 bp, Cannabis sativa
  misc_feature 1..20: note = CsMLO2 position 10993 targeted gRNA
  source 1..20: mol_type = genomic DNA
tgacgtagctgcacagaatc

SEQ ID NO: 528 - DNA, 20 bp, Cannabis sativa
  misc_feature 1..20: note = CsMLO2 position 11016 targeted gRNA
  source 1..20: mol_type = genomic DNA
aacaagggcgtagagaggga

SEQ ID NO: 529 - DNA, 20 bp, Cannabis sativa
  misc_feature 1..20: note = CsMLO2 position 11017 targeted gRNA
  source 1..20: mol_type = genomic DNA
taacaagggcgtagagaggg

SEQ ID NO: 530 - DNA, 20 bp, Cannabis sativa
  misc_feature 1..20: note = CsMLO2 position 11020 targeted gRNA
  source 1..20: mol_type = genomic DNA
gtgtaacaagggcgtagaga

SEQ ID NO: 531 - DNA, 20 bp, Cannabis sativa
  misc_feature 1..20: note = CsMLO2 position 11021 targeted gRNA
  source 1..20: mol_type = genomic DNA
tgtgtaacaagggcgtagag

SEQ ID NO: 532 - DNA, 20 bp, Cannabis sativa
  misc_feature 1..20: note = CsMLO2 position 11031 targeted gRNA
  source 1..20: mol_type = genomic DNA
tgtaattacttgtgtaacaa

SEQ ID NO: 533 - DNA, 20 bp, Cannabis sativa
  misc_feature 1..20: note = CsMLO2 position 11032 targeted gRNA
  source 1..20: mol_type = genomic DNA
gtgtaattacttgtgtaaca

SEQ ID NO: 534 - DNA, 20 bp, Cannabis sativa
  misc_feature 1..20: note = CsMLO2 position 11159 targeted gRNA
  source 1..20: mol_type = genomic DNA
agattttatatatttaatta

SEQ ID NO: 535 - DNA, 20 bp, Cannabis sativa
  misc_feature 1..20: note = CsMLO2 position 11160 targeted gRNA
  source 1..20: mol_type = genomic DNA
tagattttatatatttaatt

SEQ ID NO: 536 - DNA, 20 bp, Cannabis sativa
  misc_feature 1..20: note = CsMLO2 position 11524 targeted gRNA
  source 1..20: mol_type = genomic DNA
cggactatattttaattaaa

SEQ ID NO: 537 - DNA, 20 bp, Cannabis sativa
  misc_feature 1..20: note = CsMLO2 position 11544 targeted gRNA
  source 1..20: mol_type = genomic DNA
taattaaataaaattctaaa

SEQ ID NO: 538 - DNA, 20 bp, Cannabis sativa
  misc_feature 1..20: note = CsMLO2 position 11580 targeted gRNA
  source 1..20: mol_type = genomic DNA
taaaaaatattgtcatagtt

SEQ ID NO: 539 - DNA, 20 bp, Cannabis sativa
  misc_feature 1..20: note = CsMLO2 position 11581 targeted gRNA
  source 1..20: mol_type = genomic DNA
aaaaaatattgtcatagttt

SEQ ID NO: 540 - DNA, 20 bp, Cannabis sativa
  misc_feature 1..20: note = CsMLO2 position 11782 targeted gRNA
  source 1..20: mol_type = genomic DNA
tatatatatgacacaacaga

SEQ ID NO: 541 - DNA, 20 bp, Cannabis sativa
  misc_feature 1..20: note = CsMLO2 position 11783 targeted gRNA
  source 1..20: mol_type = genomic DNA
atatatatgacacaacagat

SEQ ID NO: 542 - DNA, 20 bp, Cannabis sativa
  misc_feature 1..20: note = CsMLO2 position 11800 targeted gRNA
  source 1..20: mol_type = genomic DNA
gttgaatatagttggtttca

SEQ ID NO: 543 - DNA, 20 bp, Cannabis sativa
  misc_feature 1..20: note = CsMLO2 position 11808 targeted gRNA
  source 1..20: mol_type = genomic DNA
actttgtcgttgaatatagt

SEQ ID NO: 544 - DNA, 20 bp, Cannabis sativa
  misc_feature 1..20: note = CsMLO2 position 11824 targeted gRNA
  source 1..20: mol_type = genomic DNA
tatattcaacgacaaagtag

SEQ ID NO: 545 - DNA, 20 bp, Cannabis sativa
  misc_feature 1..20: note = CsMLO2 position 11827 targeted gRNA
  source 1..20: mol_type = genomic DNA
attcaacgacaaagtagcgg

SEQ ID NO: 546 - DNA, 20 bp, Cannabis sativa
  misc_feature 1..20: note = CsMLO2 position 11839 targeted gRNA
  source 1..20: mol_type = genomic DNA
tgagtggtgccagttgcgga

SEQ ID NO: 547 - DNA, 20 bp, Cannabis sativa
  misc_feature 1..20: note = CsMLO2 position 11840 targeted gRNA
  source 1..20: mol_type = genomic DNA
ctgagtggtgccagttgcgg

SEQ ID NO: 548 - DNA, 20 bp, Cannabis sativa
  misc_feature 1..20: note = CsMLO2 position 11841 targeted gRNA
  source 1..20: mol_type = genomic DNA
tagcggaggccctccgcaac

SEQ ID NO: 549 - DNA, 20 bp, Cannabis sativa
  misc_feature 1..20: note = CsMLO2 position 11843 targeted gRNA
  source 1..20: mol_type = genomic DNA
gggctgagtggtgccagttg

SEQ ID NO: 550 - DNA, 20 bp, Cannabis sativa
  misc_feature 1..20: note = CsMLO2 position 11855 targeted gRNA
  source 1..20: mol_type = genomic DNA
tgatgtgctttcgggctgag

SEQ ID NO: 551 - DNA, 20 bp, Cannabis sativa
  misc_feature 1..20: note = CsMLO2 position 11863 targeted gRNA
  source 1..20: mol_type = genomic DNA
ttggtgtttgatgtgctttc

SEQ ID NO: 552 - DNA, 20 bp, Cannabis sativa
  misc_feature 1..20: note = CsMLO2 position 11864 targeted gRNA
  source 1..20: mol_type = genomic DNA
tttggtgtttgatgtgcttt

SEQ ID NO: 553 - DNA, 20 bp, Cannabis sativa
  misc_feature 1..20: note = CsMLO2 position 11881 targeted gRNA
  source 1..20: mol_type = genomic DNA
gcacatcaaacaccaaaaca

SEQ ID NO: 554 - DNA, 20 bp, Cannabis sativa
  misc_feature 1..20: note = CsMLO2 position 11882 targeted gRNA
  source 1..20: mol_type = genomic DNA
ctgaccccgccgccttgttt

SEQ ID NO: 555 - DNA, 20 bp, Cannabis sativa
  misc_feature 1..20: note = CsMLO2 position 11884 targeted gRNA
  source 1..20: mol_type = genomic DNA
catcaaacaccaaaacaagg

SEQ ID NO: 556 - DNA, 20 bp, Cannabis sativa
  misc_feature 1..20: note = CsMLO2 position 11887 targeted gRNA
  source 1..20: mol_type = genomic DNA
caaacaccaaaacaaggcgg

SEQ ID NO: 557 - DNA, 20 bp, Cannabis sativa
  misc_feature 1..20: note = CsMLO2 position 11888 targeted gRNA
  source 1..20: mol_type = genomic DNA
aaacaccaaaacaaggcggc

SEQ ID NO: 558 - DNA, 20 bp, Cannabis sativa
  misc_feature 1..20: note = CsMLO2 position 11889 targeted gRNA
  source 1..20: mol_type = genomic DNA
aacaccaaaacaaggcggcg

SEQ ID NO: 559 - DNA, 20 bp, Cannabis sativa
  misc_feature 1..20: note = CsMLO2 position 11910 targeted gRNA
  source 1..20: mol_type = genomic DNA
gtcgtcggccggcttgacag

SEQ ID NO: 560 - DNA, 20 bp, Cannabis sativa
  misc_feature 1..20: note = CsMLO2 position 11913 targeted gRNA
  source 1..20: mol_type = genomic DNA
cagtgacgccgctgtcaagc

SEQ ID NO: 561 - DNA, 20 bp, Cannabis sativa
  misc_feature 1..20: note = CsMLO2 position 11921 targeted gRNA
  source 1..20: mol_type = genomic DNA
gatgtgtgggtgtcgtcggc

SEQ ID NO: 562 - DNA, 20 bp, Cannabis sativa
  misc_feature 1..20: note = CsMLO2 position 11925 targeted gRNA
  source 1..20: mol_type = genomic DNA
atgtgatgtgtgggtgtcgt

SEQ ID NO: 563 - DNA, 20 bp, Cannabis sativa
  misc_feature 1..20: note = CsMLO2 position 11934 targeted gRNA
  source 1..20: mol_type = genomic DNA
accggggacatgtgatgtgt

SEQ ID NO: 564 - DNA, 20 bp, Cannabis sativa
  misc_feature 1..20: note = CsMLO2 position 11935 targeted gRNA
  source 1..20: mol_type = genomic DNA
gaccggggacatgtgatgtg

SEQ ID NO: 565 - DNA, 20 bp, Cannabis sativa
  misc_feature 1..20: note = CsMLO2 position 11944 targeted gRNA
  source 1..20: mol_type = genomic DNA
acccacacatcacatgtccc

SEQ ID NO: 566 - DNA, 20 bp, Cannabis sativa
  misc_feature 1..20: note = CsMLO2 position 11950 targeted gRNA
  source 1..20: mol_type = genomic DNA
gtggcgcaagaggtggaccg

SEQ ID NO: 567 - DNA, 20 bp, Cannabis sativa
  misc_feature 1..20: note = CsMLO2 position 11951 targeted gRNA
  source 1..20: mol_type = genomic DNA
agtggcgcaagaggtggacc

SEQ ID NO: 568 - DNA, 20 bp, Cannabis sativa
  misc_feature 1..20: note = CsMLO2 position 11952 targeted gRNA
  source 1..20: mol_type = genomic DNA
tagtggcgcaagaggtggac

SEQ ID NO: 569 - DNA, 20 bp, Cannabis sativa
  misc_feature 1..20: note = CsMLO2 position 11957 targeted gRNA
  source 1..20: mol_type = genomic DNA
tgcggtagtggcgcaagagg

SEQ ID NO: 570 - DNA, 20 bp, Cannabis sativa
  misc_feature 1..20: note = CsMLO2 position 11960 targeted gRNA
  source 1..20: mol_type = genomic DNA
cactgcggtagtggcgcaag

SEQ ID NO: 571 - DNA, 20 bp, Cannabis sativa
  misc_feature 1..20: note = CsMLO2 position 11969 targeted gRNA
  source 1..20: mol_type = genomic DNA
ctgctgcctcactgcggtag

SEQ ID NO: 572 - DNA, 20 bp, Cannabis sativa
  misc_feature 1..20: note = CsMLO2 position 11974 targeted gRNA
  source 1..20: mol_type = genomic DNA
cttgcgccactaccgcagtg

SEQ ID NO: 573 - DNA, 20 bp, Cannabis sativa
  misc_feature 1..20: note = CsMLO2 position 11975 targeted gRNA
  source 1..20: mol_type = genomic DNA
ggctgtctgctgcctcactg

SEQ ID NO: 574 - DNA, 20 bp, Cannabis sativa
  misc_feature 1..20: note = CsMLO2 position 11996 targeted gRNA
  source 1..20: mol_type = genomic DNA
agcgccttggggagttttgg

SEQ ID NO: 575 - DNA, 20 bp, Cannabis sativa
  misc_feature 1..20: note = CsMLO2 position 11999 targeted gRNA
  source 1..20: mol_type = genomic DNA
ttgagcgccttggggagttt

SEQ ID NO: 576 - DNA, 20 bp, Cannabis sativa
  misc_feature 1..20: note = CsMLO2 position 12003 targeted gRNA
  source 1..20: mol_type = genomic DNA
acagcctccaaaactcccca

SEQ ID NO: 577 - DNA, 20 bp, Cannabis sativa
  misc_feature 1..20: note = CsMLO2 position 12007 targeted gRNA
  source 1..20: mol_type = genomic DNA
atcaaagtttgagcgccttg

SEQ ID NO: 578 - DNA, 20 bp, Cannabis sativa
  misc_feature 1..20: note = CsMLO2 position 12008 targeted gRNA
  source 1..20: mol_type = genomic DNA
catcaaagtttgagcgcctt

SEQ ID NO: 579 - DNA, 20 bp, Cannabis sativa
  misc_feature 1..20: note = CsMLO2 position 12009 targeted gRNA
  source 1..20: mol_type = genomic DNA
ccatcaaagtttgagcgcct

SEQ ID NO: 580 - DNA, 20 bp, Cannabis sativa
  misc_feature 1..20: note = CsMLO2 position 12020 targeted gRNA
  source 1..20: mol_type = genomic DNA
ccaaggcgctcaaactttga

SEQ ID NO: 581 - DNA, 20 bp, Cannabis sativa
  misc_feature 1..20: note = CsMLO2 position 12033 targeted gRNA
  source 1..20: mol_type = genomic DNA
actttgatggcgccactgaa

SEQ ID NO: 582 - DNA, 20 bp, Cannabis sativa
  misc_feature 1..20: note = CsMLO2 position 12034 targeted gRNA
  source 1..20: mol_type = genomic DNA
atctgtctcccaccgttcag

SEQ ID NO: 583 - DNA, 20 bp, Cannabis sativa
  misc_feature 1..20: note = CsMLO2 position 12036 targeted gRNA
  source 1..20: mol_type = genomic DNA
ttgatggcgccactgaacgg

SEQ ID NO: 584 - DNA, 20 bp, Cannabis sativa
  misc_feature 1..20: note = CsMLO2 position 12037 targeted gRNA
  source 1..20: mol_type = genomic DNA
tgatggcgccactgaacggt

SEQ ID NO: 585 - DNA, 20 bp, Cannabis sativa
  misc_feature 1..20: note = CsMLO2 position 12059 targeted gRNA
  source 1..20: mol_type = genomic DNA
tggtggtggtgagatggaga

SEQ ID NO: 586 - DNA, 20 bp, Cannabis sativa
  misc_feature 1..20: note = CsMLO2 position 12065 targeted gRNA
  source 1..20: mol_type = genomic DNA
cggccgtggtggtggtgaga

SEQ ID NO: 587 - DNA, 20 bp, Cannabis sativa
  misc_feature 1..20: note = CsMLO2 position 12073 targeted gRNA
  source 1..20: mol_type = genomic DNA
tctccatctcaccaccacca

SEQ ID NO: 588 - DNA, 20 bp, Cannabis sativa
  misc_feature 1..20: note = CsMLO2 position 12073,strand -1, targeted gRNA
  source 1..20: mol_type = genomic DNA
tcgcgaagcggccgtggtgg

SEQ ID NO: 589 - DNA, 20 bp, Cannabis sativa
  misc_feature 1..20: note = CsMLO2 position 12076 targeted gRNA
  source 1..20: mol_type = genomic DNA
cggtcgcgaagcggccgtgg

SEQ ID NO: 590 - DNA, 20 bp, Cannabis sativa
  misc_feature 1..20: note = CsMLO2 position 12079 targeted gRNA
  source 1..20: mol_type = genomic DNA
cctcggtcgcgaagcggccg

SEQ ID NO: 591 - DNA, 20 bp, Cannabis sativa
  misc_feature 1..20: note = CsMLO2 position 12085 targeted gRNA
  source 1..20: mol_type = genomic DNA
aggaaccctcggtcgcgaag

SEQ ID NO: 592 - DNA, 20 bp, Cannabis sativa
  misc_feature 1..20: note = CsMLO2 position 12090 targeted gRNA
  source 1..20: mol_type = genomic DNA
ccacggccgcttcgcgaccg

SEQ ID NO: 593 - DNA, 20 bp, Cannabis sativa
  misc_feature 1..20: note = CsMLO2 position 12091 targeted gRNA
  source 1..20: mol_type = genomic DNA
cacggccgcttcgcgaccga

SEQ ID NO: 594 - DNA, 20 bp, Cannabis sativa
  misc_feature 1..20: note = CsMLO2 position 12096 targeted gRNA
  source 1..20: mol_type = genomic DNA
atgatgagaggaggaaccct

SEQ ID NO: 595 - DNA, 20 bp, Cannabis sativa
  misc_feature 1..20: note = CsMLO2 position 12105 targeted gRNA
  source 1..20: mol_type = genomic DNA
attattactatgatgagagg

SEQ ID NO: 596 - DNA, 20 bp, Cannabis sativa
  misc_feature 1..20: note = CsMLO2 position 12108 targeted gRNA
  source 1..20: mol_type = genomic DNA
attattattactatgatgag

SEQ ID NO: 597 - DNA, 20 bp, Cannabis sativa
  misc_feature 1..20: note = CsMLO2 position 12150 targeted gRNA
  source 1..20: mol_type = genomic DNA
taaaaatcagcaaattgaat

SEQ ID NO: 598 - DNA, 20 bp, Cannabis sativa
  misc_feature 1..20: note = CsMLO2 position 12151 targeted gRNA
  source 1..20: mol_type = genomic DNA
aaaaatcagcaaattgaatt

SEQ ID NO: 599 - DNA, 20 bp, Cannabis sativa
  misc_feature 1..20: note = CsMLO2 position 12162 targeted gRNA
  source 1..20: mol_type = genomic DNA
aattgaattgggacaaataa

SEQ ID NO: 600 - DNA, 20 bp, Cannabis sativa
  misc_feature 1..20: note = CsMLO2 position 12181 targeted gRNA
  source 1..20: mol_type = genomic DNA
atggaacaacatcatcttca

SEQ ID NO: 601 - DNA, 20 bp, Cannabis sativa
  misc_feature 1..20: note = CsMLO2 position 12188 targeted gRNA
  source 1..20: mol_type = genomic DNA
aacatcatcttcatggagat

SEQ ID NO: 602 - DNA, 20 bp, Cannabis sativa
  misc_feature 1..20: note = CsMLO2 position 12204 targeted gRNA
  source 1..20: mol_type = genomic DNA
ggtttgaggaggaagctcat

SEQ ID NO: 603 - DNA, 20 bp, Cannabis sativa
  misc_feature 1..20: note = CsMLO2 position 12215 targeted gRNA
  source 1..20: mol_type = genomic DNA
cttaatgtagtggtttgagg

SEQ ID NO: 604 - DNA, 20 bp, Cannabis sativa
  misc_feature 1..20: note = CsMLO2 position 12218 targeted gRNA
  source 1..20: mol_type = genomic DNA
tttcttaatgtagtggtttg

SEQ ID NO: 605 - DNA, 20 bp, Cannabis sativa
  misc_feature 1..20: note = CsMLO2 position 12225 targeted gRNA
  source 1..20: mol_type = genomic DNA
agcttgatttcttaatgtag

SEQ ID NO: 606 - DNA, 20 bp, Cannabis sativa
  misc_feature 1..20: note = CsMLO2 position 12267 targeted gRNA
  source 1..20: mol_type = genomic DNA
tgatcaatcagcagcagcac

SEQ ID NO: 607 - DNA, 20 bp, Cannabis sativa
  misc_feature 1..20: note = CsMLO2 position 12272 targeted gRNA
  source 1..20: mol_type = genomic DNA
aatcagcagcagcacaggtg

SEQ ID NO: 608 - DNA, 20 bp, Cannabis sativa
  misc_feature 1..20: note = CsMLO2 position 12284 targeted gRNA
  source 1..20: mol_type = genomic DNA
ttaatttcatggtggggcgg

SEQ ID NO: 609 - DNA, 20 bp, Cannabis sativa
  misc_feature 1..20: note = CsMLO2 position 12287 targeted gRNA
  source 1..20: mol_type = genomic DNA
atattaatttcatggtgggg

SEQ ID NO: 610 - DNA, 20 bp, Cannabis sativa
  misc_feature 1..20: note = CsMLO2 position 12290 targeted gRNA
  source 1..20: mol_type = genomic DNA
ccaatattaatttcatggtg

SEQ ID NO: 611 - DNA, 20 bp, Cannabis sativa
  misc_feature 1..20: note = CsMLO2 position 12291 targeted gRNA
  source 1..20: mol_type = genomic DNA
tccaatattaatttcatggt

SEQ ID NO: 612 - DNA, 20 bp, Cannabis sativa
  misc_feature 1..20: note = CsMLO2 position 12292 targeted gRNA
  source 1..20: mol_type = genomic DNA
gtccaatattaatttcatgg

SEQ ID NO: 613 - DNA, 20 bp, Cannabis sativa
  misc_feature 1..20: note = CsMLO2 position 12295 targeted gRNA
  source 1..20: mol_type = genomic DNA
tgtgtccaatattaatttca

SEQ ID NO: 614 - DNA, 20 bp, Cannabis sativa
  misc_feature 1..20: note = CsMLO2 position 12301 targeted gRNA
  source 1..20: mol_type = genomic DNA
ccccaccatgaaattaatat

SEQ ID NO: 615 - DNA, 20 bp, Cannabis sativa
  misc_feature 1..20: note = CsMLO2 position 12326 targeted gRNA
  source 1..20: mol_type = genomic DNA
acagagatttctcttttgaa

SEQ ID NO: 616 - DNA, 20 bp, Cannabis sativa
  misc_feature 1..20: note = CsMLO2 position 12350 targeted gRNA
  source 1..20: mol_type = genomic DNA
ctctctcgtcatcaaacgct

SEQ ID NO: 617 - DNA, 20 bp, Cannabis sativa
  misc_feature 1..20: note = CsMLO2 position 12351targeted gRNA
  source 1..20: mol_type = genomic DNA
tctctctcgtcatcaaacgc

SEQ ID NO: 618 - DNA, 20 bp, Cannabis sativa
  misc_feature 1..20: note = CsMLO2 position 12376 targeted gRNA
  source 1..20: mol_type = genomic DNA
cgagagagaattccgttatt

SEQ ID NO: 619 - DNA, 20 bp, Cannabis sativa
  misc_feature 1..20: note = CsMLO2 position 12377 targeted gRNA
  source 1..20: mol_type = genomic DNA
ttaacattataaccaaataa

SEQ ID NO: 620 - DNA, 20 bp, Cannabis sativa
  misc_feature 1..20: note = CsMLO2 position 12392 targeted gRNA
  source 1..20: mol_type = genomic DNA
tatttggttataatgttaat

SEQ ID NO: 621 - DNA, 20 bp, Cannabis sativa
  misc_feature 1..20: note = CsMLO2 position 12396 targeted gRNA
  source 1..20: mol_type = genomic DNA
tggttataatgttaatcgga

SEQ ID NO: 622 - DNA, 20 bp, Cannabis sativa
  misc_feature 1..20: note = CsMLO2 position 12411 targeted gRNA
  source 1..20: mol_type = genomic DNA
tcggacggttctcattgtct

SEQ ID NO: 623 - DNA, 20 bp, Cannabis sativa
  misc_feature 1..20: note = CsMLO2 position 12423 targeted gRNA
  source 1..20: mol_type = genomic DNA
tctagctcgttgatcatcag

SEQ ID NO: 624 - DNA, 20 bp, Cannabis sativa
  source 1..20: mol_type = genomic DNA
ataattaaaccgctcattat

SEQ ID NO: 625 - DNA, 20 bp, Cannabis sativa
  misc_feature 1..20: note = CsMLO2 position 12501 targeted gRNA
  source 1..20: mol_type = genomic DNA
taagcagctccaataatgag

SEQ ID NO: 626 - DNA, 20 bp, Cannabis sativa
  misc_feature 1..20: note = CsMLO3 position 777 targeted gRNA
  source 1..20: mol_type = genomic DNA
tgaaactcaaactaaaatca

SEQ ID NO: 627 - DNA, 20 bp, Cannabis sativa
  misc_feature 1..20: note = CsMLO3 position 801 targeted gRNA
  source 1..20: mol_type = genomic DNA
tctaacagttggtatcagag

SEQ ID NO: 628 - DNA, 20 bp, Cannabis sativa
  misc_feature 1..20: note = CsMLO3 position 812 targeted gRNA
  source 1..20: mol_type = genomic DNA
atatataaatgtctaacagt

SEQ ID NO: 629 - DNA, 20 bp, Cannabis sativa
  misc_feature 1..20: note = CsMLO3 position 860 targeted gRNA
  source 1..20: mol_type = genomic DNA
atatgtttaagtattaactg

SEQ ID NO: 630 - DNA, 20 bp, Cannabis sativa
  misc_feature 1..20: note = CsMLO3 position 894 targeted gRNA
  source 1..20: mol_type = genomic DNA
tatatacactatataactta

SEQ ID NO: 631 - DNA, 20 bp, Cannabis sativa
  misc_feature 1..20: note = CsMLO3 position 915 targeted gRNA
  source 1..20: mol_type = genomic DNA
gctcaagaatcaatggctgg

SEQ ID NO: 632 - DNA, 20 bp, Cannabis sativa
  misc_feature 1..20: note = CsMLO3 position 918 targeted gRNA
  source 1..20: mol_type = genomic DNA
gaagctcaagaatcaatggc

SEQ ID NO: 633 - DNA, 20 bp, Cannabis sativa
  misc_feature 1..20: note = CsMLO3 position 922 targeted gRNA
  source 1..20: mol_type = genomic DNA
gtttgaagctcaagaatcaa

SEQ ID NO: 634 - DNA, 20 bp, Cannabis sativa
  misc_feature 1..20: note = CsMLO3 position 944 targeted gRNA
  source 1..20: mol_type = genomic DNA
ttgcagatcaaagcttatgt

SEQ ID NO: 635 - DNA, 20 bp, Cannabis sativa
  misc_feature 1..20: note = CsMLO3 position 945 targeted gRNA
  source 1..20: mol_type = genomic DNA
cttgcagatcaaagcttatg

SEQ ID NO: 636 - DNA, 20 bp, Cannabis sativa
  misc_feature 1..20: note = CsMLO3 position 957 targeted gRNA
  source 1..20: mol_type = genomic DNA
cacataagctttgatctgca

SEQ ID NO: 637 - DNA, 20 bp, Cannabis sativa
  misc_feature 1..20: note = CsMLO3 position 958 targeted gRNA
  source 1..20: mol_type = genomic DNA
acataagctttgatctgcaa

SEQ ID NO: 638 - DNA, 20 bp, Cannabis sativa
  misc_feature 1..20: note = CsMLO3 position 965 targeted gRNA
  source 1..20: mol_type = genomic DNA
ctttgatctgcaagggaaac

SEQ ID NO: 639 - DNA, 20 bp, Cannabis sativa
  misc_feature 1..20: note = CsMLO3 position 974 targeted gRNA
  source 1..20: mol_type = genomic DNA
gcaagggaaactggttgatg

SEQ ID NO: 640 - DNA, 20 bp, Cannabis sativa
  misc_feature 1..20: note = CsMLO3 position 975 targeted gRNA
  source 1..20: mol_type = genomic DNA
caagggaaactggttgatgt

SEQ ID NO: 641 - DNA, 20 bp, Cannabis sativa
  misc_feature 1..20: note = CsMLO3 position 982 targeted gRNA
  source 1..20: mol_type = genomic DNA
aactggttgatgtgggtaat

SEQ ID NO: 642 - DNA, 20 bp, Cannabis sativa
  misc_feature 1..20: note = CsMLO3 position 983 targeted gRNA
  source 1..20: mol_type = genomic DNA
actggttgatgtgggtaatc

SEQ ID NO: 643 - DNA, 20 bp, Cannabis sativa
  misc_feature 1..20: note = CsMLO3 position 998 targeted gRNA
  source 1..20: mol_type = genomic DNA
taaagagagttgagagagcg

SEQ ID NO: 644 - DNA, 20 bp, Cannabis sativa
  misc_feature 1..20: note = CsMLO3 position 1014 targeted gRNA
  source 1..20: mol_type = genomic DNA
ctctctcaactctctttaga

SEQ ID NO: 645 - DNA, 20 bp, Cannabis sativa
  misc_feature 1..20: note = CsMLO3 position 1044 targeted gRNA
  source 1..20: mol_type = genomic DNA
tgttatgaacagaatgagtg

SEQ ID NO: 646 - DNA, 20 bp, Cannabis sativa
  misc_feature 1..20: note = CsMLO3 position 1051 targeted gRNA
  source 1..20: mol_type = genomic DNA
aacagaatgagtgaggagct

SEQ ID NO: 647 - DNA, 20 bp, Cannabis sativa
  misc_feature 1..20: note = CsMLO3 position1052 targeted gRNA
  source 1..20: mol_type = genomic DNA
acagaatgagtgaggagctc

SEQ ID NO: 648 - DNA, 20 bp, Cannabis sativa
  misc_feature 1..20: note = CsMLO3 position 1053 targeted gRNA
  source 1..20: mol_type = genomic DNA
cagaatgagtgaggagctcg

SEQ ID NO: 649 - DNA, 20 bp, Cannabis sativa
  misc_feature 1..20: note = CsMLO3 position 1066 targeted gRNA
  source 1..20: mol_type = genomic DNA
cacctataaatatagggtct

SEQ ID NO: 650 - DNA, 20 bp, Cannabis sativa
  misc_feature 1..20: note = CsMLO3 position 1072 targeted gRNA
  source 1..20: mol_type = genomic DNA
gtatctcacctataaatata

SEQ ID NO: 651 - DNA, 20 bp, Cannabis sativa
  misc_feature 1..20: note = CsMLO3 position 1073 targeted gRNA
  source 1..20: mol_type = genomic DNA
agtatctcacctataaatat

SEQ ID NO: 652 - DNA, 20 bp, Cannabis sativa
  misc_feature 1..20: note = CsMLO3 position 1075 targeted gRNA
  source 1..20: mol_type = genomic DNA
gaccgagaccctatatttat

SEQ ID NO: 653 - DNA, 20 bp, Cannabis sativa
  misc_feature 1..20: note = CsMLO3 position 1096 targeted gRNA
  source 1..20: mol_type = genomic DNA
taatgtggcacagatactga

SEQ ID NO: 654 - DNA, 20 bp, Cannabis sativa
  misc_feature 1..20: note = CsMLO3 position 1111 targeted gRNA
  source 1..20: mol_type = genomic DNA
aaatattctgacaattaatg

SEQ ID NO: 655 - DNA, 20 bp, Cannabis sativa
  misc_feature 1..20: note = CsMLO3 position 1138 targeted gRNA
  source 1..20: mol_type = genomic DNA
aatattttgacaattaattc

SEQ ID NO: 656 - DNA, 20 bp, Cannabis sativa
  misc_feature 1..20: note = CsMLO3 position 1151 targeted gRNA
  source 1..20: mol_type = genomic DNA
ttaattcaggaaatcaaatc

SEQ ID NO: 657 - DNA, 20 bp, Cannabis sativa
  misc_feature 1..20: note = CsMLO3 position 1183 targeted gRNA
  source 1..20: mol_type = genomic DNA
attatgtaatattctatata

SEQ ID NO: 658 - DNA, 20 bp, Cannabis sativa
  misc_feature 1..20: note = CsMLO3 position 4585 targeted gRNA
  source 1..20: mol_type = genomic DNA
gttctcactatcagttatta

SEQ ID NO: 659 - DNA, 20 bp, Cannabis sativa
  misc_feature 1..20: note = CsMLO3 position 4595 targeted gRNA
  source 1..20: mol_type = genomic DNA
tcagttattatggttattta

SEQ ID NO: 660 - DNA, 20 bp, Cannabis sativa
  misc_feature 1..20: note = CsMLO3 position 4615 targeted gRNA
  source 1..20: mol_type = genomic DNA
tggttatttatcttttttag

SEQ ID NO: 661 - DNA, 20 bp, Cannabis sativa
  misc_feature 1..20: note = CsMLO3 position 4634 targeted gRNA
  source 1..20: mol_type = genomic DNA
cctgaagggctttttgtgtt

SEQ ID NO: 662 - DNA, 20 bp, Cannabis sativa
  misc_feature 1..20: note = CsMLO3 position 4645 targeted gRNA
  source 1..20: mol_type = genomic DNA
ccaaacacaaaaagcccttc

SEQ ID NO: 663 - DNA, 20 bp, Cannabis sativa
  misc_feature 1..20: note = CsMLO3 position 4648 targeted gRNA
  source 1..20: mol_type = genomic DNA
cttctcaagcgcttcctgaa

SEQ ID NO: 664 - DNA, 20 bp, Cannabis sativa
  misc_feature 1..20: note = CsMLO3 position 4649 targeted gRNA
  source 1..20: mol_type = genomic DNA
tcttctcaagcgcttcctga

SEQ ID NO: 665 - DNA, 20 bp, Cannabis sativa
  misc_feature 1..20: note = CsMLO3 position 4670 targeted gRNA
  source 1..20: mol_type = genomic DNA
gcgcttgagaagattaaatt

SEQ ID NO: 666 - DNA, 20 bp, Cannabis sativa
  misc_feature 1..20: note = CsMLO3 position 4736 targeted gRNA
  source 1..20: mol_type = genomic DNA
ttattagtattttttttttt

SEQ ID NO: 667 - DNA, 20 bp, Cannabis sativa
  misc_feature 1..20: note = CsMLO3 position 4751 targeted gRNA
  source 1..20: mol_type = genomic DNA
ttttttggtctaattttaat

SEQ ID NO: 668 - DNA, 20 bp, Cannabis sativa
  misc_feature 1..20: note = CsMLO3 position 4752 targeted gRNA
  source 1..20: mol_type = genomic DNA
tttttggtctaattttaatt

SEQ ID NO: 669 - DNA, 20 bp, Cannabis sativa
  misc_feature 1..20: note = CsMLO3 position 4802 targeted gRNA
  source 1..20: mol_type = genomic DNA
tgttgcagagcttatgctat

SEQ ID NO: 670 - DNA, 20 bp, Cannabis sativa
  misc_feature 1..20: note = CsMLO3 position 4803 targeted gRNA
  source 1..20: mol_type = genomic DNA
gttgcagagcttatgctatt

SEQ ID NO: 671 - DNA, 20 bp, Cannabis sativa
  misc_feature 1..20: note = CsMLO3 position 4842 targeted gRNA
  source 1..20: mol_type = genomic DNA
atatgtcagcaatgtaatct

SEQ ID NO: 672 - DNA, 20 bp, Cannabis sativa
  misc_feature 1..20: note = CsMLO3 position 4870targeted gRNA
  source 1..20: mol_type = genomic DNA
caagtgtttgctgcactttt

SEQ ID NO: 673 - DNA, 20 bp, Cannabis sativa
  misc_feature 1..20: note = CsMLO3 position 4882 targeted gRNA
  source 1..20: mol_type = genomic DNA
caaaaagtgcagcaaacact

SEQ ID NO: 674 - DNA, 20 bp, Cannabis sativa
  misc_feature 1..20: note = CsMLO3 position 4897 targeted gRNA
  source 1..20: mol_type = genomic DNA
tcttcattttggtatgggca

SEQ ID NO: 675 - DNA, 20 bp, Cannabis sativa
  misc_feature 1..20: note = CsMLO3 position 4902 targeted gRNA
  source 1..20: mol_type = genomic DNA
ttttctcttcattttggtat

SEQ ID NO: 676 - DNA, 20 bp, Cannabis sativa
  misc_feature 1..20: note = CsMLO3 position 4903 targeted gRNA
  source 1..20: mol_type = genomic DNA
tttttctcttcattttggta

SEQ ID NO: 677 - DNA, 20 bp, Cannabis sativa
  misc_feature 1..20: note = CsMLO3 position 4908 targeted gRNA
  source 1..20: mol_type = genomic DNA
tagcctttttctcttcattt

SEQ ID NO: 678 - DNA, 20 bp, Cannabis sativa
  misc_feature 1..20: note = CsMLO3 position 4916 targeted gRNA
  source 1..20: mol_type = genomic DNA
ataccaaaatgaagagaaaa

SEQ ID NO: 679 - DNA, 20 bp, Cannabis sativa
  misc_feature 1..20: note = CsMLO3 position 4922 targeted gRNA
  source 1..20: mol_type = genomic DNA
aaatgaagagaaaaaggcta

SEQ ID NO: 680 - DNA, 20 bp, Cannabis sativa
  misc_feature 1..20: note = CsMLO3 position 4945 targeted gRNA
  source 1..20: mol_type = genomic DNA
taatcaattgtttttgattt

SEQ ID NO: 681 - DNA, 20 bp, Cannabis sativa
  misc_feature 1..20: note = CsMLO3 position 5012 targeted gRNA
  source 1..20: mol_type = genomic DNA
tgtaattatgtcttaatgat

SEQ ID NO: 682 - DNA, 20 bp, Cannabis sativa
  misc_feature 1..20: note = CsMLO3 position 5033 targeted gRNA
  source 1..20: mol_type = genomic DNA
ggacgtatactaaaagtgtg

SEQ ID NO: 683 - DNA, 20 bp, Cannabis sativa
  misc_feature 1..20: note = CsMLO3 position 5078 targeted gRNA
  source 1..20: mol_type = genomic DNA
aatgagttctgaatttttga

SEQ ID NO: 684 - DNA, 20 bp, Cannabis sativa
  misc_feature 1..20: note = CsMLO3 position 5098 targeted gRNA
  source 1..20: mol_type = genomic DNA
aggactttttgaatattgta

SEQ ID NO: 685 - DNA, 20 bp, Cannabis sativa
  misc_feature 1..20: note = CsMLO3 position 5139 targeted gRNA
  source 1..20: mol_type = genomic DNA
taatataaaattaatatata

SEQ ID NO: 686 - DNA, 20 bp, Cannabis sativa
  misc_feature 1..20: note = CsMLO3 position 5181 targeted gRNA
  source 1..20: mol_type = genomic DNA
tgatttgtgtgttttgtgtg

SEQ ID NO: 687 - DNA, 20 bp, Cannabis sativa
  misc_feature 1..20: note = CsMLO3 position 5187 targeted gRNA
  source 1..20: mol_type = genomic DNA
gtgtgttttgtgtgaggtgc

SEQ ID NO: 688 - DNA, 20 bp, Cannabis sativa
  misc_feature 1..20: note = CsMLO3 position 5188 targeted gRNA
  source 1..20: mol_type = genomic DNA
tgtgttttgtgtgaggtgca

SEQ ID NO: 689 - DNA, 20 bp, Cannabis sativa
  misc_feature 1..20: note = CsMLO3 position 5214 targeted gRNA
  source 1..20: mol_type = genomic DNA
agttctttagtgtctaaata

SEQ ID NO: 690 - DNA, 20 bp, Cannabis sativa
  misc_feature 1..20: note = CsMLO3 position 5215 targeted gRNA
  source 1..20: mol_type = genomic DNA
gttctttagtgtctaaatat

SEQ ID NO: 691 - DNA, 20 bp, Cannabis sativa
  misc_feature 1..20: note = CsMLO3 position 5232 targeted gRNA
  source 1..20: mol_type = genomic DNA
caaatatgaagatatgaagc

SEQ ID NO: 692 - DNA, 20 bp, Cannabis sativa
  misc_feature 1..20: note = CsMLO3 position 5249 targeted gRNA
  source 1..20: mol_type = genomic DNA
tcatatcttcatatttgtct

SEQ ID NO: 693 - DNA, 20 bp, Cannabis sativa
  misc_feature 1..20: note = CsMLO3 position 5268 targeted gRNA
  source 1..20: mol_type = genomic DNA
tagtaatgcaatatataata

SEQ ID NO: 694 - DNA, 20 bp, Cannabis sativa
  misc_feature 1..20: note = CsMLO3 position 5285 targeted gRNA
  source 1..20: mol_type = genomic DNA
tatatattgcattactacct

SEQ ID NO: 695 - DNA, 20 bp, Cannabis sativa
  misc_feature 1..20: note = CsMLO3 position 5291 targeted gRNA
  source 1..20: mol_type = genomic DNA
tttggttctgccaatagcca

SEQ ID NO: 696 - DNA, 20 bp, Cannabis sativa
  misc_feature 1..20: note = CsMLO3 position 5292 targeted gRNA
  source 1..20: mol_type = genomic DNA
tgcattactaccttggctat

SEQ ID NO: 697 - DNA, 20 bp, Cannabis sativa
  misc_feature 1..20: note = CsMLO3 position 5309 targeted gRNA
  source 1..20: mol_type = genomic DNA
aacttaaaaactactcactt

SEQ ID NO: 698 - DNA, 20 bp, Cannabis sativa
  misc_feature 1..20: note = CsMLO3 position 5361 targeted gRNA
  source 1..20: mol_type = genomic DNA
catattctataaaattaata

SEQ ID NO: 699 - DNA, 20 bp, Cannabis sativa
  misc_feature 1..20: note = CsMLO3 position 5401 targeted gRNA
  source 1..20: mol_type = genomic DNA
ttgaattgcagatgagaaaa

SEQ ID NO: 700 - DNA, 20 bp, Cannabis sativa
  misc_feature 1..20: note = CsMLO3 position 5410 targeted gRNA
  source 1..20: mol_type = genomic DNA
agatgagaaaatggaaagtt

SEQ ID NO: 701 - DNA, 20 bp, Cannabis sativa
  misc_feature 1..20: note = CsMLO3 position 5411 targeted gRNA
  source 1..20: mol_type = genomic DNA
gatgagaaaatggaaagttt

SEQ ID NO: 702 - DNA, 20 bp, Cannabis sativa
  misc_feature 1..20: note = CsMLO3 position 5414 targeted gRNA
  source 1..20: mol_type = genomic DNA
gagaaaatggaaagtttggg

SEQ ID NO: 703 - DNA, 20 bp, Cannabis sativa
  misc_feature 1..20: note = CsMLO3 position 5450 targeted gRNA
  source 1..20: mol_type = genomic DNA
attgagtacatatatagtaa

SEQ ID NO: 704 - DNA, 20 bp, Cannabis sativa
  misc_feature 1..20: note = CsMLO3 position 5537 targeted gRNA
  source 1..20: mol_type = genomic DNA
ttgtataattaattattttt

SEQ ID NO: 705 - DNA, 20 bp, Cannabis sativa
  misc_feature 1..20: note = CsMLO3 position 5563 targeted gRNA
  source 1..20: mol_type = genomic DNA
cactacaacttatctaactc

SEQ ID NO: 706 - DNA, 20 bp, Cannabis sativa
  misc_feature 1..20: note = CsMLO3 position 6711 targeted gRNA
  source 1..20: mol_type = genomic DNA
atctttacattcttactttt

SEQ ID NO: 707 - DNA, 20 bp, Cannabis sativa
  misc_feature 1..20: note = CsMLO3 position 6785 targeted gRNA
  source 1..20: mol_type = genomic DNA
tatataaatattcaatcaaa

SEQ ID NO: 708 - DNA, 20 bp, Cannabis sativa
  misc_feature 1..20: note = CsMLO3 position 6789 targeted gRNA
  source 1..20: mol_type = genomic DNA
taaatattcaatcaaatggt

SEQ ID NO: 709 - DNA, 20 bp, Cannabis sativa
  misc_feature 1..20: note = CsMLO3 position 6811 targeted gRNA
  source 1..20: mol_type = genomic DNA
cttgtaaatctaaatctctc

SEQ ID NO: 710 - DNA, 20 bp, Cannabis sativa
  misc_feature 1..20: note = CsMLO3 position 6837 targeted gRNA
  source 1..20: mol_type = genomic DNA
tttacaagagacacatcatt

SEQ ID NO: 711 - DNA, 20 bp, Cannabis sativa
  misc_feature 1..20: note = CsMLO3 position 6859 targeted gRNA
  source 1..20: mol_type = genomic DNA
gaagaagacatttgaacatt

SEQ ID NO: 712 - DNA, 20 bp, Cannabis sativa
  misc_feature 1..20: note = CsMLO3 position 6873 targeted gRNA
  source 1..20: mol_type = genomic DNA
tccaaagtgaaattggtgat

SEQ ID NO: 713 - DNA, 20 bp, Cannabis sativa
  misc_feature 1..20: note = CsMLO3 position 6880 targeted gRNA
  source 1..20: mol_type = genomic DNA
cttacaatccaaagtgaaat

SEQ ID NO: 714 - DNA, 20 bp, Cannabis sativa
  misc_feature 1..20: note = CsMLO3 position 6883 targeted gRNA
  source 1..20: mol_type = genomic DNA
gccaatcaccaatttcactt

SEQ ID NO: 715 - DNA, 20 bp, Cannabis sativa
  misc_feature 1..20: note = CsMLO3 position 6927 targeted gRNA
  source 1..20: mol_type = genomic DNA
ttgttttcttctctataata

SEQ ID NO: 716 - DNA, 20 bp, Cannabis sativa
  misc_feature 1..20: note = CsMLO3 position 6973 targeted gRNA
  source 1..20: mol_type = genomic DNA
tcaaaagttttttattatat

SEQ ID NO: 717 - DNA, 20 bp, Cannabis sativa
  misc_feature 1..20: note = CsMLO3 position 7030 targeted gRNA
  source 1..20: mol_type = genomic DNA
ttcttgtttatcaaatgatc

SEQ ID NO: 718 - DNA, 20 bp, Cannabis sativa
  misc_feature 1..20: note = CsMLO3 position 7055 targeted gRNA
  source 1..20: mol_type = genomic DNA
tgctttttcagacaattctt

SEQ ID NO: 719 - DNA, 20 bp, Cannabis sativa
  misc_feature 1..20: note = CsMLO3 position 7056 targeted gRNA
  source 1..20: mol_type = genomic DNA
gctttttcagacaattcttc

SEQ ID NO: 720 - DNA, 20 bp, Cannabis sativa
  misc_feature 1..20: note = CsMLO3 position 7069 targeted gRNA
  source 1..20: mol_type = genomic DNA
attcttcgggtcagtcacta

SEQ ID NO: 721 - DNA, 20 bp, Cannabis sativa
  misc_feature 1..20: note = CsMLO3 position 7089 targeted gRNA
  source 1..20: mol_type = genomic DNA
aggttgattacatgacactg

SEQ ID NO: 722 - DNA, 20 bp, Cannabis sativa
  misc_feature 1..20: note = CsMLO3 position 7094 targeted gRNA
  source 1..20: mol_type = genomic DNA
gattacatgacactgaggca

SEQ ID NO: 723 - DNA, 20 bp, Cannabis sativa
  misc_feature 1..20: note = CsMLO3 position 7105 targeted gRNA
  source 1..20: mol_type = genomic DNA
actgaggcatggatttgtaa

SEQ ID NO: 724 - DNA, 20 bp, Cannabis sativa
  misc_feature 1..20: note = CsMLO3 position 7126 targeted gRNA
  source 1..20: mol_type = genomic DNA
ggtatgttgcacaatgatct

SEQ ID NO: 725 - DNA, 20 bp, Cannabis sativa
  misc_feature 1..20: note = CsMLO3 position 7137 targeted gRNA
  source 1..20: mol_type = genomic DNA
caatgatcttggcctgaaaa

SEQ ID NO: 726 - DNA, 20 bp, Cannabis sativa
  misc_feature 1..20: note = CsMLO3 position 7138 targeted gRNA
  source 1..20: mol_type = genomic DNA
tgtaatttgaagccattttc

SEQ ID NO: 727 - DNA, 20 bp, Cannabis sativa
  misc_feature 1..20: note = CsMLO3 position 7203 targeted gRNA
  source 1..20: mol_type = genomic DNA
agctatgcttttcccatttc

SEQ ID NO: 728 - DNA, 20 bp, Cannabis sativa
  misc_feature 1..20: note = CsMLO3 position 7204 targeted gRNA
  source 1..20: mol_type = genomic DNA
gagccaaatgtgcctgaaat

SEQ ID NO: 729 - DNA, 20 bp, Cannabis sativa
  misc_feature 1..20: note = CsMLO3 position 7205 targeted gRNA
  source 1..20: mol_type = genomic DNA
ggagccaaatgtgcctgaaa

SEQ ID NO: 730 - DNA, 20 bp, Cannabis sativa
  misc_feature 1..20: note = CsMLO3 position 7212 targeted gRNA
  source 1..20: mol_type = genomic DNA
tttcccatttcaggcacatt

SEQ ID NO: 731 - DNA, 20 bp, Cannabis sativa
  misc_feature 1..20: note = CsMLO3 position 7226 targeted gRNA
  source 1..20: mol_type = genomic DNA
tcaaatcttgtttcactttc

SEQ ID NO: 732 - DNA, 20 bp, Cannabis sativa
  misc_feature 1..20: note = CsMLO3 position 7272 targeted gRNA
  source 1..20: mol_type = genomic DNA
catcagcaaatcacttgatc

SEQ ID NO: 733 - DNA, 20 bp, Cannabis sativa
  misc_feature 1..20: note = CsMLO3 position 7291 targeted gRNA
  source 1..20: mol_type = genomic DNA
caggattttgtagtaattgt

SEQ ID NO: 734 - DNA, 20 bp, Cannabis sativa
  misc_feature 1..20: note = CsMLO3 position 7292 targeted gRNA
  source 1..20: mol_type = genomic DNA
aggattttgtagtaattgtt

SEQ ID NO: 735 - DNA, 20 bp, Cannabis sativa
  misc_feature 1..20: note = CsMLO3 position 7323 targeted gRNA
  source 1..20: mol_type = genomic DNA
atattataagctgatttcaa

SEQ ID NO: 736 - DNA, 20 bp, Cannabis sativa
  misc_feature 1..20: note = CsMLO3 position 7419 targeted gRNA
  source 1..20: mol_type = genomic DNA
cggcaacgaaccaaattact

SEQ ID NO: 737 - DNA, 20 bp, Cannabis sativa
  misc_feature 1..20: note = CsMLO3 position 7420 targeted gRNA
  source 1..20: mol_type = genomic DNA
atatatgcagcccagtaatt

SEQ ID NO: 738 - DNA, 20 bp, Cannabis sativa
  misc_feature 1..20: note = CsMLO3 position 7420, strand -1, targeted gRNA
  source 1..20: mol_type = genomic DNA
acggcaacgaaccaaattac

SEQ ID NO: 739 - DNA, 20 bp, Cannabis sativa
  misc_feature 1..20: note = CsMLO3 position 7439 targeted gRNA
  source 1..20: mol_type = genomic DNA
gttggacagtagaaacaata

SEQ ID NO: 740 - DNA, 20 bp, Cannabis sativa
  misc_feature 1..20: note = CsMLO3 position 7457 targeted gRNA
  source 1..20: mol_type = genomic DNA
caataacttaccatatgtgt

SEQ ID NO: 741 - DNA, 20 bp, Cannabis sativa
  misc_feature 1..20: note = CsMLO3 position 7458 targeted gRNA
  source 1..20: mol_type = genomic DNA
tttctactgtccaacacata

SEQ ID NO: 742 - DNA, 20 bp, Cannabis sativa
  misc_feature 1..20: note = CsMLO3 position 7519 targeted gRNA
  source 1..20: mol_type = genomic DNA
caacatttcagtcactgaaa

SEQ ID NO: 743 - DNA, 20 bp, Cannabis sativa
  misc_feature 1..20: note = CsMLO3 position 7549 targeted gRNA
  source 1..20: mol_type = genomic DNA
gttgttcttttttaattaac

SEQ ID NO: 744 - DNA, 20 bp, Cannabis sativa
  misc_feature 1..20: note = CsMLO3 position 7568 targeted gRNA
  source 1..20: mol_type = genomic DNA
caggaatatactcttatttg

SEQ ID NO: 745 - DNA, 20 bp, Cannabis sativa
  misc_feature 1..20: note = CsMLO3 position 7583 targeted gRNA
  source 1..20: mol_type = genomic DNA
cttacaatcaaaggtagaaa

SEQ ID NO: 746 - DNA, 20 bp, Cannabis sativa
  misc_feature 1..20: note = CsMLO3 position 7592 targeted gRNA
  source 1..20: mol_type = genomic DNA
tgtgttgtacttacaatcaa

SEQ ID NO: 747 - DNA, 20 bp, Cannabis sativa
  misc_feature 1..20: note = CsMLO3 position 7660 targeted gRNA
  source 1..20: mol_type = genomic DNA
ttccacacattagcaaatgt

SEQ ID NO: 748 - DNA, 20 bp, Cannabis sativa
  misc_feature 1..20: note = CsMLO3 position 7661 targeted gRNA
  source 1..20: mol_type = genomic DNA
tttccacacattagcaaatg

SEQ ID NO: 749 - DNA, 20 bp, Cannabis sativa
  misc_feature 1..20: note = CsMLO3 position 7669 targeted gRNA
  source 1..20: mol_type = genomic DNA
gtcccacatttgctaatgtg

SEQ ID NO: 750 - DNA, 20 bp, Cannabis sativa
  misc_feature 1..20: note = CsMLO3 position 7699 targeted gRNA
  source 1..20: mol_type = genomic DNA
ttgtgatatataagatgaat

SEQ ID NO: 751 - DNA, 20 bp, Cannabis sativa
  misc_feature 1..20: note = CsMLO3 position 7715 targeted gRNA
  source 1..20: mol_type = genomic DNA
gaataggctactccttttat

SEQ ID NO: 752 - DNA, 20 bp, Cannabis sativa
  misc_feature 1..20: note = CsMLO3 position 7716 targeted gRNA
  source 1..20: mol_type = genomic DNA
aataggctactccttttata

SEQ ID NO: 753 - DNA, 20 bp, Cannabis sativa
  misc_feature 1..20: note = CsMLO3 position 7716, strand -1, targeted gRNA
  source 1..20: mol_type = genomic DNA
ccatttgaaaaccctataaa

SEQ ID NO: 754 - DNA, 20 bp, Cannabis sativa
  misc_feature 1..20: note = CsMLO3 position 7727 targeted gRNA
  source 1..20: mol_type = genomic DNA
ccttttatagggttttcaaa

SEQ ID NO: 755 - DNA, 20 bp, Cannabis sativa
  misc_feature 1..20: note = CsMLO3 position 7741 targeted gRNA
  source 1..20: mol_type = genomic DNA
atttaggaataagatgaatg

SEQ ID NO: 756 - DNA, 20 bp, Cannabis sativa
  misc_feature 1..20: note = CsMLO3 position 7742 targeted gRNA
  source 1..20: mol_type = genomic DNA
aatttaggaataagatgaat

SEQ ID NO: 757 - DNA, 20 bp, Cannabis sativa
  misc_feature 1..20: note = CsMLO3 position 7743 targeted gRNA
  source 1..20: mol_type = genomic DNA
gaatttaggaataagatgaa

SEQ ID NO: 758 - DNA, 20 bp, Cannabis sativa
  misc_feature 1..20: note = CsMLO3 position 7757 targeted gRNA
  source 1..20: mol_type = genomic DNA
gacataccatgttagaattt

SEQ ID NO: 759 - DNA, 20 bp, Cannabis sativa
  misc_feature 1..20: note = CsMLO3 position 7762 targeted gRNA
  source 1..20: mol_type = genomic DNA
cttattcctaaattctaaca

SEQ ID NO: 760 - DNA, 20 bp, Cannabis sativa
  misc_feature 1..20: note = CsMLO3 position 7788 targeted gRNA
  source 1..20: mol_type = genomic DNA
aaaaacccaacactggaaag

SEQ ID NO: 761 - DNA, 20 bp, Cannabis sativa
  misc_feature 1..20: note = CsMLO3 position 7793 targeted gRNA
  source 1..20: mol_type = genomic DNA
tgtgtgccactttccagtgt

SEQ ID NO: 762 - DNA, 20 bp, Cannabis sativa
  misc_feature 1..20: note = CsMLO3 position 7794 targeted gRNA
  source 1..20: mol_type = genomic DNA
gtgtgccactttccagtgtt

SEQ ID NO: 763 - DNA, 20 bp, Cannabis sativa
  misc_feature 1..20: note = CsMLO3 position 7795 targeted gRNA
  source 1..20: mol_type = genomic DNA
acaggtcaaaaacccaacac

SEQ ID NO: 764 - DNA, 20 bp, Cannabis sativa
  misc_feature 1..20: note = CsMLO3 position 7813 targeted gRNA
  source 1..20: mol_type = genomic DNA
aaatttgtagattttgaaac

SEQ ID NO: 765 - DNA, 20 bp, Cannabis sativa
  misc_feature 1..20: note = CsMLO3 position 7849 targeted gRNA
  source 1..20: mol_type = genomic DNA
ccaaatatcggaaaatttgt

SEQ ID NO: 766 - DNA, 20 bp, Cannabis sativa
  misc_feature 1..20: note = CsMLO3 position 7850 targeted gRNA
  source 1..20: mol_type = genomic DNA
gccaaatatcggaaaatttg

SEQ ID NO: 767 - DNA, 20 bp, Cannabis sativa
  misc_feature 1..20: note = CsMLO3 position 7860 targeted gRNA
  source 1..20: mol_type = genomic DNA
cccacaaattttccgatatt

SEQ ID NO: 768 - DNA, 20 bp, Cannabis sativa
  misc_feature 1..20: note = CsMLO3 position 7861 targeted gRNA
  source 1..20: mol_type = genomic DNA
aatctcacaaggccaaatat

SEQ ID NO: 769 - DNA, 20 bp, Cannabis sativa
  misc_feature 1..20: note = CsMLO3 position 7872 targeted gRNA
  source 1..20: mol_type = genomic DNA
acatttgaaagaatctcaca

SEQ ID NO: 770 - DNA, 20 bp, Cannabis sativa
  misc_feature 1..20: note = CsMLO3 position 7892 targeted gRNA
  source 1..20: mol_type = genomic DNA
attctttcaaatgtcacgtt

SEQ ID NO: 771 - DNA, 20 bp, Cannabis sativa
  misc_feature 1..20: note = CsMLO3 position 7900 targeted gRNA
  source 1..20: mol_type = genomic DNA
aaatgtcacgttcggtcctg

SEQ ID NO: 772 - DNA, 20 bp, Cannabis sativa
  misc_feature 1..20: note = CsMLO3 position 7905 targeted gRNA
  source 1..20: mol_type = genomic DNA
aacgacctttcagagaccac

SEQ ID NO: 773 - DNA, 20 bp, Cannabis sativa
  misc_feature 1..20: note = CsMLO3 position 7911 targeted gRNA
  source 1..20: mol_type = genomic DNA
tcggtcctgtggtctctgaa

SEQ ID NO: 774 - DNA, 20 bp, Cannabis sativa
  misc_feature 1..20: note = CsMLO3 position 7935 targeted gRNA
  source 1..20: mol_type = genomic DNA
cgtttgggcctgaaaagtgt

SEQ ID NO: 775 - DNA, 20 bp, Cannabis sativa
  misc_feature 1..20: note = CsMLO3 position 7936 targeted gRNA
  source 1..20: mol_type = genomic DNA
acgtttgggcctgaaaagtg

SEQ ID NO: 776 - DNA, 20 bp, Cannabis sativa
  misc_feature 1..20: note = CsMLO3 position 7938 targeted gRNA
  source 1..20: mol_type = genomic DNA
tcgttatacccacacttttc

SEQ ID NO: 777 - DNA, 20 bp, Cannabis sativa
  misc_feature 1..20: note = CsMLO3 position 7950 targeted gRNA
  source 1..20: mol_type = genomic DNA
ttaatacactcctcacgttt

SEQ ID NO: 778 - DNA, 20 bp, Cannabis sativa
  misc_feature 1..20: note = CsMLO3 position 7951 targeted gRNA
  source 1..20: mol_type = genomic DNA
acttttcaggcccaaacgtg

SEQ ID NO: 779 - DNA, 20 bp, Cannabis sativa
  misc_feature 1..20: note = CsMLO3 position 7951, strand -1, targeted gRNA
  source 1..20: mol_type = genomic DNA
cttaatacactcctcacgtt

SEQ ID NO: 780 - DNA, 20 bp, Cannabis sativa
  misc_feature 1..20: note = CsMLO3 position 7991 targeted gRNA
  source 1..20: mol_type = genomic DNA
agtctcacattgctaatgta

SEQ ID NO: 781 - DNA, 20 bp, Cannabis sativa
  misc_feature 1..20: note = CsMLO3 position 8020 targeted gRNA
  source 1..20: mol_type = genomic DNA
attgtgatatataaaatgaa

SEQ ID NO: 782 - DNA, 20 bp, Cannabis sativa
  misc_feature 1..20: note = CsMLO3 position 8021 targeted gRNA
  source 1..20: mol_type = genomic DNA
ttgtgatatataaaatgaat

SEQ ID NO: 783 - DNA, 20 bp, Cannabis sativa
  misc_feature 1..20: note = CsMLO3 position 8038 targeted gRNA
  source 1..20: mol_type = genomic DNA
taaaactaattggctgtggg

SEQ ID NO: 784 - DNA, 20 bp, Cannabis sativa
  misc_feature 1..20: note = CsMLO3 position 8041 targeted gRNA
  source 1..20: mol_type = genomic DNA
tcttaaaactaattggctgt

SEQ ID NO: 785 - DNA, 20 bp, Cannabis sativa
  misc_feature 1..20: note = CsMLO3 position 8042 targeted gRNA
  source 1..20: mol_type = genomic DNA
atcttaaaactaattggctg

SEQ ID NO: 786 - DNA, 20 bp, Cannabis sativa
  misc_feature 1..20: note = CsMLO3 position 8048 targeted gRNA
  source 1..20: mol_type = genomic DNA
ggttttatcttaaaactaat

SEQ ID NO: 787 - DNA, 20 bp, Cannabis sativa
  misc_feature 1..20: note = CsMLO3 position 8069 targeted gRNA
  source 1..20: mol_type = genomic DNA
atttagggataagatgaatg

SEQ ID NO: 788 - DNA, 20 bp, Cannabis sativa
  misc_feature 1..20: note = CsMLO3 position 8070 targeted gRNA
  source 1..20: mol_type = genomic DNA
aatttagggataagatgaat

SEQ ID NO: 789 - DNA, 20 bp, Cannabis sativa
  misc_feature 1..20: note = CsMLO3 position 8071 targeted gRNA
  source 1..20: mol_type = genomic DNA
gaatttagggataagatgaa

SEQ ID NO: 790 - DNA, 20 bp, Cannabis sativa
  misc_feature 1..20: note = CsMLO3 position 8084 targeted gRNA
  source 1..20: mol_type = genomic DNA
attaagcatgttagaattta

SEQ ID NO: 791 - DNA, 20 bp, Cannabis sativa
  misc_feature 1..20: note = CsMLO3 position 8085 targeted gRNA
  source 1..20: mol_type = genomic DNA
gattaagcatgttagaattt

SEQ ID NO: 792 - DNA, 20 bp, Cannabis sativa
  misc_feature 1..20: note = CsMLO3 position 8144 targeted gRNA
  source 1..20: mol_type = genomic DNA
caaattgcagataatattac

SEQ ID NO: 793 - DNA, 20 bp, Cannabis sativa
  misc_feature 1..20: note = CsMLO3 position 8147 targeted gRNA
  source 1..20: mol_type = genomic DNA
attgcagataatattactgg

SEQ ID NO: 794 - DNA, 20 bp, Cannabis sativa
  misc_feature 1..20: note = CsMLO3 position 8148 targeted gRNA
  source 1..20: mol_type = genomic DNA
ttgcagataatattactggt

SEQ ID NO: 795 - DNA, 20 bp, Cannabis sativa
  misc_feature 1..20: note = CsMLO3 position 8180 targeted gRNA
  source 1..20: mol_type = genomic DNA
tcaagtaatcataacaaaga

SEQ ID NO: 796 - DNA, 20 bp, Cannabis sativa
  misc_feature 1..20: note = CsMLO3 position 8181 targeted gRNA
  source 1..20: mol_type = genomic DNA
caagtaatcataacaaagat

SEQ ID NO: 797 - DNA, 20 bp, Cannabis sativa
  misc_feature 1..20: note = CsMLO3 position 8202 targeted gRNA
  source 1..20: mol_type = genomic DNA
ggattaagcattcaagagag

SEQ ID NO: 798 - DNA, 20 bp, Cannabis sativa
  misc_feature 1..20: note = CsMLO3 position 8210 targeted gRNA
  source 1..20: mol_type = genomic DNA
cattcaagagagaggagatg

SEQ ID NO: 799 - DNA, 20 bp, Cannabis sativa
  misc_feature 1..20: note = CsMLO3 position 8217 targeted gRNA
  source 1..20: mol_type = genomic DNA
gagagaggagatgtggtaaa

SEQ ID NO: 800 - DNA, 20 bp, Cannabis sativa
  misc_feature 1..20: note = CsMLO3 position 8228 targeted gRNA
  source 1..20: mol_type = genomic DNA
tgtggtaaaaggtgcaccat

SEQ ID NO: 801 - DNA, 20 bp, Cannabis sativa
  misc_feature 1..20: note = CsMLO3 position 8233 targeted gRNA
  source 1..20: mol_type = genomic DNA
tcatctcctggttgaaccaa

SEQ ID NO: 802 - DNA, 20 bp, Cannabis sativa
  misc_feature 1..20: note = CsMLO3 position 8238 targeted gRNA
  source 1..20: mol_type = genomic DNA
ggtgcaccattggttcaacc

SEQ ID NO: 803 - DNA, 20 bp, Cannabis sativa
  misc_feature 1..20: note = CsMLO3 position 8245 targeted gRNA
  source 1..20: mol_type = genomic DNA
aaccagaagaggtcatctcc

SEQ ID NO: 804 - DNA, 20 bp, Cannabis sativa
  misc_feature 1..20: note = CsMLO3 position 8254 targeted gRNA
  source 1..20: mol_type = genomic DNA
aaccaggagatgacctcttc

SEQ ID NO: 805 - DNA, 20 bp, Cannabis sativa
  misc_feature 1..20: note = CsMLO3 position 8256 targeted gRNA
  source 1..20: mol_type = genomic DNA
taggccgtccgaaccagaag

SEQ ID NO: 806 - DNA, 20 bp, Cannabis sativa
  misc_feature 1..20: note = CsMLO3 position 8259 targeted gRNA
  source 1..20: mol_type = genomic DNA
ggagatgacctcttctggtt

SEQ ID NO: 807 - DNA, 20 bp, Cannabis sativa
  misc_feature 1..20: note = CsMLO3 position 8263 targeted gRNA
  source 1..20: mol_type = genomic DNA
atgacctcttctggttcgga

SEQ ID NO: 808 - DNA, 20 bp, Cannabis sativa
  misc_feature 1..20: note = CsMLO3 position 8275 targeted gRNA
  source 1..20: mol_type = genomic DNA
atgagaaagagcattaattt

SEQ ID NO: 809 - DNA, 20 bp, Cannabis sativa
  misc_feature 1..20: note = CsMLO3 position 8301 targeted gRNA
  source 1..20: mol_type = genomic DNA
taagtacctgaaagagaaca

SEQ ID NO: 810 - DNA, 20 bp, Cannabis sativa
  misc_feature 1..20: note = CsMLO3 position 8306 targeted gRNA
  source 1..20: mol_type = genomic DNA
cattcaccttgttctctttc

SEQ ID NO: 811 - DNA, 20 bp, Cannabis sativa
  misc_feature 1..20: note = CsMLO3 position 8413 targeted gRNA
  source 1..20: mol_type = genomic DNA
aaaatgatatcttttctgct

SEQ ID NO: 812 - DNA, 20 bp, Cannabis sativa
  misc_feature 1..20: note = CsMLO3 position 8429 targeted gRNA
  source 1..20: mol_type = genomic DNA
tgcttggtactaattaatgc

SEQ ID NO: 813 - DNA, 20 bp, Cannabis sativa
  misc_feature 1..20: note = CsMLO3 position 8487 targeted gRNA
  source 1..20: mol_type = genomic DNA
tactgtactccatgcaaaaa

SEQ ID NO: 814 - DNA, 20 bp, Cannabis sativa
  misc_feature 1..20: note = CsMLO3 position 8489 targeted gRNA
  source 1..20: mol_type = genomic DNA
ttcaacttgccttttttgca

SEQ ID NO: 815 - DNA, 20 bp, Cannabis sativa
  misc_feature 1..20: note = CsMLO3 position 8531 targeted gRNA
  source 1..20: mol_type = genomic DNA
tgccttgaaaccaaaaatca

SEQ ID NO: 816 - DNA, 20 bp, Cannabis sativa
  misc_feature 1..20: note = CsMLO3 position 8532 targeted gRNA
  source 1..20: mol_type = genomic DNA
atttgactttccttgatttt

SEQ ID NO: 817 - DNA, 20 bp, Cannabis sativa
  misc_feature 1..20: note = CsMLO3 position 8540 targeted gRNA
  source 1..20: mol_type = genomic DNA
ttccttgatttttggtttca

SEQ ID NO: 818 - DNA, 20 bp, Cannabis sativa
  misc_feature 1..20: note = CsMLO3 position 8559 targeted gRNA
  source 1..20: mol_type = genomic DNA
aaggcaataaaattattaca

SEQ ID NO: 819 - DNA, 20 bp, Cannabis sativa
  misc_feature 1..20: note = CsMLO3 position 8624 targeted gRNA
  source 1..20: mol_type = genomic DNA
ttcgtggaagcaagtgttca

SEQ ID NO: 820 - DNA, 20 bp, Cannabis sativa
  misc_feature 1..20: note = CsMLO3 position 8640 targeted gRNA
  source 1..20: mol_type = genomic DNA
tgatatcttcaattttttcg

SEQ ID NO: 821 - DNA, 20 bp, Cannabis sativa
  misc_feature 1..20: note = CsMLO3 position 8669 targeted gRNA
  source 1..20: mol_type = genomic DNA
tatcatcataagaatttcaa

SEQ ID NO: 822 - DNA, 20 bp, Cannabis sativa
  misc_feature 1..20: note = CsMLO3 position 8670 targeted gRNA
  source 1..20: mol_type = genomic DNA
atcatcataagaatttcaat

SEQ ID NO: 823 - DNA, 20 bp, Cannabis sativa
  misc_feature 1..20: note = CsMLO3 position 8671 targeted gRNA
  source 1..20: mol_type = genomic DNA
tcatcataagaatttcaatg

SEQ ID NO: 824 - DNA, 20 bp, Cannabis sativa
  misc_feature 1..20: note = CsMLO3 position 8805 targeted gRNA
  source 1..20: mol_type = genomic DNA
ttctctttttctttcttact

SEQ ID NO: 825 - DNA, 20 bp, Cannabis sativa
  misc_feature 1..20: note = CsMLO3 position 8819 targeted gRNA
  source 1..20: mol_type = genomic DNA
aactgcatagaacttgtatg

SEQ ID NO: 826 - DNA, 20 bp, Cannabis sativa
  misc_feature 1..20: note = CsMLO3 position 8853 targeted gRNA
  source 1..20: mol_type = genomic DNA
tgtgtgacaagagcatatag

SEQ ID NO: 827 - DNA, 20 bp, Cannabis sativa
  misc_feature 1..20: note = CsMLO3 position 8866 targeted gRNA
  source 1..20: mol_type = genomic DNA
tctatatgctcttgtcacac

SEQ ID NO: 828 - DNA, 20 bp, Cannabis sativa
  misc_feature 1..20: note = CsMLO3 position 8893 targeted gRNA
  source 1..20: mol_type = genomic DNA
gatgataatgatgatttaga

SEQ ID NO: 829 - DNA, 20 bp, Cannabis sativa
  misc_feature 1..20: note = CsMLO3 position 8954 targeted gRNA
  source 1..20: mol_type = genomic DNA
atttgatcatatattacaga

SEQ ID NO: 830 - DNA, 20 bp, Cannabis sativa
  misc_feature 1..20: note = CsMLO3 position 8955 targeted gRNA
  source 1..20: mol_type = genomic DNA
tttgatcatatattacagat

SEQ ID NO: 831 - DNA, 20 bp, Cannabis sativa
  misc_feature 1..20: note = CsMLO3 position 8956 targeted gRNA
  source 1..20: mol_type = genomic DNA
ttgatcatatattacagatg

SEQ ID NO: 832 - DNA, 20 bp, Cannabis sativa
  misc_feature 1..20: note = CsMLO3 position 8980 targeted gRNA
  source 1..20: mol_type = genomic DNA
actctgtcattgaaaattac

SEQ ID NO: 833 - DNA, 20 bp, Cannabis sativa
  misc_feature 1..20: note = CsMLO3 position 9013 targeted gRNA
  source 1..20: mol_type = genomic DNA
tagcaacagcattaaagaac

SEQ ID NO: 834 - DNA, 20 bp, Cannabis sativa
  misc_feature 1..20: note = CsMLO3 position 9027 targeted gRNA
  source 1..20: mol_type = genomic DNA
tgttcttggttttggctgaa

SEQ ID NO: 835 - DNA, 20 bp, Cannabis sativa
  misc_feature 1..20: note = CsMLO3 position 9035 targeted gRNA
  source 1..20: mol_type = genomic DNA
gtgttttttgttcttggttt

SEQ ID NO: 836 - DNA, 20 bp, Cannabis sativa
  misc_feature 1..20: note = CsMLO3 position 9041 targeted gRNA
  source 1..20: mol_type = genomic DNA
tcggttgtgttttttgttct

SEQ ID NO: 837 - DNA, 20 bp, Cannabis sativa
  misc_feature 1..20: note = CsMLO3 position 9059 targeted gRNA
  source 1..20: mol_type = genomic DNA
caaaaaacacaaccgaaatt

SEQ ID NO: 838 - DNA, 20 bp, Cannabis sativa
  misc_feature 1..20: note = CsMLO3 position 9060 targeted gRNA
  source 1..20: mol_type = genomic DNA
gcgagtttgtctccgaattt

SEQ ID NO: 839 - DNA, 20 bp, Cannabis sativa
  misc_feature 1..20: note = CsMLO3 position 9082 targeted gRNA
  source 1..20: mol_type = genomic DNA
gttgcaggcctacttgagaa

SEQ ID NO: 840 - DNA, 20 bp, Cannabis sativa
  misc_feature 1..20: note = CsMLO3 position 9085 targeted gRNA
  source 1..20: mol_type = genomic DNA
caaactcgccattctcaagt

SEQ ID NO: 841 - DNA, 20 bp, Cannabis sativa
  source 1..20: mol_type = genomic DNA
atgccatatgttggagttgc

SEQ ID NO: 842 - DNA, 20 bp, Cannabis sativa
  misc_feature 1..20: note = CsMLO3 position 9105 targeted gRNA
  source 1..20: mol_type = genomic DNA
aggcctgcaactccaacata

SEQ ID NO: 843 - DNA, 20 bp, Cannabis sativa
  misc_feature 1..20: note = CsMLO3 position 9106 targeted gRNA
  source 1..20: mol_type = genomic DNA
actggagacatgccatatgt

SEQ ID NO: 844 - DNA, 20 bp, Cannabis sativa
  misc_feature 1..20: note = CsMLO3 position 9124 targeted gRNA
  source 1..20: mol_type = genomic DNA
taattttgcagcagatgaac

SEQ ID NO: 845 - DNA, 20 bp, Cannabis sativa
  misc_feature 1..20: note = CsMLO3 position 9156 targeted gRNA
  source 1..20: mol_type = genomic DNA
tacagaagcacagcaactga

SEQ ID NO: 846 - DNA, 20 bp, Cannabis sativa
  misc_feature 1..20: note = CsMLO3 position 9165 targeted gRNA
  source 1..20: mol_type = genomic DNA
acagcaactgatggatacta

SEQ ID NO: 847 - DNA, 20 bp, Cannabis sativa
  misc_feature 1..20: note = CsMLO3 position 9175 targeted gRNA
  source 1..20: mol_type = genomic DNA
atggatactatggttctccg

SEQ ID NO: 848 - DNA, 20 bp, Cannabis sativa
  misc_feature 1..20: note = CsMLO3 position 9181 targeted gRNA
  source 1..20: mol_type = genomic DNA
ttttcgacattagacatcct

SEQ ID NO: 849 - DNA, 20 bp, Cannabis sativa
  misc_feature 1..20: note = CsMLO3 position 9213 targeted gRNA
  source 1..20: mol_type = genomic DNA
aacgattactatgagcctga

SEQ ID NO: 850 - DNA, 20 bp, Cannabis sativa
  misc_feature 1..20: note = CsMLO3 position 9214 targeted gRNA
  source 1..20: mol_type = genomic DNA
acgattactatgagcctgaa

SEQ ID NO: 851 - DNA, 20 bp, Cannabis sativa
  misc_feature 1..20: note = CsMLO3 position 9217 targeted gRNA
  source 1..20: mol_type = genomic DNA
ttgggagatggtgtcccttc

SEQ ID NO: 852 - DNA, 20 bp, Cannabis sativa
  misc_feature 1..20: note = CsMLO3 position 9229 targeted gRNA
  source 1..20: mol_type = genomic DNA
gatggtccattgttgggaga

SEQ ID NO: 853 - DNA, 20 bp, Cannabis sativa
  misc_feature 1..20: note = CsMLO3 position 9234 targeted gRNA
  source 1..20: mol_type = genomic DNA
gggacaccatctcccaacaa

SEQ ID NO: 854 - DNA, 20 bp, Cannabis sativa
  misc_feature 1..20: note = CsMLO3 position 9235 targeted gRNA
  source 1..20: mol_type = genomic DNA
gctgcagatggtccattgtt

SEQ ID NO: 855 - DNA, 20 bp, Cannabis sativa
  misc_feature 1..20: note = CsMLO3 position 9236 targeted gRNA
  source 1..20: mol_type = genomic DNA
tgctgcagatggtccattgt

SEQ ID NO: 856 - DNA, 20 bp, Cannabis sativa
  misc_feature 1..20: note = CsMLO3 position 9247 targeted gRNA
  source 1..20: mol_type = genomic DNA
tgtatttcacttgctgcaga

SEQ ID NO: 857 - DNA, 20 bp, Cannabis sativa
  misc_feature 1..20: note = CsMLO3 position 9284 targeted gRNA
  source 1..20: mol_type = genomic DNA
gaataactatgaagttgaga

SEQ ID NO: 858 - DNA, 20 bp, Cannabis sativa
  misc_feature 1..20: note = CsMLO3 position 9296 targeted gRNA
  source 1..20: mol_type = genomic DNA
agttgagaaggatataagtg

SEQ ID NO: 859 - DNA, 20 bp, Cannabis sativa
  misc_feature 1..20: note = CsMLO3 position 9300 targeted gRNA
  source 1..20: mol_type = genomic DNA
gagaaggatataagtgagga

SEQ ID NO: 860 - DNA, 20 bp, Cannabis sativa
  misc_feature 1..20: note = CsMLO3 position 9311 targeted gRNA
  source 1..20: mol_type = genomic DNA
aagtgaggaaggacagccaa

SEQ ID NO: 861 - DNA, 20 bp, Cannabis sativa
  misc_feature 1..20: note = CsMLO3 position 9316 targeted gRNA
  source 1..20: mol_type = genomic DNA
gagcttggttcctgaaccat

SEQ ID NO: 862 - DNA, 20 bp, Cannabis sativa
  misc_feature 1..20: note = CsMLO3 position 9317 targeted gRNA
  source 1..20: mol_type = genomic DNA
ggaaggacagccaatggttc

SEQ ID NO: 863 - DNA, 20 bp, Cannabis sativa
  misc_feature 1..20: note = CsMLO3 position 9331 targeted gRNA
  source 1..20: mol_type = genomic DNA
ttttgctgtgaggaggagct

SEQ ID NO: 864 - DNA, 20 bp, Cannabis sativa
  misc_feature 1..20: note = CsMLO3 position 9338 targeted gRNA
  source 1..20: mol_type = genomic DNA
gacctcattttgctgtgagg

SEQ ID NO: 865 - DNA, 20 bp, Cannabis sativa
  misc_feature 1..20: note = CsMLO3 position 9341 targeted gRNA
  source 1..20: mol_type = genomic DNA
cttgacctcattttgctgtg

SEQ ID NO: 866 - DNA, 20 bp, Cannabis sativa
  misc_feature 1..20: note = CsMLO3 position 9347 targeted gRNA
  source 1..20: mol_type = genomic DNA
ctcctcctcacagcaaaatg

SEQ ID NO: 867 - DNA, 20 bp, Cannabis sativa
  misc_feature 1..20: note = CsMLO3 position 9368 targeted gRNA
  source 1..20: mol_type = genomic DNA
cctaaatgagaagtgagata

SEQ ID NO: 868 - DNA, 20 bp, Cannabis sativa
  misc_feature 1..20: note = CsMLO3 position 9379 targeted gRNA
  source 1..20: mol_type = genomic DNA
ccttatctcacttctcattt

SEQ ID NO: 869 - DNA, 20 bp, Cannabis sativa
  misc_feature 1..20: note = CsMLO3 position 9450 targeted gRNA
  source 1..20: mol_type = genomic DNA
ctttatttcttattatcttt

SEQ ID NO: 870 - DNA, 20 bp, Cannabis sativa
  misc_feature 1..20: note = CsMLO3 position 9498 targeted gRNA
  source 1..20: mol_type = genomic DNA
aatatgtataagcttgaatt

SEQ ID NO: 871 - DNA, 21 bp, Cannabis sativa
  misc_feature 1..21: note = Forward Primer nucleotides 4-24 of SEQ ID NO:1
  source 1..21: mol_type = genomic DNA
gagtggaactagaagaaatgc

SEQ ID NO: 872 - DNA, 20 bp, Cannabis sativa
  misc_feature 1..20: note = Reverse nucleotides 763-782 of SEQ ID NO:1
  misc_feature 1..20: note = Reverse Primer nucleotides 763-782 of SEQ ID NO:1
  source 1..20: mol_type = genomic DNA
acctccaaacacaacagtga

SEQ ID NO: 873 - DNA, 779 bp, Cannabis sativa
  misc_feature 1..779: note = CsMLO1 Wt, exone 1, nucleotides 4-782 of SEQ ID NO:1
  source 1..779: mol_type = genomic DNA
gagtggaactagaagaaatgctggagtgacattgatgagtgagtgaatgagagcaagaaggaactaaagaagaataatccatctgaattcgaaatcggaatgagtggcggtggagaaggtgagtccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcataaggtctgatctgatctaacccatatctttagttgttgttgtttttttaggggttaaacaaattttgaatctttttctataaatatactttcccaaaagggactattcgctttaatctaattgtatagtacaaatatgtttgtttcactttttttctttattgtagaaaaaagtaccccttttattccgttgaagcattgcaccatttgcatatctttatcttcgtcctagccattgtccatgtcactttctgtgttctcactgttgtgtttggaggt

SEQ ID NO: 874 - protein, 134 aa, Cannabis sativa
MSGGGEGESLEYTPTWIVAVVCSVIVAISLAAERLLHYLGQVLNNKNQKPLYEALQKIKEELMLLGFISLLLTVSQNTISKFCVPASLVDNLLPCKRPHQEANDSSSSTSHHISGSFRRLLEETSTAHLGYCAH

SEQ ID NO: 875 - DNA, 332 bp, Cannabis sativa
  misc_feature 1..332: note = CsMLO1 65-L4 Deletion of 447 nucleotides (position 109-556) ofSEQ ID NO:1
  source 1..332: mol_type = genomic DNA
gagtggaactagaagaaatgctggagtgacattgatgagtgagtgaatgagagcaagaaggaactaaagaagaataatccatctgaattcgaaatcggaatgagtggttaaacaaattttgaatctttttctataaatatactttcccaaaagggactattcgctttaatctaattgtatagtacaaatatgtttgtttcactttttttctttattgtagaaaaaagtaccccttttattccgttgaagcattgcaccatttgcatatctttatcttcgtcctagccattgtccatgtcactttctgtgttctcactgttgtgtttggaggt

SEQ ID NO: 876 - DNA, 428 bp, Cannabis sativa
  misc_feature 1..428: note = CsMLO1 65- L4 Deletion
  source 1..428: mol_type = genomic DNA
ccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcataaggtctgatctgatctaacccatatctttagttgttgttgtttttttagg

SEQ ID NO: 877 - DNA, 425 bp, Cannabis sativa
  misc_feature 1..425: note = CsMLO1 65-L5 Deletion of 373 nucleotides, position 128-501 ofSEQ ID NO:1
  source 1..425: mol_type = genomic DNA
gagtggaactagaagaaatgctggagtgacattgatgagtgagtgaatgagagcaagaaggaactaaagaagaataatccatctgaattcgaaatcggaatgagtggcggtggagaaggtgagtctcatttgggttattgcgctcataaggtctgatctgatctaacccatatctttagttgttgttgtttttttaggggttaaacaaattttgaatctttttctataaatatactttcccaaaagggactattcgctttaatctaattgtatagtacaaatatgtttgtttcactttttttctttattgtagaaaaaagtaccccttttattccgttgaagcattgcaccatttgcatatctttatcttcgtcctagccattgtccatgtcactttctgtgttctcactgttgtgtttggaggt

SEQ ID NO: 878 - protein, 8 aa, Cannabis sativa
MSGGGEGE

SEQ ID NO: 879 - DNA, 354 bp, Cannabis sativa
  misc_feature 1..354: note = CsMLO1 65-L5 373 Deletion
  source 1..354: mol_type = genomic DNA
ccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactg

SEQ ID NO: 880 - DNA, 323 bp, Cannabis sativa
  misc_feature 1..323: note = CsMLO185-4 Deletion of 456 nucleotides positions 96-552 of SEQID NO:1
  source 1..323: mol_type = genomic DNA
gagtggaactagaagaaatgctggagtgacattgatgagtgagtgaatgagagcaagaaggaactaaagaagaataatccatctgaattcgataggggttaaacaaattttgaatctttttctataaatatactttcccaaaagggactattcgctttaatctaattgtatagtacaaatatgtttgtttcactttttttctttattgtagaaaaaagtaccccttttattccgttgaagcattgcaccatttgcatatctttatcttcgtcctagccattgtccatgtcactttctgtgttctcactgttgtgtttggaggt

SEQ ID NO: 881 - DNA, 456 bp, Cannabis sativa
  misc_feature 1..456: note = CsMLO1 85-4 456 Deletion
  source 1..456: mol_type = genomic DNA
aatcggaatgagtggcggtggagaaggtgagtccttggagtatactcccacatggatcgtcgccgtcgtctgctctgtcatcgtcgccatttctctcgccgcggagcgtcttctccactatctcggtcaggttttgaataataagaaccagaagcctctatacgaggccttgcagaagatcaaggaggagttgatgcttttgggtttcatttcgcttctcctcactgtttcgcagaacacaatttcaaagttttgcgtacctgcttctttggtcgacaatctcctcccttgtaagcgccctcatcaggaagctaatgattcgtcttcttcaacctctcatcacatctccggctctttcagacgccttcttgaagaaacttccactgctcatttgggttattgcgctcataaggtctgatctgatctaacccatatctttagttgttgttgtttttt

SEQ ID NO: 882 - DNA, 123 bp, Cannabis sativa
  misc_feature 1..123: note = CsMLO1_378-500
  source 1..123: mol_type = genomic DNA
cctcccttgtaagcgccctcatcaggaagctaatgattcgtcttcttcaacctctcatcacatctccggctctttcagacgccttcttgaagaaacttccactgctcatttgggttattgcgc

SEQ ID NO: 883 - DNA, 14 bp, Cannabis sativa
  misc_feature 1..14: note = Indel_d14
  source 1..14: mol_type = genomic DNA
agcgccctcatcag

SEQ ID NO: 884 - DNA, 109 bp, Cannabis sativa
  misc_feature 1..109: note = Csmlo1_d14
  source 1..109: mol_type = genomic DNA
cctcccttgtagaagctaatgattcgtcttcttcaacctctcatcacatctccggctctttcagacgccttcttgaagaaacttccactgctcatttgggttattgcgc

SEQ ID NO: 885 - DNA, 124 bp, Cannabis sativa
  misc_feature 1..124: note = Csmlo1_i1
  source 1..124: mol_type = genomic DNA
cctcccttgtaagcgccctcatcaggaagctaatgattcgtcttcttcaacctctcatcacatctccggctctttcagacgccttcttgaagaaacttccactgactcatttgggttattgcgc

SEQ ID NO: 886 - DNA, 110 bp, Cannabis sativa
  misc_feature 1..110: note = Csmlo1_d14i1
  source 1..110: mol_type = genomic DNA
cctcccttgtagaagctaatgattcgtcttcttcaacctctcatcacatctccggctctttcagacgccttcttgaagaaacttccactgactcatttgggttattgcgc